(19) United States
(12) Patent Application Publication
(10) Pub. No.: US 2024/0246983 A1
(43) Pub. Date: Jul. 25, 2024

(54) NITROGENOUS HETEROCYCLIC COMPOUNDS FOR ORGANIC ELECTROLUMINESCENT DEVICES

(71) Applicant: Merck Patent GmbH, Darmstadt (DE)

(72) Inventors: Philipp STOESSEL, Darmstadt (DE); Amir Hossain PARHAM, Darmstadt (DE); Rouven LINGE, Darmstadt (DE)

(21) Appl. No.: 18/556,948

(22) PCT Filed: Apr. 27, 2022
(86) PCT No.: PCT/EP2022/061126

§ 371 Date: Oct. 24, 2023

(30) Foreign Application Priority Data
Apr. 30, 2021 (EP) 21171569.3

(51) Int. Cl. C07D 487/04; C07D 487/14; C07D 487/22; C07D 491/147; C07D 491/22; C07D 495/14; C07D 519/00; C09K 11/06; H10K 85/30; H10K 85/60

(52) U.S. Cl. CPC C07D 487/04; C07D 487/14; C07D 487/22; C07D 491/147; C07D 491/22; C07D 495/14; C07D 519/00; C09K 11/06; H10K 85/342; H10K 85/626; H10K 85/633; H10K 85/654; H10K 85/6572; H10K 85/6574; C09K 2211/1022

(57) ABSTRACT

The present invention relates to nitrogen-containing heterocyclic compounds suitable for use in electronic devices, and to electronic devices, especially organic electroluminescent devices, comprising these compounds.

The present invention relates to nitrogen-containing heterocyclic compounds for use in electronic devices, especially in organic electroluminescent devices, and to electronic devices, especially organic electroluminescent devices comprising these heterocyclic compounds.

Emitting materials used in organic electroluminescent devices are frequently phosphorescent organometallic complexes or fluorescent compounds. There is generally still a need for improvement in electroluminescent devices.

U.S. Pat. No. 6,322,908 and WO 03/001569 A2 disclose polycyclic compounds that can be used in organic electroluminescent devices. There is no disclosure of compounds according to the present invention. In addition, the publication J. Org. Chem. 1995, 60, 2344-2352 describes photochemical properties of compounds including nitrogen-containing heterocyclic compounds. The synthesis of nitrogen-containing heterocyclic compounds is detailed in Chin. J. Chem. 2011, 29, 2769. However, there is no description of the use of these compounds in organic electroluminescent devices in the publications cited.

In general terms, there is still a need for improvement in these nitrogen-containing heterocyclic compounds, for example for use as emitters, especially as fluorescent emitters, particularly in relation to lifetime and colour purity, but also in relation to the efficiency and operating voltage of the device.

It is therefore an object of the present invention to provide compounds which are suitable for use in an organic electronic device, especially in an organic electroluminescent device, and which lead to good device properties when used in this device, and to provide the corresponding electronic device.

More particularly, the problem addressed by the present invention is that of providing compounds which lead to a high lifetime, good efficiency and low operating voltage.

In addition, the compounds should have excellent processibility, and the compounds should especially show good solubility.

A further problem addressed by the present invention can be considered that of providing compounds suitable for use in phosphorescent or fluorescent electroluminescent devices, especially as emitter. More particularly, a problem addressed by the present invention is that of providing emitters suitable for red, green or blue electroluminescent devices.

In addition, the compounds, especially when they are used as emitters in organic electroluminescent devices, should lead to devices having excellent colour purity.

A further problem addressed by the present invention can be considered that of providing compounds suitable for use in phosphorescent or fluorescent electroluminescent devices, especially as a matrix material.

More particularly, a problem addressed by the present invention is that of providing matrix materials suitable for red-, yellow- and blue-phosphorescing electroluminescent devices.

In addition, the compounds, especially when they are used as matrix materials or as electron transport materials in organic electroluminescent devices, should lead to devices having excellent colour purity.

A further problem can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, this object is achieved by particular compounds described in detail below that are of very good suitability for use in electroluminescent devices and lead to organic electroluminescent devices that show very good properties, especially in relation to lifetime, colour purity, efficiency and operating voltage. The present invention therefore provides these compounds and electronic devices, especially organic electroluminescent devices, comprising such compounds.

The present invention provides a compound comprising at least one structure of the formula (I), preferably a compound of the formula (I),

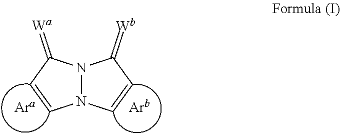

where the ring Ar^{a} is the same or different at each instance and is an aromatic or heteroaromatic ring system which has 5 to 60 aromatic ring atoms and may be substituted by one or more Ar or Ra radicals, the ring Ar^{b} is the same or different at each instance and is an aromatic or heteroaromatic ring system which has 5 to 60 aromatic ring atoms and may be substituted by one or more Ar or R^{b} radicals;
and where the further symbols and indices used are as follows:
W^{a}, W^{b} is the same or different at each instance and is O or S, preferably O;
Ar is the same or different at each instance and is an aromatic or heteroaromatic ring system which has 5 to 60 aromatic ring atoms and may be substituted by one or more R radicals; the Ar group here may form a ring system with at least one Ar, R, R^{a}, R^{b} group or a further group;
R, R^{a}, R^{b} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; or heteroarylthio group which has 5 to 60 aromatic ring atoms and may be substituted by one or more R¹ radicals, or a diarylamino, arylheteroarylamino, diheteroarylamino group which has 5 to 60 aromatic ring atoms and may be substituted by one or more R¹ radicals, or an aralkyl or heteroarylalkyl group which has 5 to 60 aromatic ring atoms and 1 to 10 carbon atoms in the alkyl radical and may be substituted by one or more R¹ radicals; at the same time, two R, R^{a}, R^{b} radicals together or with a further group may also form a ring system;
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more, preferably adjacent R¹ radicals together may form a ring system; at the same time, one or more R¹ radicals may form a ring system with a further part of the compound;
Ar″ is the same or different at each instance and is an aromatic or heteroaromatic ring system which has 5 to 30 aromatic ring atoms and may be substituted by one or more R² radicals; at the same time, it is possible for two Ar″ radicals bonded to the same carbon atom, silicon atom, nitrogen atom, phosphorus atom or boron atom also to be joined together via a bridge by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more, preferably adjacent substituents R² together may form a ring system;
with exclusion of compounds of the formulae (A), (B) and (C)

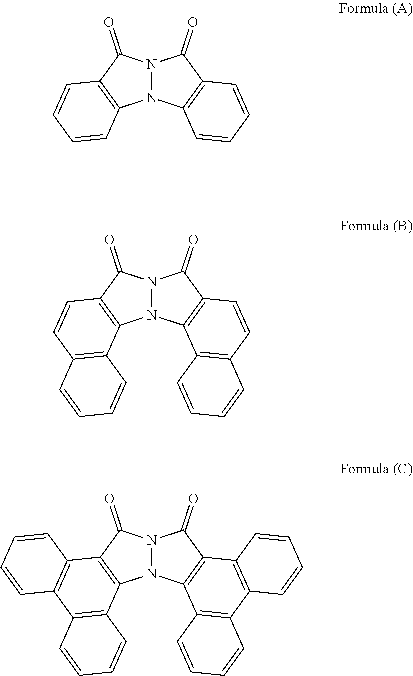 [SMILES: O=c1c2ccccc2n2c3ccccc3c(=O)n12 ; O=c1c2ccc3ccccc3c2n2c3c(ccc4ccccc43)c(=O)n12 ; O=c1c2c3ccccc3c3ccccc3c2n2c3c4ccccc4c4ccccc4c3c(=O)n12]

from protection.

Compounds of the formulae (A), (B) and (C) are excluded from protection. Compound (A) having CAS No. 125213-40-3, compound (B) having CAS No. 131847-09-1, and compound (C) having CAS No. 166042-85-9 are known from the prior art, but not the use thereof in an electronic device.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatic systems joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, further preferably F or CN, especially preferably CN.

An aromatic or heteroaromatic ring system which has 5-60 or 5-40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

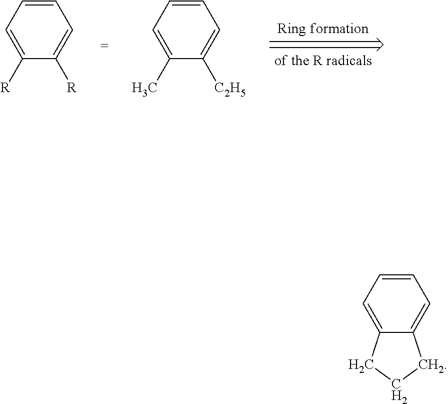

In addition, however, the abovementioned wording shall also be understood to mean that, if one of the two radicals is hydrogen, the second radical binds to the position to which the hydrogen atom was bonded, forming a ring. This will be illustrated by the following scheme:

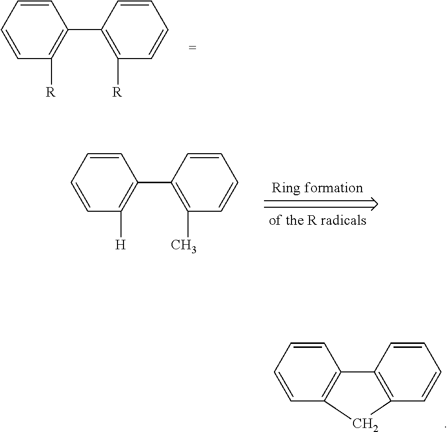

In a preferred configuration, the compounds of the invention may comprise a structure of the formulae (II-1) to (II-42); more preferably, the compounds of the invention may be selected from the compounds of the formulae (II-1) to (II-42):

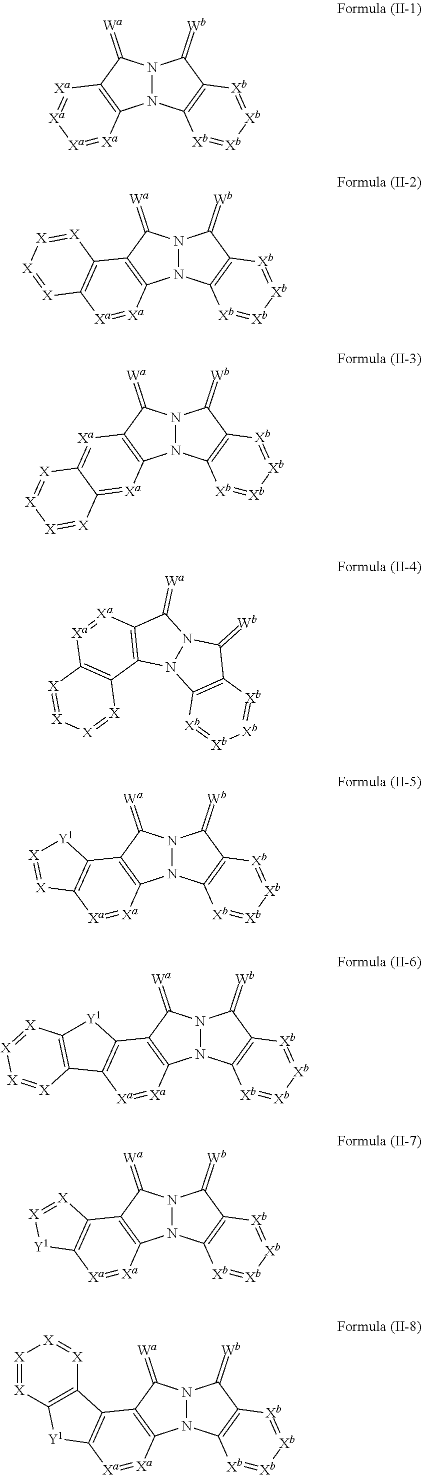

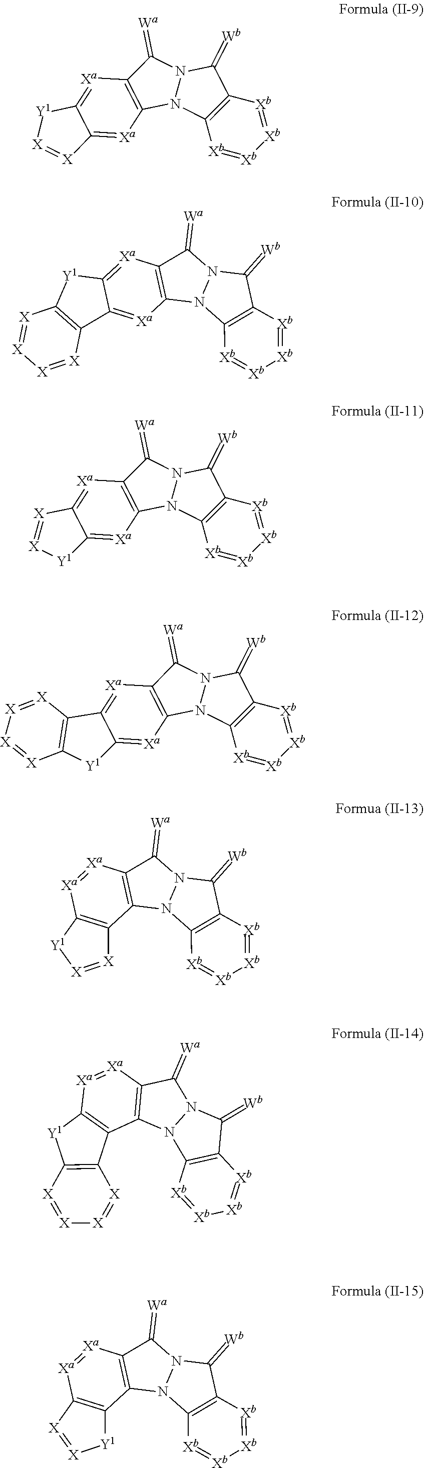

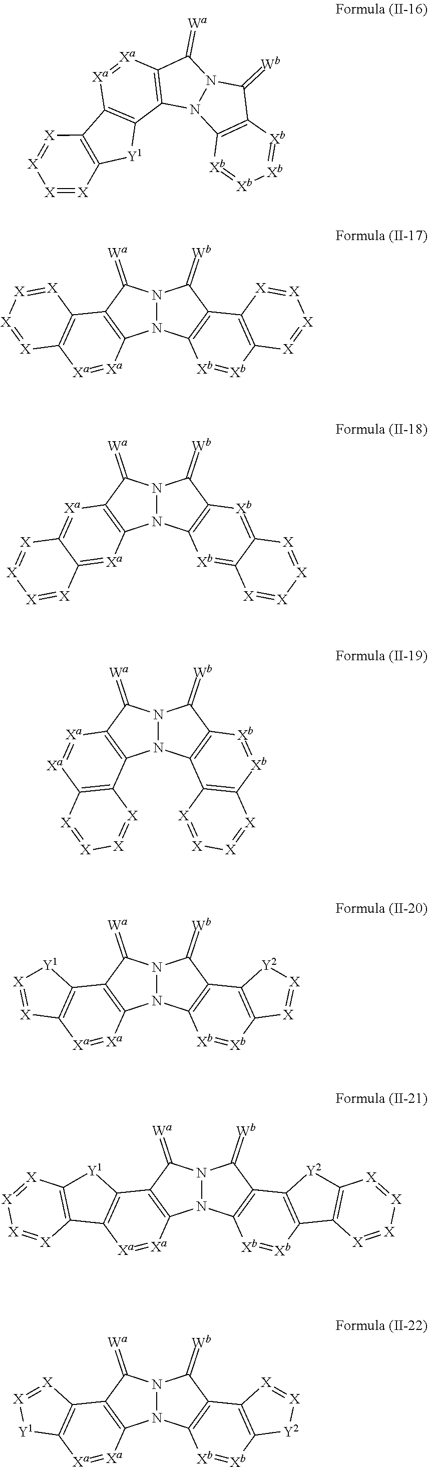

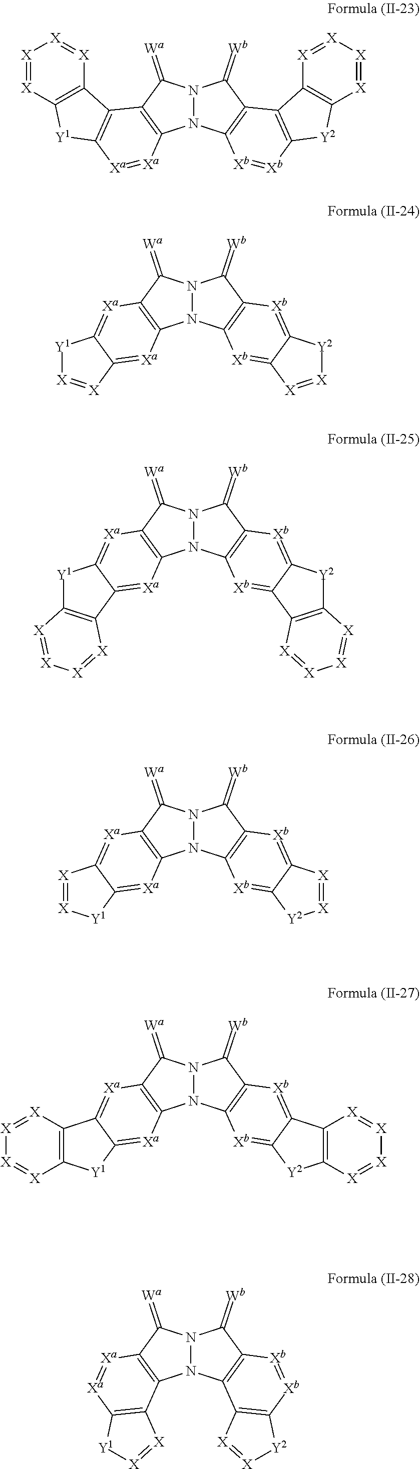

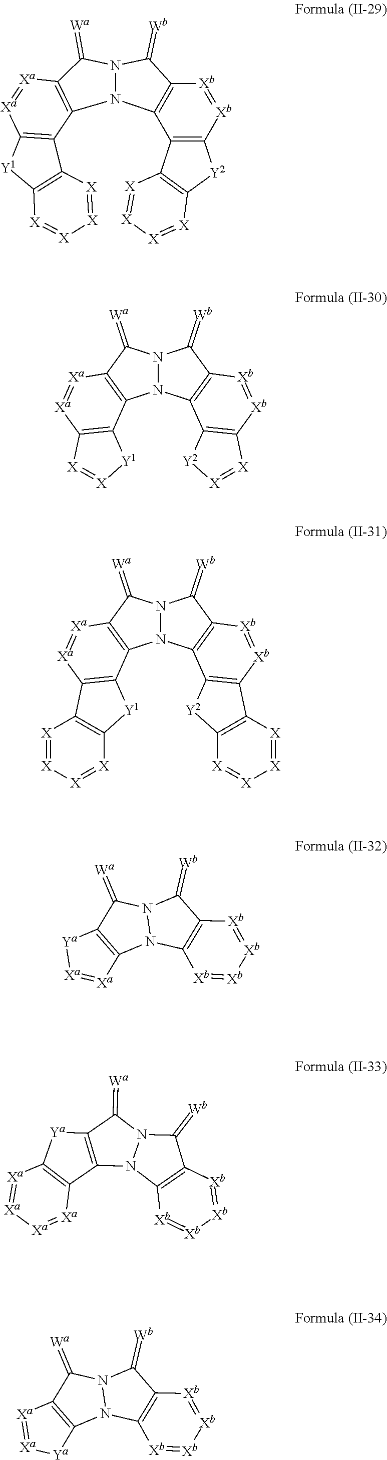

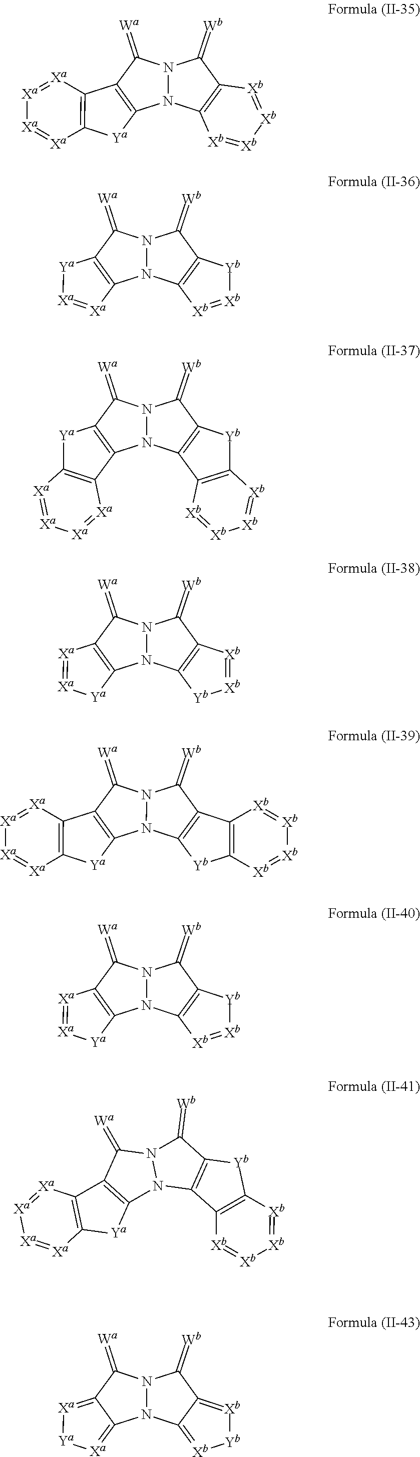

where the symbols W^{a} and W^{b} have the definition given above, especially for formula (I), and the further symbols are as follows:
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, preferably N(Ar), N(R^{a}), B(Ar), B(R^{a}), P(═O)R^{a}, P(═O)Ar, C═O, C(R^{a})₂, C═C(R^{a})₂, C═C(R^{a})(Ar), Si(R^{a})₂, O, S, Se, S═O or SO₂, more preferably N(Ar), C(R^{a})₂, O or S, where R^{a} has the definition detailed above, especially for formula (I);
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, preferably N(Ar), N(R^{b}), B(Ar), B(R^{b}), P(═O)R^{b}, P(═O)Ar, C═O, C(R^{b})₂, C═C(R^{b})₂, C═C(R^{b})(Ar), Si(R^{b})₂, O, S, Se, S═O or SO₂, more preferably N(Ar), C(R^{b})₂, O or S, where R^{b} has the definition detailed above, especially for formula (I);
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, preferably N(Ar), N(R), B(Ar), B(R), P(═O)R, P(═O)Ar, C═O, C(R)₂, C═C(R)₂, C═C(R)(Ar), Si(R)₂, O, S, Se, S═O or SO₂, more preferably N(Ar), C(R)₂, O or S, where R has the definition detailed above, especially for formula (I);
X is the same or different at each instance and is N or CR, preferably CR, with the proviso that not more than two of the X groups in one cycle are N, where R has the definition detailed above, especially for formula (I);
X^{a} is the same or different at each instance and is N or CR^{a}, preferably CR^{a}, with the proviso that not more than two of the X^{a} groups in one cycle are N, where R^{a} has the definition detailed above, especially for formula (I);
X^{b} is the same or different at each instance and is N or CR^{b}, preferably CR^{b}, with the proviso that not more than two of the X^{b} groups in one cycle are N, where R^{b} has the definition detailed above, especially for formula (I).

Preference is given here to structures of the formulae (II-1) to (II-31), and particular preference to structures of the formulae (II-1) to (II-4), (II-6), (II-8), (II-10), (II-12), (II-14), (II-16) to (II-19), (II-21), (II-23), (II-25), (II-27), (II-29), (II-31).

In addition, especially to compounds including structures of the formulae (I) and (II-1) to (II-42), at least one of the W^{a}, W^{b} groups is O; preferably, both W^{a}, W^{b} groups are O.

If two or more nitrogen atoms are present in a compound, these nitrogen atoms are preferably not adjacent, and so no N—N bonds are present.

In a further preferred embodiment, it may be the case that the compounds of the invention include a structure of the formulae (III-1) to (III-41), where the compounds of the invention may more preferably be selected from the compounds of the formulae (III-1) to (III-41)

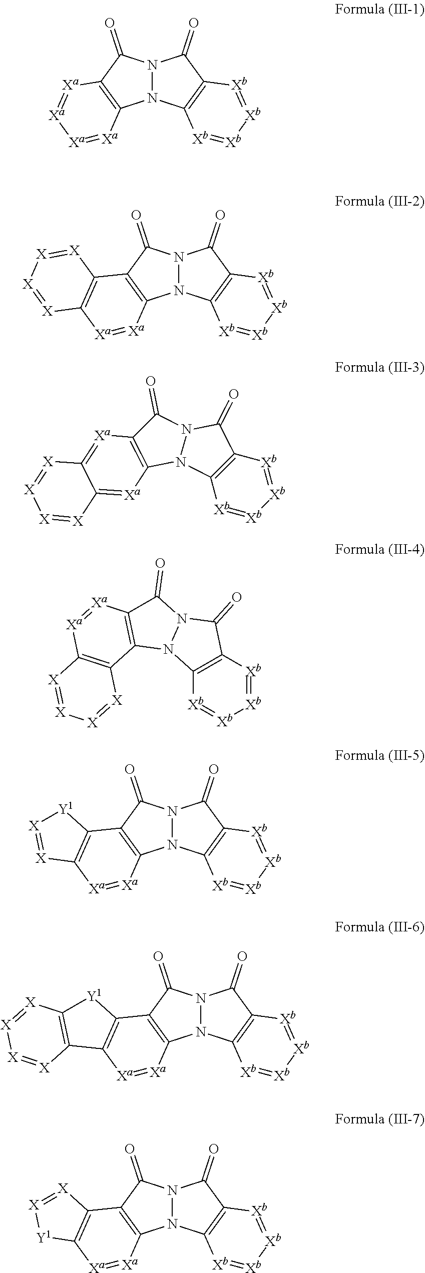

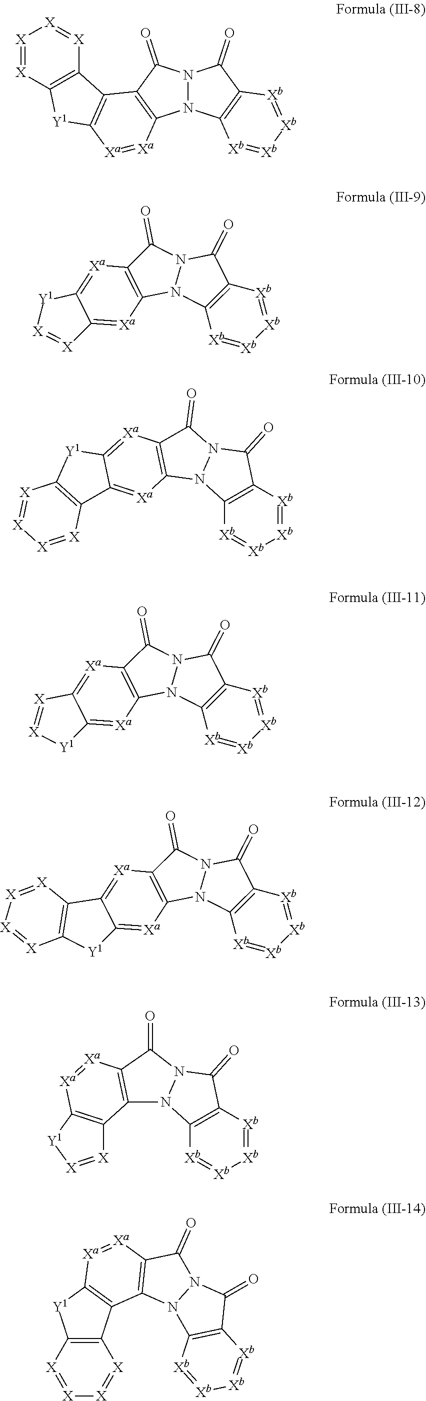

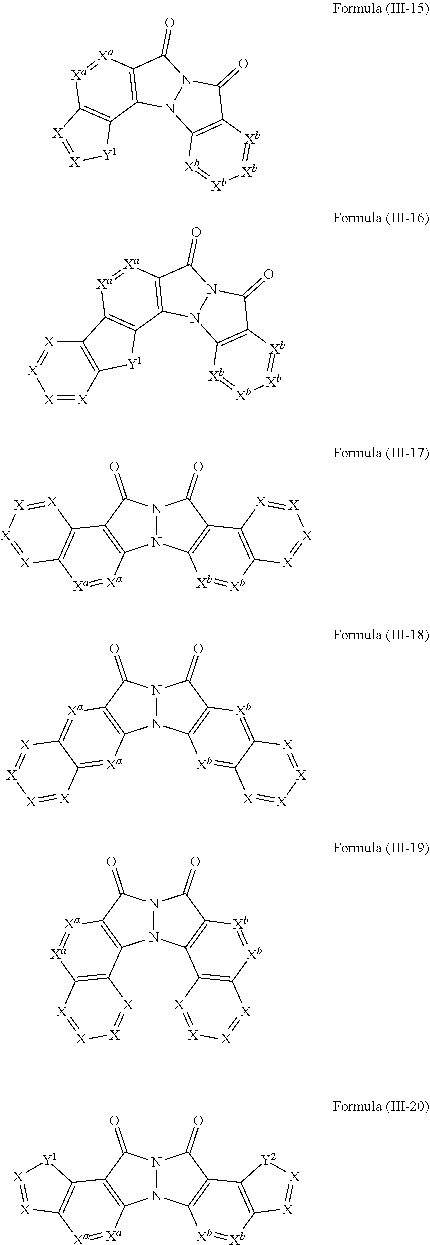

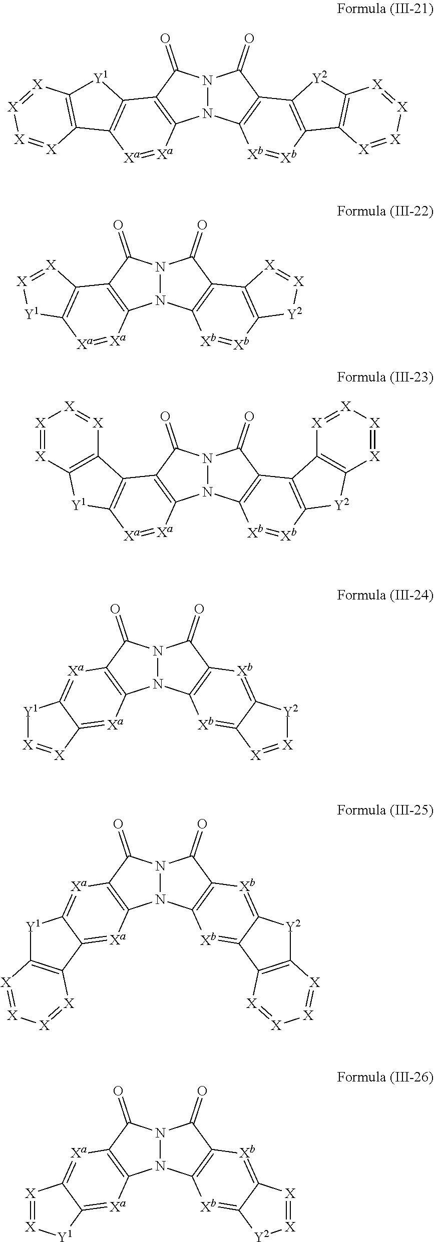

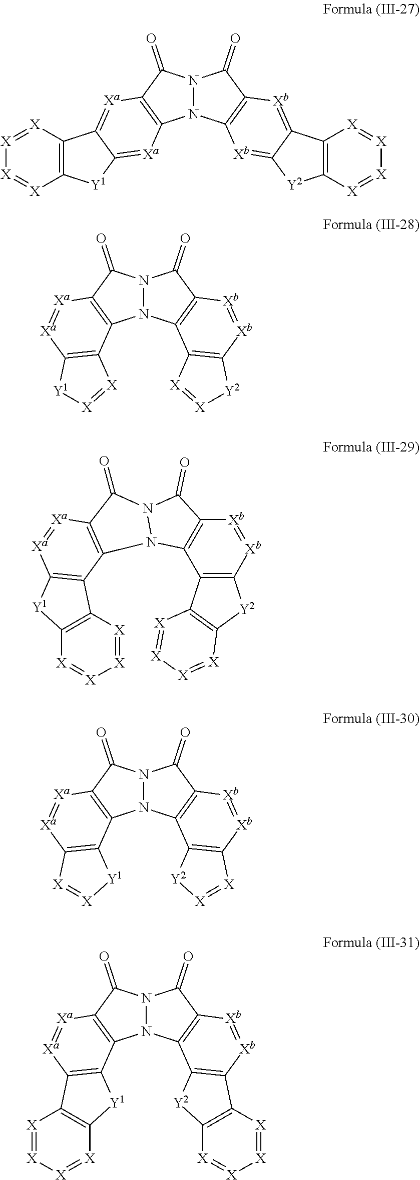

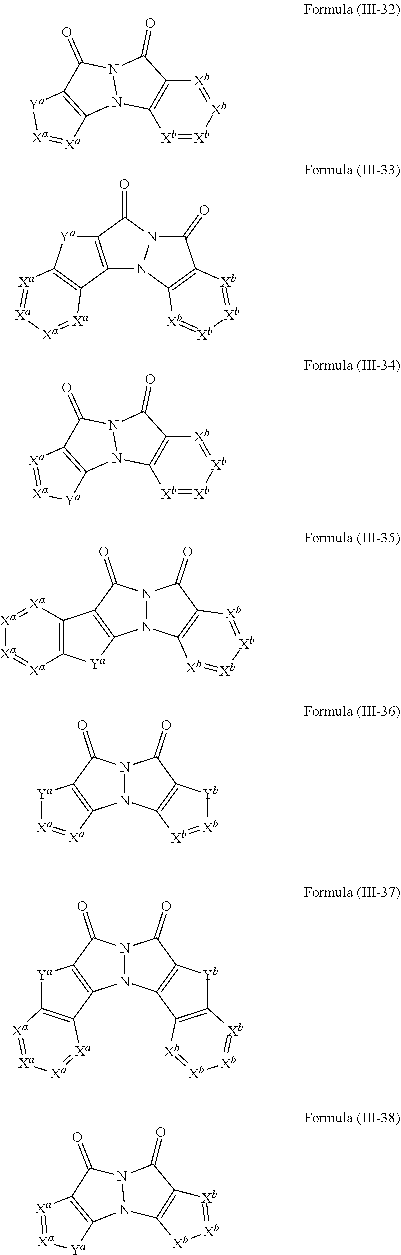

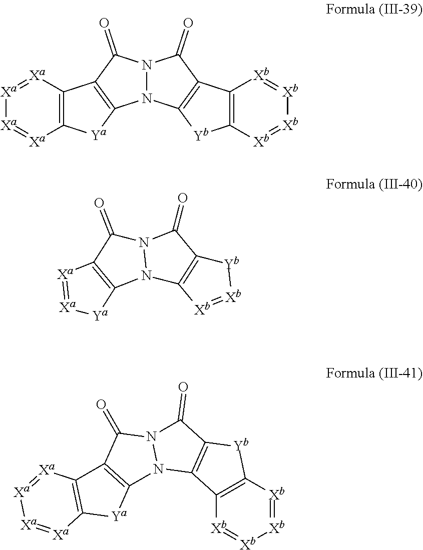

where the symbols Y¹, Y², Y^{a}, Y^{b}, X, X^{a} and X^{b} have the definitions given above, especially for formulae (II-1) to (II-42).

Preference is given here to structures of the formulae (II-1) to (II-31), and particular preference to structures of the formulae (III-1) to (III-4), (III-6), (III-8), (III-10), (III-12), (III-14), (III-16) to (III-19), (III-21), (III-23), (III-25), (III-27), (III-29), (III-31).

It may preferably be the case that, in formulae (II-1) to (II-42) and/or (III-1) to (III-41), not more than four and preferably not more than two X, X^{a} and X^{b} groups are N; more preferably, all X, X^{a} and X^{b} groups are CR, CR^{a} or CR^{b}.

In a further preferred embodiment, it may be the case that the compounds of the invention include a structure of the formulae (IV-1) to (IV-41), where the compounds of the invention may more preferably be selected from the compounds of the formulae (IV-1) to (IV-41)

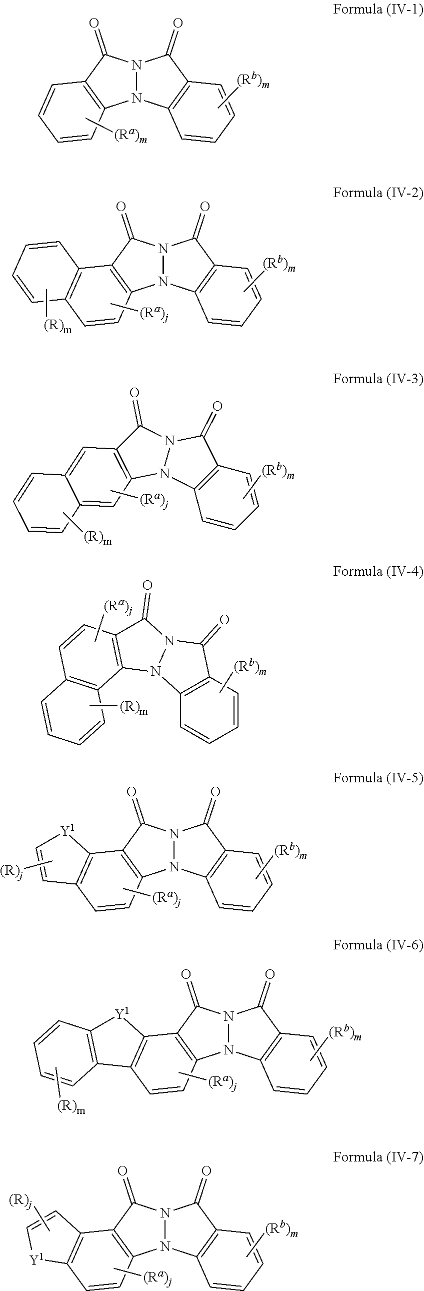

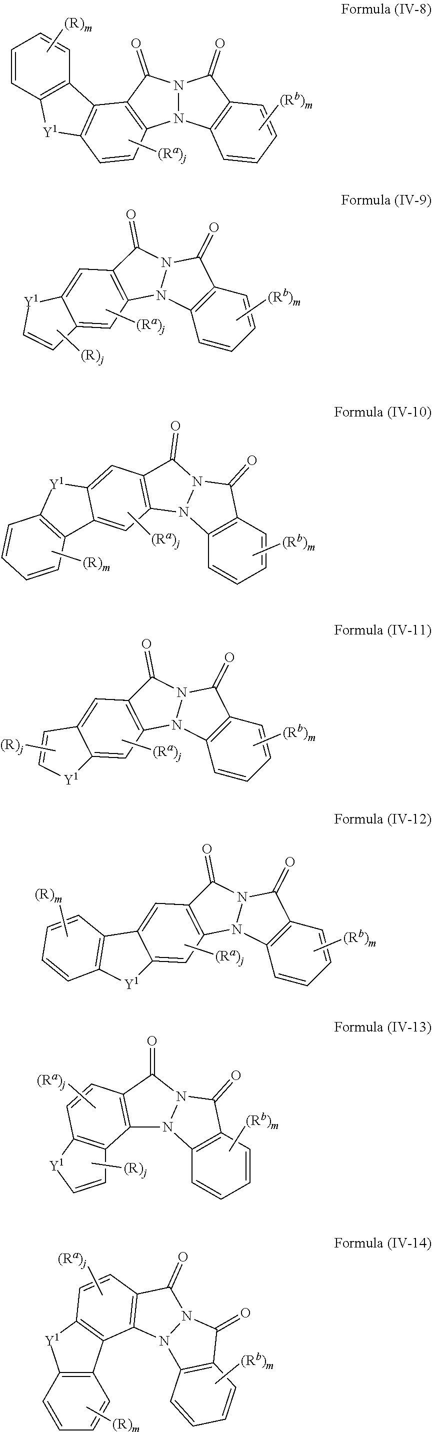

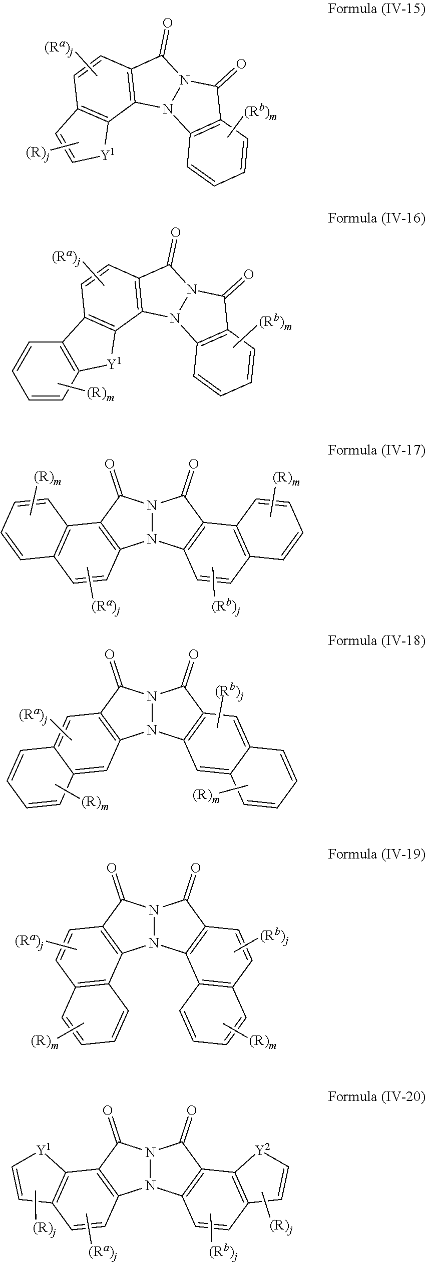

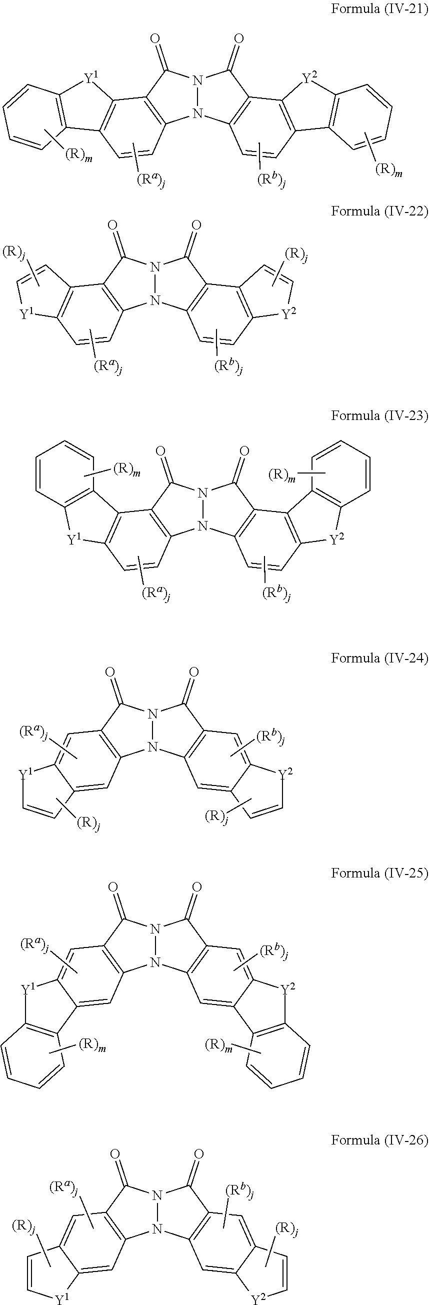

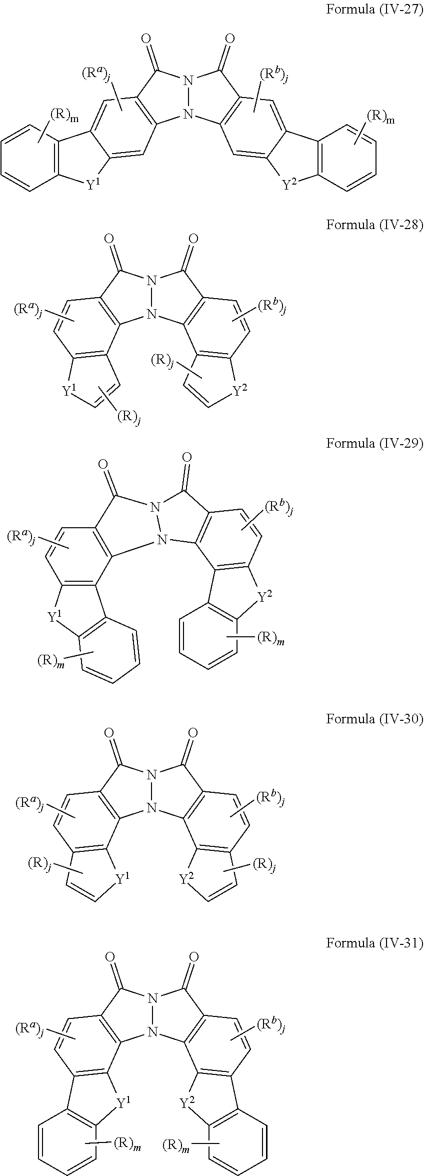

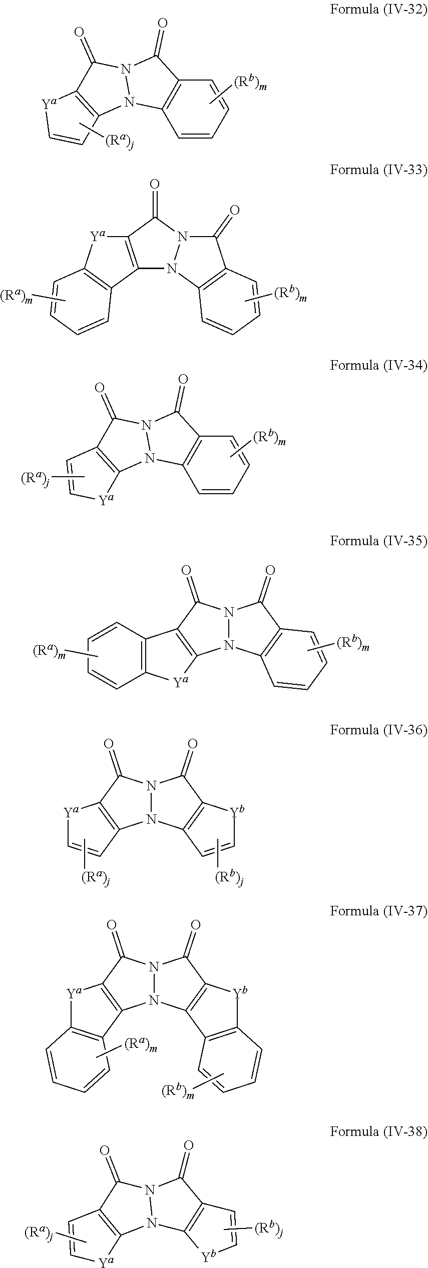

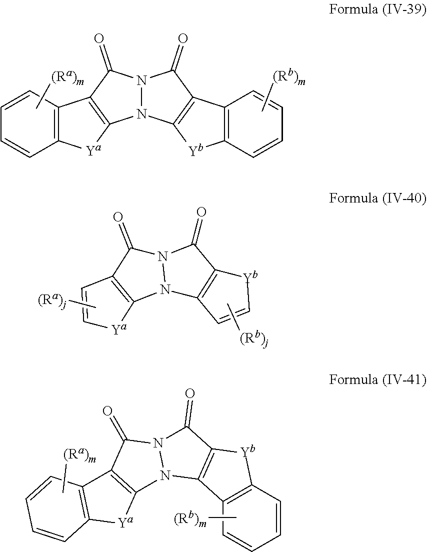

where the symbols R, R^{a} and R^{b} have the definitions given above, especially for formula (I), the symbols Y¹, Y², Y^{a} and Y^{b} have the definitions given above, especially for formulae (II-1) to (II-42), and the further symbols have the following definition:
m is 0, 1, 2, 3 or 4, preferably 0, 1 or 2;
j is 0, 1 or 2, preferably 0 or 1.

Preference is given here to structures/compounds of the formulae (IV-1) to (IV-31), and particular preference to structures/compounds of the formulae (IV-1) to (IV-4), (IV-6), (IV-8), (IV-10), (IV-12), (IV-14), (IV-16) to (IV-19), (IV-21), (IV-23), (IV-25), (IV-27), (IV-29), (IV-31).

It may further be the case that the Y^{a} and Y^{b} radicals are the same. In addition, the Y^{a} and Y^{b} radicals may be different.

In a further configuration, it may be the case that one of the Y^{a} and Y^{b} radicals is O and one of the Y^{a} and Y^{b} radicals is C(R^{a})₂ or C(R^{b})₂.

In a further embodiment, it may be the case that one of the Y^{a} and Y^{b} radicals is NAr and one of the Y^{a} and Y^{b} radicals is C(R^{a})₂ or C(R^{b})₂.

It may also be the case that the Y¹ and Y² radicals are the same. In addition, the Y¹ and Y² radicals may be different.

In a further configuration, it may be the case that one of the Y¹ and Y² radicals is O and one of the Y¹ and Y² radicals is C(R^{a})₂ or C(R^{b})₂.

In a further embodiment, it may be the case that one of the Y¹ and Y² radicals is NAr and one of the Y¹ and Y² radicals is C(R^{a})₂ or C(R^{b})₂.

In a further preferred embodiment, it may be the case that the compounds of the invention include a structure of the formulae (V-1) to (V-8), where the compounds of the invention may more preferably be selected from the compounds of the formulae (V-1) to (V-8)

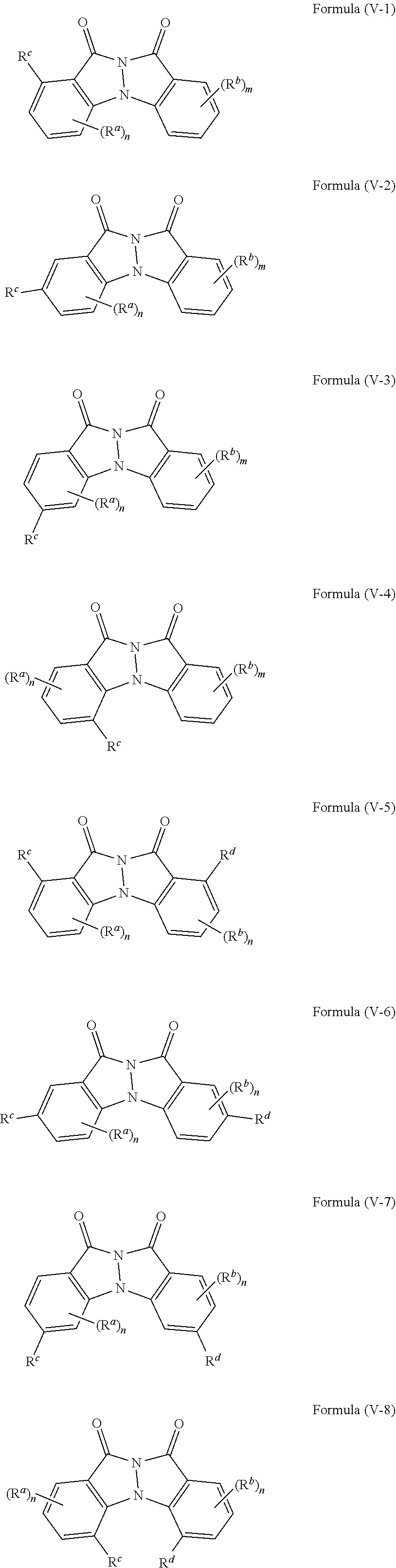

where the symbols R^{a} and R^{b} have the definition given above, especially for formula (I), and the further symbols have the following definition:
R^{c}, R^{d} is the same or different at each instance and is N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; or heteroarylthio group which has 5 to 60 aromatic ring atoms and may be substituted by one or more R¹ radicals, or a diarylamino, arylheteroarylamino, diheteroarylamino group which has 5 to 60 aromatic ring atoms and may be substituted by one or more R¹ radicals, or an arylalkyl or heteroarylalkyl group which has 5 to 60 aromatic ring atoms and 1 to 10 carbon atoms in the alkyl radical and may be substituted by one or more R¹ radicals; at the same time, one R^{c} radical may form a ring system with an R, R^{a}, R^{b}, R^{d} radical or a further group; at the same time, an R^{d} radical may form a ring system with an R, R^{a}, R^{b}, R^{c} radical or a further group;
m is 0, 1, 2, 3 or 4, preferably 0, 1 or 2;
n is 0, 1, 2 or 3, preferably 0, 1 or 2.

In addition, especially for structures/compounds of the formulae (V-1) to (V-8), it may be the case that the R^{c} and R^{d} radicals are the same. In addition, the R^{c} and R^{d} radicals may be different.

The sum total of the indices j, m and n, especially in structures/compounds of the formulae (IV-1) to (IV-41) and/or (V-1) to (V-8), is preferably at most 10, preferably not more than 8, especially preferably not more than 6 and more preferably not more than 4.

In addition, in formulae including (I), (II-1) to (II-42), (Ill-1) to (Ill-42), (IV-1) to (IV-41), (V-1) to (V-8) and/or the preferred embodiments of these formulae detailed hereinafter, it may be the case that at least one R, R^{a}, R^{b}, R^{c}, R^{d} radical is a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or is an aromatic or heteroaromatic ring system which has 5 to 60 aromatic ring atoms and may be substituted in each case by one or more R¹ radicals, or is an aryl or heteroaryloxy group which has 5 to 60 aromatic ring atoms and may be substituted by one or more R¹ radicals, or is a heteroarylthio group which has 5 to 60 aromatic ring atoms and may be substituted by one or more R¹ radicals, or is a diarylamino, arylheteroarylamino, diheteroarylamino group which has 5 to 60 aromatic ring atoms and may be substituted by one or more R¹ radicals, or is an arylalkyl or heteroarylalkyl group which has 5 to 60 aromatic ring atoms and 1 to 10 carbon atoms in the alkyl radical and may be substituted by one or more R¹ radicals.

Preferably, in formulae including (I), (II-1) to (II-42), (III-1) to (III-42), (IV-1) to (IV-41), (V-1) to (V-8) and/or the preferred embodiments of these formulae detailed hereinafter, it may be the case that at least one R, R^{a}, R^{b}, R^{c}, R^{d} radical is an aromatic or heteroaromatic ring system which has 5 to 60 aromatic ring atoms and may be substituted in each case by one or more R¹ radicals, or is an aryl or heteroaryloxy group which has 5 to 60 aromatic ring atoms and may be substituted by one or more R¹ radicals, or is a heteroarylthio group which has 5 to 60 aromatic ring atoms and may be substituted by one or more R¹ radicals, or is a diarylamino, arylheteroarylamino, diheteroarylamino group which has 5 to 60 aromatic ring atoms and may be substituted by one or more R¹ radicals, or is an arylalkyl or heteroarylalkyl group which has 5 to 60 aromatic ring atoms and 1 to 10 carbon atoms in the alkyl radical and may be substituted by one or more R¹ radicals, more preferably an aromatic or heteroaromatic ring system which has 5 to 60 aromatic ring atoms and may be substituted in each case by one or more R¹ radicals.

In a preferred development of the present invention, it may be the case that at least two R, R^{a}, R^{b}, R^{c}, R^{d} radicals together with the further groups to which the two R, R^{a}, R^{b}, R^{c}, R^{d} radicals bind form a fused ring, where the two R, R^{a}, R^{b}, R^{c}, R^{d} radicals form at least one structure of the formulae (RA-1) to (RA-12):

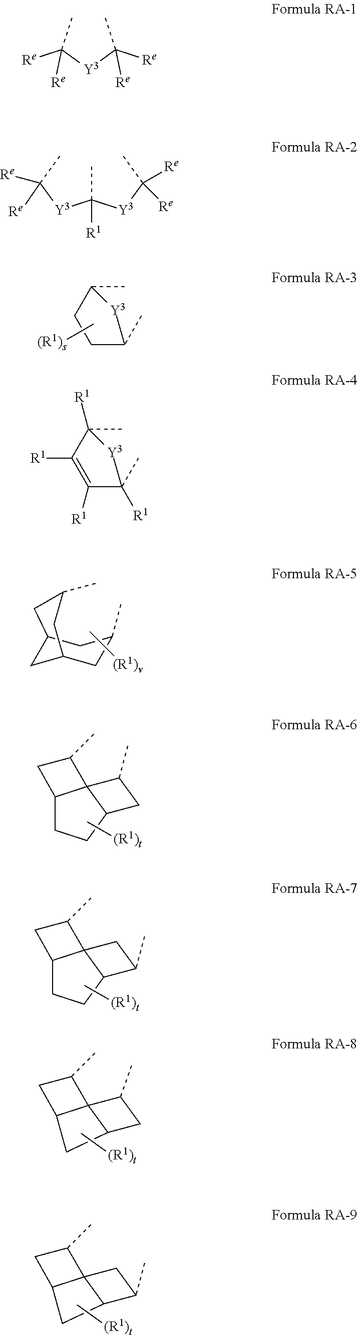

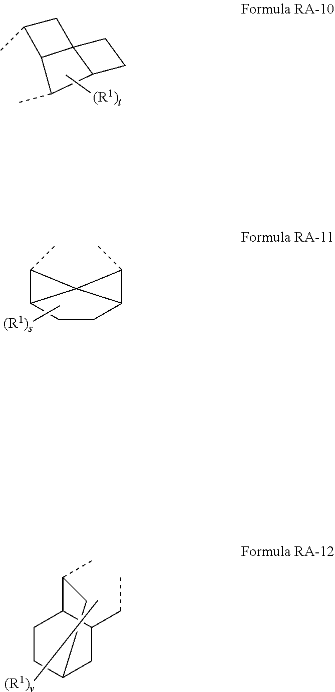

where R has the definition detailed above, the dotted bonds represent the sites of attachment via which the two R, R^{a}, R^{b}, R^{c}, R^{d} radicals bind to the further groups, and the further symbols have the following definition:
Y³ is the same or different at each instance and is C(R¹)₂, (R¹)₂C—C(R¹)₂, (R¹)C═C(R¹), NR¹, NAr′, O or S, preferably C(R¹)₂, (R¹)₂C—C(R¹)₂, (R¹)C═C(R¹), O or S;
R^{e} is the same or different at each instance and is F, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may be substituted in each case by one or more R² radicals, where one or more adjacent CH₂ groups may be replaced by R²C═CR², C≡C, Si(R²)₂, C═O, C═S, C═Se, C═NR², —C(═O)O—, —C(═O)NR²—, NR², P(═O)(R¹), —O—, —S—, SO or SO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals; at the same time, it is also possible for two Re radicals together or one Re radical together with an R¹ radical or together with a further group to form a ring system;
s is 0, 1, 2, 3, 4, 5 or 6, preferably 0, 1, 2, 3 or 4, more preferably 0, 1 or 2;
t is 0, 1, 2, 3, 4, 5, 6, 7 or 8, preferably 0, 1, 2, 3 or 4, more preferably 0, 1 or 2;
v is 0, 1, 2, 3, 4, 5, 6, 7, 8 or 9, preferably 0, 1, 2, 3 or 4, more preferably 0, 1 or 2.

In a preferred embodiment of the invention, the at least two R, R^{a}, R^{b}, R^{c}, R^{d} radicals together with the further groups to which the two R, R^{a}, R^{b}, R^{c}, R^{d} radicals bind form a fused ring, where the two R, R^{a}, R^{b}, R^{c}, R^{d} radicals preferably form at least one of the structures of the formulae (RA-1a) to (RA-4f):

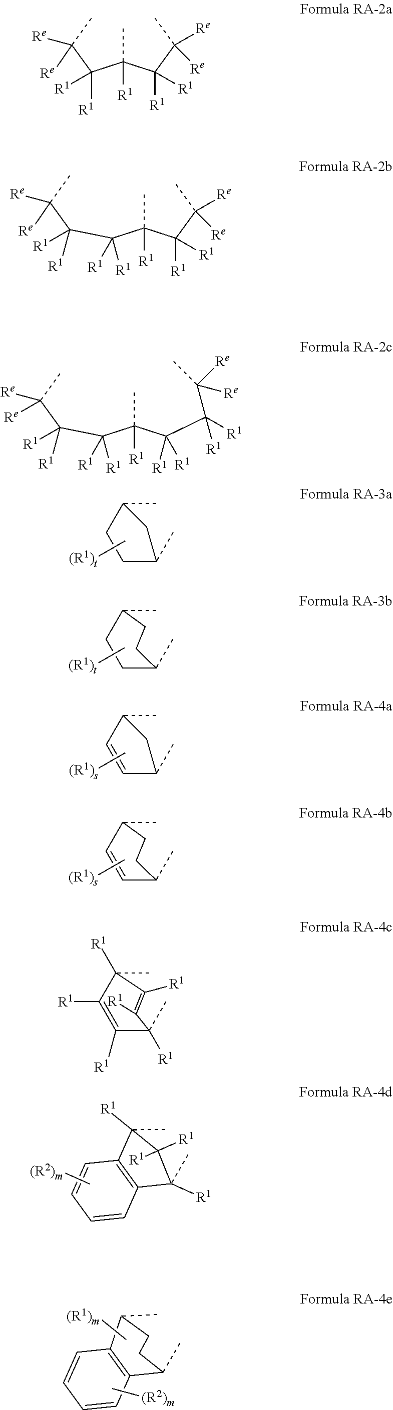

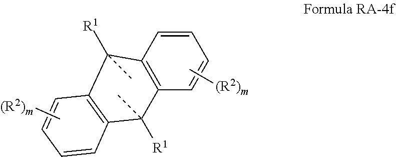

where the dotted bonds represent the sites of attachment via which the two R, R^{a}, R^{b}, R^{c}, R^{d} radicals bind to the further groups, the index m is 0, 1, 2, 3 or 4, preferably 0, 1 or 2, and the symbols R¹, R², Re and indices s and t have the definition detailed above, especially for formula (I) and/or formulae (RA-1) to (RA-12).

It may further be the case that the at least two R, R^{a}, R^{b}, R^{c}, R^{d} radicals form the structures of the formulae (RA-1) to (RA-12) and/or (RA-1a) to (RA-4f) and form a fused ring, represent R, R^{a}, R^{b}, R^{c}, R^{d} radicals from adjacent X, X^{a}, X^{b} groups, or represent R, R^{a}, R^{b}, R^{c}, R^{d} radicals that each bind to adjacent carbon atoms, where these carbon atoms are preferably connected via a bond.

In a further-preferred configuration, at least two R, R^{a}, R^{b}, R^{c}, R^{d} radicals together with the further groups to which the two R, R^{a}, R^{b}, R^{c}, R^{d} radicals bind form a fused ring, where the two R, R^{a}, R^{b}, R^{c}, R^{d} radicals form structures of the formula (RB),

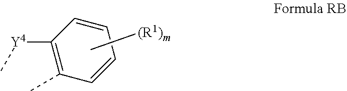

where R¹ has the definition set out above, especially for formula (I), the dotted bonds represent the bonding sites via which the two R, R^{a}, R^{b}, R^{c}, R^{d} radicals bind to the further groups, the index m is 0, 1, 2, 3 or 4, preferably 0, 1 or 2, and Y⁴ is C(R¹)₂, NR¹, NAr′, BR¹, BAr′, O or S, preferably C(R¹)₂, NAr′ or O.

It may be the case here that the at least two R, R^{a}, R^{b}, R^{c}, R^{d} radicals form the structures of the formula (RB) and form a fused ring, represent R, R^{a}, R^{b}, R^{c}, R^{d} radicals from adjacent X, X^{a}, X^{b} groups, or represent R, R^{a}, R^{b}, R^{c}, R^{d} radicals that each bind to adjacent carbon atoms, where these carbon atoms are preferably connected to one another via a bond.

More preferably, the compounds include at least one structure of the formulae (VI-1) to (VI-22); more preferably, the compounds are selected from compounds of the formulae (VI-1) to (VI-22), where the compounds have at least one fused ring:

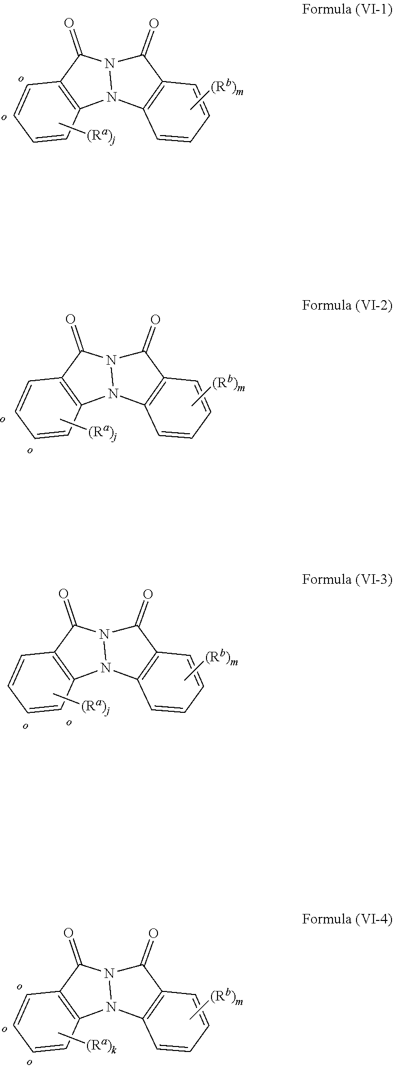

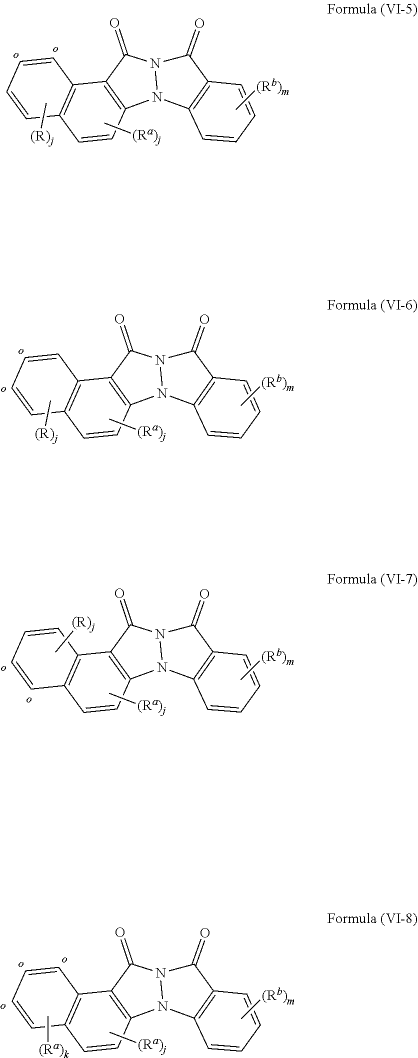

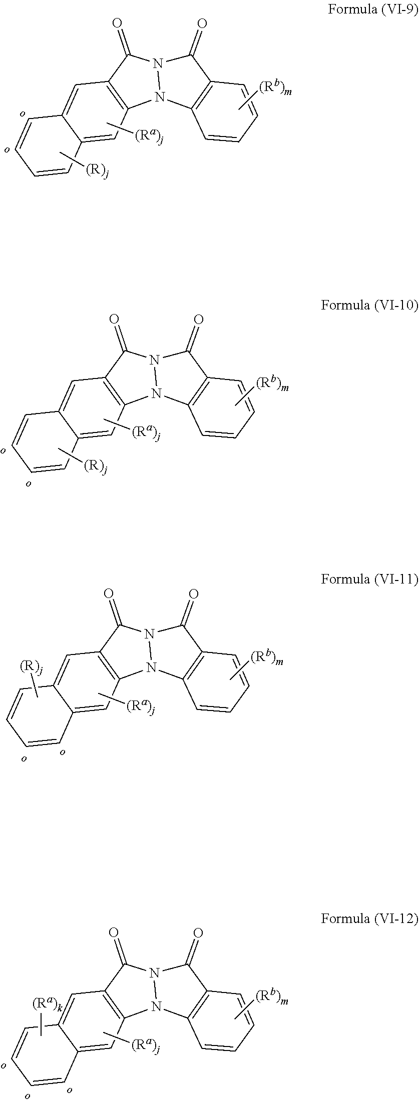

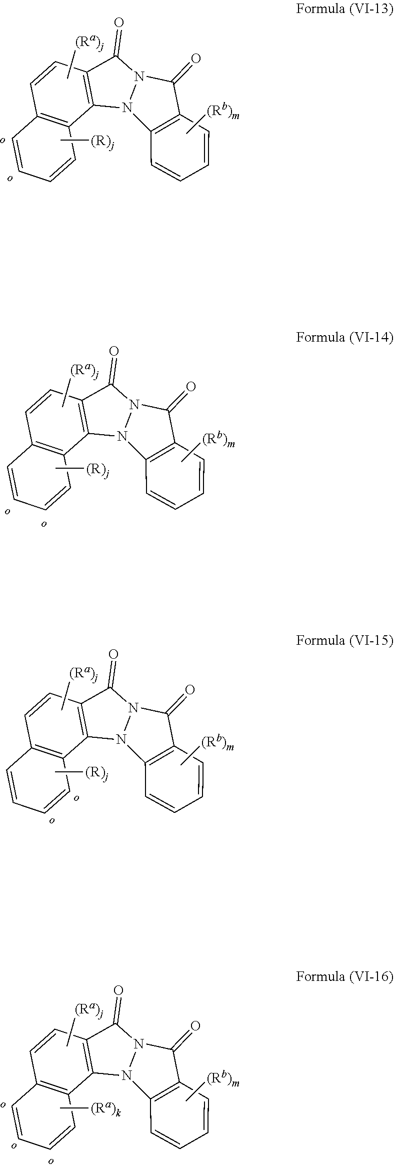

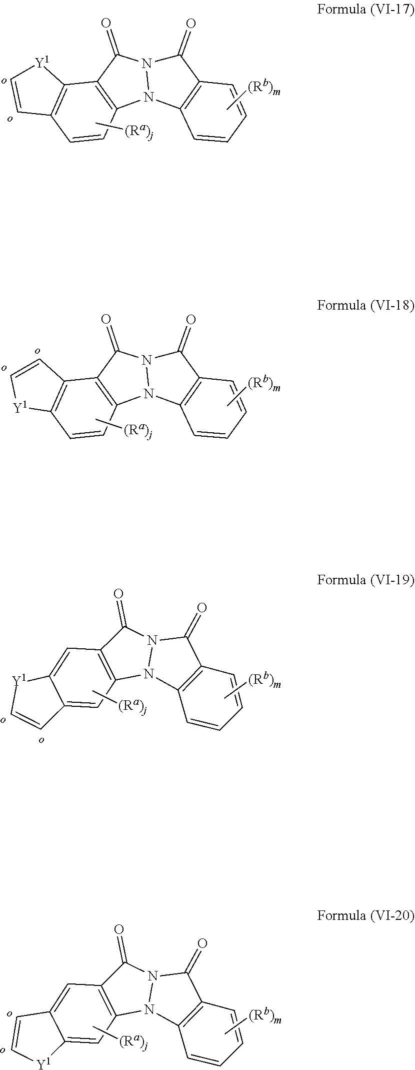

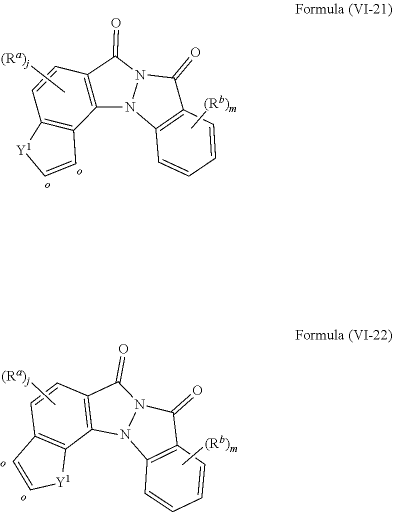

where the symbols R, R^{a}, R^{b}, Y¹ and Y² have the definitions given above, especially for formula (I) and/or formulae (II-1) to (II-42), the symbol o represents the sites of attachment, and the further symbols have the following definition:
m is 0, 1, 2, 3 or 4, preferably 0, 1 or 2;
j is 0, 1 or 2, preferably 0 or 1;
k is 0 or 1.

Preference is given here to structures/compounds of the formula (VI-1) to (VI-4).

More preferably, the compounds include at least one structure of the formulae (VII-1) to (VII-4); more preferably, the compounds are selected from compounds of the formulae (VII-1) to (VII-4), where the compounds have at least one fused ring:

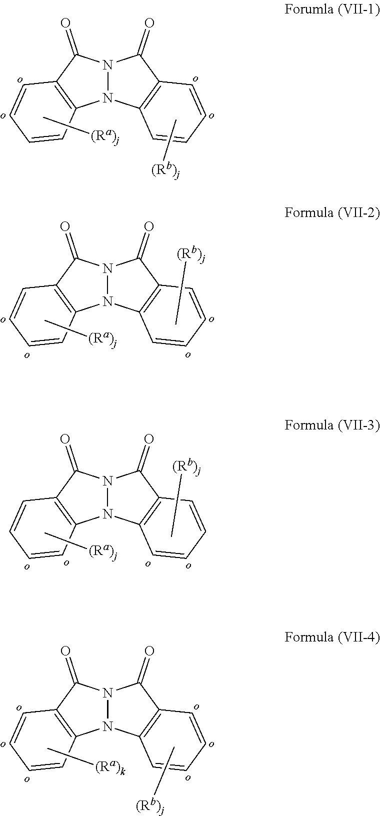

where the symbols R, R^{a} and R^{b} have the definition given above, especially for formula (I), the symbol o represents the sites of attachment of the fused ring, and the further symbols have the following definition:
j is 0, 1 or 2, preferably 0 or 1;
k is 0 or 1.

Preferably, the fused ring, especially in formulae (VI-1) to (VI-22) and/or (VII-1) to (VII-4), is formed by at least two R, R^{a}, R^{b}, R^{c}, R^{d} radicals and the further groups to which the two R, R^{a}, R^{b}, R^{c}, R^{d} radicals bind, where the at least two R, R^{a}, R^{b}, R^{c}, R^{d} radicals form structures of the formulae (RA-1) to (RA-12) and/or of the formula (RB), preferably structures of the formulae (RA-1) to (RA-12).

It may preferably be the case that the compounds have at least two fused rings, where at least one fused ring is formed by structures of the formulae (RA-1) to (RA-12) and/or (RA-1a) to (RA-4f) and a further ring by structures of the formulae (RA-1) to (RA-12), (RA-1a) to (RA-4f) or (RB).

Especially in the formulae (VI-1) to (VI-22) and/or (VII-1) to (VII-4), the sum total of the indices k, j and m is preferably 0, 1, 2 or 3, more preferably 1 or 2.

It may additionally be the case that the substituents R, R^{a}, R^{b}, R^{c}, R^{d}, R^{e}, R¹ and R² according to the above formulae do not form a fused aromatic or heteroaromatic ring system with the ring atoms of the ring system to which the substituents R, R^{a}, R^{b}, R^{c}, R^{d}, Re, R¹ and R² bind. This includes the formation of a fused aromatic or heteroaromatic ring system with possible substituents R¹ and R² that may be bonded to the R, R^{a}, R^{b}, R^{c}, R^{d}, R^{e} and R¹ radicals.

When two radicals that may especially be selected from R, R^{a}, R^{b}, R^{c}, R^{d}, R^{e}, R¹ and/or R² form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R, R^{a}, R^{b}, R^{c}, R^{d}, R^{e}, R¹ and/or R² may also be joined to one another via a bond, such that this can bring about a ring closure. In this case, each of the corresponding bonding sites has preferably been provided with a substituent R, R^{a}, R^{b}, R^{c}, R^{d}, R^{e}, R¹ and/or R².

In a preferred configuration, a compound of the invention can be represented by at least one of the structures of formulae (I), (II-1) to (II-42), (III-1) to (III-42), (IV-1) to (IV-41), (V-1) to (V-8), (VI-1) to (VI-22) and/or (VII-1) to (VII-4). Preferably, compounds of the invention, preferably comprising structures of formulae (I), (II-1) to (II-42), (III-1) to (III-42), (IV-1) to (IV-41), (V-1) to (V-8), (VI-1) to (VI-22) and/or (VII-1) to (VII-4) have a molecular weight of not more than 5000 g/mol, preferably not more than 4000 g/mol, particularly preferably not more than 3000 g/mol, especially preferably not more than 2000 g/mol and most preferably not more than 1200 g/mol.

In addition, it is a feature of preferred compounds of the invention that they are sublimable. These compounds generally have a molar mass of less than about 1200 g/mol.

Preferred aromatic or heteroaromatic ring systems R, R^{a}, R^{b}, R^{c}, R^{d}, R^{e}, Ar′ and/or Ar are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3, 4 or 9 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹ or R radicals.

It may preferably be the case that at least one substituent R, R^{a}, R^{b} is the same or different at each instance and is selected from the group consisting of H, D, a branched or cyclic alkyl, alkoxy or thioalkoxy group having 3 to 20 carbon atoms or an aromatic or heteroaromatic ring system selected from the groups of the following formulae Ar-1 to Ar-78; preferably, the substituents R, R^{a}, R^{b} either form a fused ring, preferably according to the structures of the formulae (RA-1) to (RA-12) or (RB), or the substituent R, R^{a}, R^{b} is the same or different at each instance and is selected from the group consisting of H, D or an aromatic heteroaromatic ring system selected from the groups of the following formulae Ar-1 to Ar-78, and/or the Ar′ group is the same or different at each instance and is selected from the groups of the following formulae Ar-1 to Ar-78:

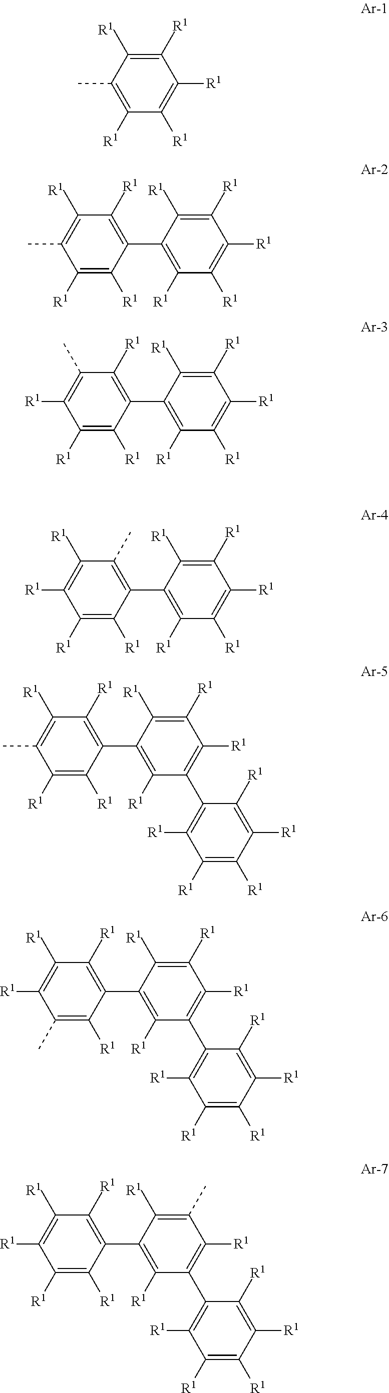

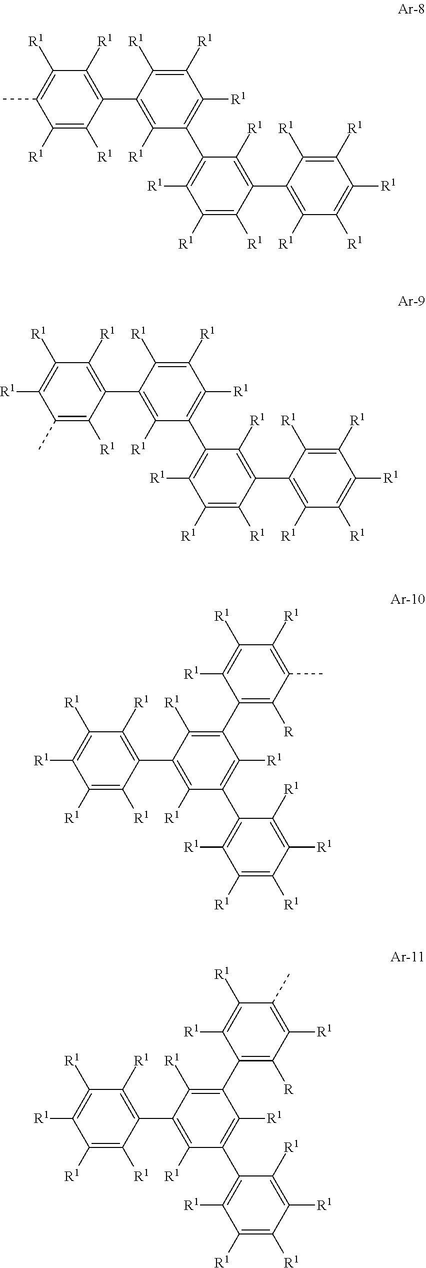

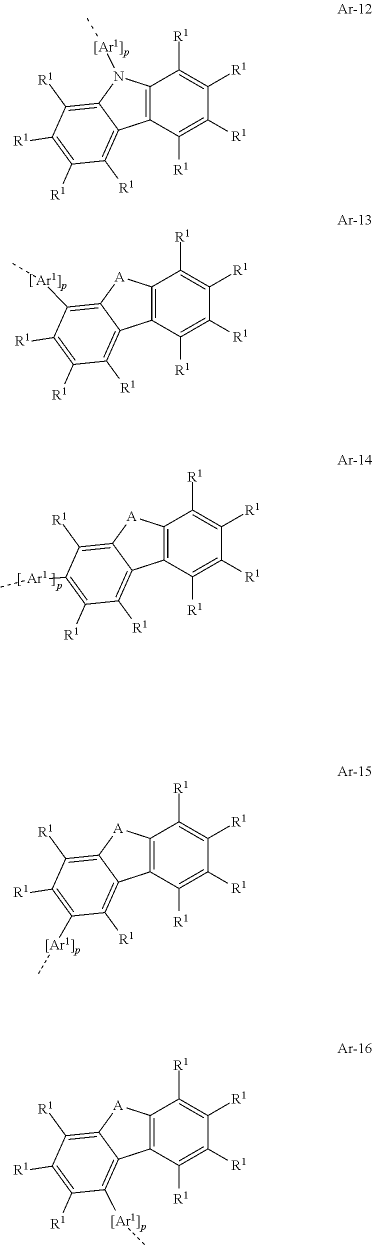

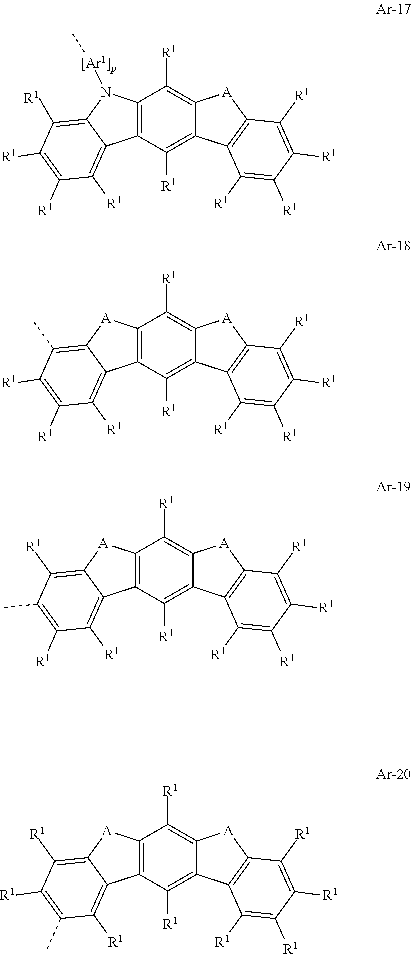

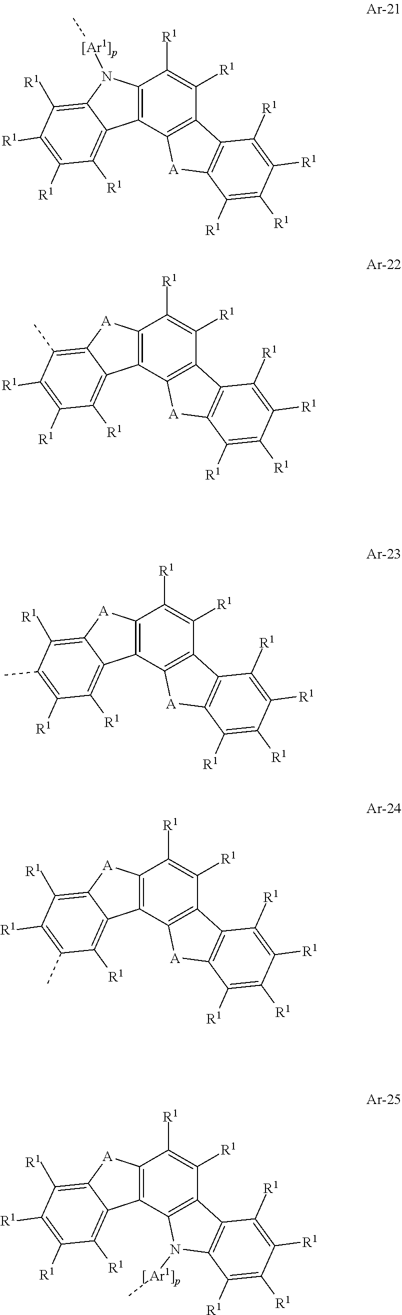

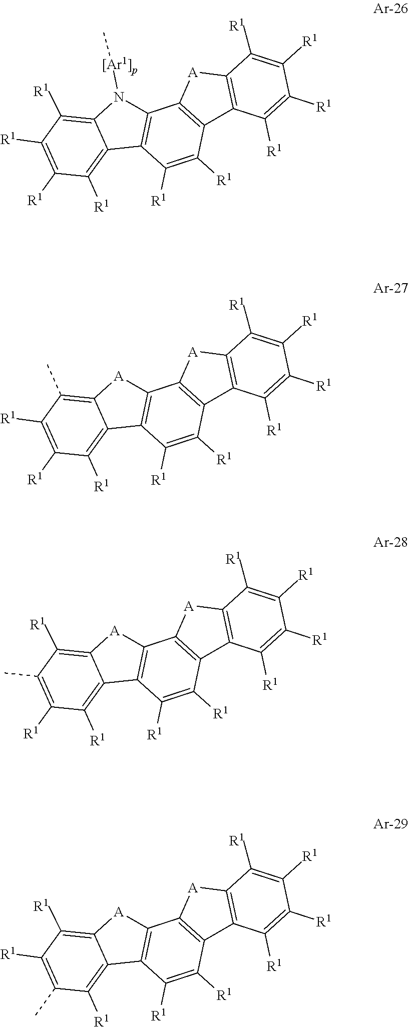

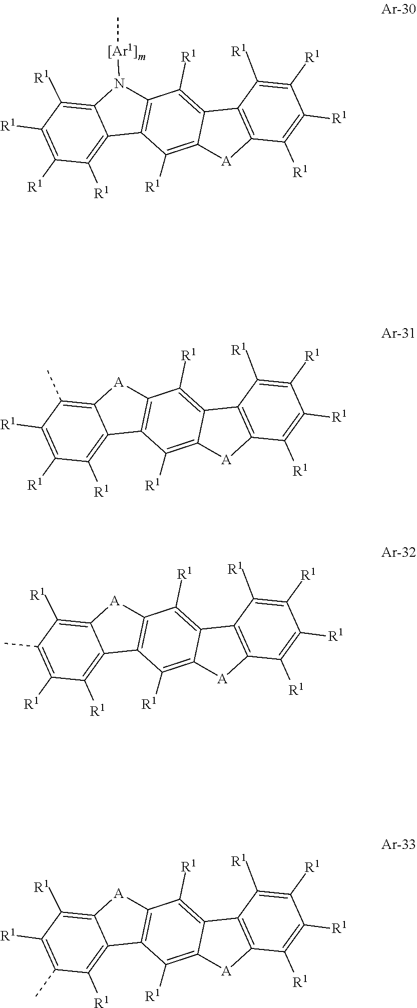

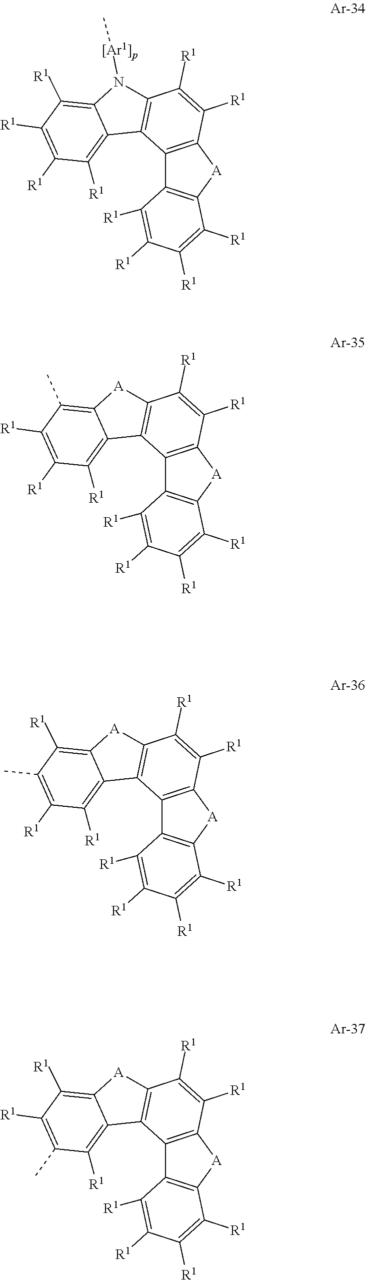

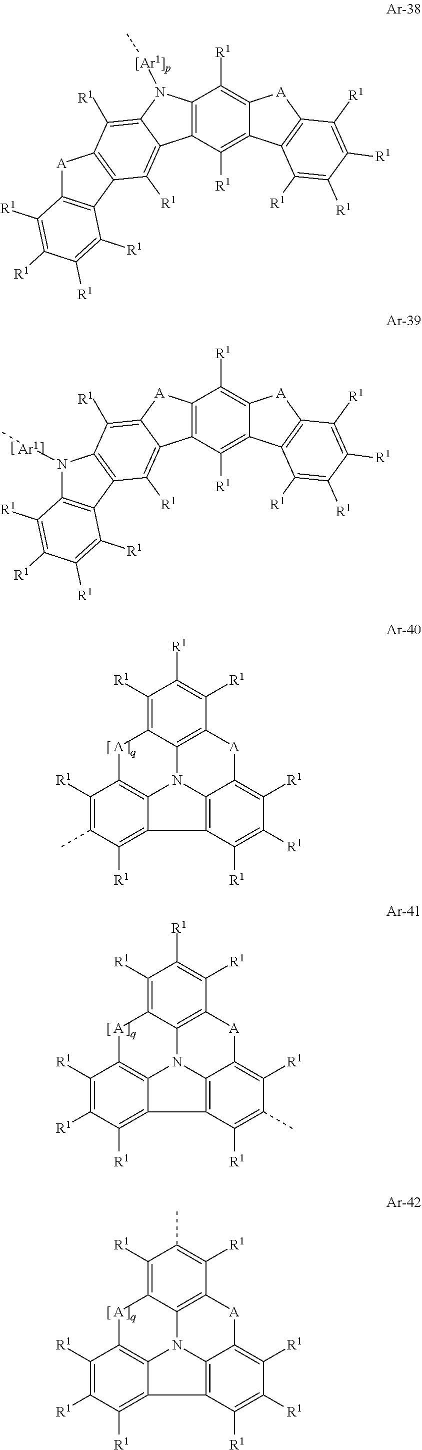

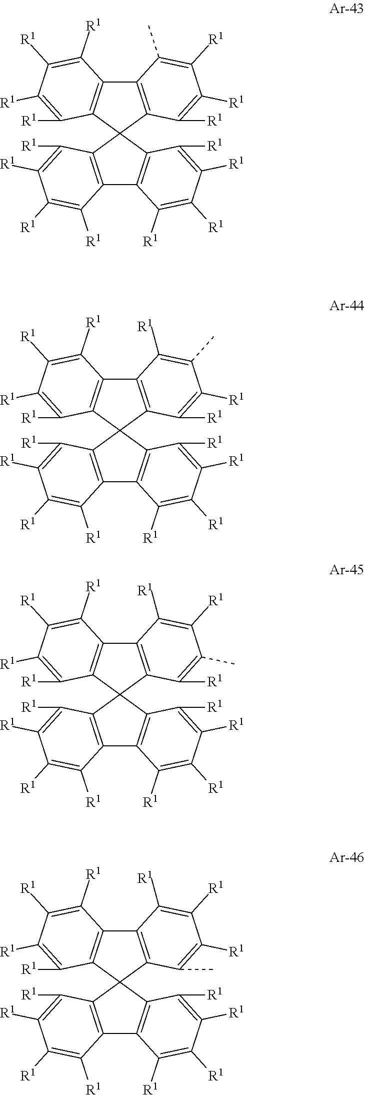

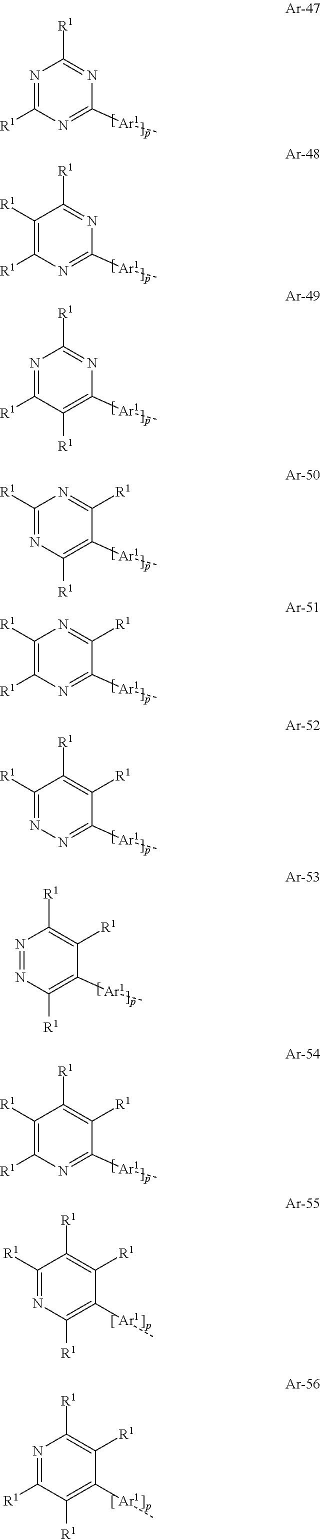

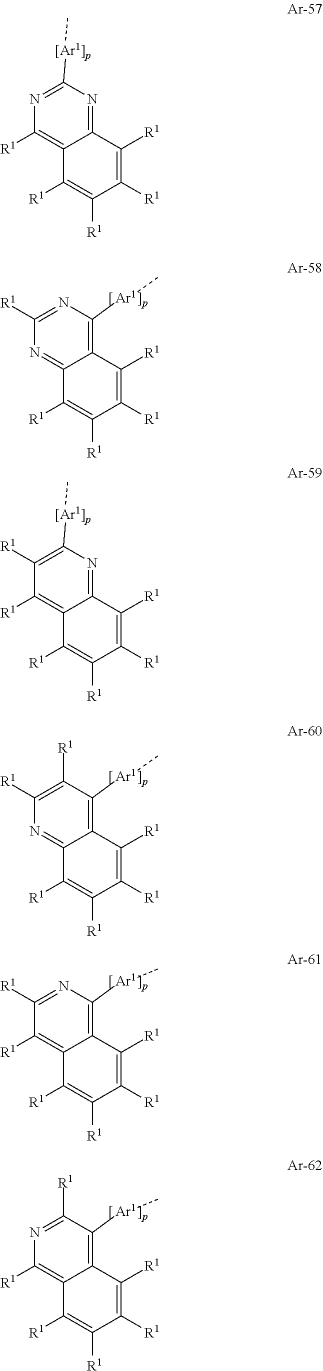

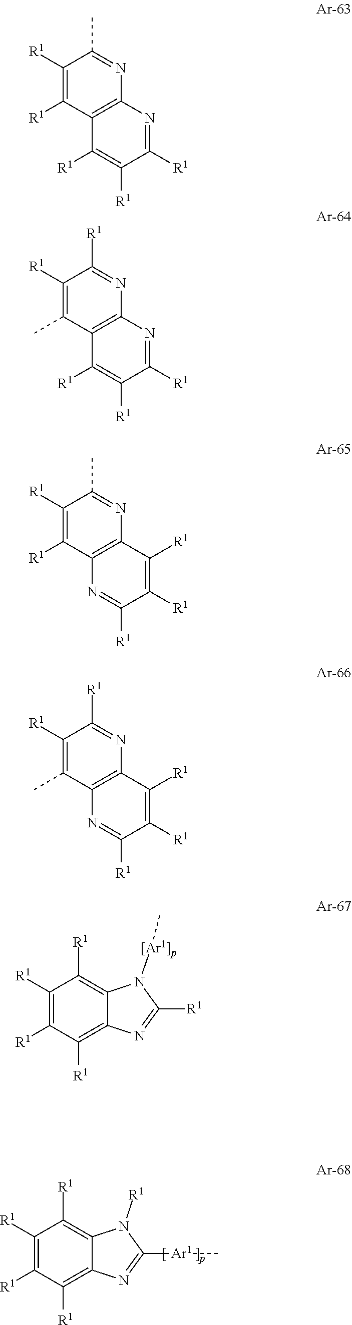

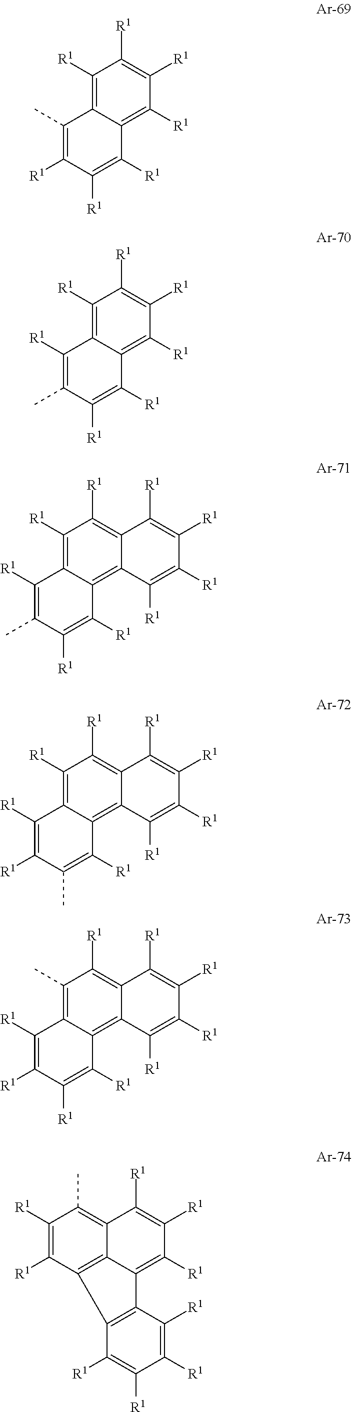

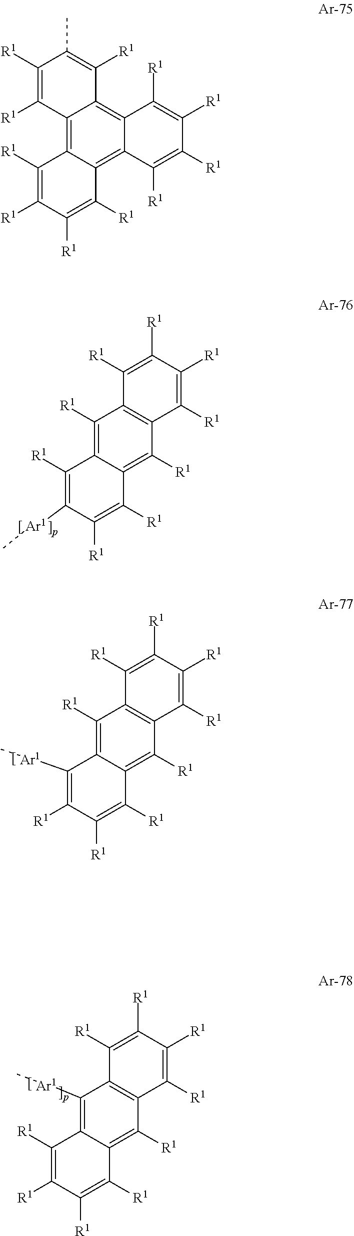

where R¹ is as defined above, the dotted bond represents the attachment site and, in addition:
Ar¹ is the same or different at each instance and is a bivalent aromatic or heteroaromatic ring system which has 6 to 18 aromatic ring atoms and may be substituted in each case by one or more R¹ radicals;
A is the same or different at each instance and is C(R¹)₂, NR¹, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R¹ radicals are bonded to the corresponding carbon atoms instead.

Preference is given here to structures of the formulae (Ar-1), (Ar-2), (Ar-3), (Ar-12), (Ar-13), (Ar-14), (Ar-15), (Ar-16), (Ar-40), (Ar-41), (Ar-42), (Ar-43), (Ar-45), (Ar-47), (Ar-57), (Ar-69), (Ar-70), (Ar-75), (Ar-78), and particular preference to structures of the formulae (Ar-1), (Ar-2), (Ar-3), (Ar-12), (Ar-13), (Ar-14), (Ar-15), (Ar-16).

When the abovementioned groups for structures of the formulae (Ar-1) to (Ar-78) have two or more A groups, possible options for these include all combinations from the definition of A. Preferred embodiments in that case are those in which one A group is NR¹ and the other A group is C(R¹)₂ or in which both A groups are NR¹ or in which both A groups are O.

When A is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R² radicals. Preference is given to phenyl, biphenyl, terphenyl and quaterphenyl having bonding patterns as listed above for Ar-1 to Ar-11, where these structures, rather than by R¹, may be substituted by one or more R² radicals, but are preferably unsubstituted. Preference is further given to triazine, pyrimidine and quinazoline as listed above for Ar-47 to Ar-50, Ar-57 and Ar-58, where these structures, rather than by R¹, may be substituted by one or more R² radicals.

There follows a description of preferred substituents R, R^{a}, R^{b}, R^{c}, R^{d} and R^{e}.

In a preferred embodiment of the invention, R, R^{a}, R^{b} are the same or different at each instance and are selected from the group consisting of H, D, F, CN, NO₂, Si(R¹)₃, B(OR¹)₂, a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R¹ radicals.

In a further-preferred embodiment of the invention, substituent R, R^{a}, R^{b} is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R¹ radicals.

It may further be the case that at least one substituent R, R^{a}, R^{b} is the same or different at each instance and is selected from the group consisting of H, D, an aromatic or heteroaromatic ring system which has 6 to 30 aromatic ring atoms and may be substituted by one or more R¹ radicals, or an N(Ar′)₂ group. In a further-preferred embodiment of the invention, the substituents R, R^{a}, R^{b}, R^{c}, R^{d} either form a ring according to the structures of the formulae (RA-1) to (RA-12), (RA-1a) to (RA-4f) or (RB), or R, R^{a}, R^{b} is the same or different at each instance and is selected from the group consisting of H, D, an aromatic or heteroaromatic ring system which has 6 to 30 aromatic ring atoms and may be substituted by one or more R¹ radicals, or an N(Ar′)₂ group. More preferably, substituent R, R^{a}, R^{b} is the same or different at each instance and is selected from the group consisting of H or an aromatic or heteroaromatic ring system having 6 to 24 aromatic ring atoms, preferably having 6 to 18 aromatic ring atoms, more preferably having 6 to 13 aromatic ring atoms, each of which may be substituted by one or more R¹ radicals.

It may preferably be the case that at least one substituent R, R^{a}, R^{b}, R^{c}, R^{d} is selected from phenyl, biphenyl, terphenyl, quaterphenyl, fluorene, spirobifluorene, naphthalene, indole, benzofuran, benzothiophene, carbazole, dibenzofuran, dibenzothiophene, indenocarbazole, indolocarbazole, pyridine, pyrimidine, pyrazine, pyridazine, triazine, quinoline, isoquinoline, quinazoline, quinoxaline, phenanthrene and triphenylene, each of which may be substituted by one or more R¹ radicals. What is meant more particularly here by the expression “substituent” is that R, R^{a}, R^{b}, R^{c}, R^{d} are not H. In addition, the substituents R, R^{a}, R^{b}, R^{c}, R^{d} may be the same or different if two or more substituents selected from the aromatic or heteroaromatic groups mentioned are present.

In a further embodiment, it may be the case that at least one substituent R, R^{a}, R^{b}, R^{c}, R^{d} is selected from o-biphenyl, o,o′-terphenyl, o,o′,p-quaterphenyl, 4,6-diphenylpyrimidin-2-yl, 4,6-diphenyltriazin-2-yl, naphthalene, phenanthrene, chrysene, spirobifluorene, triphenylene, anthracene, benzanthracene, fluorene and/or pyrene, each of which may be substituted by one or more R¹ radicals. Preference is given here to spirobifluorene, o-biphenyl, o,o′-terphenyl, o,o′,p-quaterphenyl, 4,6-diphenylpyrimidin-2-yl, 4,6-diphenyltriazin-2-yl radicals. The substituents R, R^{a}, R^{b}, R^{c}, R^{d} may be the same or different if two or more substituents selected from the aromatic groups mentioned are present. Structures/compounds having a group selected from o-biphenyl, o,o′-terphenyl, o,o′,p-quaterphenyl, 4,6-diphenylpyrimidin-2-yl, 4,6-diphenyltriazin-2-yl, naphthalene, phenanthrene, chrysene, spirobifluorene, triphenylene, anthracene, benzanthracene, fluorene and/or pyrene are especially suitable for use as electron transport material and/or as matrix material.

In respect of the substituents R^{c}, R^{d}, the same is correspondingly applicable as was stated above for the preferences set out in relation to the substituents R, R^{a}, R^{b} with the aforementioned restrictions. It may especially be the case that at least one substituent R^{c}, R^{d} is the same or different at each instance and is selected from the group consisting of an aromatic or heteroaromatic ring system which has 6 to 30 aromatic ring atoms and may be substituted by one or more R¹ radicals, and an N(Ar′)₂ group. It may additionally be the case that the substituents R^{c}, R^{d} either form a fused ring, preferably according to the structures of the formulae (RA-1) to (RA-12) or (RB), or the substituent R^{c}, R^{d} is the same or different at each instance and is selected from the group consisting of an aromatic or heteroaromatic ring system which has 6 to 30 aromatic ring atoms and may be substituted by one or more R¹ radicals, and an N(Ar′)₂ group.

It may more preferably be the case that the substituent R^{c}, R^{d} is the same or different at each instance and represents an aromatic or heteroaromatic ring system which has 6 to 30 aromatic ring atoms and may be substituted by one or more R¹ radicals selected from the groups of the formulae (Ar-1) to (Ar-78) shown above.

In a preferred embodiment of the invention, Re is the same or different at each instance and is selected from the group consisting of a straight-chain alkyl group having 1 to 20 carbon atoms or a branched or cyclic alkyl group having 3 to 20 carbon atoms, where the alkyl group may be substituted in each case by one or more R¹ radicals, or an aromatic or heteroaromatic ring system which has 5 to 60 aromatic ring atoms, preferably 5 to 40 aromatic ring atoms, and may be substituted in each case by one or more R² radicals.

In a further-preferred embodiment of the invention, Re is the same or different at each instance and are selected from the group consisting of a straight-chain alkyl group having 1 to 10 carbon atoms or a branched or cyclic alkyl group having 3 to 10 carbon atoms, where the alkyl group may be substituted in each case by one or more R² radicals, an aromatic or heteroaromatic ring system which has 6 to 30 aromatic ring atoms and may be substituted by one or more R² radicals. More preferably, Re is the same or different at each instance and are selected from the group consisting of a straight-chain alkyl group having 1 to 5 carbon atoms or a branched or cyclic alkyl group having 3 to 5 carbon atoms, where the alkyl group may be substituted in each case by one or more R² radicals, or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R² radicals.

In a preferred embodiment of the invention, Re is the same or different at each instance and is selected from the group consisting of a straight-chain alkyl group having 1 to 6 carbon atoms or a cyclic alkyl group having 3 to 6 carbon atoms, where the alkyl group may be substituted in each case by one or more R² radicals, or an aromatic or heteroaromatic ring system which has 6 to 24 aromatic ring atoms and may be substituted in each case by one or more R² radicals; at the same time, two Re radicals together may also form a ring system. More preferably, Re is the same or different at each instance and is selected from the group consisting of a straight-chain alkyl group having 1, 2, 3 or 4 carbon atoms or a branched or cyclic alkyl group having 3 to 6 carbon atoms, where the alkyl group may be substituted in each case by one or more R² radicals, but is preferably unsubstituted, or an aromatic ring system which has 6 to 12 aromatic ring atoms, especially 6 aromatic ring atoms, and may be substituted in each case by one or more preferably nonaromatic R² radicals, but is preferably unsubstituted; at the same time, two Re radicals together may form a ring system. Most preferably, Re is the same or different at each instance and is selected from the group consisting of a straight-chain alkyl group having 1, 2, 3 or 4 carbon atoms, or a branched alkyl group having 3 to 6 carbon atoms. Most preferably, Re is a methyl group or is a phenyl group, where two phenyl groups together may form a ring system, preference being given to a methyl group over a phenyl group.

Preferred aromatic or heteroaromatic ring systems represented by the substituent R, R^{a}, R^{b}, R^{c}, R^{d}, R^{e} or Ar or Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R, R¹ or R² radicals. The structures Ar-1 to Ar-78 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). With regard to the structures Ar-1 to Ar-78, it should be stated that these are shown with a substituent R¹. In the case of the ring systems Ar, these substituents R¹ should be replaced by R, and in the case of Re, these substituents R¹ should be replaced by R².

Further suitable R, R^{a}, R^{b}, R^{c}, R^{d} groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R¹ radicals. The total number of aromatic ring atoms in Ar², Ar³ and Ar⁴ here is not more than 60 and preferably not more than 40.

In this case, Ar⁴ and Ar² may also be bonded to one another and/or Ar² and Ar³ to one another by a group selected from C(R¹)₂, NR¹, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R¹ radicals. More preferably, Ar⁴ is selected from the group consisting of ortho-, meta- or para-phenylene or ortho-, meta- or para-biphenyl, each of which may be substituted by one or more R¹ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R¹ radicals. Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a further preferred embodiment of the invention, R¹ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R² radicals. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁵ radicals, but is preferably unsubstituted, or an aromatic or heteroaromatic ring system which has 6 to 13 aromatic ring atoms and may be substituted in each case by one or more R⁵ radicals, but is preferably unsubstituted.

In a further preferred embodiment of the invention, R² is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

It may further be the case that the compound includes exactly two or exactly three structures of formula (I), (II-1) to (II-42), (III-1) to (III-41), (IV-1) to (IV-41), (V-1) to (V-8), (VI-1) to (VI-22) and/or (VII-1) to (VII-4).

In a preferred configuration, the compounds are selected from compounds of the formula (D-1) or (D-2):

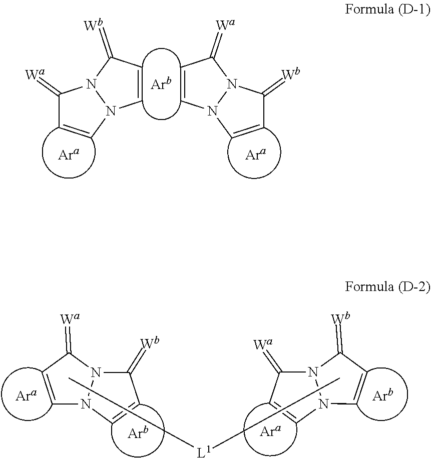

where the L¹ group is a connecting group, preferably a bond or an aromatic or heteroaromatic ring system which has 5 to 40, preferably 5 to 30, aromatic ring atoms and may be substituted by one or more R¹ radicals, and the further symbols used have the definitions given above, especially for formula (I).

In a further preferred embodiment of the invention, L¹ is a bond or an aromatic or heteroaromatic ring system which has 5 to 14 aromatic or heteroaromatic ring atoms, preferably an aromatic ring system which has 6 to 12 carbon atoms, and which may be substituted by one or more R¹ radicals, but is preferably unsubstituted, where R¹ may have the definition given above, especially for formula (I). More preferably, L¹ is an aromatic ring system having 6 to 10 aromatic ring atoms or a heteroaromatic ring system having 6 to 13 heteroaromatic ring atoms, each of which may be substituted by one or more R² radicals, but is preferably unsubstituted, where R² may have the definition given above, especially for formula (I).

Further preferably, the symbol L¹ shown in formula (D2) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L¹ group shown in formula (D2) comprises an aromatic ring system having not more than four, preferably not more than three, more preferably not more than two, fused aromatic and/or heteroaromatic 6-membered rings, and preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L¹ are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R¹ radicals, but are preferably unsubstituted.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds shown in the following table:

| | |
|---|---|
| 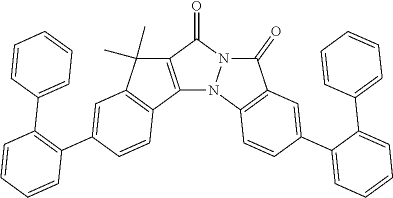 [SMILES: CC1(C)c2cc(-c3ccccc3-c3ccccc3)ccc2-c2c1c(=O)n1c(=O)c3cc(-c4ccccc4-c4ccccc4)ccc3n21] | 1 |
| 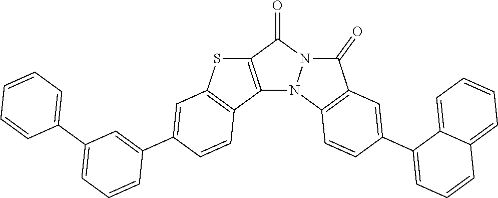 [SMILES: O=c1c2cc(-c3cccc4ccccc34)ccc2n2c3c(sc4cc(-c5cccc(-c6ccccc6)c5)ccc43)c(=O)n12] | 2 |
| 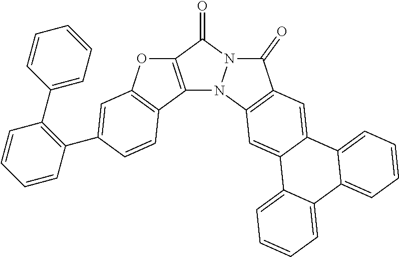 [SMILES: O=c1c2cc3c4ccccc4c4ccccc4c3cc2n2c3c(oc4cc(-c5ccccc5-c5ccccc5)ccc43)c(=O)n12] | 3 |
| 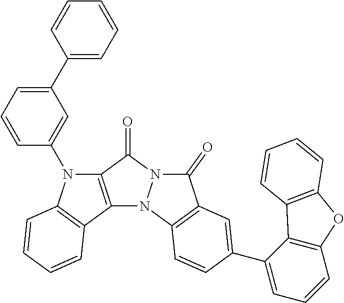 [SMILES: O=c1c2cc(-c3cccc4oc5ccccc5c34)ccc2n2c3c4ccccc4n(-c4cccc(-c5ccccc5)c4)c3c(=O)n12] | 4 |
| 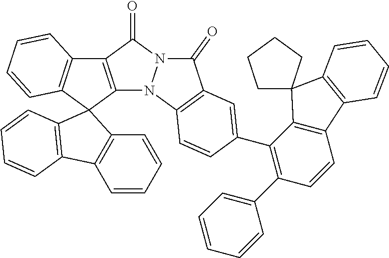 [SMILES: O=c1c2c(n3c4ccc(-c5c(-c6ccccc6)ccc6c5C5(CCCC5)c5ccccc5-6)cc4c(=O)n13)C1(c3ccccc3-c3ccccc31)c1ccccc1-2] | 5 |
| 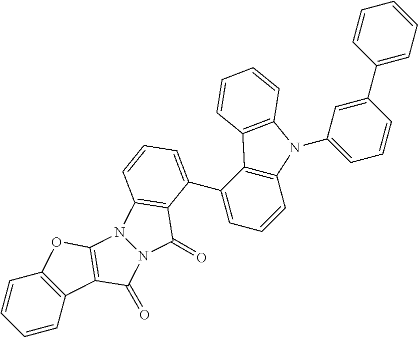 [SMILES: O=c1c2c(-c3cccc4c3c3ccccc3n4-c3cccc(-c4ccccc4)c3)cccc2n2c3oc4ccccc4c3c(=O)n12] | 6 |
| 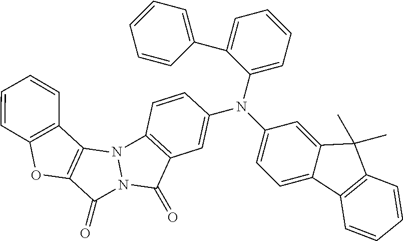 [SMILES: CC1(C)c2ccccc2-c2ccc(N(c3ccc4c(c3)c(=O)n3c(=O)c5oc6ccccc6c5n43)c3ccccc3-c3ccccc3)cc21] | 7 |
| 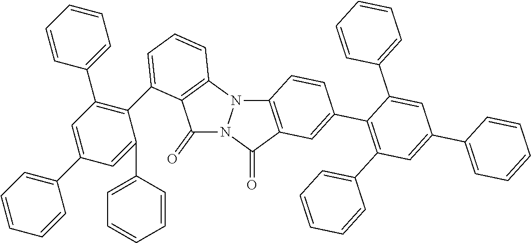 [SMILES: O=c1c2cc(-c3c(-c4ccccc4)cc(-c4ccccc4)cc3-c3ccccc3)ccc2n2c3cccc(-c4c(-c5ccccc5)cc(-c5ccccc5)cc4-c4ccccc4)c3c(=O)n12] | 8 |
| 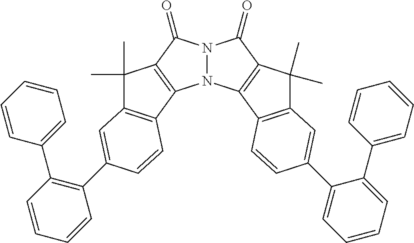 [SMILES: CC1(C)c2cc(-c3ccccc3-c3ccccc3)ccc2-c2c1c(=O)n1c(=O)c3c(n21)-c1ccc(-c2ccccc2-c2ccccc2)cc1C3(C)C] | 9 |
| 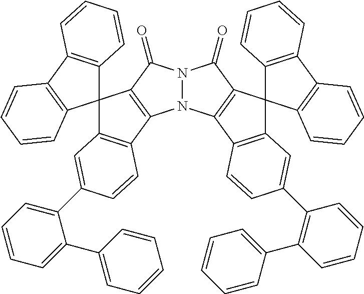 [SMILES: O=c1c2c(n3c4c(c(=O)n13)C1(c3ccccc3-c3ccccc31)c1cc(-c3ccccc3-c3ccccc3)ccc1-4)-c1ccc(-c3ccccc3-c3ccccc3)cc1C21c2ccccc2-c2ccccc21] | 10 |
| 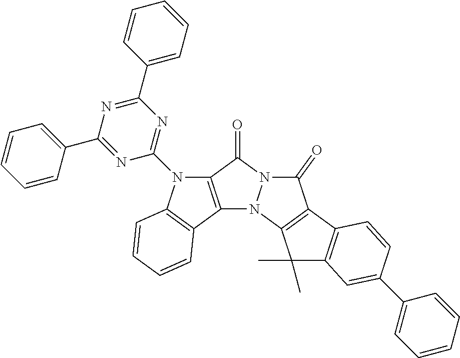 [SMILES: CC1(C)c2cc(-c3ccccc3)ccc2-c2c1n1c3c4ccccc4n(-c4nc(-c5ccccc5)nc(-c5ccccc5)n4)c3c(=O)n1c2=O] | 11 |
| 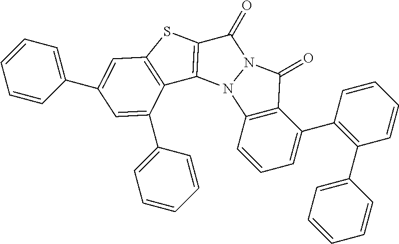 [SMILES: O=c1c2sc3cc(-c4ccccc4)cc(-c4ccccc4)c3c2n2c3cccc(-c4ccccc4-c4ccccc4)c3c(=O)n12] | 12 |
| 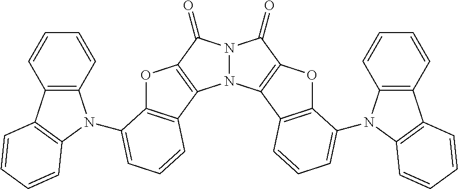 [SMILES: O=c1c2oc3c(-n4c5ccccc5c5ccccc54)cccc3c2n2c3c(oc4c(-n5c6ccccc6c6ccccc65)cccc43)c(=O)n12] | 13 |
| 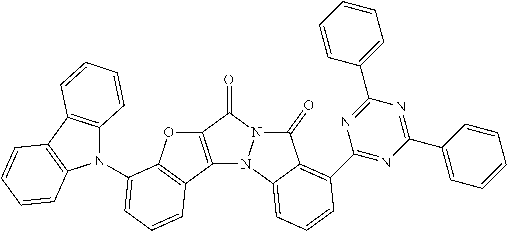 [SMILES: O=c1c2oc3c(-n4c5ccccc5c5ccccc54)cccc3c2n2c3cccc(-c4nc(-c5ccccc5)nc(-c5ccccc5)n4)c3c(=O)n12] | 14 |
| 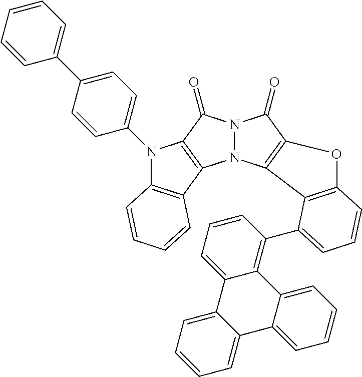 [SMILES: O=c1c2oc3cccc(-c4cccc5c6ccccc6c6ccccc6c45)c3c2n2c3c4ccccc4n(-c4ccc(-c5ccccc5)cc4)c3c(=O)n12] | 15 |
| 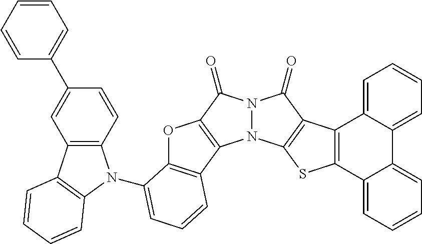 [SMILES: O=c1c2oc3c(-n4c5ccccc5c5cc(-c6ccccc6)ccc54)cccc3c2n2c3sc4c5ccccc5c5ccccc5c4c3c(=O)n12] | 16 |
| 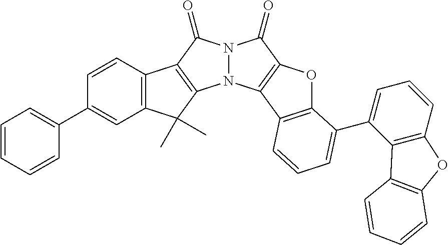 [SMILES: CC1(C)c2cc(-c3ccccc3)ccc2-c2c1n1c3c(oc4c(-c5cccc6oc7ccccc7c56)cccc43)c(=O)n1c2=O] | 17 |
| 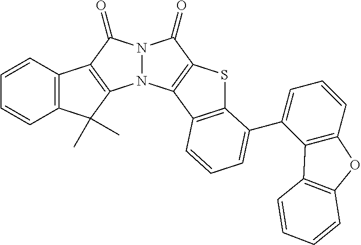 [SMILES: CC1(C)c2ccccc2-c2c1n1c3c(sc4c(-c5cccc6oc7ccccc7c56)cccc43)c(=O)n1c2=O] | 18 |
| 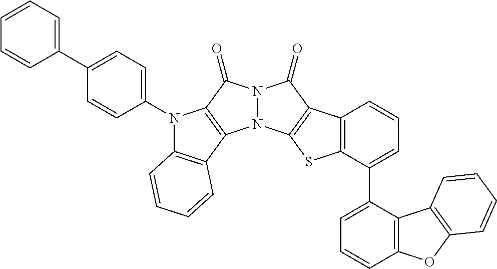 [SMILES: O=c1c2c3cccc(-c4cccc5oc6ccccc6c45)c3sc2n2c3c4ccccc4n(-c4ccc(-c5ccccc5)cc4)c3c(=O)n12] | 19 |
| 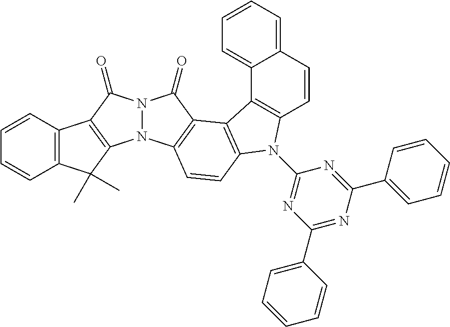 [SMILES: CC1(C)c2ccccc2-c2c1n1c3ccc4c(c3c(=O)n1c2=O)c1c2ccccc2ccc1n4-c1nc(-c2ccccc2)nc(-c2ccccc2)n1] | 20 |
| 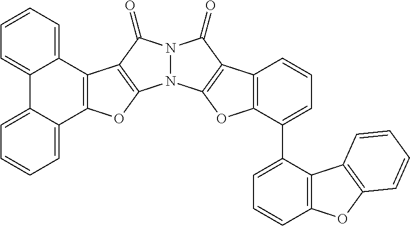 [SMILES: O=c1c2c3cccc(-c4cccc5oc6ccccc6c45)c3oc2n2c3oc4c5ccccc5c5ccccc5c4c3c(=O)n12] | 21 |
| 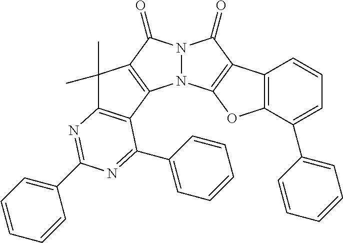 [SMILES: CC1(C)c2nc(-c3ccccc3)nc(-c3ccccc3)c2-c2c1c(=O)n1c(=O)c3c4cccc(-c5ccccc5)c4oc3n21] | 22 |
| 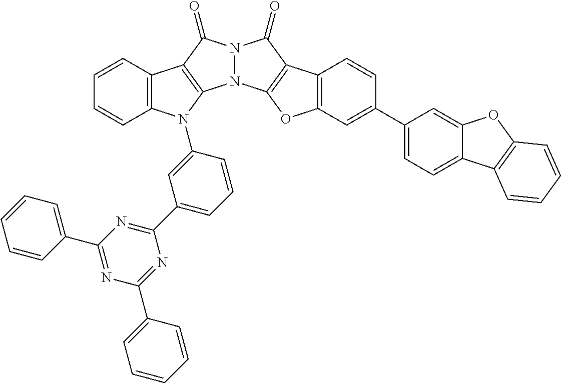 [SMILES: O=c1c2c3ccc(-c4ccc5c(c4)oc4ccccc45)cc3oc2n2c3c(c(=O)n12)c1ccccc1n3-c1cccc(-c2nc(-c3ccccc3)nc(-c3ccccc3)n2)c1] | 23 |
| 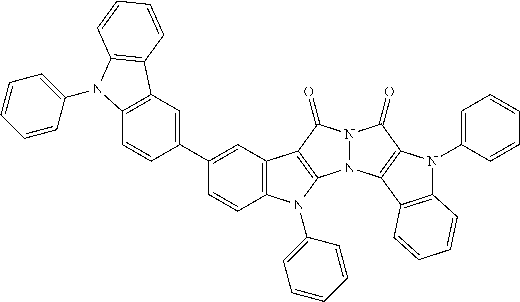 [SMILES: O=c1c2c3cc(-c4ccc5c(c4)c4ccccc4n5-c4ccccc4)ccc3n(-c3ccccc3)c2n2c3c4ccccc4n(-c4ccccc4)c3c(=O)n12] | 24 |
| 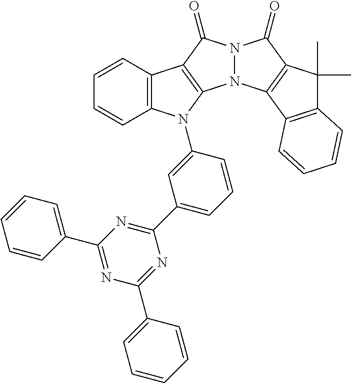 [SMILES: CC1(C)c2ccccc2-c2c1c(=O)n1c(=O)c3c4ccccc4n(-c4cccc(-c5nc(-c6ccccc6)nc(-c6ccccc6)n5)c4)c3n21] | 25 |
| 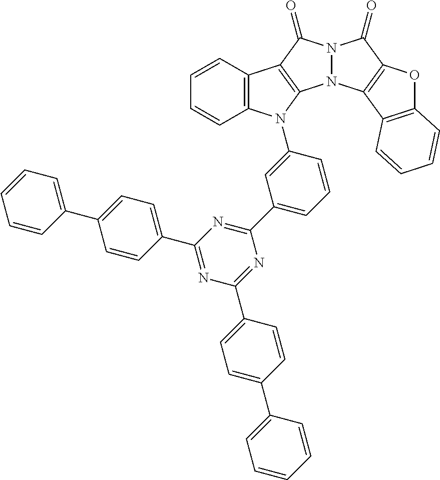 [SMILES: O=c1c2oc3ccccc3c2n2c3c(c(=O)n12)c1ccccc1n3-c1cccc(-c2nc(-c3ccc(-c4ccccc4)cc3)nc(-c3ccc(-c4ccccc4)cc3)n2)c1] | 26 |
| 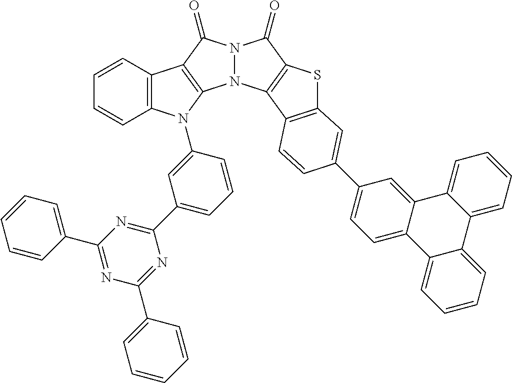 [SMILES: O=c1c2sc3cc(-c4ccc5c6ccccc6c6ccccc6c5c4)ccc3c2n2c3c(c(=O)n12)c1ccccc1n3-c1cccc(-c2nc(-c3ccccc3)nc(-c3ccccc3)n2)c1] | 27 |
| 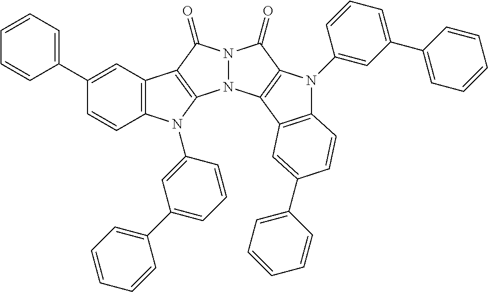 [SMILES: O=c1c2c3cc(-c4ccccc4)ccc3n(-c3cccc(-c4ccccc4)c3)c2n2c3c4cc(-c5ccccc5)ccc4n(-c4cccc(-c5ccccc5)c4)c3c(=O)n12] | 28 |
| 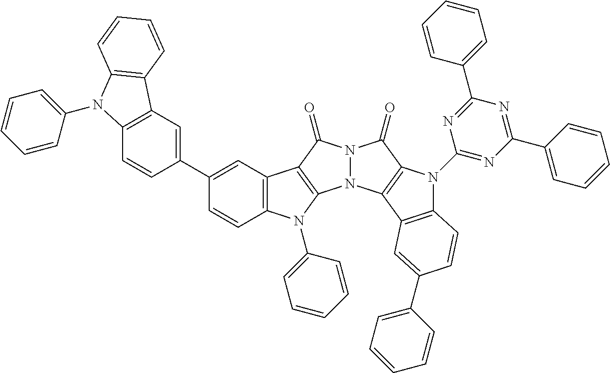 [SMILES: O=c1c2c3cc(-c4ccc5c(c4)c4ccccc4n5-c4ccccc4)ccc3n(-c3ccccc3)c2n2c3c4cc(-c5ccccc5)ccc4n(-c4nc(-c5ccccc5)nc(-c5ccccc5)n4)c3c(=O)n12] | 29 |
| 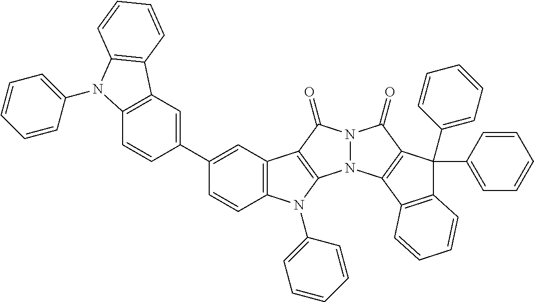 [SMILES: O=c1c2c(n3c4c(c(=O)n13)c1cc(-c3ccc5c(c3)c3ccccc3n5-c3ccccc3)ccc1n4-c1ccccc1)-c1ccccc1C2(c1ccccc1)c1ccccc1] | 30 |
| 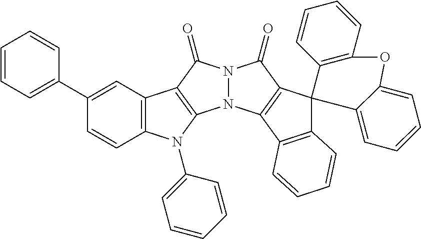 [SMILES: O=c1c2c(n3c4c(c(=O)n13)c1cc(-c3ccccc3)ccc1n4-c1ccccc1)-c1ccccc1C21c2ccccc2Oc2ccccc21] | 31 |
| 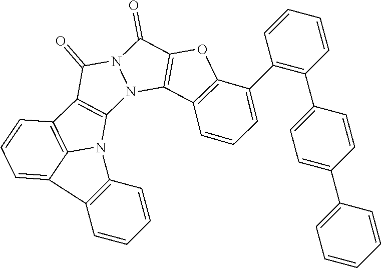 [SMILES: O=c1c2oc3c(-c4ccccc4-c4ccc(-c5ccccc5)cc4)cccc3c2n2c3c(c(=O)n12)c1cccc2c4ccccc4n3c21] | 32 |
| 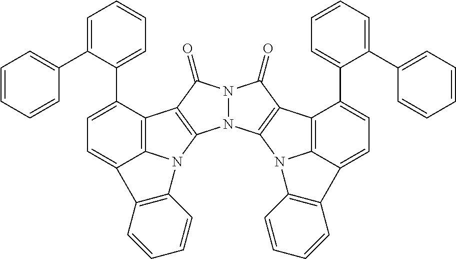 [SMILES: O=c1c2c3c(-c4ccccc4-c4ccccc4)ccc4c5ccccc5n(c43)c2n2c3c(c(=O)n12)c1c(-c2ccccc2-c2ccccc2)ccc2c4ccccc4n3c21] | 33 |
| 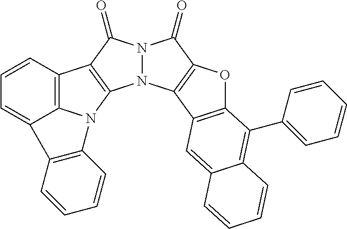 [SMILES: O=c1c2oc3c(-c4ccccc4)c4ccccc4cc3c2n2c3c(c(=O)n12)c1cccc2c4ccccc4n3c21] | 34 |
| 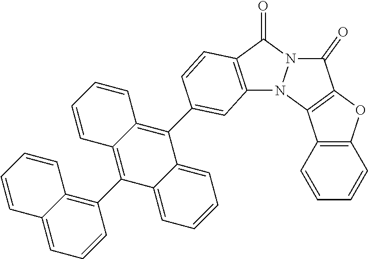 [SMILES: O=c1c2ccc(-c3c4ccccc4c(-c4cccc5ccccc45)c4ccccc34)cc2n2c3c(oc4ccccc43)c(=O)n12] | 35 |
| 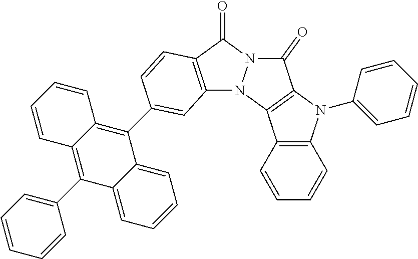 [SMILES: O=c1c2ccc(-c3c4ccccc4c(-c4ccccc4)c4ccccc34)cc2n2c3c4ccccc4n(-c4ccccc4)c3c(=O)n12] | 36 |
| 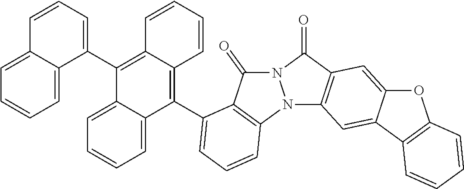 [SMILES: O=c1c2cc3oc4ccccc4c3cc2n2c3cccc(-c4c5ccccc5c(-c5cccc6ccccc56)c5ccccc45)c3c(=O)n12] | 37 |
| 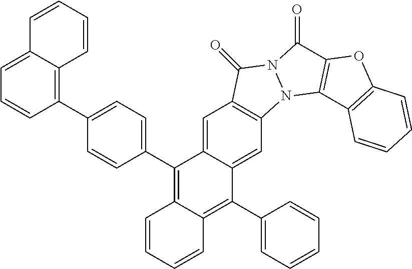 [SMILES: O=c1c2cc3c(-c4ccc(-c5cccc6ccccc56)cc4)c4ccccc4c(-c4ccccc4)c3cc2n2c3c(oc4ccccc43)c(=O)n12] | 38 |
| 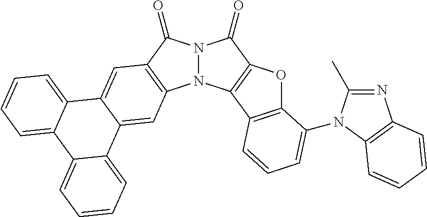 [SMILES: Cc1nc2ccccc2n1-c1cccc2c1oc1c(=O)n3c(=O)c4cc5c6ccccc6c6ccccc6c5cc4n3c12] | 39 |
| 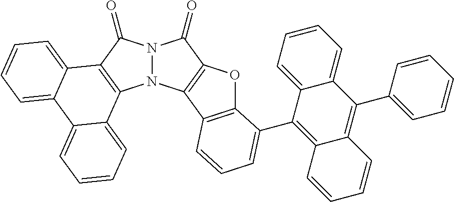 [SMILES: O=c1c2oc3c(-c4c5ccccc5c(-c5ccccc5)c5ccccc45)cccc3c2n2c3c4ccccc4c4ccccc4c3c(=O)n12] | 40 |
| 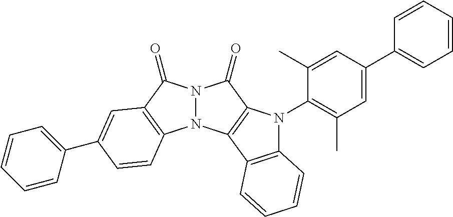 [SMILES: Cc1cc(-c2ccccc2)cc(C)c1-n1c2ccccc2c2c1c(=O)n1c(=O)c3cc(-c4ccccc4)ccc3n21] | 41 |
| 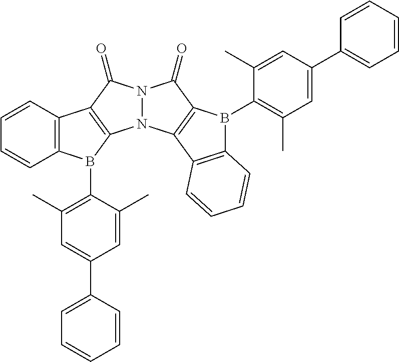 [SMILES: Cc1cc(-c2ccccc2)cc(C)c1B1c2ccccc2-c2c1c(=O)n1c(=O)c3c(n21)B(c1c(C)cc(-c2ccccc2)cc1C)c1ccccc1-3] | 42 |
| 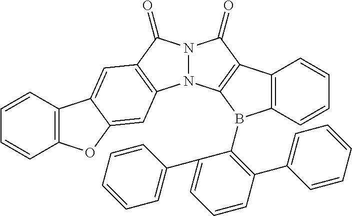 [SMILES: O=c1c2c(n3c4cc5oc6ccccc6c5cc4c(=O)n13)B(c1c(-c3ccccc3)cccc1-c1ccccc1)c1ccccc1-2] | 43 |
| 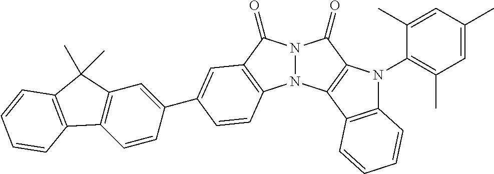 [SMILES: Cc1cc(C)c(-n2c3ccccc3c3c2c(=O)n2c(=O)c4cc(-c5ccc6c(c5)C(C)(C)c5ccccc5-6)ccc4n32)c(C)c1] | 44 |
| 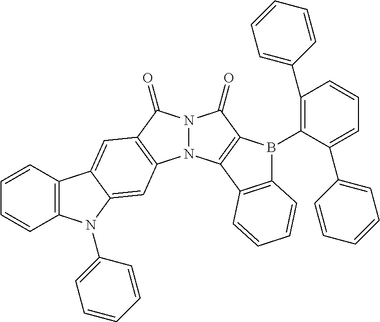 [SMILES: O=c1c2c(n3c4cc5c(cc4c(=O)n13)c1ccccc1n5-c1ccccc1)-c1ccccc1B2c1c(-c2ccccc2)cccc1-c1ccccc1] | 45 |
| 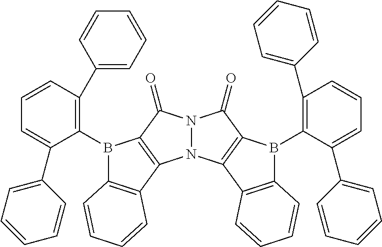 [SMILES: O=c1c2c(n3c4c(c(=O)n13)B(c1c(-c3ccccc3)cccc1-c1ccccc1)c1ccccc1-4)-c1ccccc1B2c1c(-c2ccccc2)cccc1-c1ccccc1] | 46 |
| 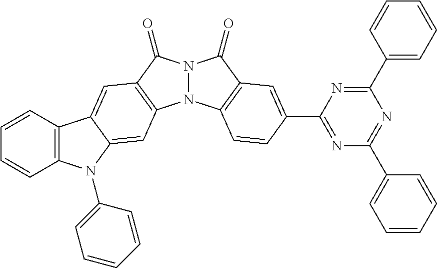 [SMILES: O=c1c2cc(-c3nc(-c4ccccc4)nc(-c4ccccc4)n3)ccc2n2c3cc4c(cc3c(=O)n12)c1ccccc1n4-c1ccccc1] | 47 |
| 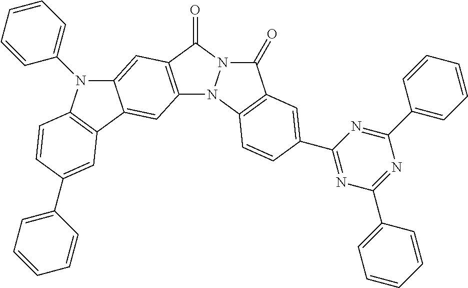 [SMILES: O=c1c2cc(-c3nc(-c4ccccc4)nc(-c4ccccc4)n3)ccc2n2c3cc4c5cc(-c6ccccc6)ccc5n(-c5ccccc5)c4cc3c(=O)n12] | 48 |
| 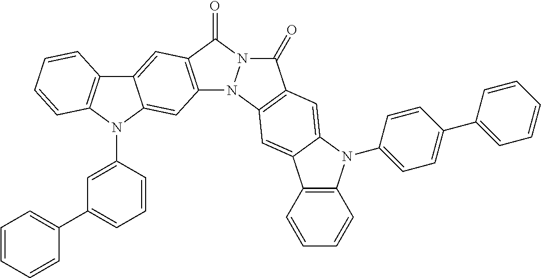 [SMILES: O=c1c2cc3c(cc2n2c4cc5c(cc4c(=O)n12)c1ccccc1n5-c1cccc(-c2ccccc2)c1)c1ccccc1n3-c1ccc(-c2ccccc2)cc1] | 49 |
| 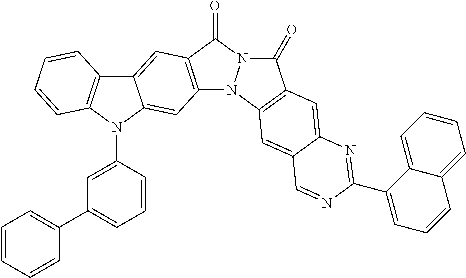 [SMILES: O=c1c2cc3nc(-c4cccc5ccccc45)ncc3cc2n2c3cc4c(cc3c(=O)n12)c1ccccc1n4-c1cccc(-c2ccccc2)c1] | 50 |
| 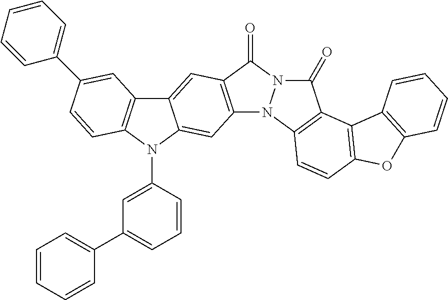 [SMILES: O=c1c2cc3c4cc(-c5ccccc5)ccc4n(-c4cccc(-c5ccccc5)c4)c3cc2n2c3ccc4oc5ccccc5c4c3c(=O)n12] | 51 |
| 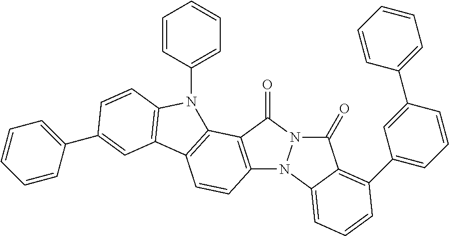 [SMILES: O=c1c2c(-c3cccc(-c4ccccc4)c3)cccc2n2c3ccc4c5cc(-c6ccccc6)ccc5n(-c5ccccc5)c4c3c(=O)n12] | 52 |
| 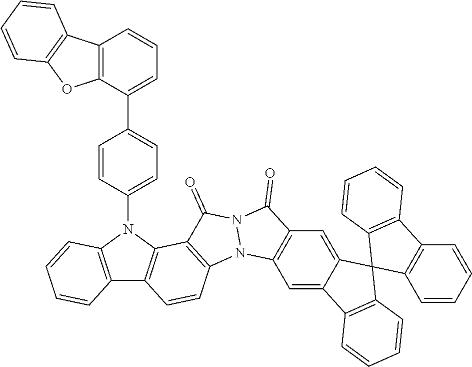 [SMILES: O=c1c2cc3c(cc2n2c4ccc5c6ccccc6n(-c6ccc(-c7cccc8c7oc7ccccc78)cc6)c5c4c(=O)n12)-c1ccccc1C31c2ccccc2-c2ccccc21] | 53 |
| 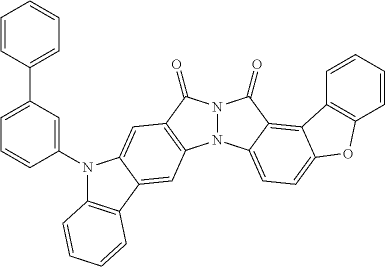 [SMILES: O=c1c2cc3c(cc2n2c4ccc5oc6ccccc6c5c4c(=O)n12)c1ccccc1n3-c1cccc(-c2ccccc2)c1] | 54 |
| 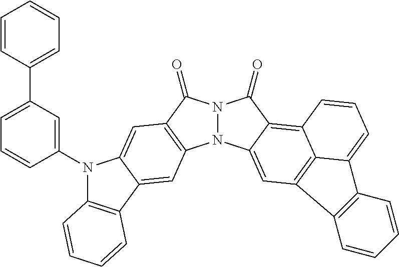 [SMILES: O=c1c2cc3c(cc2n2c4cc5c6c(cccc6c4c(=O)n12)-c1ccccc1-5)c1ccccc1n3-c1cccc(-c2ccccc2)c1] | 55 |
| 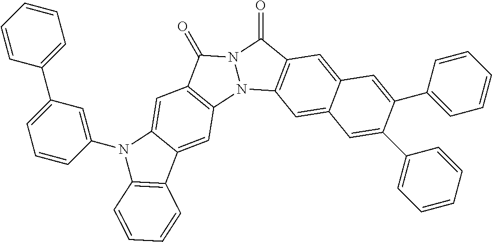 [SMILES: O=c1c2cc3cc(-c4ccccc4)c(-c4ccccc4)cc3cc2n2c3cc4c5ccccc5n(-c5cccc(-c6ccccc6)c5)c4cc3c(=O)n12] | 56 |
| 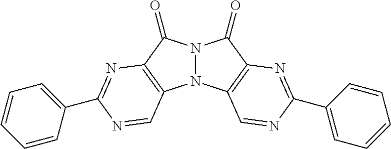 [SMILES: O=c1c2nc(-c3ccccc3)ncc2n2c3cnc(-c4ccccc4)nc3c(=O)n12] | 57 |
| 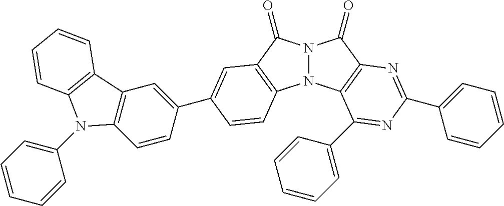 [SMILES: O=c1c2cc(-c3ccc4c(c3)c3ccccc3n4-c3ccccc3)ccc2n2c3c(-c4ccccc4)nc(-c4ccccc4)nc3c(=O)n12] | 58 |
| 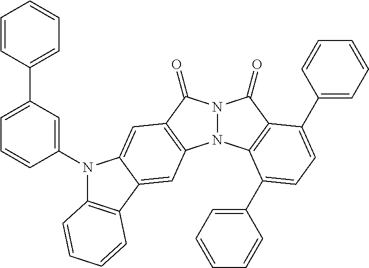 [SMILES: O=c1c2cc3c(cc2n2c4c(-c5ccccc5)ccc(-c5ccccc5)c4c(=O)n12)c1ccccc1n3-c1cccc(-c2ccccc2)c1] | 59 |
| 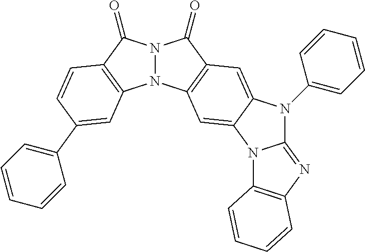 [SMILES: O=c1c2ccc(-c3ccccc3)cc2n2c3cc4c(cc3c(=O)n12)n(-c1ccccc1)c1nc2ccccc2n41] | 60 |
| 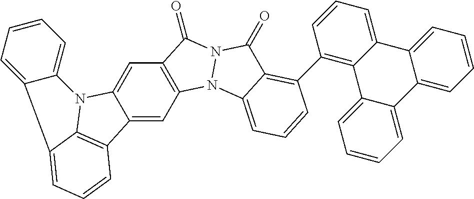 [SMILES: O=c1c2cc3c(cc2n2c4cccc(-c5cccc6c7ccccc7c7ccccc7c56)c4c(=O)n12)c1cccc2c4ccccc4n3c21] | 61 |
| 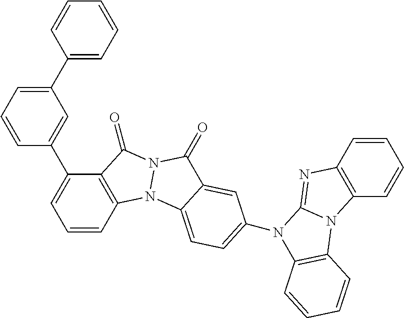 [SMILES: O=c1c2cc(-n3c4ccccc4n4c5ccccc5nc34)ccc2n2c3cccc(-c4cccc(-c5ccccc5)c4)c3c(=O)n12] | 62 |
| 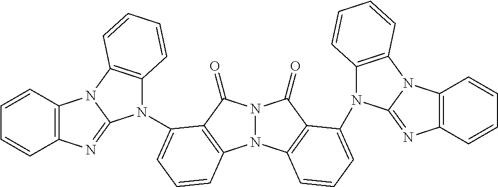 [SMILES: O=c1c2c(-n3c4ccccc4n4c5ccccc5nc34)cccc2n2c3cccc(-n4c5ccccc5n5c6ccccc6nc45)c3c(=O)n12] | 63 |
| 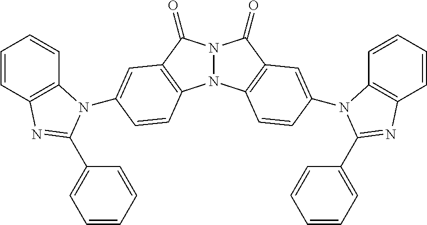 [SMILES: O=c1c2cc(-n3c(-c4ccccc4)nc4ccccc43)ccc2n2c3ccc(-n4c(-c5ccccc5)nc5ccccc54)cc3c(=O)n12] | 64 |
| 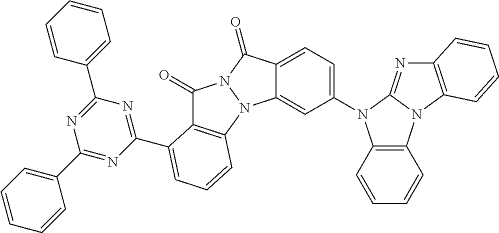 [SMILES: O=c1c2ccc(-n3c4ccccc4n4c5ccccc5nc34)cc2n2c3cccc(-c4nc(-c5ccccc5)nc(-c5ccccc5)n4)c3c(=O)n12] | 65 |
| 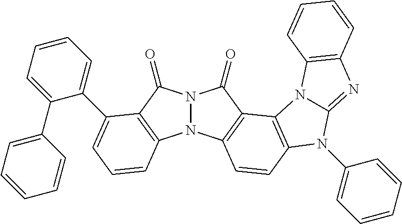 [SMILES: O=c1c2c(-c3ccccc3-c3ccccc3)cccc2n2c3ccc4c(c3c(=O)n12)n1c2ccccc2nc1n4-c1ccccc1] | 66 |
| 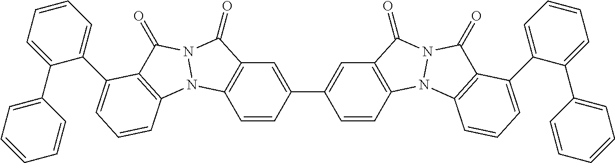 [SMILES: O=c1c2cc(-c3ccc4c(c3)c(=O)n3c(=O)c5c(-c6ccccc6-c6ccccc6)cccc5n43)ccc2n2c3cccc(-c4ccccc4-c4ccccc4)c3c(=O)n12] | 67 |
| 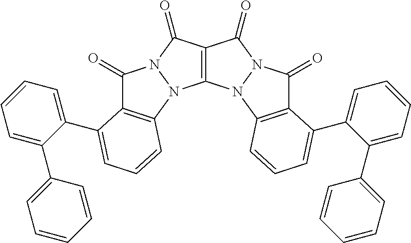 [SMILES: O=c1c2c(-c3ccccc3-c3ccccc3)cccc2n2c3c(c(=O)n12)c(=O)n1c(=O)c2c(-c4ccccc4-c4ccccc4)cccc2n31] | 68 |
| 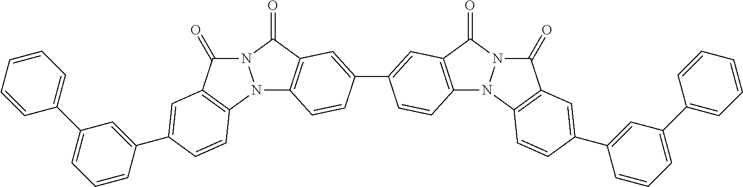 [SMILES: O=c1c2cc(-c3cccc(-c4ccccc4)c3)ccc2n2c3ccc(-c4ccc5c(c4)c(=O)n4c(=O)c6cc(-c7cccc(-c8ccccc8)c7)ccc6n54)cc3c(=O)n12] | 69 |
| 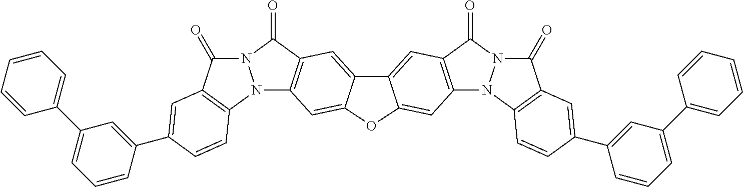 [SMILES: O=c1c2cc(-c3cccc(-c4ccccc4)c3)ccc2n2c3cc4oc5cc6c(cc5c4cc3c(=O)n12)c(=O)n1c(=O)c2cc(-c3cccc(-c4ccccc4)c3)ccc2n61] | 70 |
| 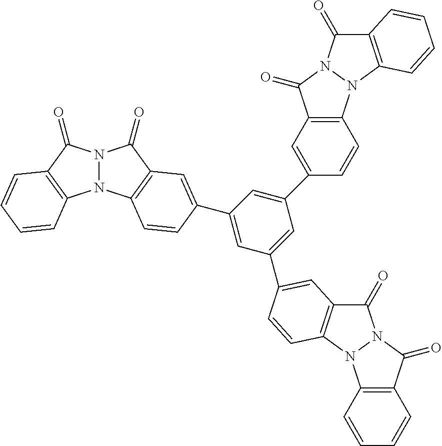 [SMILES: O=c1c2ccccc2n2c3ccc(-c4cc(-c5ccc6c(c5)c(=O)n5c(=O)c7ccccc7n65)cc(-c5ccc6c(c5)c(=O)n5c(=O)c7ccccc7n65)c4)cc3c(=O)n12] | 71 |
| 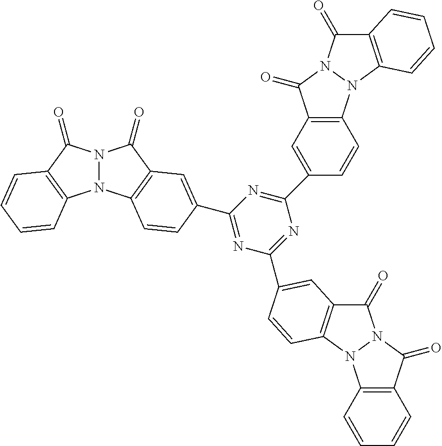 [SMILES: O=c1c2ccccc2n2c3ccc(-c4nc(-c5ccc6c(c5)c(=O)n5c(=O)c7ccccc7n65)nc(-c5ccc6c(c5)c(=O)n5c(=O)c7ccccc7n65)n4)cc3c(=O)n12] | 72 |
| 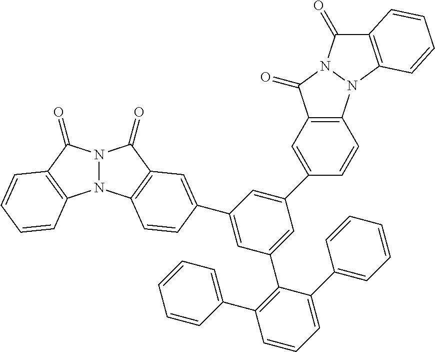 [SMILES: O=c1c2ccccc2n2c3ccc(-c4cc(-c5ccc6c(c5)c(=O)n5c(=O)c7ccccc7n65)cc(-c5c(-c6ccccc6)cccc5-c5ccccc5)c4)cc3c(=O)n12] | 73 |
| 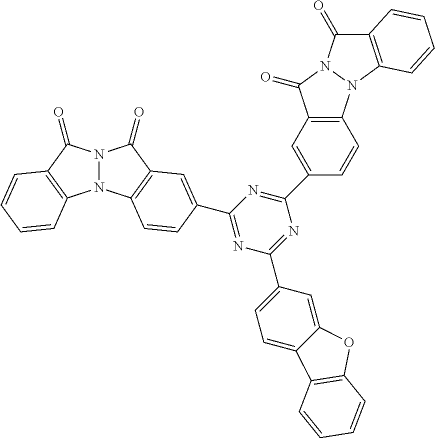 [SMILES: O=c1c2ccccc2n2c3ccc(-c4nc(-c5ccc6c(c5)oc5ccccc56)nc(-c5ccc6c(c5)c(=O)n5c(=O)c7ccccc7n65)n4)cc3c(=O)n12] | 74 |
| 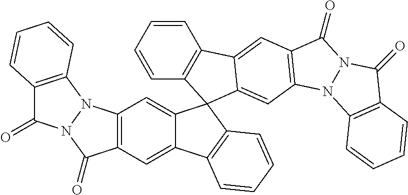 [SMILES: O=c1c2ccccc2n2c3cc4c(cc3c(=O)n12)-c1ccccc1C41c2ccccc2-c2cc3c(=O)n4c(=O)c5ccccc5n4c3cc21] | 75 |
| 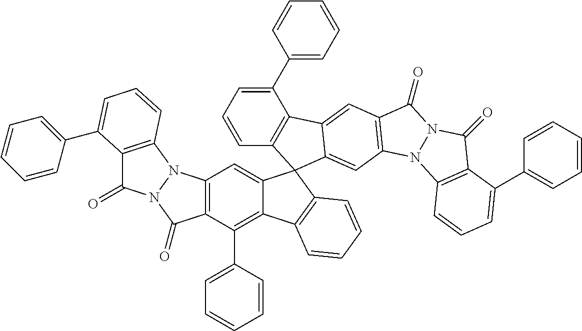 [SMILES: O=c1c2cc3c(cc2n2c4cccc(-c5ccccc5)c4c(=O)n12)C1(c2ccccc2-c2c1cc1c(c2-c2ccccc2)c(=O)n2c(=O)c4c(-c5ccccc5)cccc4n12)c1cccc(-c2ccccc2)c1-3] | 76 |
| 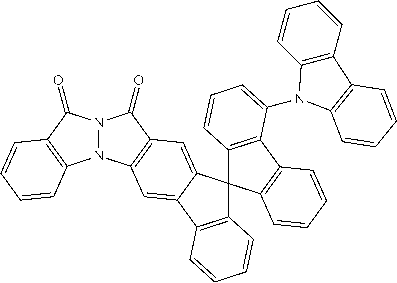 [SMILES: O=c1c2ccccc2n2c3cc4c(cc3c(=O)n12)C1(c2ccccc2-4)c2ccccc2-c2c(-n3c4ccccc4c4ccccc43)cccc21] | 77 |
| 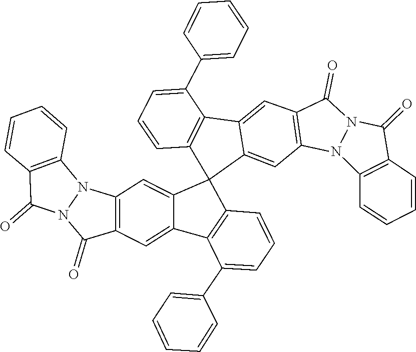 [SMILES: O=c1c2ccccc2n2c3cc4c(cc3c(=O)n12)-c1c(-c2ccccc2)cccc1C41c2cc3c(cc2-c2c(-c4ccccc4)cccc21)c(=O)n1c(=O)c2ccccc2n31] | 78 |
| 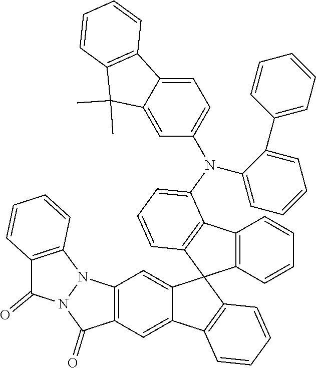 [SMILES: CC1(C)c2ccccc2-c2ccc(N(c3ccccc3-c3ccccc3)c3cccc4c3-c3ccccc3C43c4ccccc4-c4cc5c(=O)n6c(=O)c7ccccc7n6c5cc43)cc21] | 79 |
| 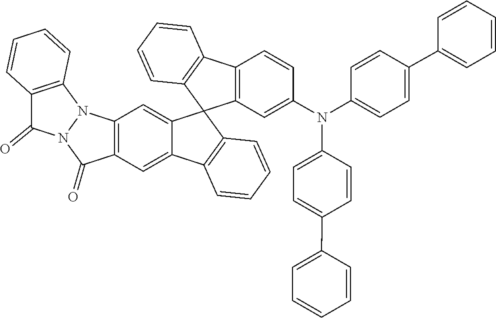 [SMILES: O=c1c2ccccc2n2c3cc4c(cc3c(=O)n12)-c1ccccc1C41c2ccccc2-c2ccc(N(c3ccc(-c4ccccc4)cc3)c3ccc(-c4ccccc4)cc3)cc21] | 80 |
| 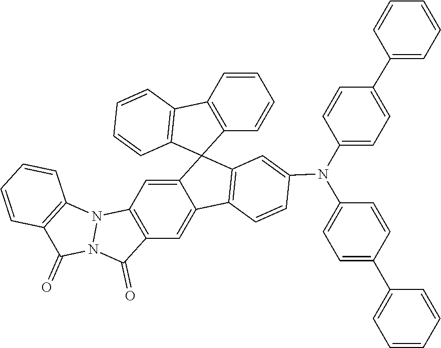 [SMILES: O=c1c2ccccc2n2c3cc4c(cc3c(=O)n12)-c1ccc(N(c2ccc(-c3ccccc3)cc2)c2ccc(-c3ccccc3)cc2)cc1C41c2ccccc2-c2ccccc21] | 81 |
| 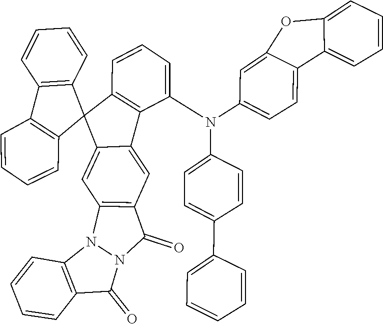 [SMILES: O=c1c2ccccc2n2c3cc4c(cc3c(=O)n12)-c1c(N(c2ccc(-c3ccccc3)cc2)c2ccc3c(c2)oc2ccccc23)cccc1C41c2ccccc2-c2ccccc21] | 82 |
| 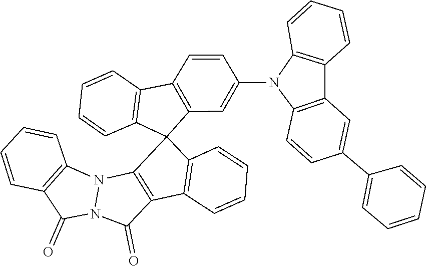 [SMILES: O=c1c2c(n3c4ccccc4c(=O)n13)C1(c3ccccc3-c3ccc(-n4c5ccccc5c5cc(-c6ccccc6)ccc54)cc31)c1ccccc1-2] | 83 |
| 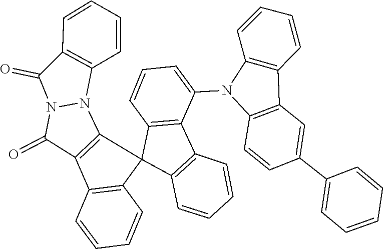 [SMILES: O=c1c2c(n3c4ccccc4c(=O)n13)C1(c3ccccc3-c3c(-n4c5ccccc5c5cc(-c6ccccc6)ccc54)cccc31)c1ccccc1-2] | 84 |
| 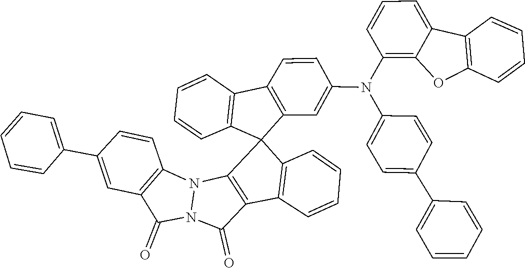 [SMILES: O=c1c2c(n3c4ccc(-c5ccccc5)cc4c(=O)n13)C1(c3ccccc3-c3ccc(N(c4ccc(-c5ccccc5)cc4)c4cccc5c4oc4ccccc45)cc31)c1ccccc1-2] | 85 |
| 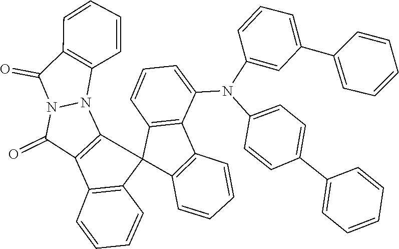 [SMILES: O=c1c2c(n3c4ccccc4c(=O)n13)C1(c3ccccc3-c3c(N(c4ccc(-c5ccccc5)cc4)c4cccc(-c5ccccc5)c4)cccc31)c1ccccc1-2] | 86 |
| 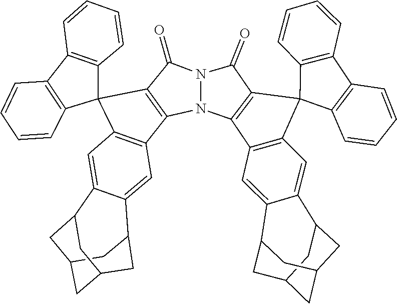 [SMILES: O=c1c2c(n3c4c(c(=O)n13)C1(c3ccccc3-c3ccccc31)c1cc3c(cc1-4)C1CC4CC(C1)CC3C4)-c1cc3c(cc1C21c2ccccc2-c2ccccc21)C1CC2CC(CC3C2)C1] | 87 |
| 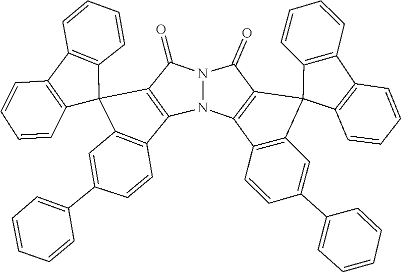 [SMILES: O=c1c2c(n3c4c(c(=O)n13)C1(c3ccccc3-c3ccccc31)c1cc(-c3ccccc3)ccc1-4)-c1ccc(-c3ccccc3)cc1C21c2ccccc2-c2ccccc21] | 88 |
| 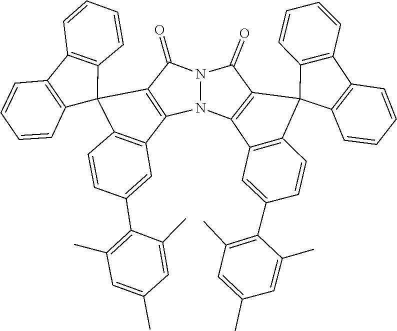 [SMILES: Cc1cc(C)c(-c2ccc3c(c2)-c2c(c(=O)n4c(=O)c5c(n24)-c2cc(-c4c(C)cc(C)cc4C)ccc2C52c4ccccc4-c4ccccc42)C32c3ccccc3-c3ccccc32)c(C)c1] | 89 |
| 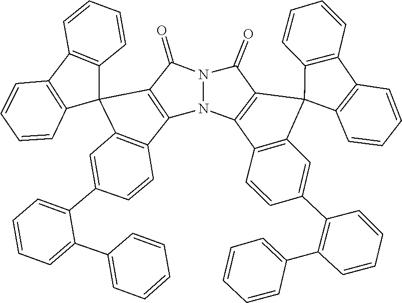 [SMILES: O=c1c2c(n3c4c(c(=O)n13)C1(c3ccccc3-c3ccccc31)c1cc(-c3ccccc3-c3ccccc3)ccc1-4)-c1ccc(-c3ccccc3-c3ccccc3)cc1C21c2ccccc2-c2ccccc21] | 90 |
| 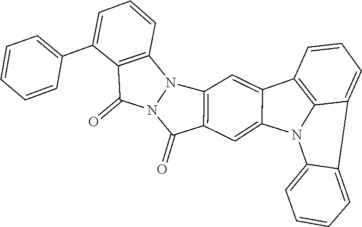 [SMILES: O=c1c2cc3c(cc2n2c4cccc(-c5ccccc5)c4c(=O)n12)c1cccc2c4ccccc4n3c21] | 91 |
| 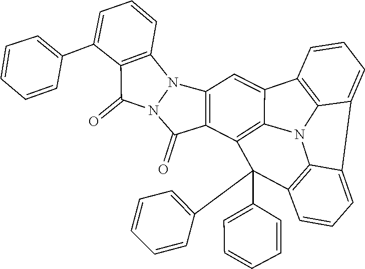 [SMILES: O=c1c2c(-c3ccccc3)cccc2n2c3cc4c5cccc6c7cccc8c7n(c4c(c3c(=O)n12)C8(c1ccccc1)c1ccccc1)c65] | 92 |
| 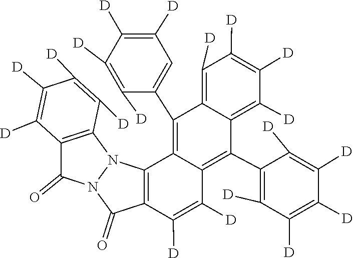 [SMILES: [2H]c1cc(-c2c3c([2H])c([2H])c([2H])c([2H])c3c(-c3c([2H])c([2H])c([2H])c([2H])c3[2H])c3c([2H])c([2H])c4c(=O)n5c(=O)c6c([2H])c([2H])c([2H])c([2H])c6n5c4c23)c([2H])c([2H])c1[2H]] | 93 |
| 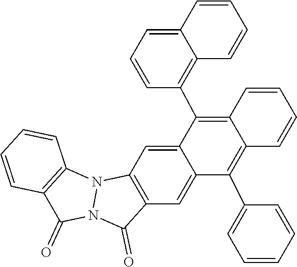 [SMILES: O=c1c2ccccc2n2c3cc4c(-c5cccc6ccccc56)c5ccccc5c(-c5ccccc5)c4cc3c(=O)n12] | 94 |
| 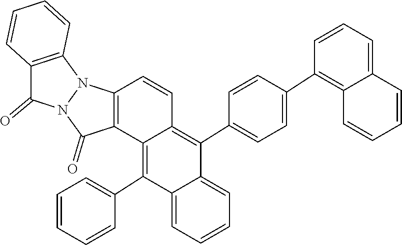 [SMILES: O=c1c2ccccc2n2c3ccc4c(-c5ccc(-c6cccc7ccccc67)cc5)c5ccccc5c(-c5ccccc5)c4c3c(=O)n12] | 95 |
| 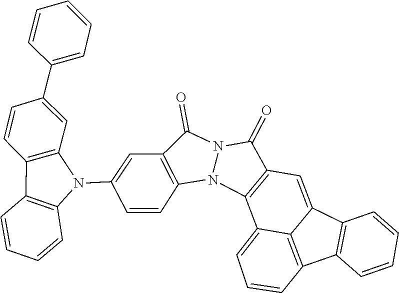 [SMILES: O=c1c2cc(-n3c4ccccc4c4ccc(-c5ccccc5)cc43)ccc2n2c3c(cc4c5c(cccc53)-c3ccccc3-4)c(=O)n12] | 96 |
| 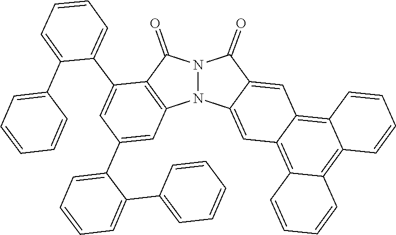 [SMILES: O=c1c2cc3c4ccccc4c4ccccc4c3cc2n2c3cc(-c4ccccc4-c4ccccc4)cc(-c4ccccc4-c4ccccc4)c3c(=O)n12] | 97 |
| 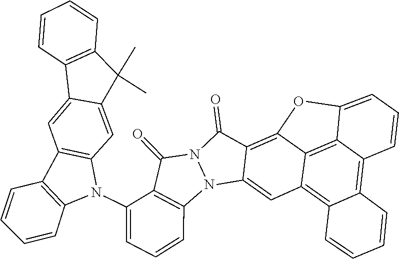 [SMILES: CC1(C)c2ccccc2-c2cc3c4ccccc4n(-c4cccc5c4c(=O)n4c(=O)c6c7oc8cccc9c%10ccccc%10c(cc6n54)c7c89)c3cc21] | 98 |
| 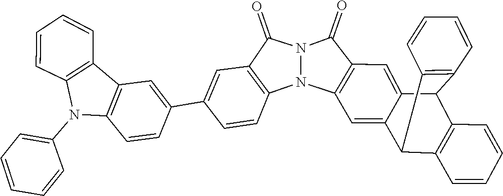 [SMILES: O=c1c2cc(-c3ccc4c(c3)c3ccccc3n4-c3ccccc3)ccc2n2c3cc4c(cc3c(=O)n12)C1c2ccccc2C4c2ccccc21] | 99 |
| 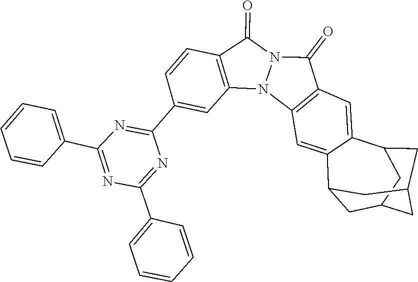 [SMILES: O=c1c2ccc(-c3nc(-c4ccccc4)nc(-c4ccccc4)n3)cc2n2c3cc4c(cc3c(=O)n12)C1CC2CC(C1)CC4C2] | 100 |
| 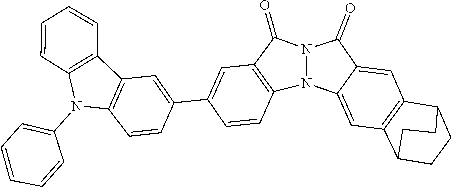 [SMILES: O=c1c2cc(-c3ccc4c(c3)c3ccccc3n4-c3ccccc3)ccc2n2c3cc4c(cc3c(=O)n12)C1CCC4CC1] | 101 |
| 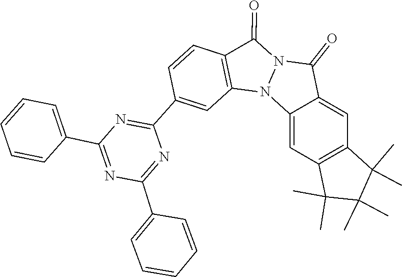 [SMILES: CC1(C)c2cc3c(=O)n4c(=O)c5ccc(-c6nc(-c7ccccc7)nc(-c7ccccc7)n6)cc5n4c3cc2C(C)(C)C1(C)C] | 102 |
| 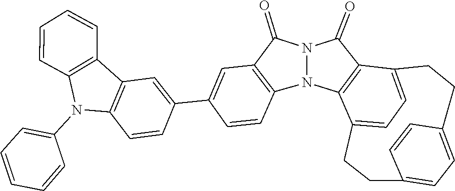 [SMILES: O=c1c2cc(-c3ccc4c(c3)c3ccccc3n4-c3ccccc3)ccc2n2c3c4ccc(c3c(=O)n12)CCc1ccc(cc1)CC4] | 103 |
| 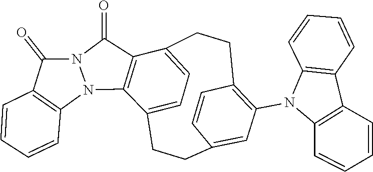 [SMILES: O=c1c2ccccc2n2c3c4ccc(c3c(=O)n12)CCc1ccc(cc1-n1c2ccccc2c2ccccc21)CC4] | 104 |
| 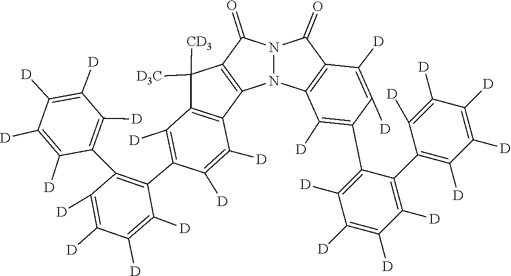 [SMILES: [2H]c1c([2H])c([2H])c(-c2c([2H])c([2H])c([2H])c([2H])c2-c2c([2H])c([2H])c3c(c2[2H])C(C([2H])([2H])[2H])(C([2H])([2H])[2H])c2c-3n3c4c([2H])c(-c5c([2H])c([2H])c([2H])c([2H])c5-c5c([2H])c([2H])c([2H])c([2H])c5[2H])c([2H])c([2H])c4c(=O)n3c2=O)c([2H])c1[2H]] | 105 |
| 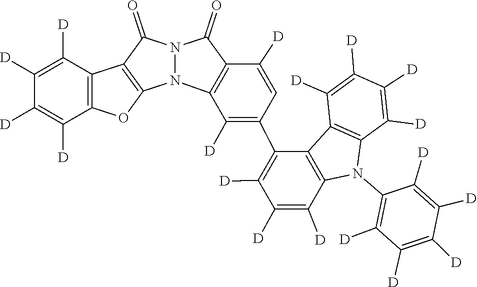 [SMILES: [2H]c1c([2H])c([2H])c(-n2c3c([2H])c([2H])c([2H])c([2H])c3c3c(-c4cc([2H])c5c(=O)n6c(=O)c7c8c([2H])c([2H])c([2H])c([2H])c8oc7n6c5c4[2H])c([2H])c([2H])c([2H])c32)c([2H])c1[2H]] | 106 |
| 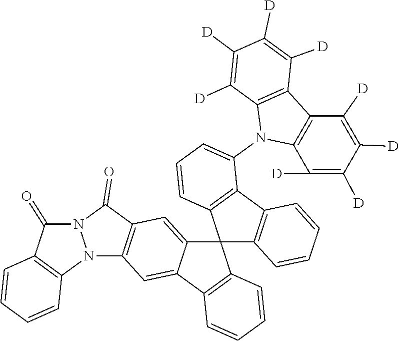 [SMILES: [2H]c1c([2H])c([2H])c2c(c1[2H])c1c([2H])c([2H])c([2H])c([2H])c1n2-c1cccc2c1-c1ccccc1C21c2ccccc2-c2cc3c(cc21)c(=O)n1c(=O)c2ccccc2n31] | 107 |
| 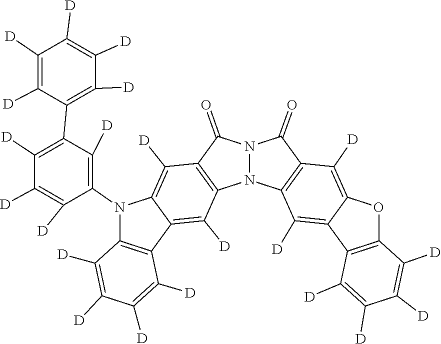 [SMILES: [2H]c1c([2H])c([2H])c(-c2c([2H])c([2H])c([2H])c(-n3c4c([2H])c([2H])c([2H])c([2H])c4c4c([2H])c5c(c([2H])c43)c(=O)n3c(=O)c4c([2H])c6oc7c([2H])c([2H])c([2H])c([2H])c7c6c([2H])c4n53)c2[2H])c([2H])c1[2H]] | 108 |
| 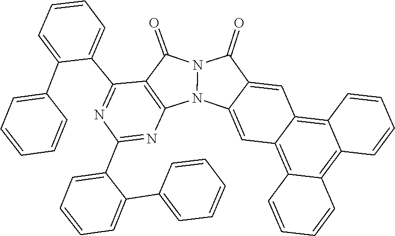 [SMILES: O=c1c2cc3c4ccccc4c4ccccc4c3cc2n2c3nc(-c4ccccc4-c4ccccc4)nc(-c4ccccc4-c4ccccc4)c3c(=O)n12] | 109 |
| 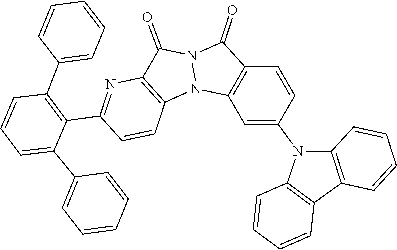 [SMILES: O=c1c2ccc(-n3c4ccccc4c4ccccc43)cc2n2c3ccc(-c4c(-c5ccccc5)cccc4-c4ccccc4)nc3c(=O)n12] | 110 |
| 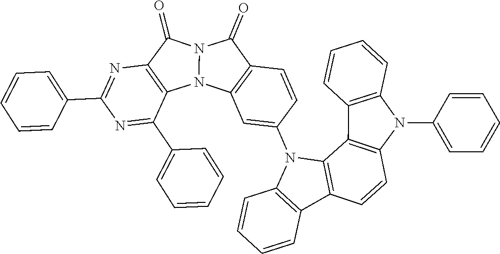 [SMILES: O=c1c2ccc(-n3c4ccccc4c4ccc5c(c6ccccc6n5-c5ccccc5)c43)cc2n2c3c(-c4ccccc4)nc(-c4ccccc4)nc3c(=O)n12] | 111 |
| 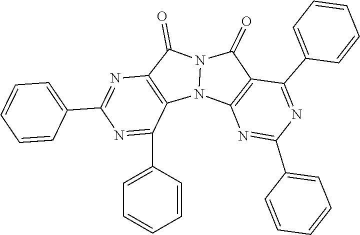 [SMILES: O=c1c2nc(-c3ccccc3)nc(-c3ccccc3)c2n2c3nc(-c4ccccc4)nc(-c4ccccc4)c3c(=O)n12] | 112 |
| 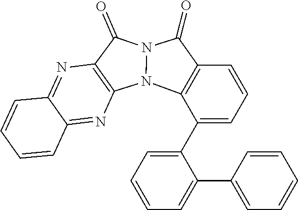 [SMILES: O=c1c2cccc(-c3ccccc3-c3ccccc3)c2n2c3nc4ccccc4nc3c(=O)n12] | 113 |
| 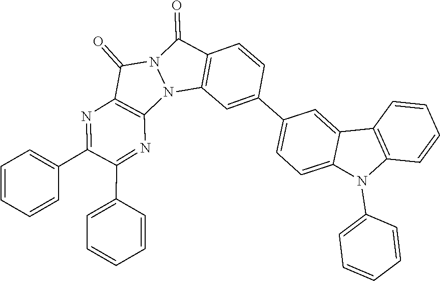 [SMILES: O=c1c2ccc(-c3ccc4c(c3)c3ccccc3n4-c3ccccc3)cc2n2c3nc(-c4ccccc4)c(-c4ccccc4)nc3c(=O)n12] | 114 |
| 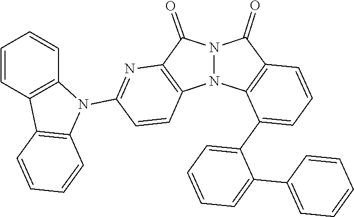 [SMILES: O=c1c2cccc(-c3ccccc3-c3ccccc3)c2n2c3ccc(-n4c5ccccc5c5ccccc54)nc3c(=O)n12] | 115 |
| 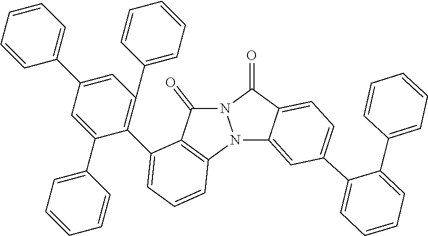 [SMILES: O=c1c2ccc(-c3ccccc3-c3ccccc3)cc2n2c3cccc(-c4c(-c5ccccc5)cc(-c5ccccc5)cc4-c4ccccc4)c3c(=O)n12] | 116 |
| 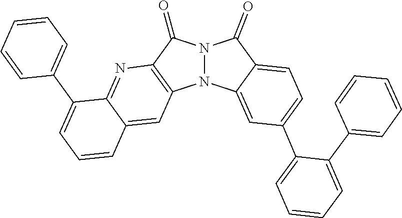 [SMILES: O=c1c2ccc(-c3ccccc3-c3ccccc3)cc2n2c3cc4cccc(-c5ccccc5)c4nc3c(=O)n12] | 117 |
| 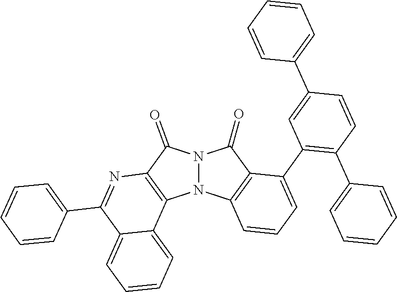 [SMILES: O=c1c2nc(-c3ccccc3)c3ccccc3c2n2c3cccc(-c4cc(-c5ccccc5)ccc4-c4ccccc4)c3c(=O)n12] | 118 |
| 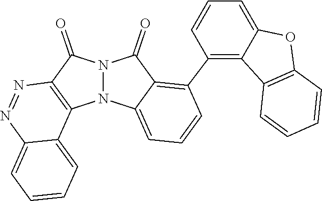 [SMILES: O=c1c2nnc3ccccc3c2n2c3cccc(-c4cccc5oc6ccccc6c45)c3c(=O)n12] | 119 |
| 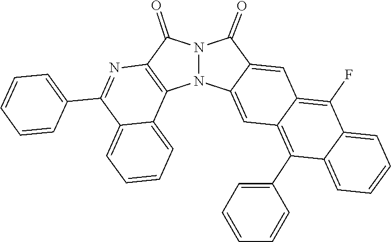 [SMILES: O=c1c2cc3c(F)c4ccccc4c(-c4ccccc4)c3cc2n2c3c(nc(-c4ccccc4)c4ccccc43)c(=O)n12] | 120 |
| 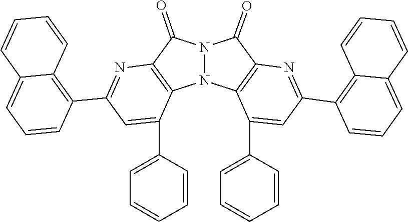 [SMILES: O=c1c2nc(-c3cccc4ccccc34)cc(-c3ccccc3)c2n2c3c(-c4ccccc4)cc(-c4cccc5ccccc45)nc3c(=O)n12] | 121 |
| 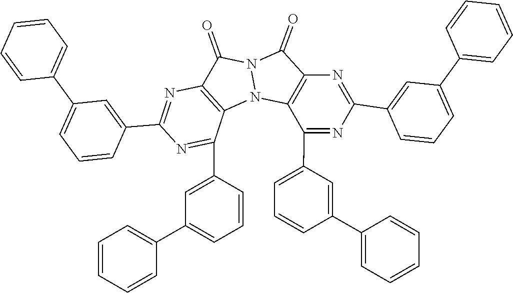 [SMILES: O=c1c2nc(-c3cccc(-c4ccccc4)c3)nc(-c3cccc(-c4ccccc4)c3)c2n2c3c(-c4cccc(-c5ccccc5)c4)nc(-c4cccc(-c5ccccc5)c4)nc3c(=O)n12] | 122 |
| 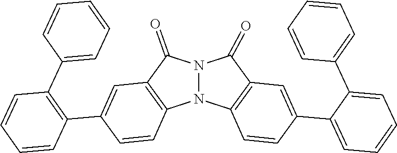 [SMILES: O=c1c2cc(-c3ccccc3-c3ccccc3)ccc2n2c3ccc(-c4ccccc4-c4ccccc4)cc3c(=O)n12] | 123 |
| 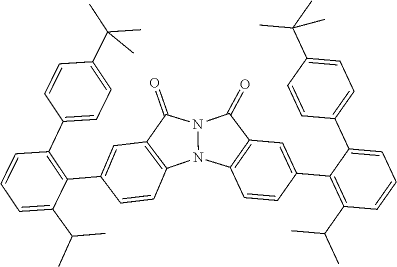 [SMILES: CC(C)c1cccc(-c2ccc(C(C)(C)C)cc2)c1-c1ccc2c(c1)c(=O)n1c(=O)c3cc(-c4c(-c5ccc(C(C)(C)C)cc5)cccc4C(C)C)ccc3n21] | 124 |
| 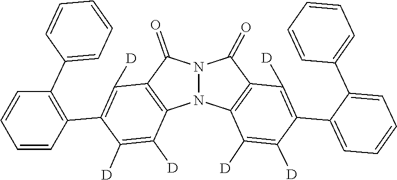 [SMILES: [2H]c1c(-c2ccccc2-c2ccccc2)c([2H])c2c(=O)n3c(=O)c4c([2H])c(-c5ccccc5-c5ccccc5)c([2H])c([2H])c4n3c2c1[2H]] | 125 |
| 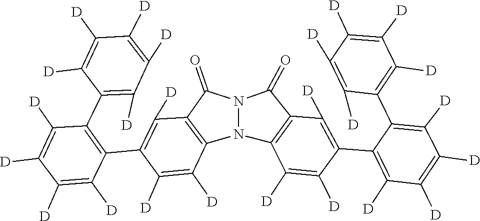 [SMILES: [2H]c1c([2H])c([2H])c(-c2c([2H])c([2H])c([2H])c([2H])c2-c2c([2H])c([2H])c3c(c2[2H])c(=O)n2c(=O)c4c([2H])c(-c5c([2H])c([2H])c([2H])c([2H])c5-c5c([2H])c([2H])c([2H])c([2H])c5[2H])c([2H])c([2H])c4n32)c([2H])c1[2H]] | 126 |
| 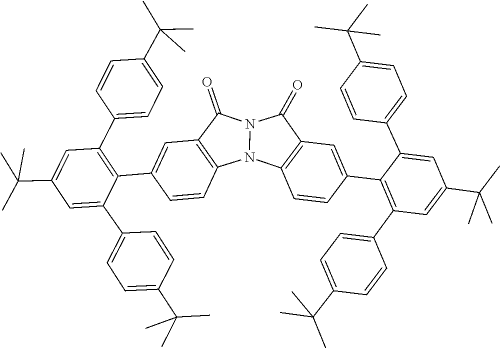 [SMILES: CC(C)(C)c1ccc(-c2cc(C(C)(C)C)cc(-c3ccc(C(C)(C)C)cc3)c2-c2ccc3c(c2)c(=O)n2c(=O)c4cc(-c5c(-c6ccc(C(C)(C)C)cc6)cc(C(C)(C)C)cc5-c5ccc(C(C)(C)C)cc5)ccc4n32)cc1] | 127 |
| 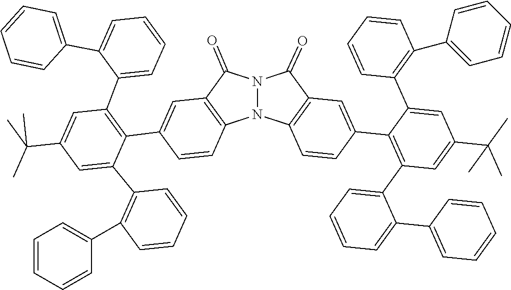 [SMILES: CC(C)(C)c1cc(-c2ccccc2-c2ccccc2)c(-c2ccc3c(c2)c(=O)n2c(=O)c4cc(-c5c(-c6ccccc6-c6ccccc6)cc(C(C)(C)C)cc5-c5ccccc5-c5ccccc5)ccc4n32)c(-c2ccccc2-c2ccccc2)c1] | 128 |
| 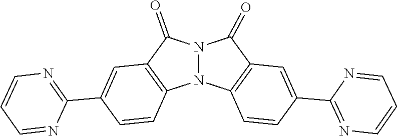 [SMILES: O=c1c2cc(-c3ncccn3)ccc2n2c3ccc(-c4ncccn4)cc3c(=O)n12] | 129 |
| 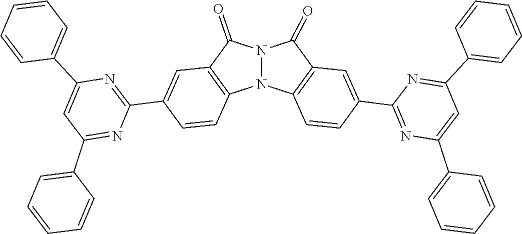 [SMILES: O=c1c2cc(-c3nc(-c4ccccc4)cc(-c4ccccc4)n3)ccc2n2c3ccc(-c4nc(-c5ccccc5)cc(-c5ccccc5)n4)cc3c(=O)n12] | 130 |
| 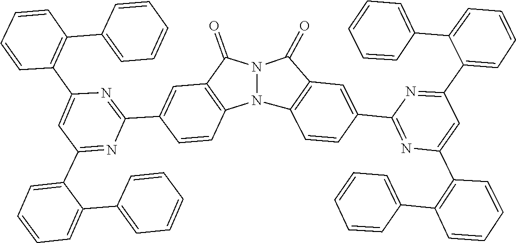 [SMILES: O=c1c2cc(-c3nc(-c4ccccc4-c4ccccc4)cc(-c4ccccc4-c4ccccc4)n3)ccc2n2c3ccc(-c4nc(-c5ccccc5-c5ccccc5)cc(-c5ccccc5-c5ccccc5)n4)cc3c(=O)n12] | 131 |
| 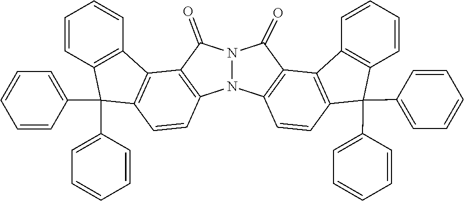 [SMILES: O=c1c2c3c(ccc2n2c4ccc5c(c4c(=O)n12)-c1ccccc1C5(c1ccccc1)c1ccccc1)C(c1ccccc1)(c1ccccc1)c1ccccc1-3] | 132 |
| 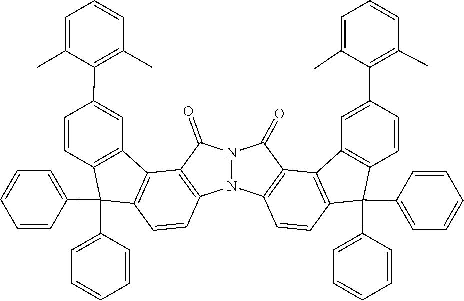 [SMILES: Cc1cccc(C)c1-c1ccc2c(c1)-c1c(ccc3c1c(=O)n1c(=O)c4c5c(ccc4n31)C(c1ccccc1)(c1ccccc1)c1ccc(-c3c(C)cccc3C)cc1-5)C2(c1ccccc1)c1ccccc1] | 133 |
| 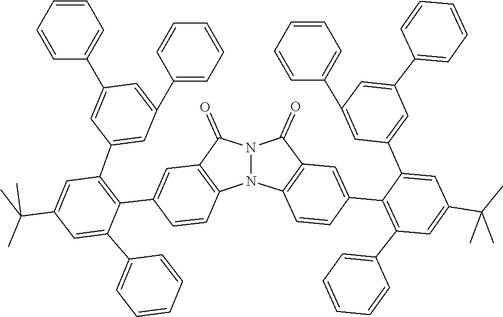 [SMILES: CC(C)(C)c1cc(-c2ccccc2)c(-c2ccc3c(c2)c(=O)n2c(=O)c4cc(-c5c(-c6ccccc6)cc(C(C)(C)C)cc5-c5cc(-c6ccccc6)cc(-c6ccccc6)c5)ccc4n32)c(-c2cc(-c3ccccc3)cc(-c3ccccc3)c2)c1] | 134 |
| 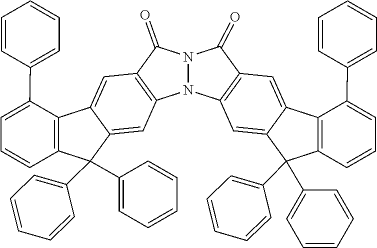 [SMILES: O=c1c2cc3c(cc2n2c4cc5c(cc4c(=O)n12)-c1c(-c2ccccc2)cccc1C5(c1ccccc1)c1ccccc1)C(c1ccccc1)(c1ccccc1)c1cccc(-c2ccccc2)c1-3] | 135 |
| 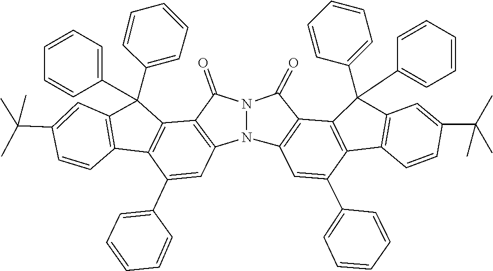 [SMILES: CC(C)(C)c1ccc2c(c1)C(c1ccccc1)(c1ccccc1)c1c-2c(-c2ccccc2)cc2c1c(=O)n1c(=O)c3c4c(c(-c5ccccc5)cc3n21)-c1ccc(C(C)(C)C)cc1C4(c1ccccc1)c1ccccc1] | 136 |
| 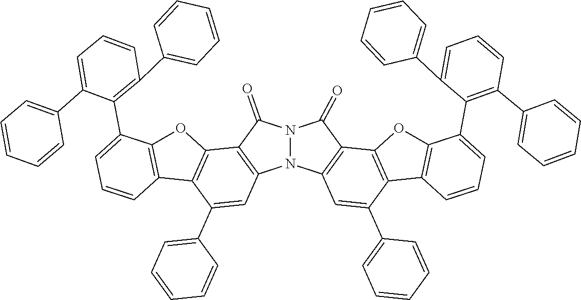 [SMILES: O=c1c2c3oc4c(-c5c(-c6ccccc6)cccc5-c5ccccc5)cccc4c3c(-c3ccccc3)cc2n2c3cc(-c4ccccc4)c4c5cccc(-c6c(-c7ccccc7)cccc6-c6ccccc6)c5oc4c3c(=O)n12] | 137 |
| 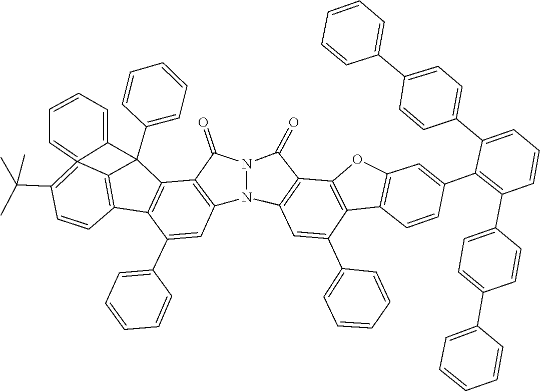 | 138 |
| 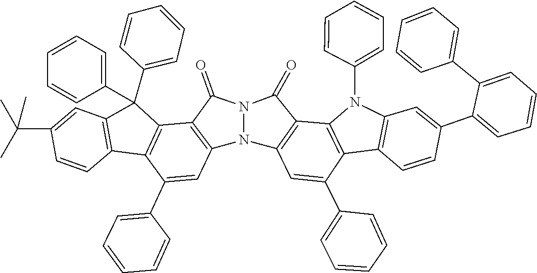 [SMILES: CC(C)(C)c1ccc2c(c1)C(c1ccccc1)(c1ccccc1)c1c-2c(-c2ccccc2)cc2c1c(=O)n1c(=O)c3c4c(c(-c5ccccc5)cc3n21)c1ccc(-c2ccccc2-c2ccccc2)cc1n4-c1ccccc1] | 139 |
| 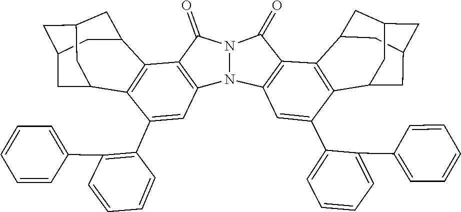 [SMILES: O=c1c2c3c(c(-c4ccccc4-c4ccccc4)cc2n2c4cc(-c5ccccc5-c5ccccc5)c5c(c4c(=O)n12)C1CC2CC(CC5C2)C1)C1CC2CC(C1)CC3C2] | 140 |
| 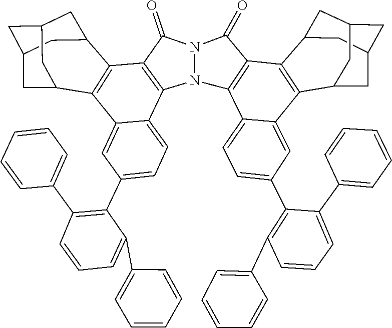 [SMILES: O=c1c2c3c(c4cc(-c5c(-c6ccccc6)cccc5-c5ccccc5)ccc4c2n2c4c(c5c(c6cc(-c7c(-c8ccccc8)cccc7-c7ccccc7)ccc64)C4CC6CC(C4)CC5C6)c(=O)n12)C1CC2CC(C1)CC3C2] | 141 |
| 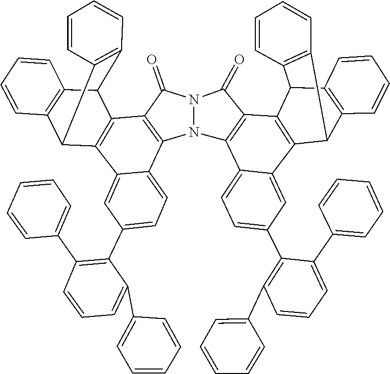 [SMILES: O=c1c2c3c(c4cc(-c5c(-c6ccccc6)cccc5-c5ccccc5)ccc4c2n2c4c(c5c(c6cc(-c7c(-c8ccccc8)cccc7-c7ccccc7)ccc64)C4c6ccccc6C5c5ccccc54)c(=O)n12)C1c2ccccc2C3c2ccccc21] | 142 |
| 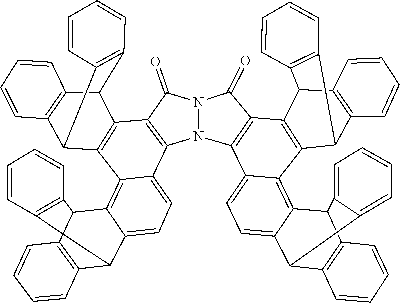 [SMILES: O=c1c2c3c(c4c5c(ccc4c2n2c4c(c6c(c7c8c(ccc74)C4c7ccccc7C47c4ccccc4C87)C4c7ccccc7C6c6ccccc64)c(=O)n12)C1c2ccccc2C5c2ccccc21)C1c2ccccc2C3c2ccccc21] | 143 |
| 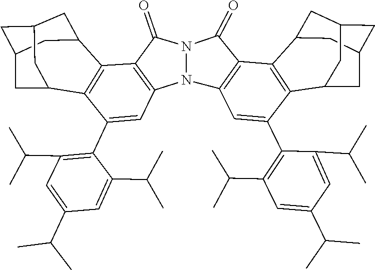 [SMILES: CC(C)c1cc(C(C)C)c(-c2cc3c(c4c2C2CC5CC(C2)CC4C5)c(=O)n2c(=O)c4c5c(c(-c6c(C(C)C)cc(C(C)C)cc6C(C)C)cc4n32)C2CC3CC(C2)CC5C3)c(C(C)C)c1] | 144 |
| 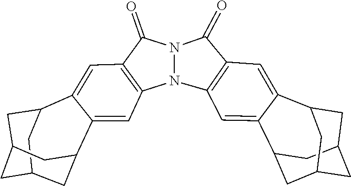 [SMILES: O=c1c2cc3c(cc2n2c4cc5c(cc4c(=O)n12)C1CC2CC(C1)CC5C2)C1CC2CC(CC3C2)C1] | 145 |
| 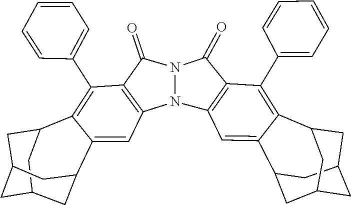 [SMILES: O=c1c2c(-c3ccccc3)c3c(cc2n2c4cc5c(c(-c6ccccc6)c4c(=O)n12)C1CC2CC(CC5C2)C1)C1CC2CC(C1)CC3C2] | 146 |
| 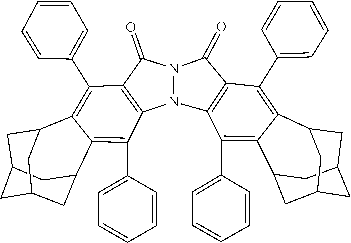 [SMILES: O=c1c2c(-c3ccccc3)c3c(c(-c4ccccc4)c2n2c4c(-c5ccccc5)c5c(c(-c6ccccc6)c4c(=O)n12)C1CC2CC(C1)CC5C2)C1CC2CC(CC3C2)C1] | 147 |
| 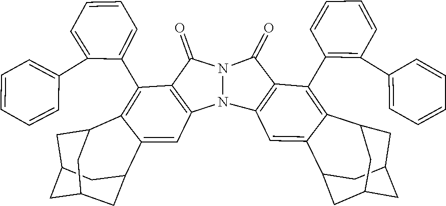 [SMILES: O=c1c2c(-c3ccccc3-c3ccccc3)c3c(cc2n2c4cc5c(c(-c6ccccc6-c6ccccc6)c4c(=O)n12)C1CC2CC(CC5C2)C1)C1CC2CC(C1)CC3C2] | 148 |
| 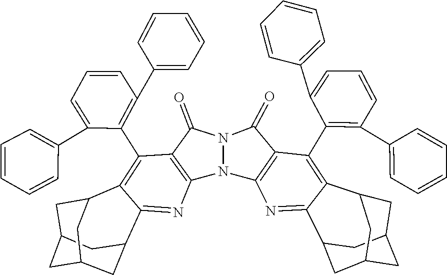 [SMILES: O=c1c2c(-c3c(-c4ccccc4)cccc3-c3ccccc3)c3c(nc2n2c4nc5c(c(-c6c(-c7ccccc7)cccc6-c6ccccc6)c4c(=O)n12)C1CC2CC(CC5C2)C1)C1CC2CC(C1)CC3C2] | 149 |
| 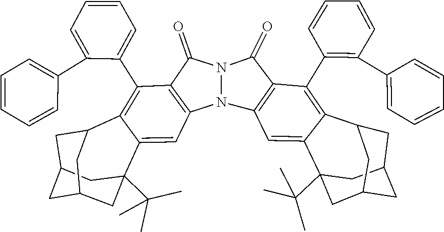 [SMILES: CC(C)(C)C12CC3CC(CC(C3)c3c1cc1c(c3-c3ccccc3-c3ccccc3)c(=O)n3c(=O)c4c(-c5ccccc5-c5ccccc5)c5c(cc4n13)C1(C(C)(C)C)CC3CC(CC5C3)C1)C2] | 150 |
| 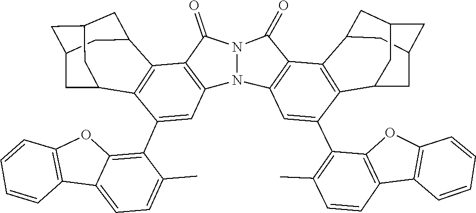 [SMILES: Cc1ccc2c(oc3ccccc32)c1-c1cc2c(c3c1C1CC4CC(C1)CC3C4)c(=O)n1c(=O)c3c4c(c(-c5c(C)ccc6c5oc5ccccc56)cc3n21)C1CC2CC(C1)CC4C2] | 151 |
| 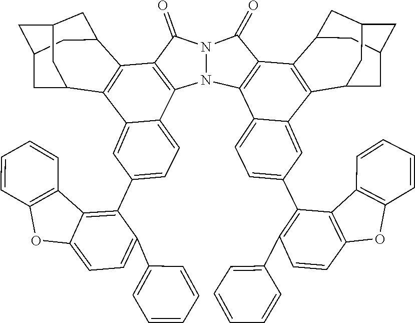 [SMILES: O=c1c2c3c(c4cc(-c5c(-c6ccccc6)ccc6oc7ccccc7c56)ccc4c2n2c4c(c5c(c6cc(-c7c(-c8ccccc8)ccc8oc9ccccc9c78)ccc64)C4CC6CC(C4)CC5C6)c(=O)n12)C1CC2CC(C1)CC3C2] | 152 |

Preferred embodiments of compounds of the invention are recited in detail in the examples, these compounds being usable alone or in combination with further compounds for all purposes of the invention.

Provided that the conditions specified in Claim 1 are met, the abovementioned preferred embodiments can be combined with one another as desired. In a particularly preferred embodiment of the invention, the abovementioned preferred embodiments apply simultaneously.

The compounds of the invention are preparable in principle by various processes. However, the processes described hereinafter have been found to be particularly suitable.

Therefore, the present invention further provides a process for preparing the compounds of the invention, in which a base skeleton having at least one of the W^{a} groups or a precursor of one of the W^{a} groups is synthesized, and then an aromatic or heteroaromatic radical is introduced by means of a nucleophilic aromatic substitution reaction or a coupling reaction.

Suitable compounds comprising a base skeleton having a W^{a} group are in many cases commercially available, and the starting compounds detailed in the examples are obtainable by known processes, and so reference is made thereto.

These compounds can be reacted with further compounds by known coupling reactions, the necessary conditions for this purpose being known to the person skilled in the art, and detailed specifications in the examples giving support to the person skilled in the art in conducting these reactions.

Particularly suitable and preferred coupling reactions which all lead to C—C bond formations and/or C—N bond formations are those according to BUCHWALD, SUZUKI, YAMAMOTO, STILLE, HECK, NEGISHI, SONOGASHIRA and HIYAMA. These reactions are widely known, and the examples will provide the person skilled in the art with further pointers.

The principles of the preparation processes detailed above are known in principle from the literature for similar compounds and can be adapted easily by the person skilled in the art for the preparation of the compounds of the invention. Further information can be found in the examples.

It is possible by these methods, if necessary followed by purification, for example recrystallization or sublimation, to obtain the compounds of the invention in high purity, preferably more than 99% (determined by means of ¹H NMR and/or HPLC).

The compounds of the invention may also be mixed with a polymer. It is likewise possible to incorporate these compounds covalently into a polymer. This is especially possible with compounds substituted by reactive leaving groups such as bromine, iodine, chlorine, boronic acid or boronic ester, or by reactive polymerizable groups such as olefins or oxetanes. These may find use as monomers for production of corresponding oligomers, dendrimers or polymers. The oligomerization or polymerization is preferably effected via the halogen functionality or the boronic acid functionality or via the polymerizable group. It is additionally possible to crosslink the polymers via groups of this kind. The compounds and polymers of the invention may be used in the form of a crosslinked or uncrosslinked layer.

The invention therefore further provides oligomers, polymers or dendrimers containing one or more of the above-detailed structures of the formula (I) and preferred embodiments of this formula or compounds of the invention, wherein one or more bonds of the compounds of the invention or of the structures of the formula (I) and preferred embodiments of that formula to the polymer, oligomer or dendrimer are present. According to the linkage of the structures of the formula (I) and preferred embodiments of this formula or of the compounds, these therefore form a side chain of the oligomer or polymer or are bonded within the main chain. The polymers, oligomers or dendrimers may be conjugated, partly conjugated or nonconjugated. The oligomers or polymers may be linear, branched or dendritic. For the repeat units of the compounds of the invention in oligomers, dendrimers and polymers, the same preferences apply as described above.

For preparation of the oligomers or polymers, the monomers of the invention are homopolymerized or copolymerized with further monomers. Preference is given to copolymers wherein the units of formula (I) or the preferred embodiments recited above and hereinafter are present to an extent of 0.01 to 99.9 mol %, preferably 5 to 90 mol %, more preferably 20 to 80 mol %. Suitable and preferred comonomers which form the polymer base skeleton are chosen from fluorenes (for example according to EP 842208 or WO 2000/022026), spirobifluorenes (for example according to EP 707020, EP 894107 or WO 2006/061181), paraphenylenes (for example according to WO 92/18552), carbazoles (for example according to WO 2004/070772 or WO 2004/113468), thiophenes (for example according to EP 1028136), dihydrophenanthrenes (for example according to WO 2005/014689), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers may contain still further units, for example hole transport units, especially those based on triarylamines, and/or electron transport units.

Additionally of particular interest are compounds of the invention which feature a high glass transition temperature. In this connection, preference is given especially to compounds of the invention comprising structures of the formula (I) or the preferred embodiments recited above and hereinafter which have a glass transition temperature of at least 70° C., more preferably of at least 110° C., even more preferably of at least 125° C. and especially preferably of at least 150° C., determined in accordance with DIN 51005 (2005-08 version).

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, a-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitter and/or a matrix material, where these compounds differ from the compounds of the invention. Suitable emitters and matrix materials are listed at the back in connection with the organic electroluminescent device. The further compound may also be polymeric.

The present invention therefore still further provides a composition comprising at least one compound of the formula (I)

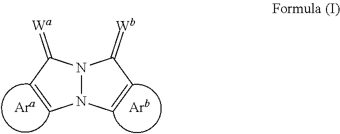

where the symbols used have the definitions given above, preferably at least one compound of the invention or an oligomer, polymer or dendrimer comprising structures of formula (I) and at least one further organofunctional material. Functional materials are generally the organic or inorganic materials introduced between the anode and cathode. Preferably, the organically functional material is selected from the group consisting of fluorescent emitters, phosphorescent emitters, emitters that exhibit TADF (thermally activated delayed fluorescence), host materials, electron transport materials, electron injection materials, hole conductor materials, hole injection materials, electron blocker materials, hole blocker materials, wide bandgap materials and n-dopants.

The present invention further provides for the use of a compound of formula (I)

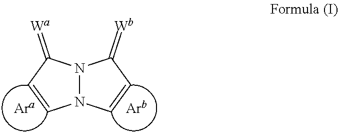

where the symbols used have the definitions given above, preferably that of a compound of the invention or an oligomer, polymer or dendrimer comprising structures of formula (I) in an electronic device, especially in an organic electroluminescent device, preferably as emitter, more preferably as green, red or blue emitter. In this case, compounds of the invention preferably exhibit fluorescent properties and thus provide preferentially fluorescent emitters.

It may preferably be the case that structures/compounds of formulae (II-2) to (II-8), (II-17) to (II-23), (III-2) to (III-8), (III-17) to (III-23), (IV-2) to (IV-8), (IV-17) to (IV-23), (V-1), (V-2), (V-5) and/or (V-6) are used as emitter.

In addition, compounds of formula (I) or an oligomer, polymer or dendrimer comprising structures of formula (I) may be used as host materials and/or electron transport materials. It may preferably be the case that structures/compounds having an anthracene group (Ar-76) to (Ar-78), preferably (Ar-78), and/or compounds of formulae (II-9), (II-10), (II-24), (II-25), (III-9), (III-10), (III-24), (III-25), (IV-9), (IV-10), (IV-24) and/or (IV-25) are used as electron transport material and/or matrix material.

The present invention still further provides an electronic device comprising at least one compound of formula (I)

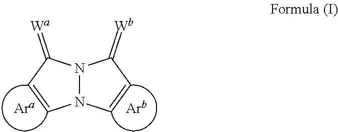

where the symbols used have the definitions given above, preferably a compound of the invention or an oligomer, polymer or dendrimer comprising structures of formula (I). An electronic device in the context of the present invention is a device comprising at least one layer comprising at least one organic compound. This component may also comprise inorganic materials or else layers formed entirely from inorganic materials.

More preferably, the electronic device is selected from the group consisting of organic electroluminescent devices (OLEDs, sOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (0-laser), organic plasmon-emitting devices (D. M. Koller et al., Nature Photonics 2008, 1-4), organic integrated circuits (0-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, sOLED, PLEDs, LECs, etc.), more preferably organic light-emitting diodes (OLEDs), organic light-emitting diodes based on small molecules (sOLEDs), organic light-emitting diodes based on polymers (PLEDs), especially phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The compound of the invention may be used in different layers, according to the exact structure. Preference is given to an organic electroluminescent device comprising a structure/compound of formula (I) or the above-detailed preferred embodiments in an emitting layer as emitter, preferably red, green or blue emitter. Very preferred is an organic electroluminescent device comprising a structure/compound of formula (I) or the above-detailed preferred embodiments in an emitting layer as fluorescent blue emitter. Preference is given here especially to structures/compounds of formulae (II-2) to (II-8), (II-17) to (II-23), (III-2) to (III-8), (III-17) to (III-23), (IV-2) to (IV-8), (IV-17) to (IV-23), (V-1), (V-2), (V-5) and/or (V-6).

When the compound of the invention is used as emitter in an emitting layer, preference is given to using a suitable matrix material (also called host material) which is known as such.

A preferred mixture of the compound of the invention and a matrix material contains between 99% and 1% by volume, preferably between 98% and 10% by volume, more preferably between 97% and 60% by volume and especially between 95% and 80% by volume of matrix material, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

In a preferred configuration, a compound containing a structure/compound of formula (I) or the above-detailed preferred embodiments which is used as emitter is preferably used in combination with one or more phosphorescent materials (triplet emitters) and/or a compound which is a TADF (thermally activated delayed fluorescence) host material. Preference is given here to forming a hyperfluorescence system as described in WO 2012/133188 and/or a hyperphosphorescence system as in US 2017271611. This combination is a preferred composition according to the present invention.

WO 2015/091716 A1 and WO 2016/193243 A1 disclose OLEDs containing both a phosphorescent compound and a fluorescent emitter in the emission layer, where the energy is transferred from the phosphorescent compound to the fluorescent emitter (hyperphosphorescence). In this context, the phosphorescent compound accordingly behaves as a host material. As the person skilled in the art knows, host materials have higher singlet and triplet energies as compared to the emitters in order that the energy from the host material can also be transferred to the emitter with maximum efficiency. The systems disclosed in the prior art have exactly such an energy relation.

Phosphorescence in the context of this invention is understood to mean luminescence from an excited state having higher spin multiplicity, i.e. a spin state>1, especially from an excited triplet state. In the context of this application, all luminescent complexes with transition metals or lanthanides, especially all iridium, platinum and copper complexes, shall be regarded as phosphorescent compounds.

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/001990, WO 2018/019687, WO 2018/019688, WO 2018/041769, WO 2018/054798, WO 2018/069196, WO 2018/069197, WO 2018/069273, WO 2018/178001, WO 2018/177981, WO 2019/020538, WO 2019/115423, WO 2019/158453 and WO 2019/179909. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

A compound of the invention may preferably be used in combination with a TADF host material and/or a TADF emitter, as set out above.

The process referred to as thermally activated delayed fluorescence (TADF) is described, for example, by B. H. Uoyama et al., Nature 2012, Vol. 492, 234. In order to enable this process, a comparatively small singlet-triplet separation ΔE(S₁−T₁) of less than about 2000 cm⁻¹, for example, is needed in the emitter. In order to open up the T₁→S₁ transition which is spin-forbidden in principle, as well as the emitter, it is possible to provide a further compound in the matrix that has strong spin-orbit coupling, such that intersystem crossing is enabled via the spatial proximity and the interaction which is thus possible between the molecules, or the spin-orbit coupling is generated by means of a metal atom present in the emitter.

Sources of further valuable information relating to hyperfluorescence systems include WO2012/133188 (Idemitsu), WO2015/022974 (Kyushu Univ.), WO2015/098975 (Idemitsu), WO2020/053150 (Merck) and DE202019005189 (Merck).

Sources of further valuable information relating to hyperphosphorescence systems include WO2015/091716 A1, WO2016/193243 A1 (BASF), WO01/08230 A1 (Princeton Univ. (Mark Thompson)), US2005/0214575A1 (Fuji), WO2012/079673 (Merck), WO2020/053314 (Merck) and WO2020/053315 (Merck).

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

Also preferred is an organic electroluminescent device comprising a structure/compound of formula (I) or the above-detailed preferred embodiments in an electron-conducting layer as electron transport material. Preference is given here especially to compounds having an anthracene group, preferably a group of formulae (Ar-76) bis (Ar-78), preferably (Ar-78), and/or structures/compounds of formulae (II-9), (II-10), (II-24), (II-25), (III-9), (III-10), (III-24), (III-25), (IV-9), (IV-10), (IV-24) and/or (IV-25).

In the further layers of the organic electroluminescent device of the invention, it is possible to use any materials as typically used according to the prior art. The person skilled in the art will therefore be able, without exercising inventive skill, to use any materials known for organic electroluminescent devices in combination with the structure/compound of formula (I) or the above-recited preferred embodiments.

Additionally preferred is an organic electroluminescent device, characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for applying a compound of formula (I) or the preferred embodiments thereof detailed above are novel. The present invention therefore further provides formulations containing at least one solvent and a compound according to formula (I) or the preferred embodiments thereof detailed above.

In addition, hybrid methods are possible, in which, for example, one or more layers are applied from solution and one or more further layers are applied by vapour deposition.

Those skilled in the art are generally aware of these methods and are able to apply them without exercising inventive skill to organic electroluminescent devices comprising the compounds of the invention.

The compounds of the invention and the organic electroluminescent devices of the invention have the particular feature of an improved lifetime over the prior art. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. Electronic devices, especially organic electroluminescent devices comprising structures/compounds of formula (I) or the preferred embodiments as emitters that have been recited above and hereinafter have very narrow emission bands having very low FWHM (Full Width Half Maximum) values, and lead to particularly pure-color emission, recognizable by the low CIE y values. What is particularly surprising here is that both blue emitters having low FWHM values and emitters having low FWHM values that emit in the green, yellow or red region of the colour spectrum are provided.
2. Electronic devices, especially organic electroluminescent devices, comprising structures/compounds of formula (I) or the preferred embodiments recited above and hereinafter, especially as emitter, as hole conductor material and/or as electron transport material, have a very good lifetime. In this context, these compounds especially bring about low roll-off, i.e. a small drop in power efficiency of the device at high luminances.
3. Electronic devices, especially organic electroluminescent devices, comprising structures/compounds of formula (I) or the preferred embodiments that have been recited above and hereinafter, have excellent efficiency as emitter and/or as electron transport material. In this context, inventive structures/compounds of formula (I) or the preferred embodiments recited above and hereinafter bring about a low operating voltage when used in electronic devices.
4. The inventive structures/compounds of formula (I) or the preferred embodiments recited above and hereinafter exhibit very high stability and lifetime.
5. With structures/compounds of formula (I) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
   Exciton energy is transmitted from a matrix or host in the emission layer to the emitter, typically either via what is called Dexter transfer or via Förster transfer. Förster energy transfer (FRET) from a host or matrix to the emitter of the invention is particularly preferred here, since it is particularly efficient, which leads to electronic devices having particularly good performance data (for example efficiency, voltage and lifetime). It is found that energy is preferably transferred from a host or matrix to the compounds of the invention via Förster transfer.
6. Structures/compounds of formula (I) or the preferred embodiments recited above and hereinafter have excellent glass film formation.
7. Structures/compounds of formula (I) or the preferred embodiments recited above and hereinafter form very good films from solutions and show excellent solubility.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The metal complexes are additionally handled with exclusion of light or under yellow light. The solvents and reagents can be purchased, for example, from Sigma-ALDRICH or ABCR. The respective figures in square brackets or the numbers quoted for individual compounds relate to the CAS numbers of the compounds known from the literature. In the case of compounds that can have multiple enantiomeric, diastereomeric or tautomeric forms, one form is shown in a representative manner.

Synthons LS Known from the Literature:

| | |
|---|---|
| 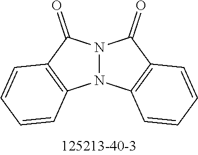 [SMILES: O=c1c2ccccc2n2c3ccccc3c(=O)n12] | LS1 |
| 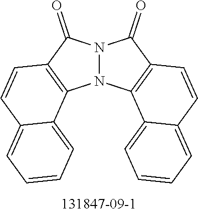 [SMILES: O=c1c2ccc3ccccc3c2n2c3c(ccc4ccccc43)c(=O)n12] | LS2 |
| 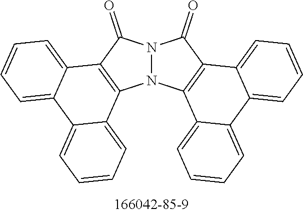 [SMILES: O=c1c2c3ccccc3c3ccccc3c2n2c3c4ccccc4c4ccccc4c3c(=O)n12] | LS3 |

### Synthesis of Synthons S:

Reactants Known from the Literature:

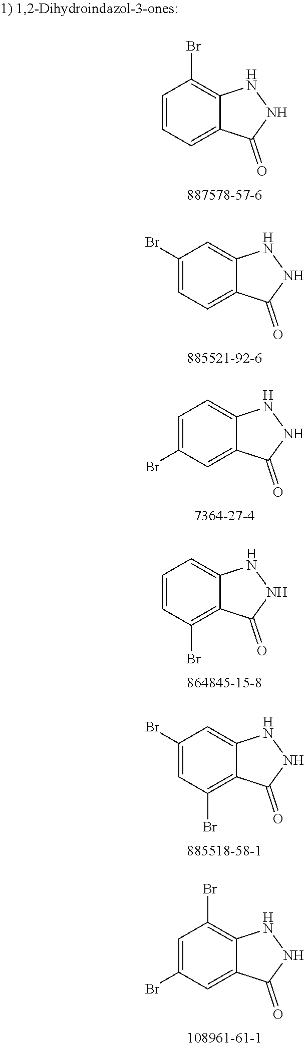 [SMILES: O=c1[nH][nH]c2c(Br)cccc12 ; O=c1[nH][nH]c2cc(Br)ccc12 ; O=c1[nH][nH]c2ccc(Br)cc12 ; O=c1[nH][nH]c2cccc(Br)c12 ; O=c1[nH][nH]c2cc(Br)cc(Br)c12 ; O=c1[nH][nH]c2c(Br)cc(Br)cc12]

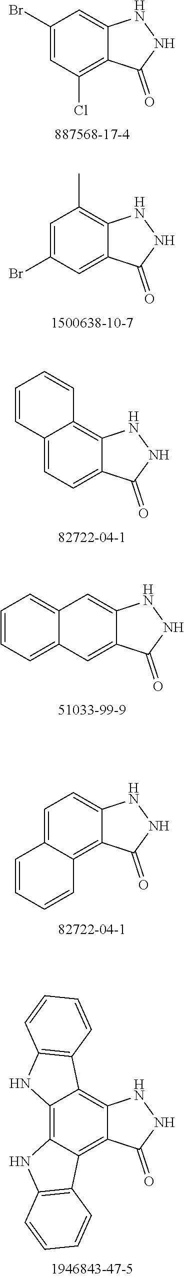 [SMILES: O=c1[nH][nH]c2cc(Br)cc(Cl)c12 ; Cc1cc(Br)cc2c(=O)[nH][nH]c12 ; O=c1[nH][nH]c2c1ccc1ccccc12 ; O=c1[nH][nH]c2cc3ccccc3cc12 ; O=c1[nH][nH]c2ccc3ccccc3c12 ; O=c1[nH][nH]c2c1c1c3ccccc3[nH]c1c1[nH]c3ccccc3c12]

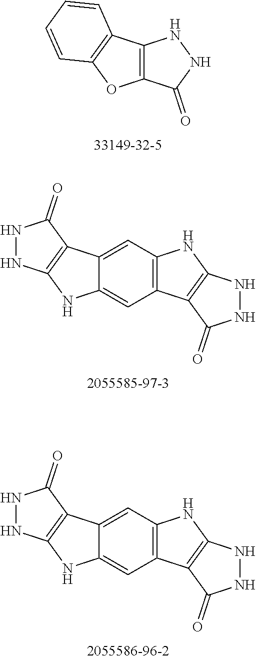 [SMILES: O=c1[nH][nH]c2c1oc1ccccc12 ; O=c1[nH][nH]c2[nH]c3cc4c(cc3c12)[nH]c1[nH][nH]c(=O)c14 ; O=c1[nH][nH]c2[nH]c3cc4c(cc3c12)[nH]c1[nH][nH]c(=O)c14]

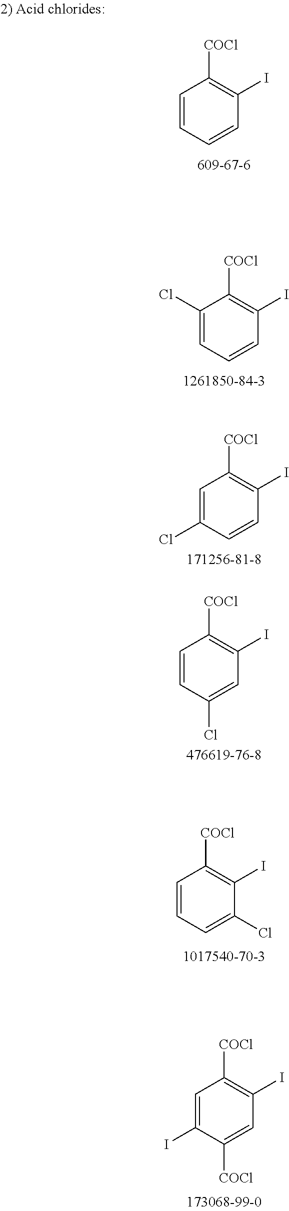 [SMILES: O=C(Cl)c1ccccc1I ; O=C(Cl)c1c(Cl)cccc1I ; O=C(Cl)c1cc(Cl)ccc1I ; O=C(Cl)c1ccc(Cl)cc1I ; O=C(Cl)c1cccc(Cl)c1I ; O=C(Cl)c1cc(I)c(C(=O)Cl)cc1I]

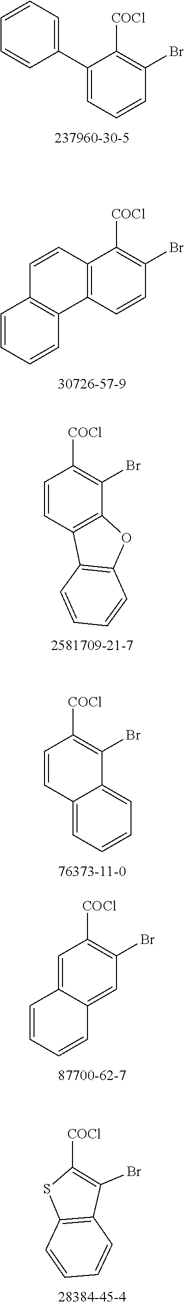 [SMILES: O=C(Cl)c1c(Br)cccc1-c1ccccc1 ; O=C(Cl)c1c(Br)ccc2c1ccc1ccccc12 ; O=C(Cl)c1ccc2c(oc3ccccc32)c1Br ; O=C(Cl)c1ccc2ccccc2c1Br ; O=C(Cl)c1cc2ccccc2cc1Br ; O=C(Cl)c1sc2ccccc2c1Br]

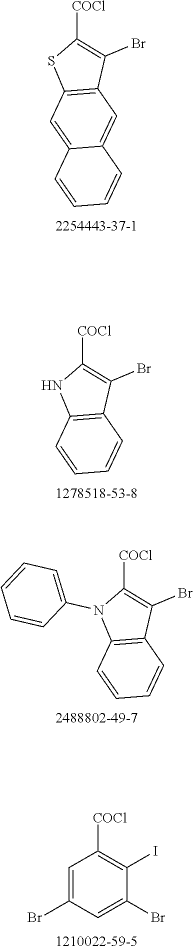 [SMILES: O=C(Cl)c1sc2cc3ccccc3cc2c1Br ; O=C(Cl)c1[nH]c2ccccc2c1Br ; O=C(Cl)c1c(Br)c2ccccc2n1-c1ccccc1 ; O=C(Cl)c1cc(Br)cc(Br)c1I]

### Example S1

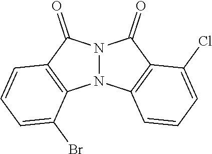 [SMILES: O=c1c2cccc(Br)c2n2c3cccc(Cl)c3c(=O)n12]

Procedure according to Z. Wang et al., Chin. J. Chem., 29, 2769, 2011. A mixture of 21.3 g (100 mmol) of 7-bromo-1,2-dihydro-3H-indazol-3-one [887578-57-6], 39.1 g (130 mmol) of 2-chloro-6-iodobenzoyl chloride [1261850-84-3], 97.8 g (300 mmol) of caesium carbonate, anhydrous, 3.6 g (20 mmol) of 1,10-phenanthroline, 1.9 g (10 mmol) of copper iodide, 200 g of glass beads (diameter 3 mm) and 800 ml of toluene is stirred at 100° C. for 16 h (TLC monitoring for complete conversion of the 7-bromo-1,2-dihydro-3H-indazol-3-one). The mixture is left to cool to 80° C. and filtered through a Celite bed in the form of a hot toluene slurry, and the filtrate is concentrated to dryness under reduced pressure. The residue is taken up in 500 ml of dichloromethane (DCM) and applied to about 400 g of silica gel, the silica gel is packed into a silica gel column in the form of a DCM slurry (800 g), and DCM is used to elute the anti-dibenzobimane regioisomer. Subsequently, the desired syn-dibenzobimane regioisomer is eluted with DCM:methyl tert-butyl ether (MTBE) 8:2, and this is recrystallized once from acetonitrile. Yield: 9.5 g (27 mmol), 27%. Purity: 97% by ¹H NMR.

The following compounds can be prepared analogously:

| Ex | Reactants | Product | Yield |
|---|---|---|---|
| S2 | 885521-92-6 1261850-84-3 | 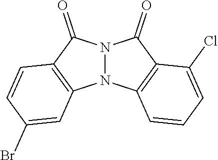 [SMILES: O=c1c2ccc(Br)cc2n2c3cccc(Cl)c3c(=O)n12] | 30% |
| S3 | 7364-27-4 1261850-84-3 | 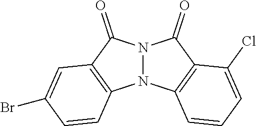 [SMILES: O=c1c2cc(Br)ccc2n2c3cccc(Cl)c3c(=O)n12] | 30% |
| S4 | 864845-15-8 1261850-84-3 | 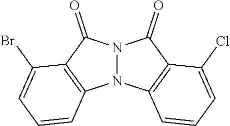 [SMILES: O=c1c2c(Cl)cccc2n2c3cccc(Br)c3c(=O)n12] | 28% |
| S5 | 887578-57-6 171256-81-8 | 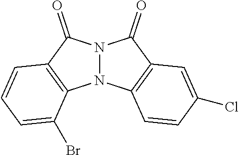 [SMILES: O=c1c2cc(Cl)ccc2n2c3c(Br)cccc3c(=O)n12] | 32% |
| S6 | 885521-92-6 171256-81-8 | 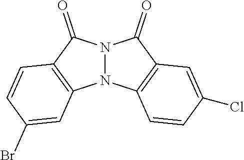 [SMILES: O=c1c2cc(Cl)ccc2n2c3cc(Br)ccc3c(=O)n12] | 35% |
| S7 | 7364-27-4 171256-81-8 | 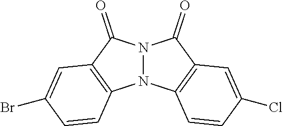 [SMILES: O=c1c2cc(Cl)ccc2n2c3ccc(Br)cc3c(=O)n12] | 33% |
| S8 | 864845-15-8 171256-81-8 | 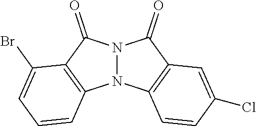 [SMILES: O=c1c2cc(Cl)ccc2n2c3cccc(Br)c3c(=O)n12] | 32% |
| S9 | 887578-57-6 476619-76-8 | 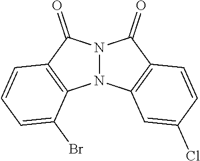 [SMILES: O=c1c2ccc(Cl)cc2n2c3c(Br)cccc3c(=O)n12] | 29% |
| S10 | 885521-92-6 476619-76-8 | 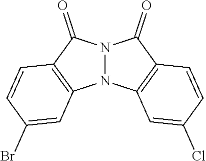 [SMILES: O=c1c2ccc(Cl)cc2n2c3cc(Br)ccc3c(=O)n12] | 38% |
| S11 | 7364-27-4 476619-76-8 | 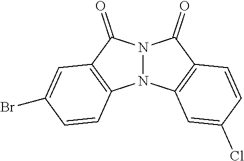 [SMILES: O=c1c2cc(Br)ccc2n2c3cc(Cl)ccc3c(=O)n12] | 35% |
| S12 | 864845-15-8 476619-76-8 | 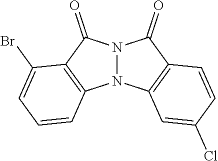 [SMILES: O=c1c2ccc(Cl)cc2n2c3cccc(Br)c3c(=O)n12] | 33% |
| S13 | 887578-57-6 1017540-70-3 | 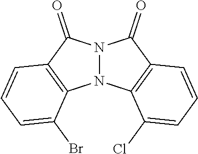 [SMILES: O=c1c2cccc(Cl)c2n2c3c(Br)cccc3c(=O)n12] | 20% |
| S14 | 885521-92-6 1017540-70-3 | 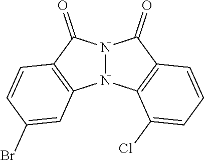 [SMILES: O=c1c2ccc(Br)cc2n2c3c(Cl)cccc3c(=O)n12] | 26% |
| S15 | 7364-27-4 1017540-70-3 | 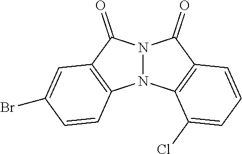 [SMILES: O=c1c2cc(Br)ccc2n2c3c(Cl)cccc3c(=O)n12] | 29% |
| S16 | 864845-15-8 1017540-70-3 | 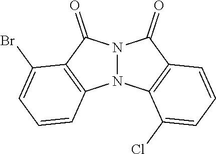 [SMILES: O=c1c2cccc(Cl)c2n2c3cccc(Br)c3c(=O)n12] | 27% |
| S17 | 1500638-10-7 476619-76-8 | 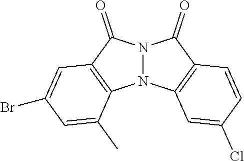 [SMILES: Cc1cc(Br)cc2c(=O)n3c(=O)c4ccc(Cl)cc4n3c12] | 30% |
| S18 | 885518-58-1 171256-81-8 | 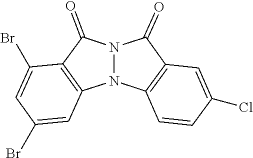 [SMILES: O=c1c2cc(Cl)ccc2n2c3cc(Br)cc(Br)c3c(=O)n12] | 34% |
| S19 | 885518-58-1 476619-76-8 | 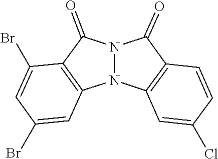 [SMILES: O=c1c2ccc(Cl)cc2n2c3cc(Br)cc(Br)c3c(=O)n12] | 31% |
| S20 | 108961-61-1 171256-81-8 | 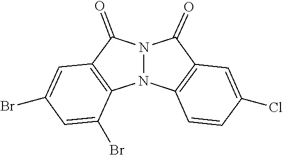 [SMILES: O=c1c2cc(Cl)ccc2n2c3c(Br)cc(Br)cc3c(=O)n12] | 27% |
| S21 | 108961-61-1 476619-76-8 | 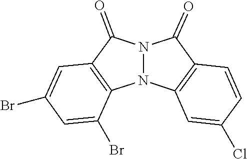 [SMILES: O=c1c2ccc(Cl)cc2n2c3c(Br)cc(Br)cc3c(=O)n12] | 26% |
| S22 | 887568-17-4 171256-81-8 | 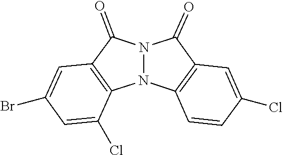 [SMILES: O=c1c2cc(Cl)ccc2n2c3c(Cl)cc(Br)cc3c(=O)n12] | 28% |
| S23 | 887568-17-4 476619-76-8 | 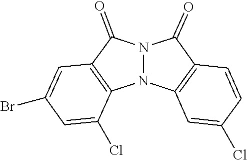 [SMILES: O=c1c2ccc(Cl)cc2n2c3c(Cl)cc(Br)cc3c(=O)n12] | 23% |
| S24 | 82722-04-1 1261850-84-3 | 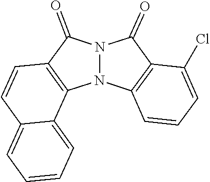 [SMILES: O=c1c2ccc3ccccc3c2n2c3cccc(Cl)c3c(=O)n12] | 30% |
| S25 | 51033-99-9 171256-81-8 | 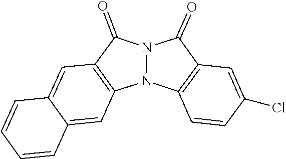 [SMILES: O=c1c2cc(Cl)ccc2n2c3cc4ccccc4cc3c(=O)n12] | 35% |
| S26 | 82722-04-1 476619-76-8 | 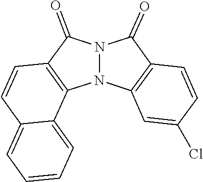 [SMILES: O=c1c2ccc(Cl)cc2n2c3c(ccc4ccccc43)c(=O)n12] | 32% |
| S27 | 33149-32-5 476619-76-8 | 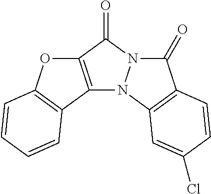 [SMILES: O=c1c2ccc(Cl)cc2n2c3c(oc4ccccc43)c(=O)n12] | 31% |
| S28 | 2379460-30-5 7364-27-4 | 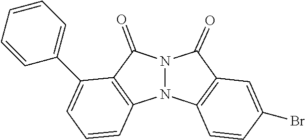 [SMILES: O=c1c2cc(Br)ccc2n2c3cccc(-c4ccccc4)c3c(=O)n12] | 32% |
| S29 | 30726-57-9 864845-15-8 | 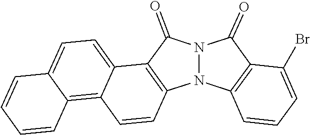 [SMILES: O=c1c2c(Br)cccc2n2c3ccc4c5ccccc5ccc4c3c(=O)n12] | 26% |
| S30 | 2581709-21-7 1500638-10-7 | 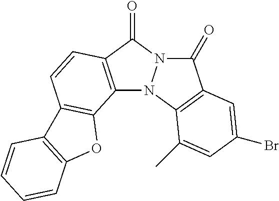 [SMILES: Cc1cc(Br)cc2c(=O)n3c(=O)c4ccc5c6ccccc6oc5c4n3c12] | 29% |
| S31 | 887578-57-6 28384-45-4 | 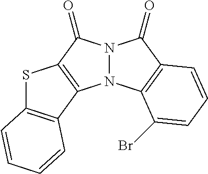 [SMILES: O=c1c2cccc(Br)c2n2c3c(sc4ccccc43)c(=O)n12] | 23% |
| S32 | 864845-15-8 173068-99-0 50 mmol | 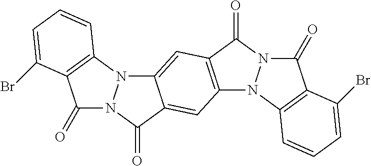 [SMILES: O=c1c2cc3c(cc2n2c4cccc(Br)c4c(=O)n12)c(=O)n1c(=O)c2c(Br)cccc2n31] | 17% |
| S33 | 885518-58-1 2254443-37-1 | 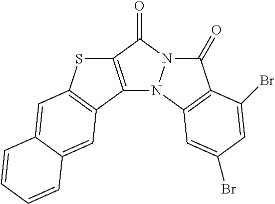 [SMILES: O=c1c2sc3cc4ccccc4cc3c2n2c3cc(Br)cc(Br)c3c(=O)n12] | 33% |
| S34 | 2488802-49-7 885521-92-6 | 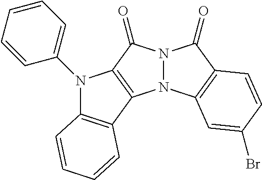 [SMILES: O=c1c2ccc(Br)cc2n2c3c4ccccc4n(-c4ccccc4)c3c(=O)n12] | 32% |
| S35 | 609-67-6 1278518-53-8 | 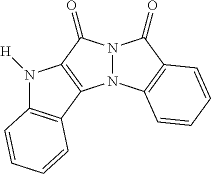 [SMILES: O=c1c2ccccc2n2c3c([nH]c4ccccc43)c(=O)n12] | 21% |
| S36 | 2055586-96-2 50 mmol 609-67-6 | 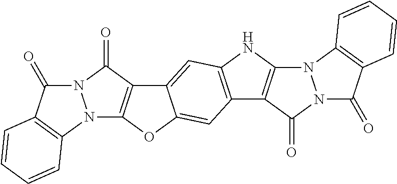 [SMILES: O=c1c2ccccc2n2c3[nH]c4cc5c(cc4c3c(=O)n12)oc1c5c(=O)n2c(=O)c3ccccc3n12] | 16% |
| S37 | 609-67-6 1946843-47-5 | 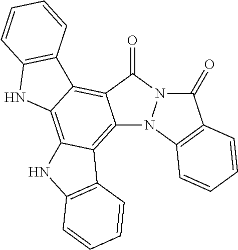 [SMILES: O=c1c2ccccc2n2c3c(c(=O)n12)c1c2ccccc2[nH]c1c1[nH]c2ccccc2c13] | 29% |
| S38 | 2055586-97-3 50 mmol 609-67-6 | 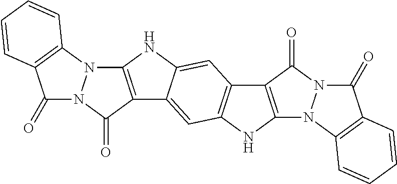 [SMILES: O=c1c2ccccc2n2c3[nH]c4cc5c(cc4c3c(=O)n12)[nH]c1c5c(=O)n2c(=O)c3ccccc3n12] | 16% |
| S39 | 887578-57-6 609-67-6 | 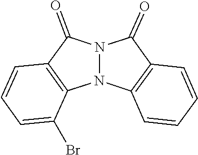 [SMILES: O=c1c2ccccc2n2c3c(Br)cccc3c(=O)n12] | 28% |
| S40 | 885521-92-6 609-67-6 | 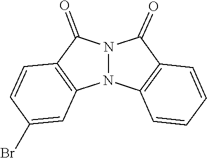 [SMILES: O=c1c2ccccc2n2c3cc(Br)ccc3c(=O)n12] | 36% |
| S41 | 7364-27-4 609-67-6 | 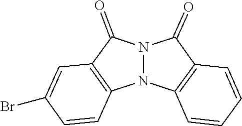 [SMILES: O=c1c2ccccc2n2c3ccc(Br)cc3c(=O)n12] | 35% |
| S42 | 864845-15-8 609-67-6 | 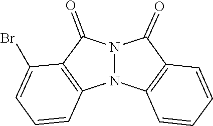 [SMILES: O=c1c2ccccc2n2c3cccc(Br)c3c(=O)n12] | 31% |
| S43 | 108961-61-1 1210022-59-5 | 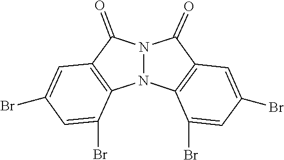 [SMILES: O=c1c2cc(Br)cc(Br)c2n2c3c(Br)cc(Br)cc3c(=O)n12] | 17% |

### Example S50

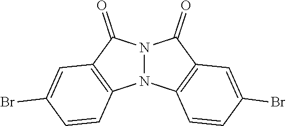 [SMILES: O=c1c2cc(Br)ccc2n2c3ccc(Br)cc3c(=O)n12]

To a well-stirred solution of 23.6 g (100 mmol) of syn-dibenzobimane [125213-40-3] LS1 in 500 ml of DCM is added in portions, in the dark at room temperature, 37.4 g (210 mmol) of N-bromosuccinimide, and then the mixture is stirred for a further 12 h. The reaction mixture is washed three times with 300 ml each time of water and once with 300 ml of saturated sodium chloride solution, and dried over sodium sulfate. The desiccant is filtered off, 100 ml of methanol is added, the filtrate is concentrated to a volume of about 80 ml on a rotary evaporator, and the crystallized product is filtered off with suction and washed twice with a little methanol. Yield: 26.7 g (68 mmol), 68%. Purity: 97% by ¹H NMR.

The following compounds can be prepared analogously:

| Ex. | Reactants | Product | Yield |
|---|---|---|---|
| S51 | LS2 | 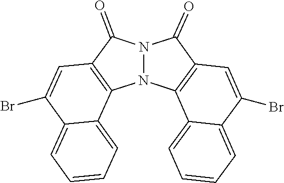 [SMILES: O=c1c2cc(Br)c3ccccc3c2n2c3c(cc(Br)c4ccccc43)c(=O)n12] | 63% |
| S52 | LS3 | 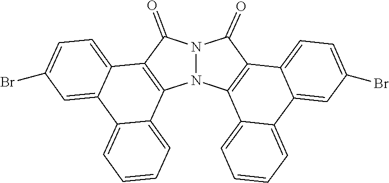 [SMILES: O=c1c2c3ccc(Br)cc3c3ccccc3c2n2c3c4ccccc4c4cc(Br)ccc4c3c(=O)n12] | 37% |
| | | Addition of 1 drop of HBr in glacial acetic acid | |
| | | (47% strength by weight) | |
| S53 | S30 110 mmol NBS | 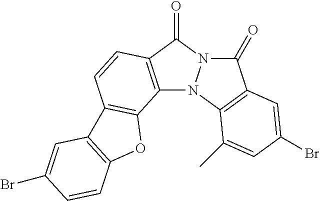 [SMILES: Cc1cc(Br)cc2c(=O)n3c(=O)c4ccc5c6cc(Br)ccc6oc5c4n3c12] | 54% |

### Example Int-D1

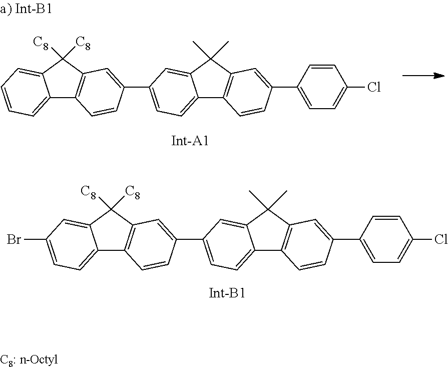

26.0 g (33.7 mmol) of Int-A1 [2171483-83-1] is dissolved in 600 ml of dichloromethane. One drop of HBr (33%, aqueous) is added in the dark, 7.2 g (44.0 mmol) of N-bromosuccinimide is added in portions, and the mixture is stirred at room temperature for 48 h. After 48 h, 200 ml of saturated aqueous sodium hydrogensulfite solution is added, and the mixture is stirred for a further 1 h. Subsequently, the two phases are separated, and the organic phase is washed three times with 200 ml each time of water and then concentrated under reduced pressure. The residue is admixed with 300 ml of n-heptane and heated under reflux for 1 h. After the mixture has been cooled, the colourless solids are filtered off, washed twice with 50 ml each time of n-heptane and dried under reduced pressure. Yield: 25.3 g (32 mmol), 97%. Purity: about 98% by HPLC.

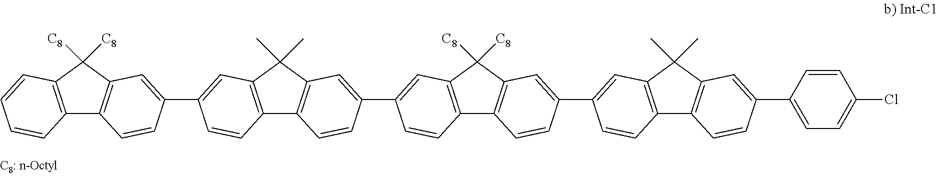

To 28.0 g (35.5 mmol) of Int-B1, 33.6 g (35.5 mmol) of [2171483-74-0] and 9.8 g (71 mmol) of potassium carbonate are added 1100 ml of THE and 550 ml of water. Subsequently, 373 mg (1.4 mmol) of triphenylphosphine and 325 mg (0.4 mmol) of tri(dibenzylideneacetone)dipalladium are added and the mixture is heated under reflux for 16 h. 500 ml of water and 500 ml of toluene are added to the cooled reaction mixture; the phases are separated. The aqueous phase is extracted twice with 200 ml each time of toluene and the combined organic phases are washed once with 500 ml of water. The organic phase is filtered through alumina and then concentrated to dryness. The product is purified by repeated crystallization from toluene/n-heptane 1:10 and obtained in solid form. Yield: 41.0 g (28.8 mmol) 81%. Purity: about 98% by HPLC.

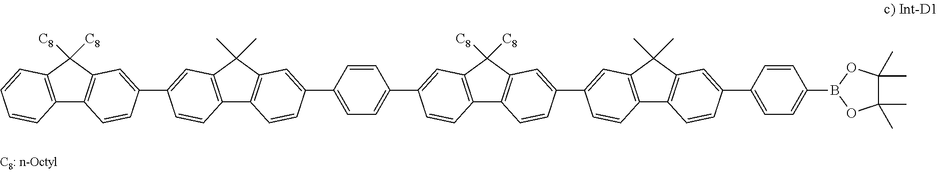

To 25.3 g (18.7 mmol) of Int-C1, 8.6 g (33.7 mmol) of bis(pinacolato)diborane, 5.5 g (56 mmol) of potassium acetate and 830 mg (1.1 mmol) of trans-dichlorobis(tricyclohexylphosphine)palladium(II) are added 750 ml of dioxane, and the mixture is heated under reflux for 48 h. Subsequently, 1000 ml of toluene and 1000 ml of water are added to the reaction mixture, and the phases are separated. The aqueous phase is extracted twice with 200 ml each time of toluene, and the combined organic phases are washed twice with 500 ml each time of water, filtered through alumina and concentrated to dryness. The residue is subjected to extraction by stirring with hot ethanol. Yield: 16.0 g (11.1 mmol), 60%. Purity: about 99% by HPLC.

### Preparation of the Inventive Compounds V:

### Example V1

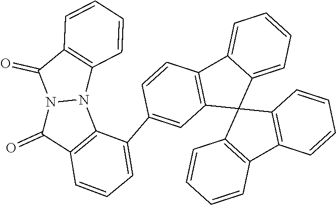 [SMILES: O=c1c2ccccc2n2c3c(-c4ccc5c(c4)C4(c6ccccc6-c6ccccc64)c4ccccc4-5)cccc3c(=O)n12]

A mixture of 27.1 g (100 mmol) of S39, 39.6 g (110 mmol) of 9,9′-spirobifluorene-2-boronic acid [236389-21-2], 21.2 g (100 mmol) of tripotassium phosphate, 1.64 g (4 mmol) of S-Phos, 499 mg (2 mmol) of palladium(II) acetate, 300 ml of toluene, 100 ml dioxane and 300 ml of water is stirred at 70° C. for 12 h (it is alternatively possible to use AmPhosPdCl₂ [887919-35-9]). After cooling, the organic phase is removed and washed twice with 300 ml each time of water and once with 300 ml of saturated sodium chloride solution. The organic phase is concentrated to dryness, the residue is taken up in dichloromethane (DCM) and filtered through a silica gel bed in the form of a DCM slurry, and the filtrate is concentrated on a rotary evaporator, with continuous replacement of the DCM removed by rotary evaporation with methanol. The crystallized product is filtered off with suction, washed twice with 50 ml each time of methanol and dried under reduced pressure. Further purification is effected by hot extraction crystallization (solvent mixtures: DCM/acetonitrile 1:3 to 3:1) and fractional sublimation. Yield: 31.5 g (57 mmol), 57%. Purity: about 99.9% by HPLC.

The following compounds can be pared analogously:

| Ex. | Reactants | Product | Yield |
|---|---|---|---|
| V2 | 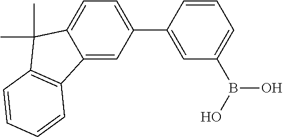 [SMILES: CC1(C)c2ccccc2-c2cc(-c3cccc(B(O)O)c3)ccc21] S40 2363139-27-7 | 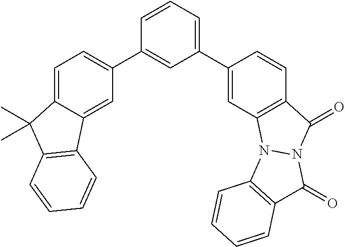 [SMILES: CC1(C)c2ccccc2-c2cc(-c3cccc(-c4ccc5c(=O)n6c(=O)c7ccccc7n6c5c4)c3)ccc21] | 67% |
| V3 | 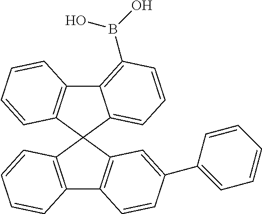 [SMILES: OB(O)c1cccc2c1-c1ccccc1C21c2ccccc2-c2ccc(-c3ccccc3)cc21] S41 2201147-30-8 | 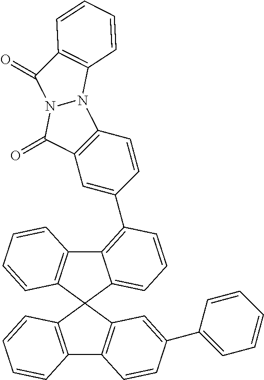 [SMILES: O=c1c2ccccc2n2c3ccc(-c4cccc5c4-c4ccccc4C54c5ccccc5-c5ccc(-c6ccccc6)cc54)cc3c(=O)n12] | 70% |
| V4 | 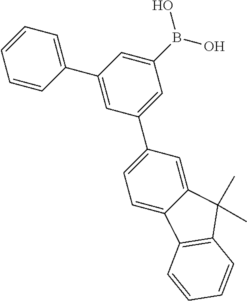 [SMILES: CC1(C)c2ccccc2-c2ccc(-c3cc(B(O)O)cc(-c4ccccc4)c3)cc21] | 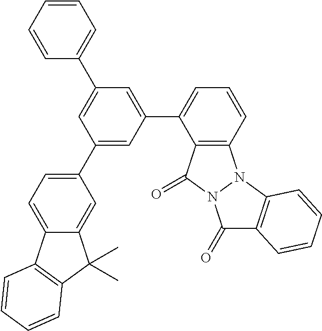 [SMILES: CC1(C)c2ccccc2-c2ccc(-c3cc(-c4ccccc4)cc(-c4cccc5c4c(=O)n4c(=O)c6ccccc6n54)c3)cc21] | 51% |
| | S42 | | |
| | 881911-75-7 | | |
| V5 | 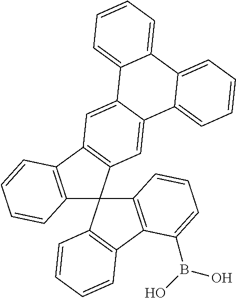 [SMILES: OB(O)c1cccc2c1-c1ccccc1C21c2ccccc2-c2cc3c4ccccc4c4ccccc4c3cc21] S40 2088929-31-9 | 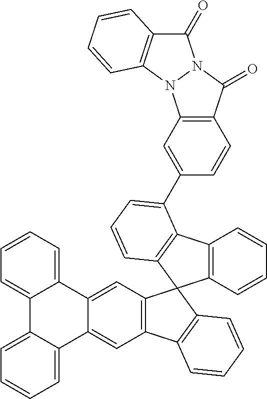 [SMILES: O=c1c2ccccc2n2c3cc(-c4cccc5c4-c4ccccc4C54c5ccccc5-c5cc6c7ccccc7c7ccccc7c6cc54)ccc3c(=O)n12] | 56% |
| V6 | 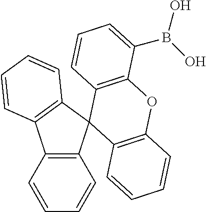 [SMILES: OB(O)c1cccc2c1Oc1ccccc1C21c2ccccc2-c2ccccc21] S41 2418007-78-8 | 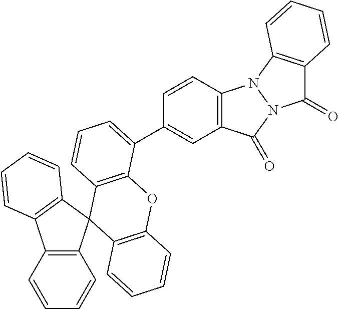 [SMILES: O=c1c2ccccc2n2c3ccc(-c4cccc5c4Oc4ccccc4C54c5ccccc5-c5ccccc54)cc3c(=O)n12] | 65% |
| V7 | 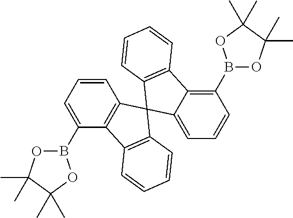 S39 1257321-47-3 50 mmol | 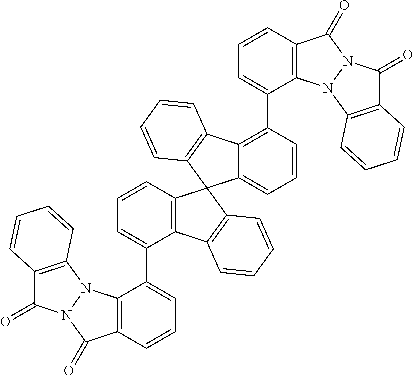 [SMILES: O=c1c2ccccc2n2c3c(-c4cccc5c4-c4ccccc4C54c5ccccc5-c5c(-c6cccc7c(=O)n8c(=O)c9ccccc9n8c67)cccc54)cccc3c(=O)n12] | 43% |
| V8 | 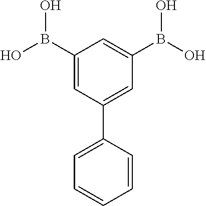 [SMILES: OB(O)c1cc(B(O)O)cc(-c2ccccc2)c1] S42 2249944-12-3 50 mmol | 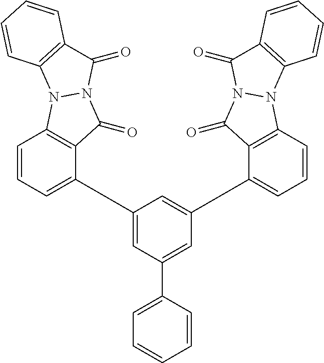 [SMILES: O=c1c2ccccc2n2c3cccc(Cc4cc(Cc5cccc6c5c(=O)n5c(=O)c7ccccc7n65)cc(-c5ccccc5)c4)c3c(=O)n12] | 58% |
| V9 | 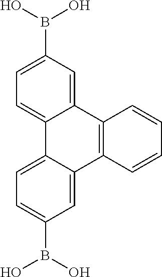 [SMILES: OB(O)c1ccc2c3ccc(B(O)O)cc3c3ccccc3c2c1] S39 1326240-20-3 50 mmol | 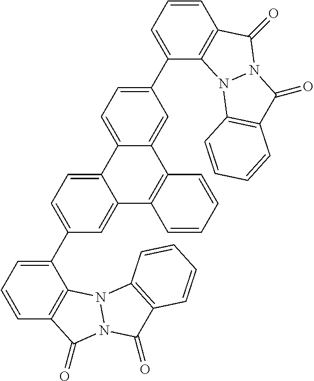 [SMILES: O=c1c2ccccc2n2c3c(-c4ccc5c6ccc(-c7cccc8c(=O)n9c(=O)c%10ccccc%10n9c78)cc6c6ccccc6c5c4)cccc3c(=O)n12] | 61% |
| V10 | 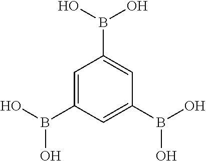 [SMILES: OB(O)c1cc(B(O)O)cc(B(O)O)c1] S41 1216638-44-6 30 mmol | 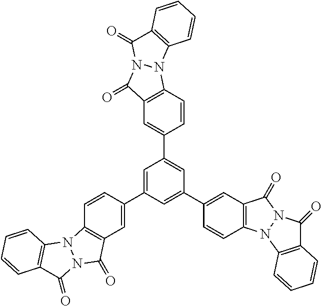 [SMILES: O=c1c2ccccc2n2c3ccc(-c4cc(-c5ccc6c(c5)c(=O)n5c(=O)c7ccccc7n65)cc(-c5ccc6c(c5)c(=O)n5c(=O)c7ccccc7n65)c4)cc3c(=O)n12] | 44% |
| V11 | 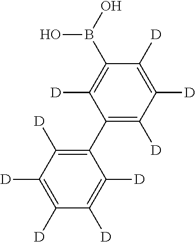 [SMILES: [2H]c1c([2H])c([2H])c(-c2c([2H])c([2H])c([2H])c(B(O)O)c2[2H])c([2H])c1[2H]] S32 2368221-45-6 220 mmol | 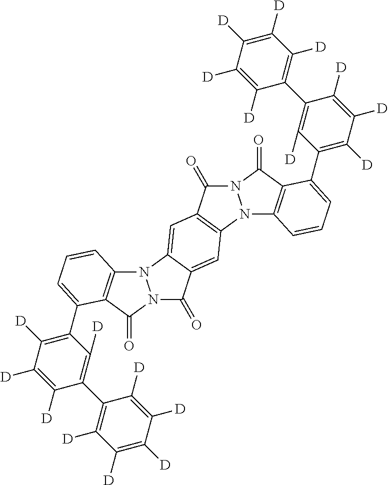 [SMILES: [2H]c1c([2H])c([2H])c(-c2c([2H])c([2H])c([2H])c(-c3cccc4c3c(=O)n3c(=O)c5cc6c(cc5n43)c(=O)n3c(=O)c4c(-c5c([2H])c([2H])c([2H])c(-c7c([2H])c([2H])c([2H])c([2H])c7[2H])c5[2H])cccc4n63)c2[2H])c([2H])c1[2H]] | 40% |
| V12 | 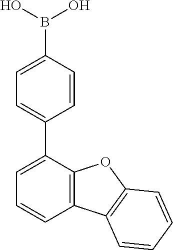 [SMILES: OB(O)c1ccc(-c2cccc3c2oc2ccccc23)cc1] S39 796071-96-0 | 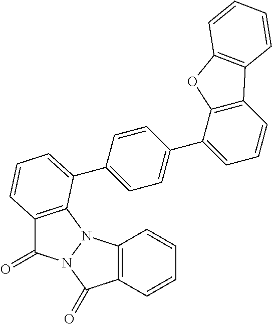 [SMILES: O=c1c2ccccc2n2c3c(-c4ccc(-c5cccc6c5oc5ccccc56)cc4)cccc3c(=O)n12] | 43% |
| V13 | 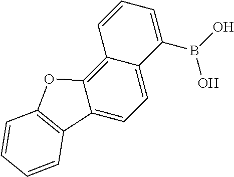 [SMILES: OB(O)c1cccc2c1ccc1c3ccccc3oc21] S40 | 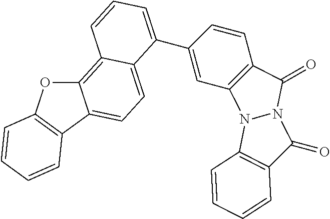 [SMILES: O=c1c2ccccc2n2c3cc(-c4cccc5c4ccc4c6ccccc6oc54)ccc3c(=O)n12] | 69% |
| | 2345673-16-5 | | |
| V14 | 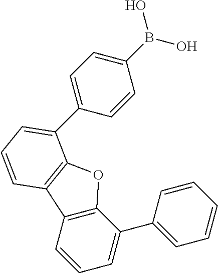 [SMILES: OB(O)c1ccc(-c2cccc3c2oc2c(-c4ccccc4)cccc23)cc1] S41 2247722-85-4 | 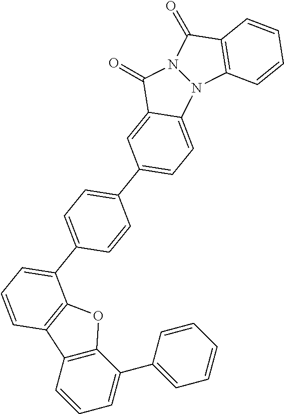 [SMILES: O=c1c2ccccc2n2c3ccc(-c4ccc(-c5cccc6c5oc5c(-c7ccccc7)cccc56)cc4)cc3c(=O)n12] | 65% |
| V15 | 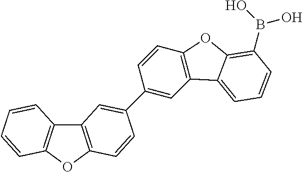 [SMILES: OB(O)c1cccc2c1oc1ccc(-c3ccc4oc5ccccc5c4c3)cc12] S42 2411326-31-1 | 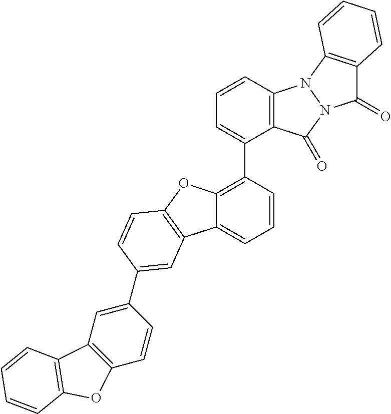 [SMILES: O=c1c2ccccc2n2c3cccc(-c4cccc5c4oc4ccc(-c6ccc7oc8ccccc8c7c6)cc45)c3c(=O)n12] | 56% |
| V16 | 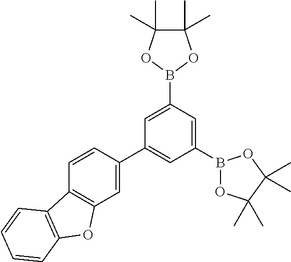 [SMILES: CC1(C)OB(c2cc(B3OC(C)(C)C(C)(C)O3)cc(-c3ccc4c(c3)oc3ccccc34)c2)OC1(C)C] S42 | 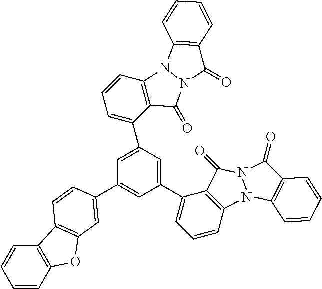 [SMILES: O=c1c2ccccc2n2c3cccc(-c4cc(-c5ccc6c(c5)oc5ccccc56)cc(-c5cccc6c5c(=O)n5c(=O)c7ccccc7n65)c4)c3c(=O)n12] | 47% |
| | 2375083-92-2 | | |
| | 50 mmol | | |
| V17 | 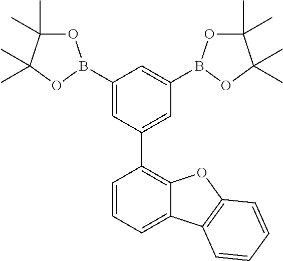 | 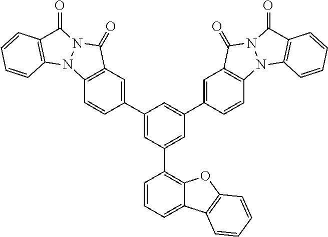 [SMILES: O=c1c2ccccc2n2c3ccc(-c4cc(-c5ccc6c(c5)c(=O)n5c(=O)c7ccccc7n65)cc(-c5cccc6c5oc5ccccc56)c4)cc3c(=O)n12] | 61% |
| | S41 | | |
| | 2375083-93-3 | | |
| | 50 mmol | | |
| V18 | 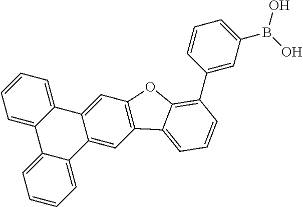 [SMILES: OB(O)c1cccc(-c2cccc3c2oc2cc4c5ccccc5c5ccccc5c4cc23)c1] | 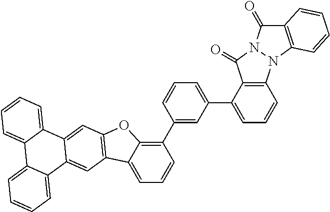 [SMILES: O=c1c2ccccc2n2c3cccc(-c4cccc(-c5cccc6c5oc5cc7c8ccccc8c8ccccc8c7cc56)c4)c3c(=O)n12] | 53% |
| | S42 | | |
| | 1644606-71-2 | | |
| V19 | 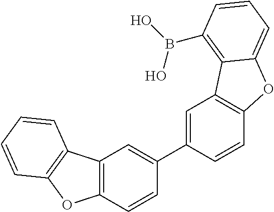 [SMILES: OB(O)c1cccc2oc3ccc(-c4ccc5oc6ccccc6c5c4)cc3c12] S40 1822320-55-7 | 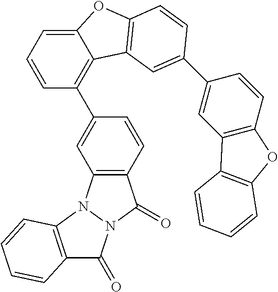 [SMILES: O=c1c2ccccc2n2c3cc(-c4cccc5oc6ccc(-c7ccc8oc9ccccc9c8c7)cc6c45)ccc3c(=O)n12] | 67% |
| V20 | 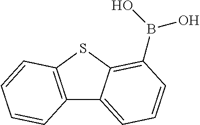 [SMILES: OB(O)c1cccc2c1sc1ccccc12] S33 108847-20-7 210 mmol | 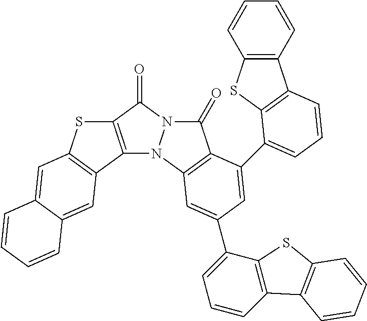 [SMILES: O=c1c2sc3cc4ccccc4cc3c2n2c3cc(-c4cccc5c4sc4ccccc45)cc(-c4cccc5c4sc4ccccc45)c3c(=O)n12] | 51% |
| V21 | 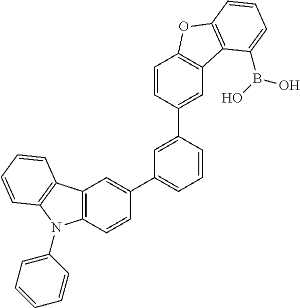 [SMILES: OB(O)c1cccc2oc3ccc(-c4cccc(-c5ccc6c(c5)c5ccccc5n6-c5ccccc5)c4)cc3c12] S41 | 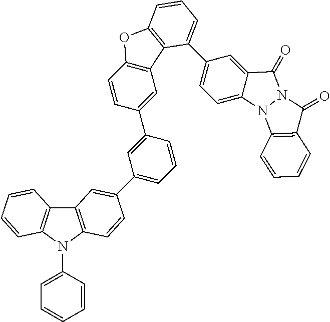 [SMILES: O=c1c2ccccc2n2c3ccc(-c4cccc5oc6ccc(-c7cccc(-c8ccc9c(c8)c8ccccc8n9-c8ccccc8)c7)cc6c45)cc3c(=O)n12] | 70% |
| | 1822311-37-4 | | |
| V22 | 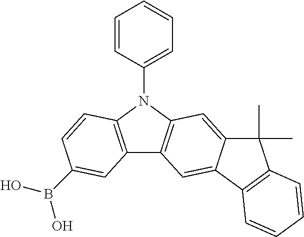 [SMILES: CC1(C)c2ccccc2-c2cc3c4cc(B(O)O)ccc4n(-c4ccccc4)c3cc21] S39 | 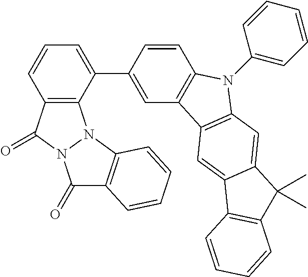 [SMILES: CC1(C)c2ccccc2-c2cc3c4cc(-c5cccc6c(=O)n7c(=O)c8ccccc8n7c56)ccc4n(-c4ccccc4)c3cc21] | 44% |
| | 1379585-25-7 | | |
| V23 | 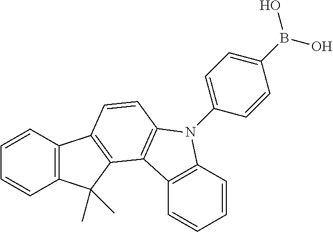 [SMILES: CC1(C)c2ccccc2-c2ccc3c(c21)c1ccccc1n3-c1ccc(B(O)O)cc1] S41 2241229-08-1 | 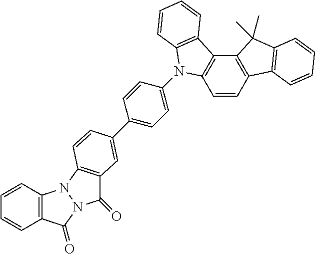 [SMILES: CC1(C)c2ccccc2-c2ccc3c(c21)c1ccccc1n3-c1ccc(-c2ccc3c(c2)c(=O)n2c(=O)c4ccccc4n32)cc1] | 69% |
| V24 | 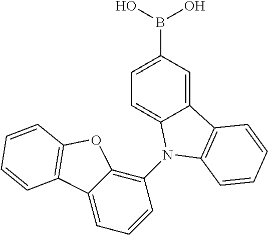 [SMILES: OB(O)c1ccc2c(c1)c1ccccc1n2-c1cccc2c1oc1ccccc12] S41 1547397-15-8 | 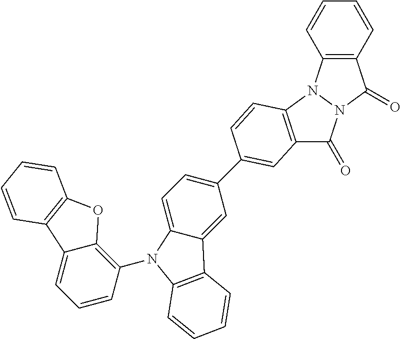 [SMILES: O=c1c2ccccc2n2c3ccc(-c4ccc5c(c4)c4ccccc4n5-c4cccc5c4oc4ccccc45)cc3c(=O)n12] | 67% |
| V25 | 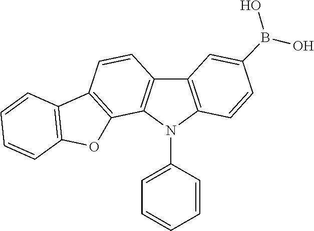 [SMILES: OB(O)c1ccc2c(c1)c1ccc3c4ccccc4oc3c1n2-c1ccccc1] S42 2128290-87-7 | 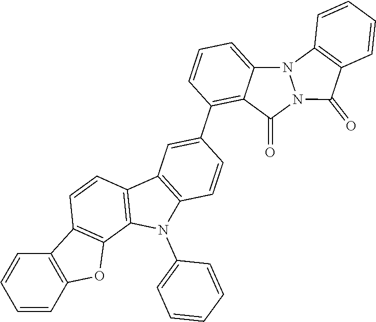 [SMILES: O=c1c2ccccc2n2c3cccc(-c4ccc5c(c4)c4ccc6c7ccccc7oc6c4n5-c4ccccc4)c3c(=O)n12] | 55% |
| V26 | 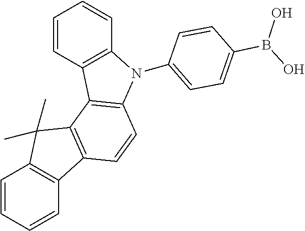 [SMILES: CC1(C)c2ccccc2-c2ccc3c(c21)c1ccccc1n3-c1ccc(B(O)O)cc1] S25 2241229-08-1 | 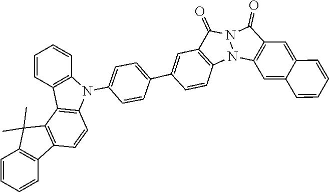 [SMILES: CC1(C)c2ccccc2-c2ccc3c(c21)c1ccccc1n3-c1ccc(-c2ccc3c(c2)c(=O)n2c(=O)c4cc5ccccc5cc4n32)cc1] | 67% |
| V27 | 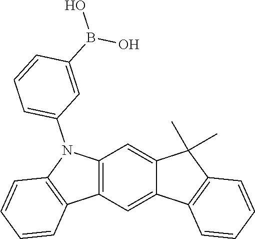 [SMILES: CC1(C)c2ccccc2-c2cc3c4ccccc4n(-c4cccc(B(O)O)c4)c3cc21] S28 1369587-64-3 | 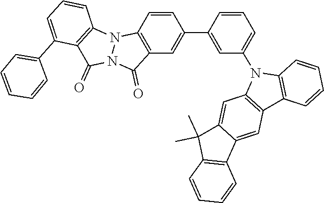 [SMILES: CC1(C)c2ccccc2-c2cc3c4ccccc4n(-c4cccc(-c5ccc6c(c5)c(=O)n5c(=O)c7c(-c8ccccc8)cccc7n65)c4)c3cc21] | 66% |
| V28 | 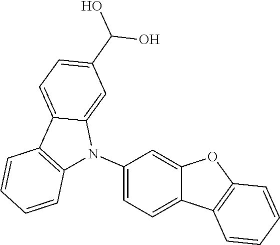 [SMILES: OC(O)c1ccc2c3ccccc3n(-c3ccc4c(c3)oc3ccccc34)c2c1] S30 1637323-05-7 | 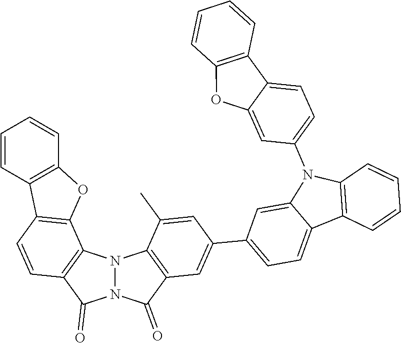 [SMILES: Cc1cc(-c2ccc3c4ccccc4n(-c4ccc5c(c4)oc4ccccc45)c3c2)cc2c(=O)n3c(=O)c4ccc5c6ccccc6oc5c4n3c12] | 53% |
| V29 | 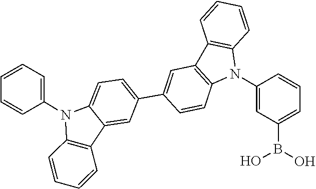 [SMILES: OB(O)c1cccc(-n2c3ccccc3c3cc(-c4ccc5c(c4)c4ccccc4n5-c4ccccc4)ccc32)c1] S34 1398394-64-3 | 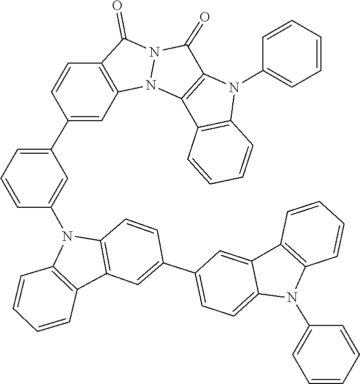 [SMILES: O=c1c2ccc(-c3cccc(-n4c5ccccc5c5cc(-c6ccc7c(c6)c6ccccc6n7-c6ccccc6)ccc54)c3)cc2n2c3c4ccccc4n(-c4ccccc4)c3c(=O)n12] | 72% |
| V30 | 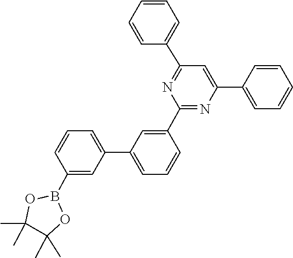 [SMILES: CC1(C)OB(c2cccc(-c3cccc(-c4nc(-c5ccccc5)cc(-c5ccccc5)n4)c3)c2)OC1(C)C] S42 1802233-05-1 | 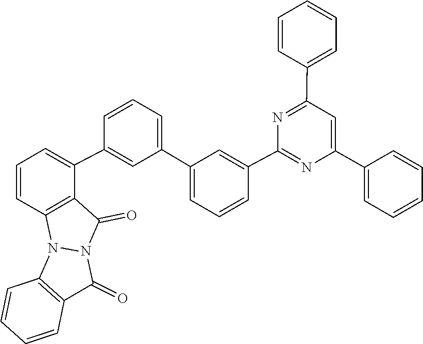 [SMILES: O=c1c2ccccc2n2c3cccc(-c4cccc(-c5cccc(-c6nc(-c7ccccc7)cc(-c7ccccc7)n6)c5)c4)c3c(=O)n12] | 56% |
| V31 | 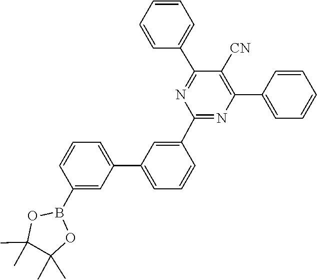 [SMILES: CC1(C)OB(c2cccc(-c3cccc(-c4nc(-c5ccccc5)c(C#N)c(-c5ccccc5)n4)c3)c2)OC1(C)C] S40 2319558-93-3 | 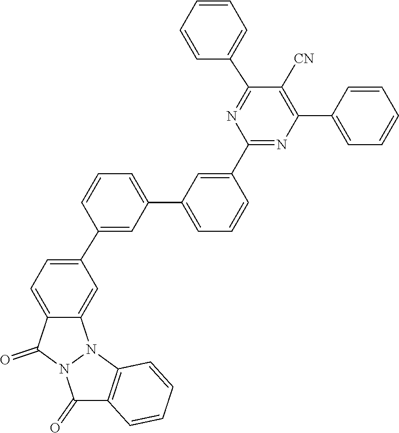 [SMILES: N#Cc1c(-c2ccccc2)nc(-c2cccc(-c3cccc(-c4ccc5c(=O)n6c(=O)c7ccccc7n6c5c4)c3)c2)nc1-c1ccccc1] | 68% |
| V32 | 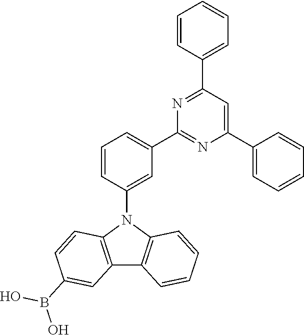 [SMILES: OB(O)c1ccc2c(c1)c1ccccc1n2-c1cccc(-c2nc(-c3ccccc3)cc(-c3ccccc3)n2)c1] S41 1813537-09-5 | 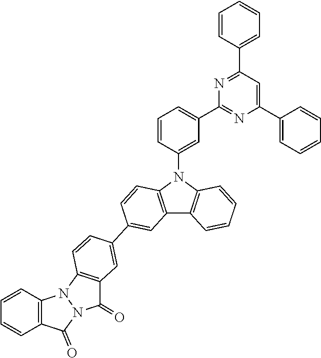 [SMILES: O=c1c2ccccc2n2c3ccc(-c4ccc5c(c4)c4ccccc4n5-c4cccc(-c5nc(-c6ccccc6)cc(-c6ccccc6)n5)c4)cc3c(=O)n12] | 64% |
| V33 | 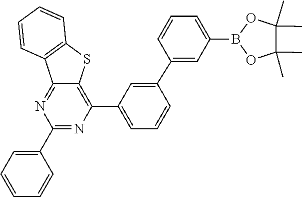 [SMILES: CC1(C)OB(c2cccc(-c3cccc(-c4nc(-c5ccccc5)nc5c4sc4ccccc45)c3)c2)OC1(C)C] S39 1801325-80-3 | 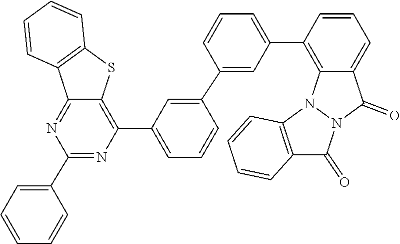 [SMILES: O=c1c2ccccc2n2c3c(-c4cccc(-c5cccc(-c6nc(-c7ccccc7)nc7c6sc6ccccc67)c5)c4)cccc3c(=O)n12] | 47% |
| V34 | 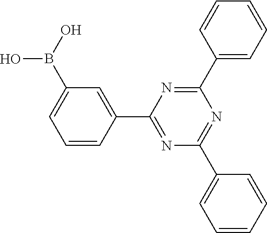 [SMILES: OB(O)c1cccc(-c2nc(-c3ccccc3)nc(-c3ccccc3)n2)c1] | 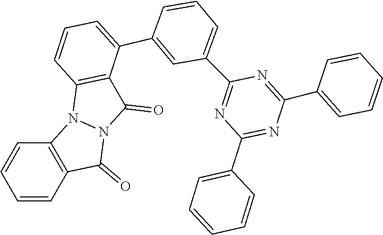 [SMILES: O=c1c2ccccc2n2c3cccc(-c4cccc(-c5nc(-c6ccccc6)nc(-c6ccccc6)n5)c4)c3c(=O)n12] | 54% |
| | S42 | | |
| | 1612243-82-9 | | |
| V35 | 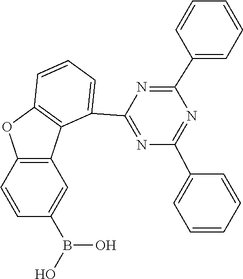 [SMILES: OB(O)c1ccc2oc3cccc(-c4nc(-c5ccccc5)nc(-c5ccccc5)n4)c3c2c1] | 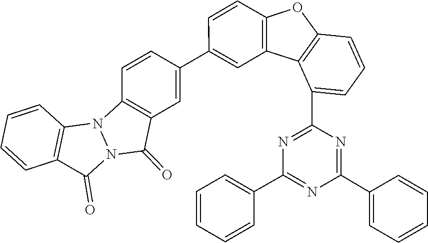 [SMILES: O=c1c2ccccc2n2c3ccc(-c4ccc5oc6cccc(-c7nc(-c8ccccc8)nc(-c8ccccc8)n7)c6c5c4)cc3c(=O)n12] | 69% |
| | S41 | | |
| | 2138490-96-5 | | |
| V36 | 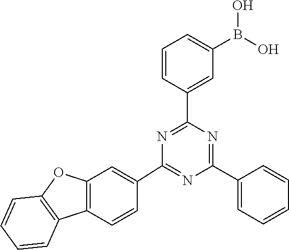 [SMILES: OB(O)c1cccc(-c2nc(-c3ccccc3)nc(-c3ccc4c(c3)oc3ccccc34)n2)c1] S42 2305626-41-7 | 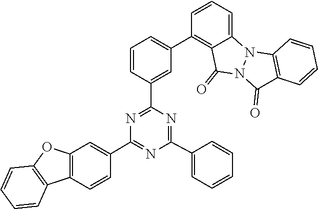 [SMILES: O=c1c2ccccc2n2c3cccc(-c4cccc(-c5nc(-c6ccccc6)nc(-c6ccc7c(c6)oc6ccccc67)n5)c4)c3c(=O)n12] | 56% |
| V37 | 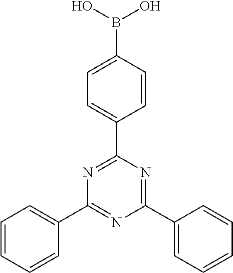 [SMILES: OB(O)c1ccc(-c2nc(-c3ccccc3)nc(-c3ccccc3)n2)cc1] | 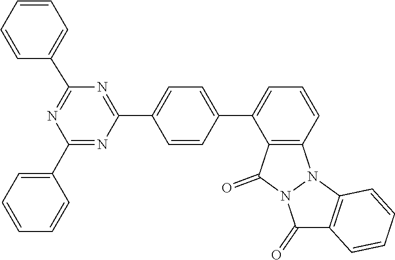 [SMILES: O=c1c2ccccc2n2c3cccc(-c4ccc(-c5nc(-c6ccccc6)nc(-c6ccccc6)n5)cc4)c3c(=O)n12] | 55% |
| | S42 | | |
| | 1313018-07-3 | | |
| V38 | 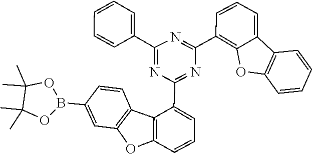 [SMILES: CC1(C)OB(c2ccc3c(c2)oc2cccc(-c4nc(-c5ccccc5)nc(-c5cccc6c5oc5ccccc56)n4)c23)OC1(C)C] S40 2268674-03-7 | 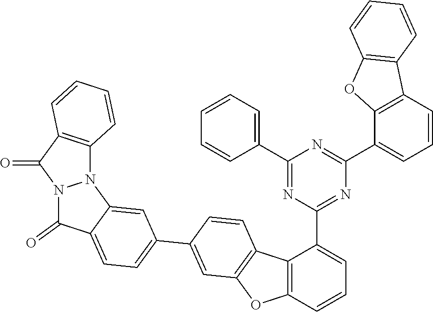 [SMILES: O=c1c2ccccc2n2c3cc(-c4ccc5c(c4)oc4cccc(-c6nc(-c7ccccc7)nc(-c7cccc8c7oc7ccccc78)n6)c45)ccc3c(=O)n12] | 71% |
| V39 | 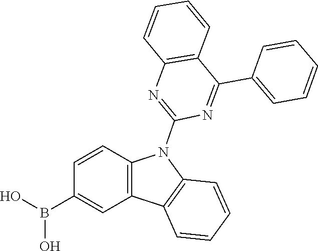 [SMILES: OB(O)c1ccc2c(c1)c1ccccc1n2-c1nc(-c2ccccc2)c2ccccc2n1] S34 1642121-58-1 | 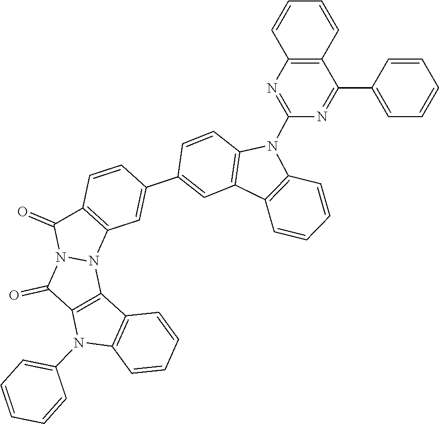 [SMILES: O=c1c2ccc(-c3ccc4c(c3)c3ccccc3n4-c3nc(-c4ccccc4)c4ccccc4n3)cc2n2c3c4ccccc4n(-c4ccccc4)c3c(=O)n12] | 71% |
| V40 | 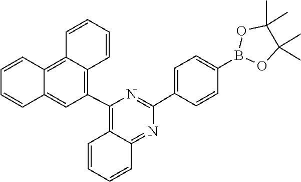 [SMILES: CC1(C)OB(c2ccc(-c3nc(-c4cc5ccccc5c5ccccc45)c4ccccc4n3)cc2)OC1(C)C] S24 1637377-61-7 | 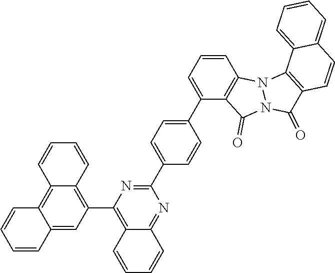 [SMILES: O=c1c2ccc3ccccc3c2n2c3cccc(-c4ccc(-c5nc(-c6cc7ccccc7c7ccccc67)c6ccccc6n5)cc4)c3c(=O)n12] | 56% |
| V41 | 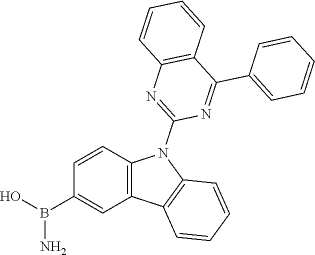 [SMILES: NB(O)c1ccc2c(c1)c1ccccc1n2-c1nc(-c2ccccc2)c2ccccc2n1] | 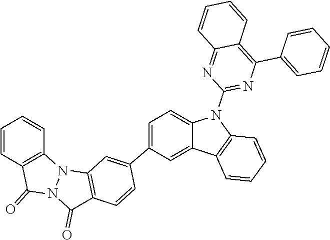 [SMILES: O=c1c2ccccc2n2c3cc(-c4ccc5c(c4)c4ccccc4n5-c4nc(-c5ccccc5)c5ccccc5n4)ccc3c(=O)n12] | 73% |
| | S40 | | |
| | 1642121-58-1 | | |
| V42 | 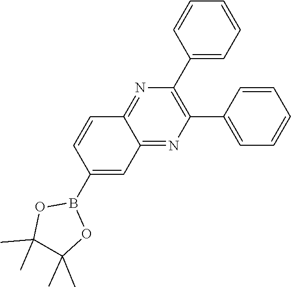 [SMILES: CC1(C)OB(c2ccc3nc(-c4ccccc4)c(-c4ccccc4)nc3c2)OC1(C)C] | 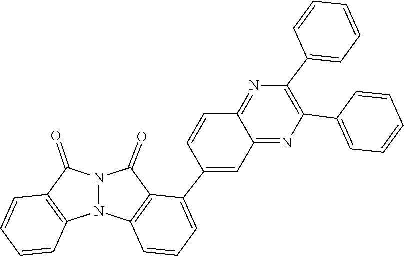 [SMILES: O=c1c2ccccc2n2c3cccc(-c4ccc5nc(-c6ccccc6)c(-c6ccccc6)nc5c4)c3c(=O)n12] | 57% |
| | S42 | | |
| | 1133318-06-5 | | |
| V43 | 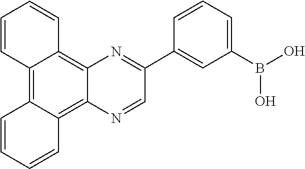 [SMILES: OB(O)c1cccc(-c2cnc3c4ccccc4c4ccccc4c3n2)c1] S40 1609021-60-4 | 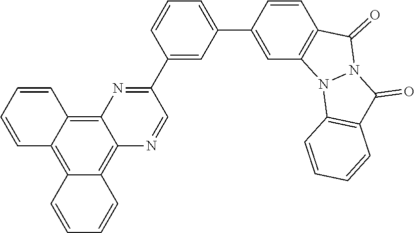 [SMILES: O=c1c2ccccc2n2c3cc(-c4cccc(-c5cnc6c7ccccc7c7ccccc7c6n5)c4)ccc3c(=O)n12] | 70% |
| V44 | 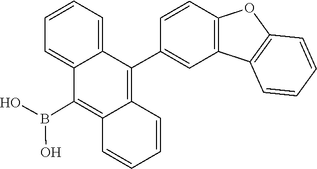 [SMILES: OB(O)c1c2ccccc2c(-c2ccc3oc4ccccc4c3c2)c2ccccc12] S42 1188095-78-4 | 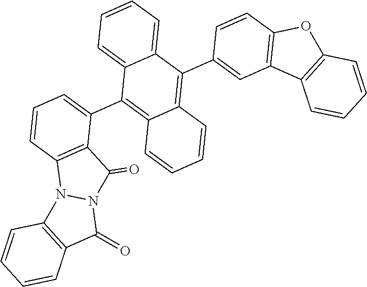 [SMILES: O=c1c2ccccc2n2c3cccc(-c4c5ccccc5c(-c5ccc6oc7ccccc7c6c5)c5ccccc45)c3c(=O)n12] | 52% |
| V45 | 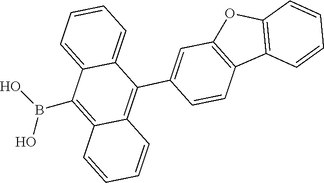 [SMILES: OB(O)c1c2ccccc2c(-c2ccc3c(c2)oc2ccccc23)c2ccccc12] S41 2241542-34-5 | 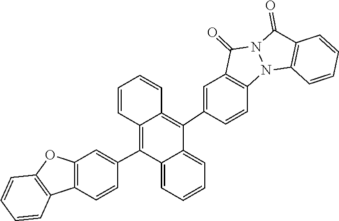 [SMILES: O=c1c2ccccc2n2c3ccc(-c4c5ccccc5c(-c5ccc6c(c5)oc5ccccc56)c5ccccc45)cc3c(=O)n12] | 67% |
| V46 | 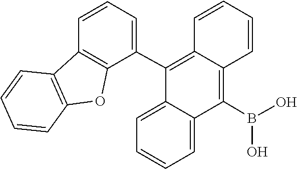 [SMILES: OB(O)c1c2ccccc2c(-c2cccc3c2oc2ccccc23)c2ccccc12] S42 1304130-07-1 | 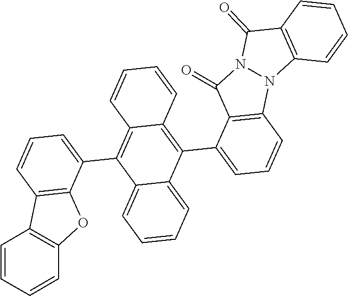 [SMILES: O=c1c2ccccc2n2c3cccc(-c4c5ccccc5c(-c5cccc6c5oc5ccccc56)c5ccccc45)c3c(=O)n12] | 55% |
| V47 | 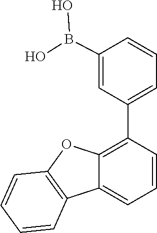 [SMILES: OB(O)c1cccc(-c2cccc3c2oc2ccccc23)c1] | 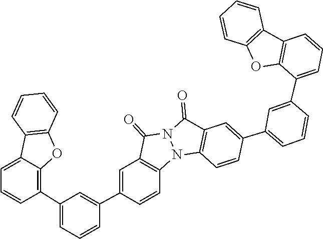 [SMILES: O=c1c2cc(-c3cccc(-c4cccc5c4oc4ccccc45)c3)ccc2n2c3ccc(-c4cccc(-c5cccc6c5oc5ccccc56)c4)cc3c(=O)n12] | 58% |
| | S7 | | |
| | 50 mmol | | |
| | 1271726-52-3 | | |
| V48 | 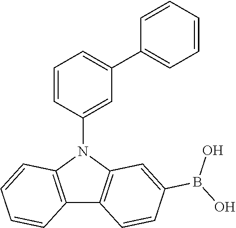 [SMILES: OB(O)c1ccc2c3ccccc3n(-c3cccc(-c4ccccc4)c3)c2c1] S6 50 mmol 1835251-21-2 | 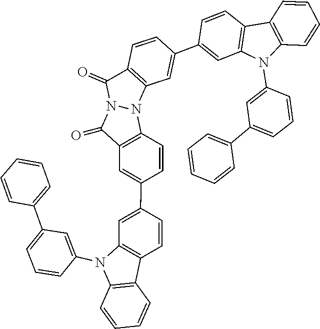 [SMILES: O=c1c2cc(-c3ccc4c5ccccc5n(-c5cccc(-c6ccccc6)c5)c4c3)ccc2n2c3cc(-c4ccc5c6ccccc6n(-c6cccc(-c7ccccc7)c6)c5c4)ccc3c(=O)n12] | 50% |
| V49 | 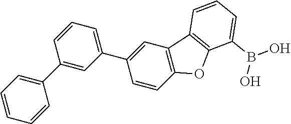 [SMILES: OB(O)c1cccc2c1oc1ccc(-c3cccc(-c4ccccc4)c3)cc12] S10 50 mmol 2305718-67-4 | 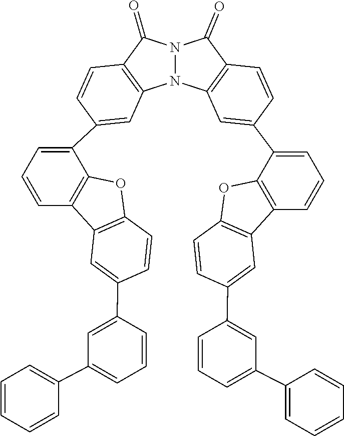 [SMILES: O=c1c2ccc(-c3cccc4c3oc3ccc(-c5cccc(-c6ccccc6)c5)cc34)cc2n2c3cc(-c4cccc5c4oc4ccc(-c6cccc(-c7ccccc7)c6)cc45)ccc3c(=O)n12] | 48% |
| V50 | 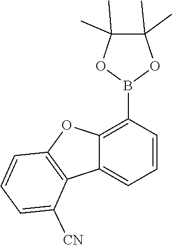 [SMILES: CC1(C)OB(c2cccc3c2oc2cccc(C#N)c23)OC1(C)C] S12 50 mmol 2458817-52-0 | 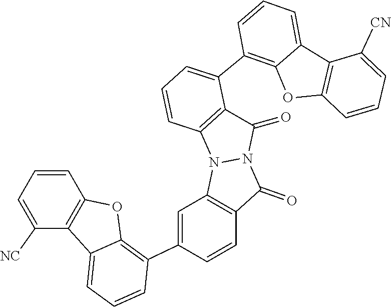 [SMILES: N#Cc1cccc2oc3c(-c4ccc5c(=O)n6c(=O)c7c(-c8cccc9c8oc8cccc(C#N)c89)cccc7n6c5c4)cccc3c12] | 53% |
| V51 | 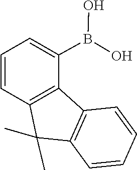 [SMILES: CC1(C)c2ccccc2-c2c(B(O)O)cccc21] S18 30 mmol 1246022-50-3 | 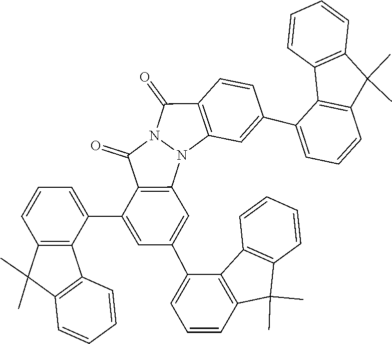 | 46% |
| V52 | 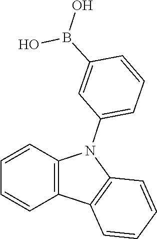 [SMILES: OB(O)c1cccc(-n2c3ccccc3c3ccccc32)c1] S23 30 mmol 864377-33-3 | 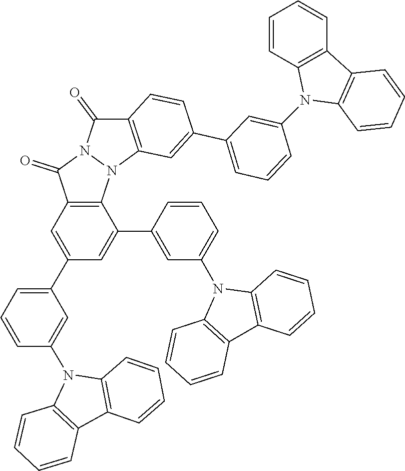 [SMILES: O=c1c2ccc(-c3cccc(-n4c5ccccc5c5ccccc54)c3)cc2n2c3c(-c4cccc(-n5c6ccccc6c6ccccc65)c4)cc(-c4cccc(-n5c6ccccc6c6ccccc65)c4)cc3c(=O)n12] | 39% |
| V53 | 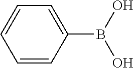 S50 50 mmol 98-80-6 | 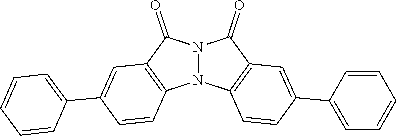 [SMILES: O=c1c2cc(-c3ccccc3)ccc2n2c3ccc(-c4ccccc4)cc3c(=O)n12] | 70% |
| V54 | 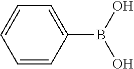 S51 50 mmol 98-80-6 | 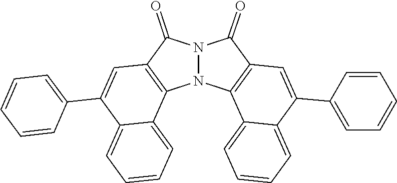 [SMILES: O=c1c2cc(-c3ccccc3)c3ccccc3c2n2c3c(cc(-c4ccccc4)c4ccccc43)c(=O)n12] | 67% |
| V55 | 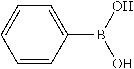 S52 50 mmol 98-80-6 | 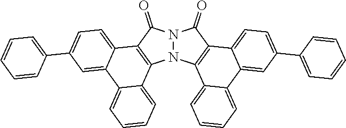 [SMILES: O=c1c2c3ccc(-c4ccccc4)cc3c3ccccc3c2n2c3c4ccccc4c4cc(-c5ccccc5)ccc4c3c(=O)n12] | 74% |
| V56 | 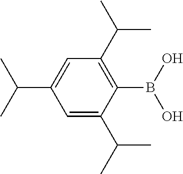 [SMILES: CC(C)c1cc(C(C)C)c(B(O)O)c(C(C)C)c1] | 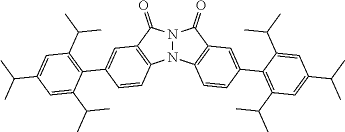 [SMILES: CC(C)c1cc(C(C)C)c(-c2ccc3c(c2)c(=O)n2c(=O)c4cc(-c5c(C(C)C)cc(C(C)C)cc5C(C)C)ccc4n32)c(C(C)C)c1] | 46% |
| | S50 | | |
| | 50 mmol | | |
| | 154549-38-9 | | |
| V57 | 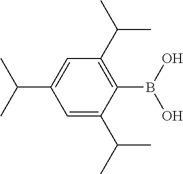 [SMILES: CC(C)c1cc(C(C)C)c(B(O)O)c(C(C)C)c1] S51 50 mmol 154549-38-9 | 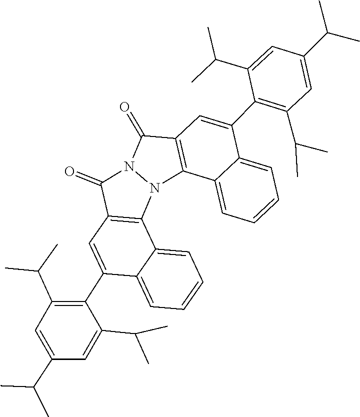 [SMILES: CC(C)c1cc(C(C)C)c(-c2cc3c(=O)n4c(=O)c5cc(-c6c(C(C)C)cc(C(C)C)cc6C(C)C)c6ccccc6c5n4c3c3ccccc23)c(C(C)C)c1] | 43% |
| V58 | 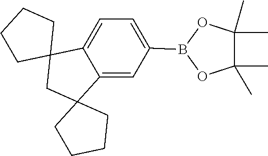 [SMILES: CC1(C)OB(c2ccc3c(c2)C2(CCCC2)CC32CCCC2)OC1(C)C] S50 50 mmol 1912467-76-5 | 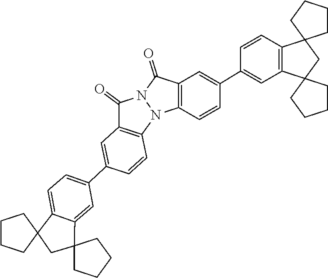 [SMILES: O=c1c2cc(-c3ccc4c(c3)C3(CCCC3)CC43CCCC3)ccc2n2c3ccc(-c4ccc5c(c4)C4(CCCC4)CC54CCCC4)cc3c(=O)n12] | 67% |
| V59 | 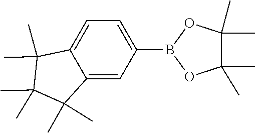 S51 50 mmol 1562418-16-9 | 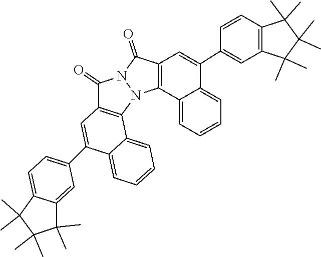 | 59% |
| V60 | 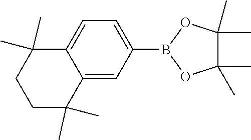 S27 169126-63-0 | 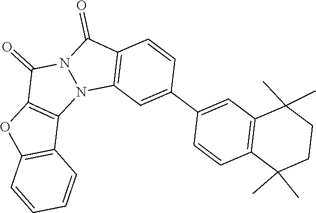 [SMILES: CC1(C)CCC(C)(C)c2cc(-c3ccc4c(=O)n5c(=O)c6oc7ccccc7c6n5c4c3)ccc21] | 60% |
| V61 | 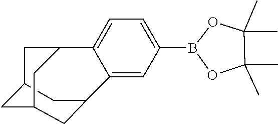 [SMILES: CC1(C)OB(c2ccc3c(c2)C2CC4CC(CC3C4)C2)OC1(C)C] S28 1801624-63-4 | 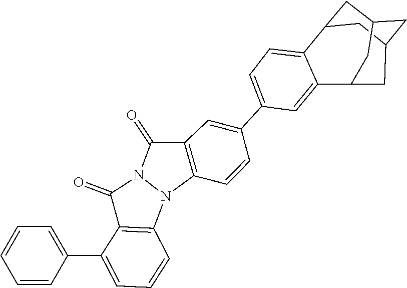 [SMILES: O=c1c2cc(-c3ccc4c(c3)C3CC5CC(CC4C5)C3)ccc2n2c3cccc(-c4ccccc4)c3c(=O)n12] | 65% |
| V62 | 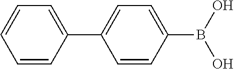 [SMILES: OB(O)c1ccc(-c2ccccc2)cc1] S50 50 mmol 5122-94-1 | 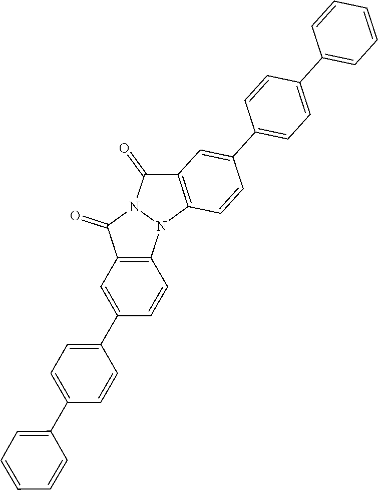 [SMILES: O=c1c2cc(-c3ccc(-c4ccccc4)cc3)ccc2n2c3ccc(-c4ccc(-c5ccccc5)cc4)cc3c(=O)n12] | 57% |
| V63 | 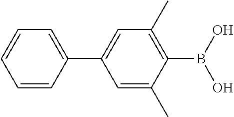 [SMILES: Cc1cc(-c2ccccc2)cc(C)c1B(O)O] S50 50 mmol 1057654-43-9 | 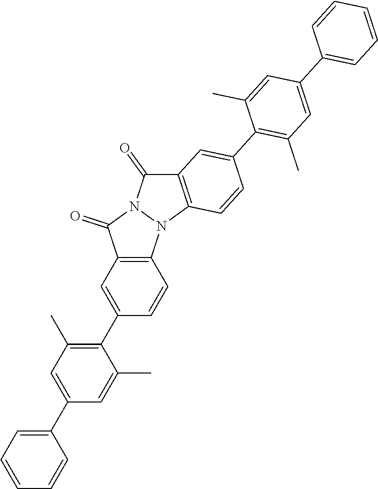 [SMILES: Cc1cc(-c2ccccc2)cc(C)c1-c1ccc2c(c1)c(=O)n1c(=O)c3cc(-c4c(C)cc(-c5ccccc5)cc4C)ccc3n21] | 53% |
| V64 | 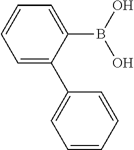 [SMILES: OB(O)c1ccccc1-c1ccccc1] S50 50 mmol 4688-76-0 | 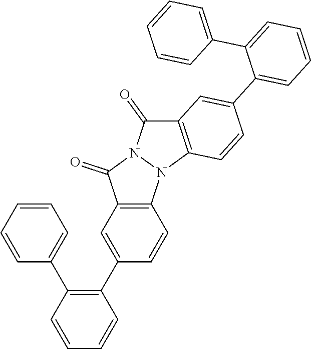 [SMILES: O=c1c2cc(-c3ccccc3-c3ccccc3)ccc2n2c3ccc(-c4ccccc4-c4ccccc4)cc3c(=O)n12] | 51% |
| V65 | 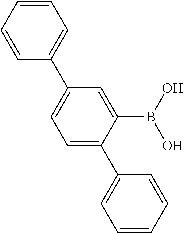 [SMILES: OB(O)c1cc(-c2ccccc2)ccc1-c1ccccc1] S50 50 mmol 1205748-34-0 | 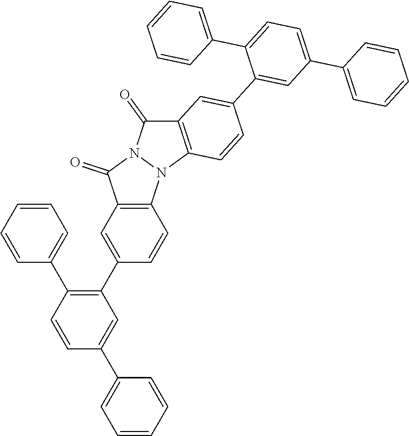 [SMILES: O=c1c2cc(-c3cc(-c4ccccc4)ccc3-c3ccccc3)ccc2n2c3ccc(-c4cc(-c5ccccc5)ccc4-c4ccccc4)cc3c(=O)n12] | 62% |
| V66 | 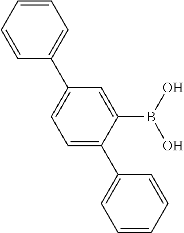 [SMILES: OB(O)c1cc(-c2ccccc2)ccc1-c1ccccc1] S51 50 mmol 1602576-91-9 | 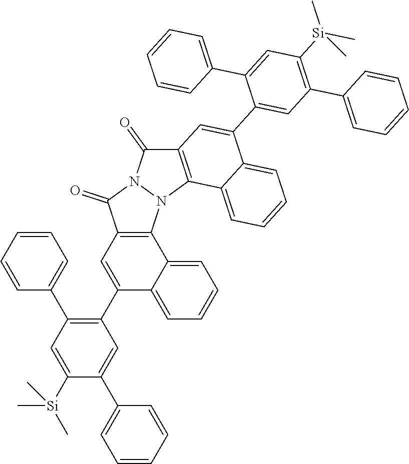 [SMILES: C[Si](C)(C)c1cc(-c2ccccc2)c(-c2cc3c(=O)n4c(=O)c5cc(-c6cc(-c7ccccc7)c([Si](C)(C)C)cc6-c6ccccc6)c6ccccc6c5n4c3c3ccccc23)cc1-c1ccccc1] | 41% |
| V67 | 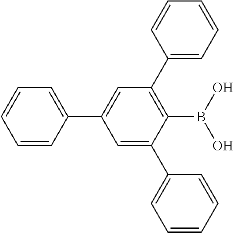 [SMILES: OB(O)c1c(-c2ccccc2)cc(-c2ccccc2)cc1-c1ccccc1] S50 50 mmol 1004321-87-2 | 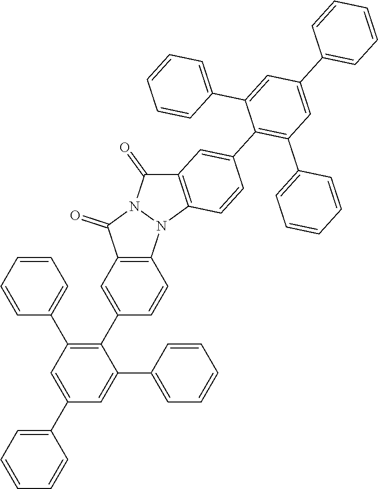 [SMILES: O=c1c2cc(-c3c(-c4ccccc4)cc(-c4ccccc4)cc3-c3ccccc3)ccc2n2c3ccc(-c4c(-c5ccccc5)cc(-c5ccccc5)cc4-c4ccccc4)cc3c(=O)n12] | 47% |
| V68 | 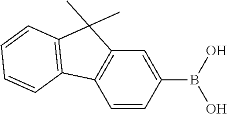 [SMILES: CC1(C)c2ccccc2-c2ccc(B(O)O)cc21] S50 50 mmol 333432-28-3 | 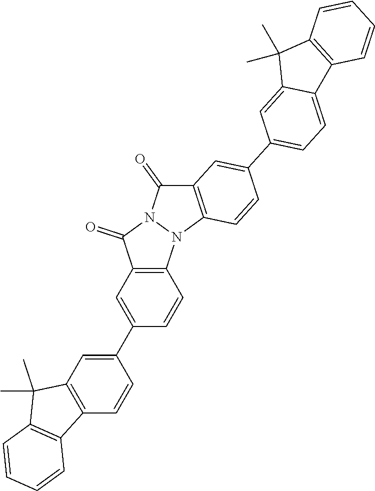 [SMILES: CC1(C)c2ccccc2-c2ccc(-c3ccc4c(c3)c(=O)n3c(=O)c5cc(-c6ccc7c(c6)C(C)(C)c6ccccc6-7)ccc5n43)cc21] | 63% |
| V69 | 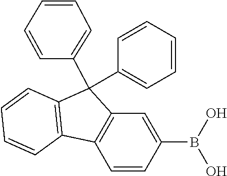 [SMILES: OB(O)c1ccc2c(c1)C(c1ccccc1)(c1ccccc1)c1ccccc1-2] S50 50 mmol 400607-31-0 | 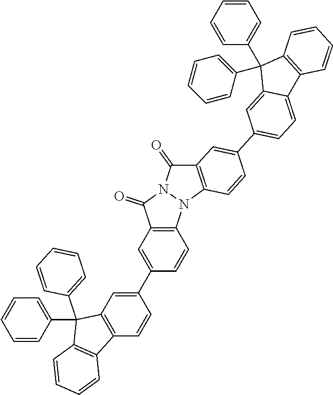 [SMILES: O=c1c2cc(-c3ccc4c(c3)C(c3ccccc3)(c3ccccc3)c3ccccc3-4)ccc2n2c3ccc(-c4ccc5c(c4)C(c4ccccc4)(c4ccccc4)c4ccccc4-5)cc3c(=O)n12] | 60% |
| V70 | 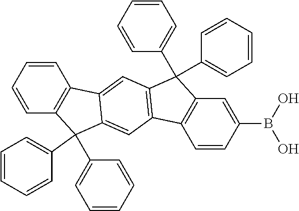 [SMILES: OB(O)c1ccc2c(c1)C(c1ccccc1)(c1ccccc1)c1cc3c(cc1-2)C(c1ccccc1)(c1ccccc1)c1ccccc1-3] S24 50 mmol 1207559-33-8 | 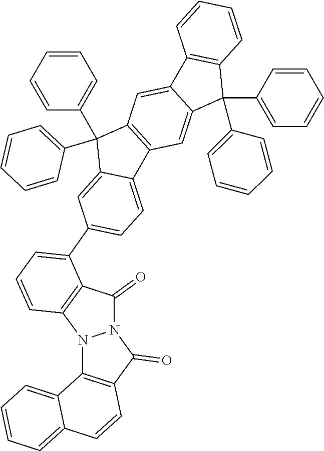 [SMILES: O=c1c2ccc3ccccc3c2n2c3cccc(-c4ccc5c(c4)C(c4ccccc4)(c4ccccc4)c4cc6c(cc4-5)C(c4ccccc4)(c4ccccc4)c4ccccc4-6)c3c(=O)n12] | 59% |
| V71 | 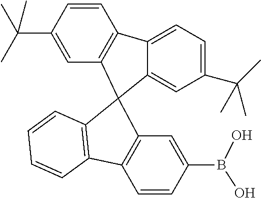 [SMILES: CC(C)(C)c1ccc2c(c1)C1(c3ccccc3-c3ccc(B(O)O)cc31)c1cc(C(C)(C)C)ccc1-2] S25 50 mmol 1241891-39-3 | 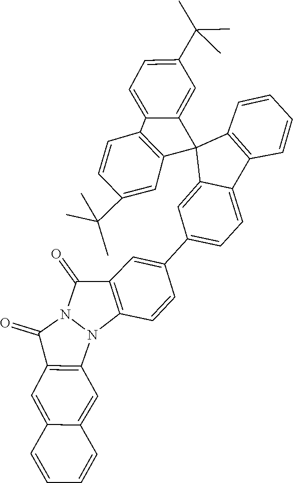 [SMILES: CC(C)(C)c1ccc2c(c1)C1(c3ccccc3-c3ccc(-c4ccc5c(c4)c(=O)n4c(=O)c6cc7ccccc7cc6n54)cc31)c1cc(C(C)(C)C)ccc1-2] | 72% |
| V72 | 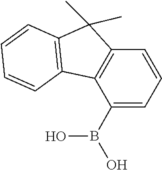 [SMILES: CC1(C)c2ccccc2-c2c(B(O)O)cccc21] S43 25 mmol 1246022-50-3 | 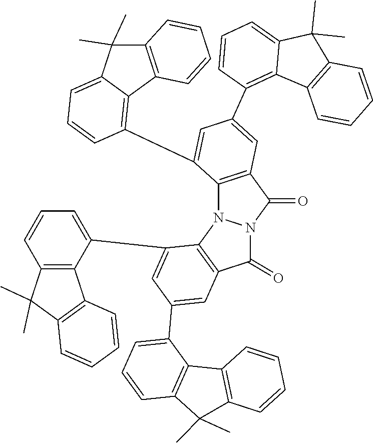 [SMILES: CC1(C)c2ccccc2-c2c(-c3cc(-c4cccc5c4-c4ccccc4C5(C)C)c4c(c3)c(=O)n3c(=O)c5cc(-c6cccc7c6-c6ccccc6C7(C)C)cc(-c6cccc7c6-c6ccccc6C7(C)C)c5n43)cccc21] | 31% |
| V73 | 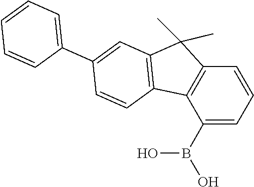 [SMILES: CC1(C)c2cc(-c3ccccc3)ccc2-c2c(B(O)O)cccc21] S50 50 mmol 2597100-63-3 | 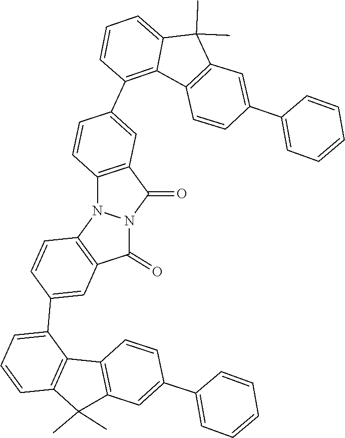 [SMILES: CC1(C)c2cc(-c3ccccc3)ccc2-c2c(-c3ccc4c(c3)c(=O)n3c(=O)c5cc(-c6cccc7c6-c6ccc(-c8ccccc8)cc6C7(C)C)ccc5n43)cccc21] | 61% |
| V74 | 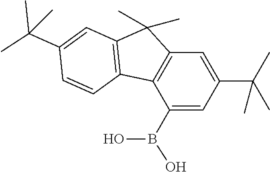 [SMILES: CC(C)(C)c1ccc2c(c1)C(C)(C)c1cc(C(C)(C)C)cc(B(O)O)c1-2] S51 50 mmol 1092576-56-1 | 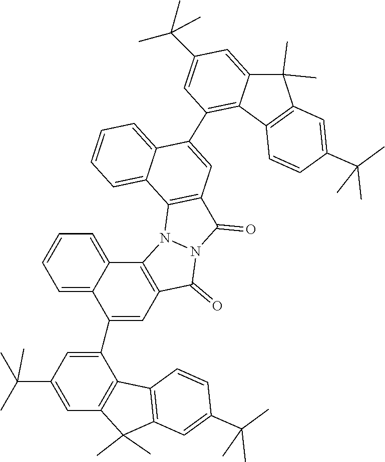 [SMILES: CC(C)(C)c1ccc2c(c1)C(C)(C)c1cc(C(C)(C)C)cc(-c3cc4c(=O)n5c(=O)c6cc(-c7cc(C(C)(C)C)cc8c7-c7ccc(C(C)(C)C)cc7C8(C)C)c7ccccc7c6n5c4c4ccccc34)c1-2] | 59% |
| V75 | 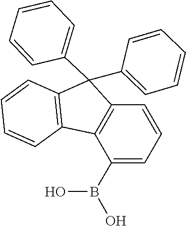 [SMILES: OB(O)c1cccc2c1-c1ccccc1C2(c1ccccc1)c1ccccc1] S53 50 mmol 1224976-40-2 | 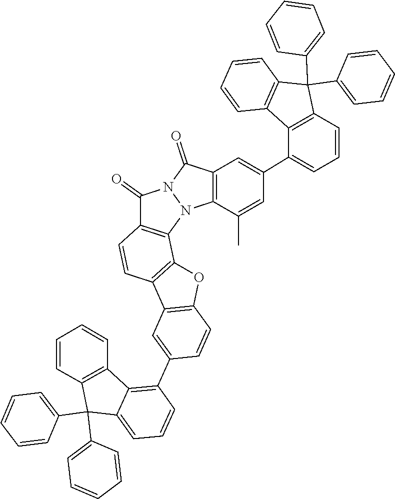 [SMILES: Cc1cc(-c2cccc3c2-c2ccccc2C3(c2ccccc2)c2ccccc2)cc2c(=O)n3c(=O)c4ccc5c6cc(-c7cccc8c7-c7ccccc7C8(c7ccccc7)c7ccccc7)ccc6oc5c4n3c12] | 51% |
| V76 | 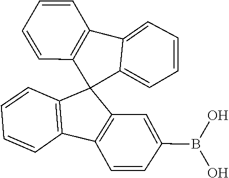 [SMILES: OB(O)c1ccc2c(c1)C1(c3ccccc3-c3ccccc31)c1ccccc1-2] S50 50 mmol 1421789-05-0 | 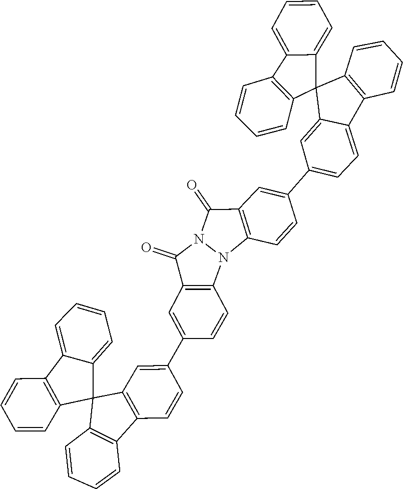 [SMILES: O=c1c2cc(-c3ccc4c(c3)C3(c5ccccc5-c5ccccc53)c3ccccc3-4)ccc2n2c3ccc(-c4ccc5c(c4)C4(c6ccccc6-c6ccccc64)c4ccccc4-5)cc3c(=O)n12] | 66% |
| V77 | 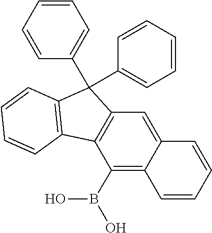 [SMILES: OB(O)c1c2c(cc3ccccc13)C(c1ccccc1)(c1ccccc1)c1ccccc1-2] S34 2540904-37-6 | 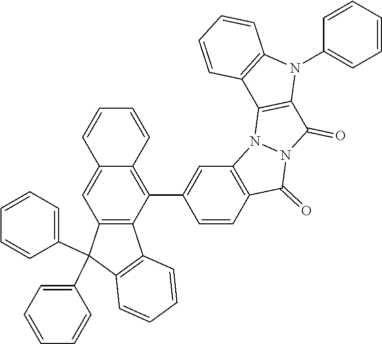 [SMILES: O=c1c2ccc(-c3c4c(cc5ccccc35)C(c3ccccc3)(c3ccccc3)c3ccccc3-4)cc2n2c3c4ccccc4n(-c4ccccc4)c3c(=O)n12] | 70% |
| V78 | 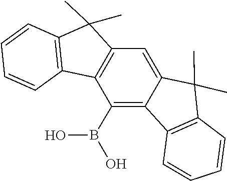 [SMILES: CC1(C)c2ccccc2-c2c1cc1c(c2B(O)O)-c2ccccc2C1(C)C] S50 50 mmol 2364629-32-1 | 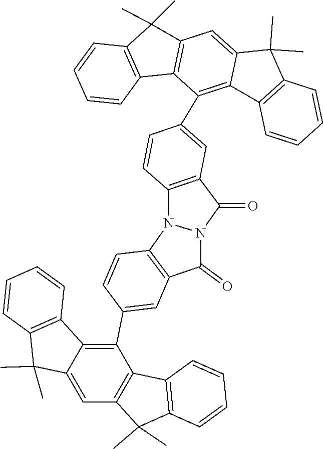 [SMILES: CC1(C)c2ccccc2-c2c1cc1c(c2-c2ccc3c(c2)c(=O)n2c(=O)c4cc(-c5c6c(cc7c5-c5ccccc5C7(C)C)C(C)(C)c5ccccc5-6)ccc4n32)-c2ccccc2C1(C)C] | 50% |
| V79 | 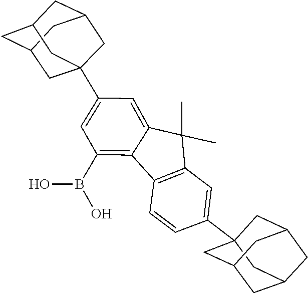 [SMILES: CC1(C)c2cc(C34CC5CC(CC(C5)C3)C4)ccc2-c2c(B(O)O)cc(C34CC5CC(CC(C5)C3)C4)cc21] S50 50 mmol 1630795-54-8 | 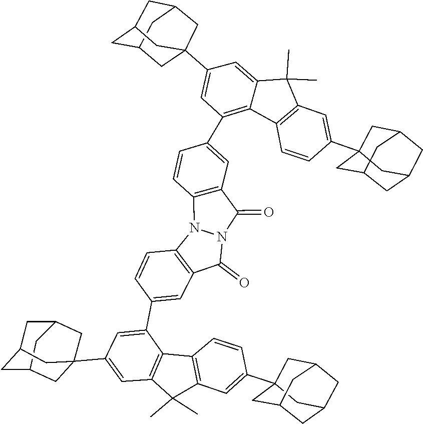 [SMILES: CC1(C)c2cc(C34CC5CC(CC(C5)C3)C4)ccc2-c2c(-c3ccc4c(c3)c(=O)n3c(=O)c5cc(-c6cc(C78CC9CC(CC(C9)C7)C8)cc7c6-c6ccc(C89CC%10CC(CC(C%10)C8)C9)cc6C7(C)C)ccc5n43)cc(C34CC5CC(CC(C5)C3)C4)cc21] | 49% |
| V80 | 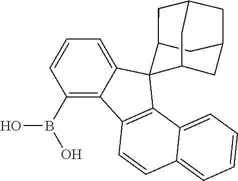 [SMILES: OB(O)c1cccc2c1-c1ccc3ccccc3c1C21C2CC3CC(C2)CC1C3] S50 50 mmol 2549197-79-5 | 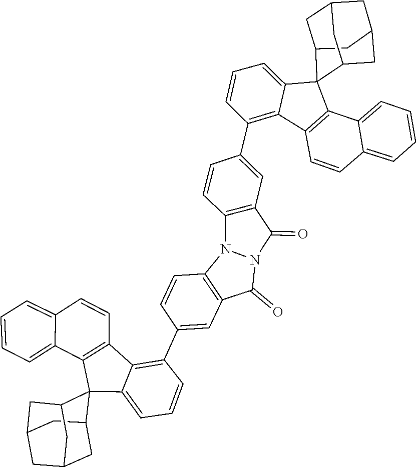 [SMILES: O=c1c2cc(-c3cccc4c3-c3ccc5ccccc5c3C43C4CC5CC(C4)CC3C5)ccc2n2c3ccc(-c4cccc5c4-c4ccc6ccccc6c4C54C5CC6CC(C5)CC4C6)cc3c(=O)n12] | 50% |
| V81 | 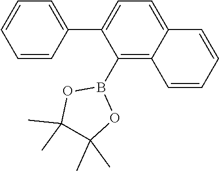 [SMILES: CC1(C)OB(c2c(-c3ccccc3)ccc3ccccc23)OC1(C)C] S43 25 mmol 612086-25-6 | 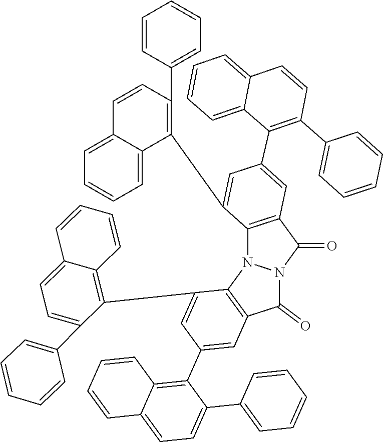 [SMILES: O=c1c2cc(-c3c(-c4ccccc4)ccc4ccccc34)cc(-c3c(-c4ccccc4)ccc4ccccc34)c2n2c3c(-c4c(-c5ccccc5)ccc5ccccc45)cc(-c4c(-c5ccccc5)ccc5ccccc45)cc3c(=O)n12] | 21% |
| V82 | 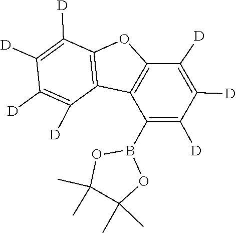 [SMILES: [2H]c1c([2H])c([2H])c2c(oc3c([2H])c([2H])c([2H])c(B4OC(C)(C)C(C)(C)O4)c32)c1[2H]] S43 25 mmol 2605856-02-6 | 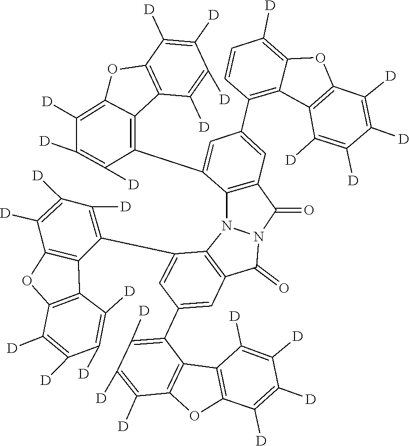 [SMILES: [2H]c1c([2H])c([2H])c2c(oc3c([2H])ccc(-c4cc(-c5c([2H])c([2H])c([2H])c6oc7c([2H])c([2H])c([2H])c([2H])c7c56)c5c(c4)c(=O)n4c(=O)c6cc(-c7c([2H])c([2H])c([2H])c8oc9c([2H])c([2H])c([2H])c([2H])c9c78)cc(-c7c([2H])c([2H])c([2H])c8oc9c([2H])c([2H])c([2H])c([2H])c9c78)c6n54)c32)c1[2H]] | 23% |
| V83 | 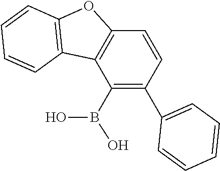 [SMILES: OB(O)c1c(-c2ccccc2)ccc2oc3ccccc3c12] S50 50 mmol 2402769-87-1 | 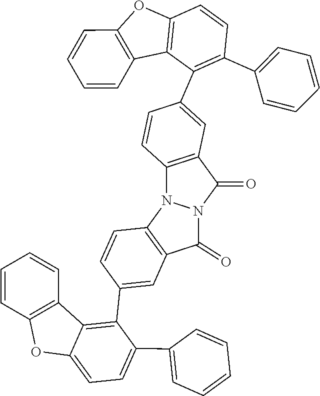 [SMILES: O=c1c2cc(-c3c(-c4ccccc4)ccc4oc5ccccc5c34)ccc2n2c3ccc(-c4c(-c5ccccc5)ccc5oc6ccccc6c45)cc3c(=O)n12] | 60% |
| V84 | 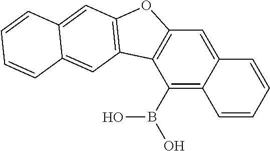 [SMILES: OB(O)c1c2ccccc2cc2oc3cc4ccccc4cc3c12] S50 50 mmol 2049003-24-7 | 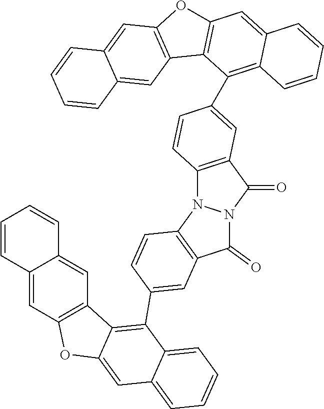 [SMILES: O=c1c2cc(-c3c4ccccc4cc4oc5cc6ccccc6cc5c34)ccc2n2c3ccc(-c4c5ccccc5cc5oc6cc7ccccc7cc6c45)cc3c(=O)n12] | 51% |
| V85 | 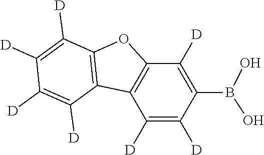 [SMILES: [2H]c1c([2H])c([2H])c2c(oc3c([2H])c(B(O)O)c([2H])c([2H])c32)c1[2H]] S29 2377545-64-5 | 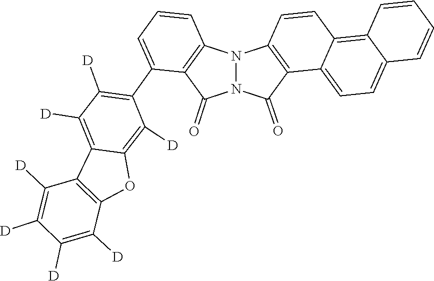 [SMILES: [2H]c1c([2H])c([2H])c2c(oc3c([2H])c(-c4cccc5c4c(=O)n4c(=O)c6c7ccc8ccccc8c7ccc6n54)c([2H])c([2H])c32)c1[2H]] | 59% |
| V86 | 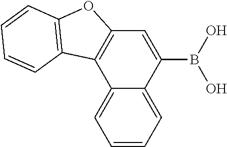 [SMILES: OB(O)c1cc2oc3ccccc3c2c2ccccc12] S30 2035080-71-6 | 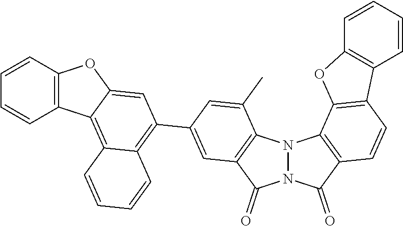 [SMILES: Cc1cc(-c2cc3oc4ccccc4c3c3ccccc23)cc2c(=O)n3c(=O)c4ccc5c6ccccc6oc5c4n3c12] | 62% |
| V87 | 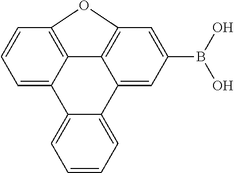 [SMILES: OB(O)c1cc2oc3cccc4c5ccccc5c(c1)c2c34] S28 2606072-21-1 | 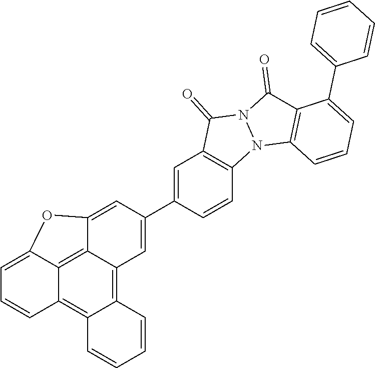 [SMILES: O=c1c2cc(-c3cc4oc5cccc6c7ccccc7c(c3)c4c56)ccc2n2c3cccc(-c4ccccc4)c3c(=O)n12] | 60% |
| V88 | 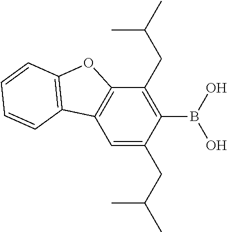 [SMILES: CC(C)Cc1cc2c(oc3ccccc32)c(CC(C)C)c1B(O)O] S50 50 mmol 1651221-10-1 | 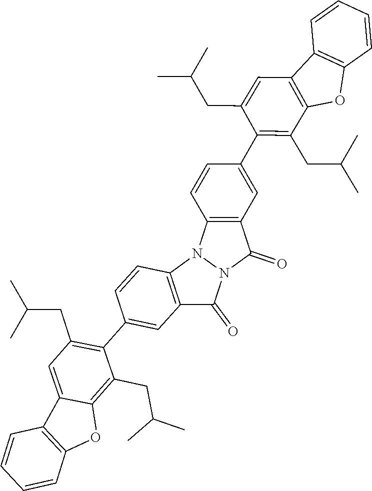 [SMILES: CC(C)Cc1cc2c(oc3ccccc32)c(CC(C)C)c1-c1ccc2c(c1)c(=O)n1c(=O)c3cc(-c4c(CC(C)C)cc5c(oc6ccccc65)c4CC(C)C)ccc3n21] | 54% |
| V89 | 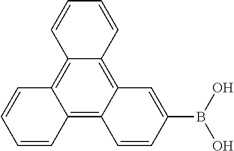 [SMILES: OB(O)c1ccc2c3ccccc3c3ccccc3c2c1] S29 654664-63-8 | 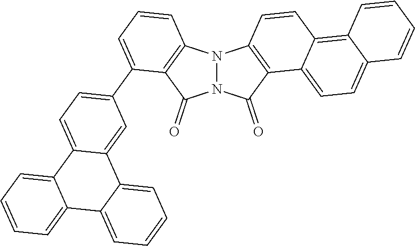 [SMILES: O=c1c2c(-c3ccc4c5ccccc5c5ccccc5c4c3)cccc2n2c3ccc4c5ccccc5ccc4c3c(=O)n12] | 59% |
| V90 | 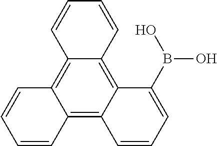 [SMILES: OB(O)c1cccc2c3ccccc3c3ccccc3c12] | 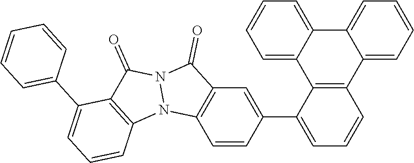 [SMILES: O=c1c2cc(-c3cccc4c5ccccc5c5ccccc5c34)ccc2n2c3cccc(-c4ccccc4)c3c(=O)n12] | 68% |
| | S28 | | |
| | 1430392-46-3 | | |
| V91 | 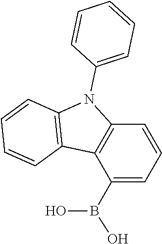 [SMILES: OB(O)c1cccc2c1c1ccccc1n2-c1ccccc1] S50 50 mmol 1370555-65-9 | 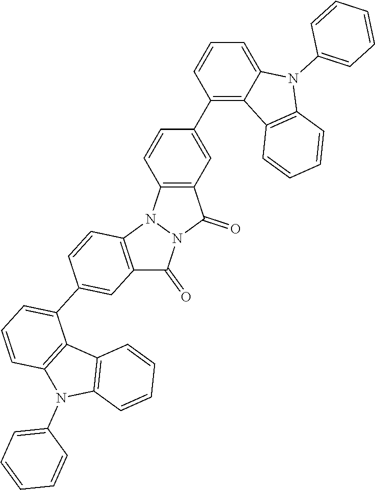 [SMILES: O=c1c2cc(-c3cccc4c3c3ccccc3n4-c3ccccc3)ccc2n2c3ccc(-c4cccc5c4c4ccccc4n5-c4ccccc4)cc3c(=O)n12] | 51% |
| V92 | 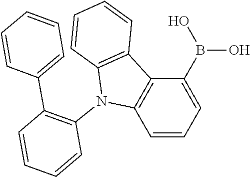 [SMILES: OB(O)c1cccc2c1c1ccccc1n2-c1ccccc1-c1ccccc1] S28 2361216-20-6 | 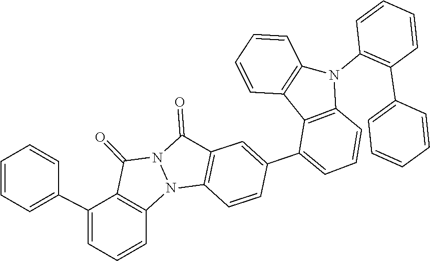 [SMILES: O=c1c2cc(-c3cccc4c3c3ccccc3n4-c3ccccc3-c3ccccc3)ccc2n2c3cccc(-c4ccccc4)c3c(=O)n12] | 62% |
| V93 | 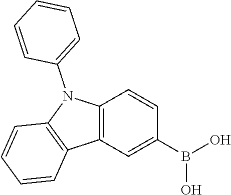 [SMILES: OB(O)c1ccc2c(c1)c1ccccc1n2-c1ccccc1] S30 854952-58-2 | 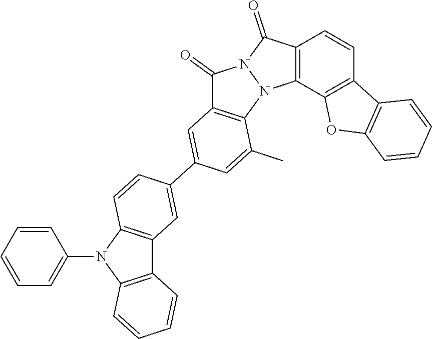 [SMILES: Cc1cc(-c2ccc3c(c2)c2ccccc2n3-c2ccccc2)cc2c(=O)n3c(=O)c4ccc5c6ccccc6oc5c4n3c12] | 67% |
| V94 | 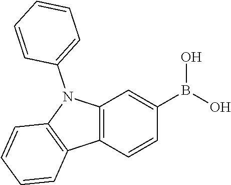 [SMILES: OB(O)c1ccc2c3ccccc3n(-c3ccccc3)c2c1] | 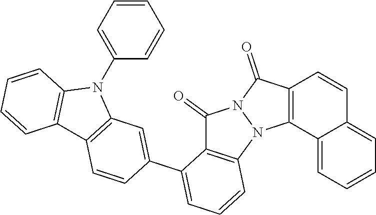 [SMILES: O=c1c2ccc3ccccc3c2n2c3cccc(-c4ccc5c6ccccc6n(-c6ccccc6)c5c4)c3c(=O)n12] | 53% |
| | S24 | | |
| | 1001911-63-2 | | |
| V95 | 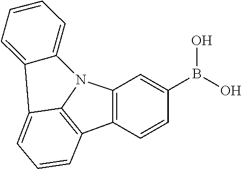 [SMILES: OB(O)c1ccc2c3cccc4c5ccccc5n(c2c1)c43] | 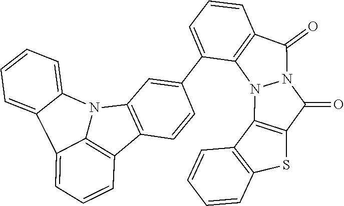 [SMILES: O=c1c2cccc(-c3ccc4c5cccc6c7ccccc7n(c4c3)c65)c2n2c3c(sc4ccccc43)c(=O)n12] | 43% |
| | S50 | | |
| | 2193827-55-1 | | |
| V96 | 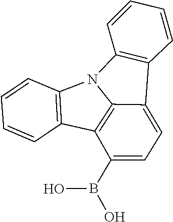 [SMILES: OB(O)c1ccc2c3ccccc3n3c4ccccc4c1c23] S50 50 mmol 2144406-20-0 | 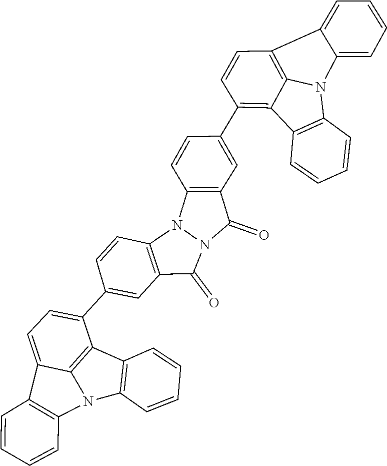 [SMILES: O=c1c2cc(-c3ccc4c5ccccc5n5c6ccccc6c3c45)ccc2n2c3ccc(-c4ccc5c6ccccc6n6c7ccccc7c4c56)cc3c(=O)n12] | 61% |
| V97 | 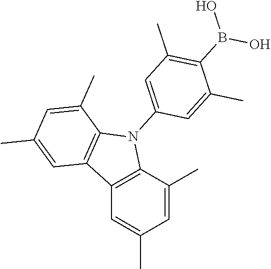 [SMILES: Cc1cc(C)c2c(c1)c1cc(C)cc(C)c1n2-c1cc(C)c(B(O)O)c(C)c1] S50 50 mmol 2595368-09-3 | 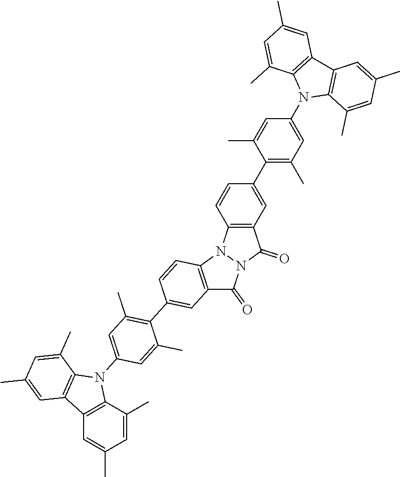 | 50% |
| V98 | 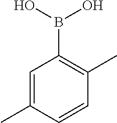 S50 50 mmol 85199-06-0 | 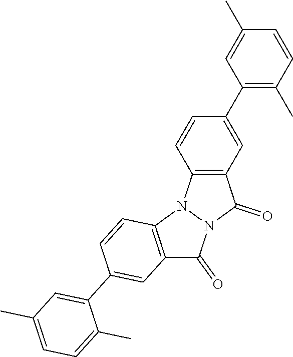 | 66% |
| V99 | 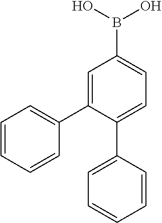 [SMILES: OB(O)c1ccc(-c2ccccc2)c(-c2ccccc2)c1] S50 50 mmol 1246022-49-0 | 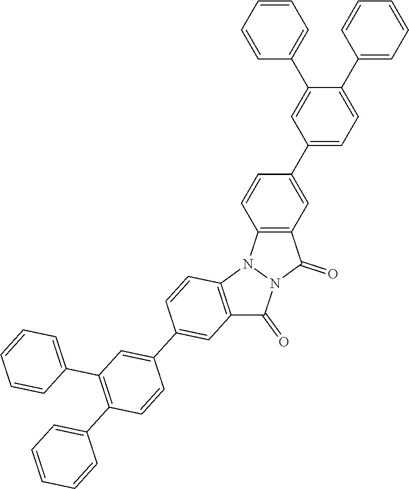 [SMILES: O=c1c2cc(-c3ccc(-c4ccccc4)c(-c4ccccc4)c3)ccc2n2c3ccc(-c4ccc(-c5ccccc5)c(-c5ccccc5)c4)cc3c(=O)n12] | 61% |

### Example V200

### Step 1: Bromine Suzuki Coupling

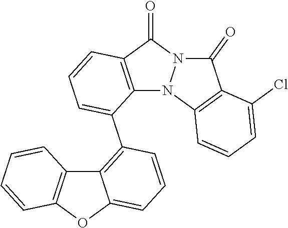 [SMILES: O=c1c2cccc(-c3cccc4oc5ccccc5c34)c2n2c3cccc(Cl)c3c(=O)n12]

A mixture of 35.0 g (100 mmol) of S1, g (110 mmol) of 1-dibenzofuranylboronic acid [162607-19-4], 10.6 g (100 mmol) of sodium carbonate, 1.83 g (6 mmol) of tri-o-tolylphosphine, 225 mg (1 mmol) of palladium(II) acetate, 400 ml of toluene, 200 ml of dioxane and 400 ml of water is heated under reflux for 24 h. After cooling, the organic phase is removed and washed twice with 300 ml each time of water and once with 300 ml of saturated sodium chloride solution. The organic phase is concentrated to dryness, the residue is taken up in dichloromethane (DCM) and filtered through a silica gel bed in the form of a DCM slurry, and the filtrate is concentrated on a rotary evaporator, with continuous replacement of the DCM removed by rotary evaporation with methanol. The crystallized product is filtered off with suction, washed twice with 50 ml each time of methanol and dried under reduced pressure. The crude product is recrystallized from acetonitrile. Yield: 30.3 g (69 mmol), 69%. Purity: about 98% by ¹H NMR.

### Step 2: Chlorine Suzuki Coupling

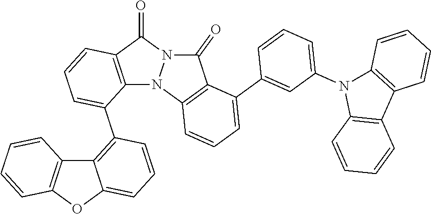 [SMILES: O=c1c2cccc(-c3cccc4oc5ccccc5c34)c2n2c3cccc(-c4cccc(-n5c6ccccc6c6ccccc65)c4)c3c(=O)n12]

Procedure analogous to Example V1, use of 3-(9H-carbazol-9-yl)phenylboronic acid [864377-33-3]. Yield: 30.3 g (47 mmol), 47%. Purity: about 99.9% by HPLC.

The following compounds can be prepared analogously:

| Ex. | Reactants | Product | Yield |
|---|---|---|---|
| V201 | 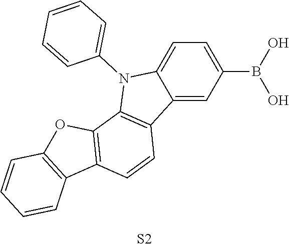 [SMILES: OB(O)c1ccc2c(c1)c1ccc3c4ccccc4oc3c1n2-c1ccccc1 ; "S2"] 2128290-87-7 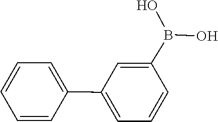 [SMILES: OB(O)c1cccc(-c2ccccc2)c1] 5122-95-2 | 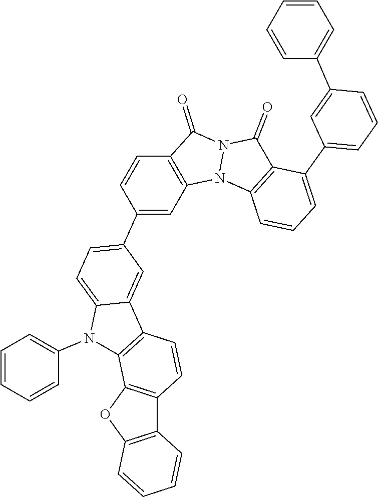 [SMILES: O=c1c2ccc(-c3ccc4c(c3)c3ccc5c6ccccc6oc5c3n4-c3ccccc3)cc2n2c3cccc(-c4cccc(-c5ccccc5)c4)c3c(=O)n12] | 35% |
| V202 | 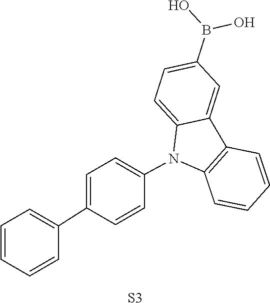 [SMILES: OB(O)c1ccc2c(c1)c1ccccc1n2-c1ccc(-c2ccccc2)cc1 ; "S3"] 1028648-22-7 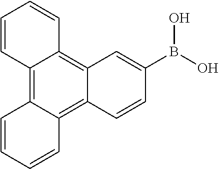 [SMILES: OB(O)c1ccc2c3ccccc3c3ccccc3c2c1] 654664-63-8 | 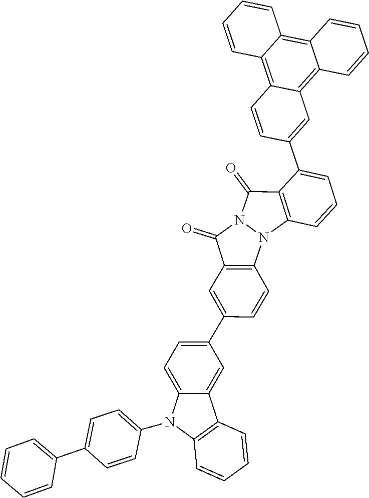 [SMILES: O=c1c2cc(-c3ccc4c(c3)c3ccccc3n4-c3ccc(-c4ccccc4)cc3)ccc2n2c3cccc(-c4ccc5c6ccccc6c6ccccc6c5c4)c3c(=O)n12] | 31% |
| V203 | 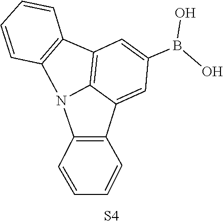 1174032-85-9 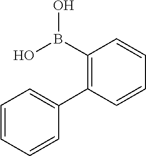 [SMILES: OB(O)c1ccccc1-c1ccccc1] 4688-76-0 | 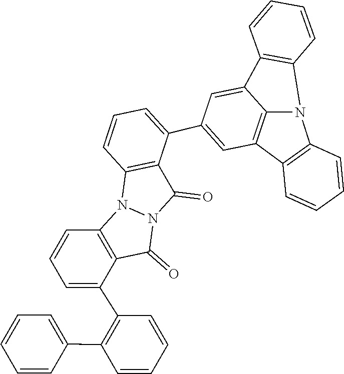 [SMILES: O=c1c2c(-c3cc4c5ccccc5n5c6ccccc6c(c3)c45)cccc2n2c3cccc(-c4ccccc4-c4ccccc4)c3c(=O)n12] | 30% |
| V204 | 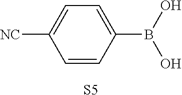 [SMILES: N#Cc1ccc(B(O)O)cc1 ; "S5"] 126747-14-6 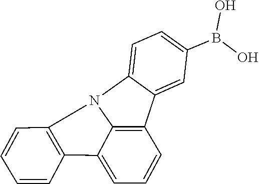 [SMILES: OB(O)c1ccc2c(c1)c1cccc3c4ccccc4n2c31] 1174032-92-8 | 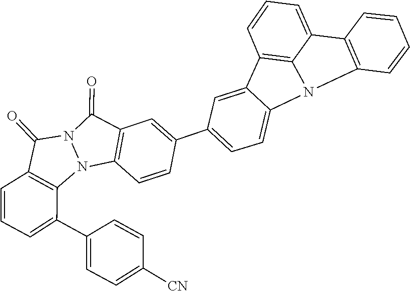 [SMILES: N#Cc1ccc(-c2cccc3c(=O)n4c(=O)c5cc(-c6ccc7c(c6)c6cccc8c9ccccc9n7c86)ccc5n4c23)cc1] | 28% |
| V205 | 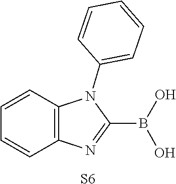 1214723-25-7 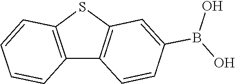 [SMILES: OB(O)c1ccc2c(c1)sc1ccccc12] 108847-24-1 | 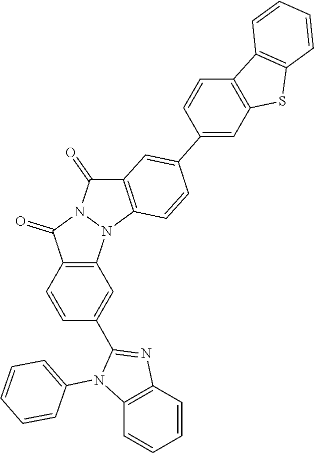 [SMILES: O=c1c2cc(-c3ccc4c(c3)sc3ccccc34)ccc2n2c3cc(-c4nc5ccccc5n4-c4ccccc4)ccc3c(=O)n12] | 46% |
| V206 | 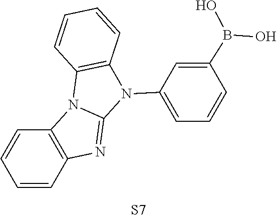 1401068-23-2 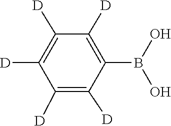 [SMILES: [2H]c1c([2H])c([2H])c(B(O)O)c([2H])c1[2H]] 215527-70-1 | 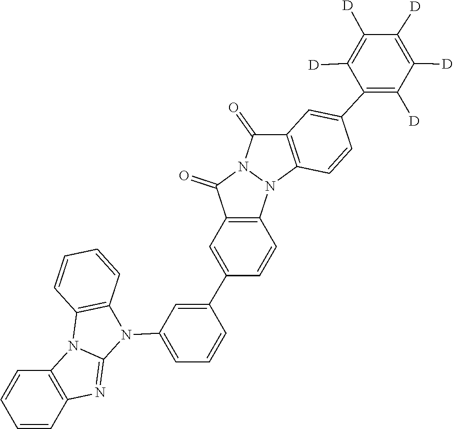 [SMILES: [2H]c1c([2H])c([2H])c(-c2ccc3c(c2)c(=O)n2c(=O)c4cc(-c5cccc(-n6c7ccccc7n7c8ccccc8nc67)c5)ccc4n32)c([2H])c1[2H]] | 45% |
| V207 | 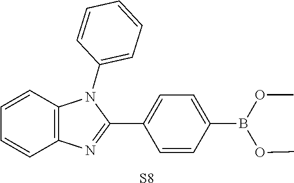 2395777-93-0 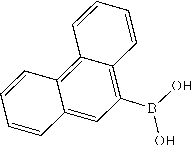 [SMILES: OB(O)c1cc2ccccc2c2ccccc12] 68572-87-2 | 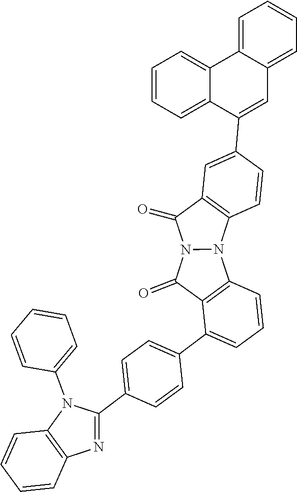 [SMILES: O=c1c2cc(-c3cc4ccccc4c4ccccc34)ccc2n2c3cccc(-c4ccc(-c5nc6ccccc6n5-c5ccccc5)cc4)c3c(=O)n12] | 41% |
| V208 | 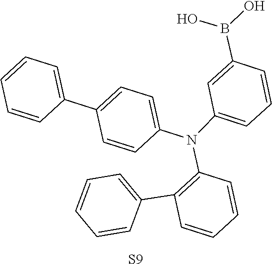 [SMILES: OB(O)c1cccc(N(c2ccc(-c3ccccc3)cc2)c2ccccc2-c2ccccc2)c1 ; "S9"] 1978347-56-6 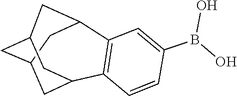 [SMILES: OB(O)c1ccc2c(c1)C1CC3CC(CC2C3)C1] 1801624-63-4 | 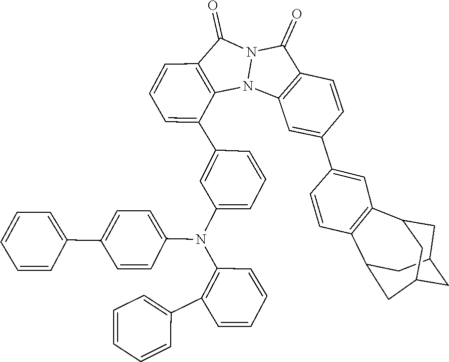 [SMILES: O=c1c2ccc(-c3ccc4c(c3)C3CC5CC(CC4C5)C3)cc2n2c3c(-c4cccc(N(c5ccc(-c6ccccc6)cc5)c5ccccc5-c5ccccc5)c4)cccc3c(=O)n12] | 36% |
| V209 | 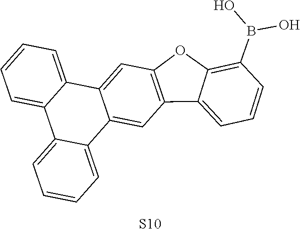 [SMILES: OB(O)c1cccc2c1oc1cc3c4ccccc4c4ccccc4c3cc12 ; "S10"] 2454395-23-2 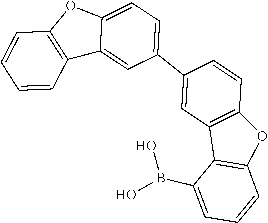 1822320-55-7 | 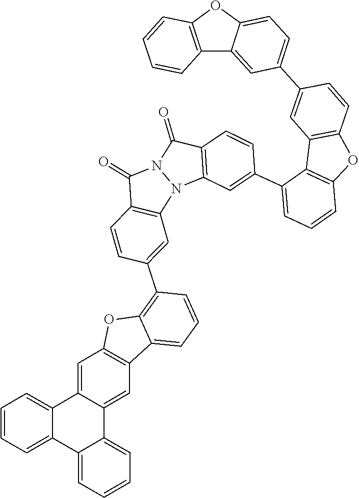 [SMILES: O=c1c2ccc(-c3cccc4c3oc3cc5c6ccccc6c6ccccc6c5cc34)cc2n2c3cc(-c4cccc5oc6ccc(-c7ccc8oc9ccccc9c8c7)cc6c45)ccc3c(=O)n12] | 45% |
| V210 | 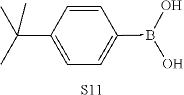 123324-71-0 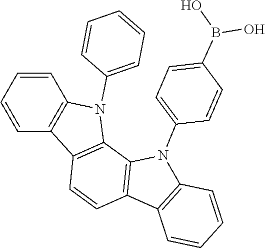 [SMILES: OB(O)c1ccc(-n2c3ccccc3c3ccc4c5ccccc5n(-c5ccccc5)c4c32)cc1] 2222276-71-1 | 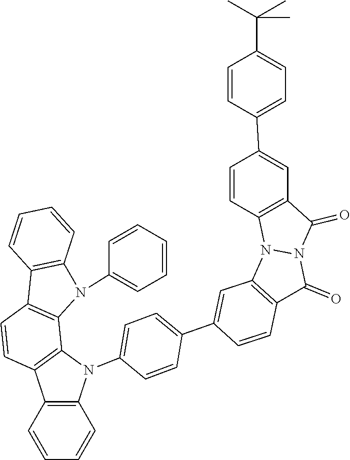 [SMILES: CC(C)(C)c1ccc(-c2ccc3c(c2)c(=O)n2c(=O)c4ccc(-c5ccc(-n6c7ccccc7c7ccc8c9ccccc9n(-c9ccccc9)c8c76)cc5)cc4n32)cc1] | 44% |
| V211 | 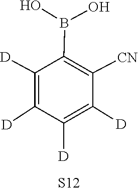 [SMILES: [2H]c1c([2H])c([2H])c(B(O)O)c(C#N)c1[2H] ; "S12"] 2241870-66-4 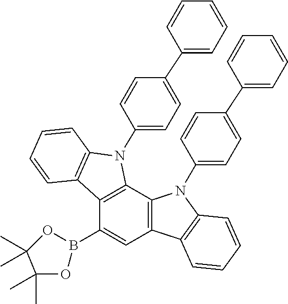 [SMILES: CC1(C)OB(c2cc3c4ccccc4n(-c4ccc(-c5ccccc5)cc4)c3c3c2c2ccccc2n3-c2ccc(-c3ccccc3)cc2)OC1(C)C] 1900681-96-0 | 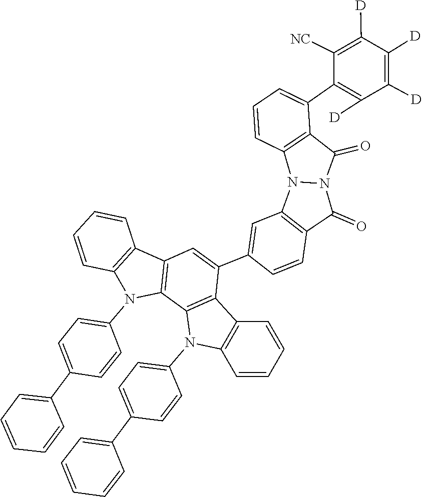 [SMILES: [2H]c1c([2H])c([2H])c(-c2cccc3c2c(=O)n2c(=O)c4ccc(-c5cc6c7ccccc7n(-c7ccc(-c8ccccc8)cc7)c6c6c5c5ccccc5n6-c5ccc(-c6ccccc6)cc5)cc4n32)c(C#N)c1[2H]] | 41% |
| V212 | 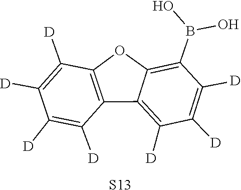 [SMILES: [2H]c1c([2H])c([2H])c2c(oc3c(B(O)O)c([2H])c([2H])c([2H])c32)c1[2H] ; "S13"] 2230311-62-1 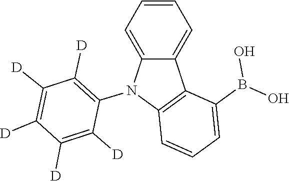 [SMILES: [2H]c1c([2H])c([2H])c(-n2c3ccccc3c3c(B(O)O)cccc32)c([2H])c1[2H]] 2271286-98-5 | 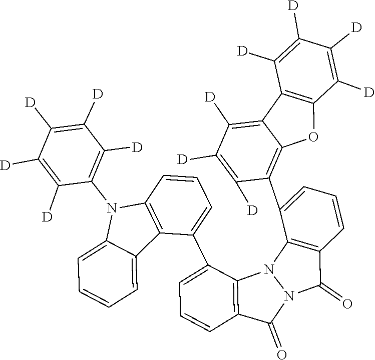 [SMILES: [2H]c1c([2H])c([2H])c(-n2c3ccccc3c3c(-c4cccc5c(=O)n6c(=O)c7cccc(-c8c([2H])c([2H])c([2H])c9c8oc8c([2H])c([2H])c([2H])c([2H])c89)c7n6c45)cccc32)c([2H])c1[2H]] | 22% |
| V213 | 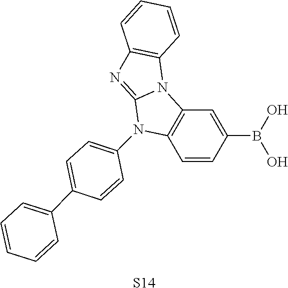 [SMILES: OB(O)c1ccc2c(c1)n1c3ccccc3nc1n2-c1ccc(-c2ccccc2)cc1 ; "S14"] 2217618-64-7 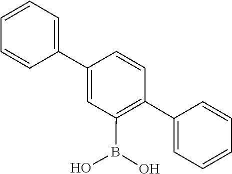 [SMILES: OB(O)c1cc(-c2ccccc2)ccc1-c1ccccc1] 1205748-34-0 | 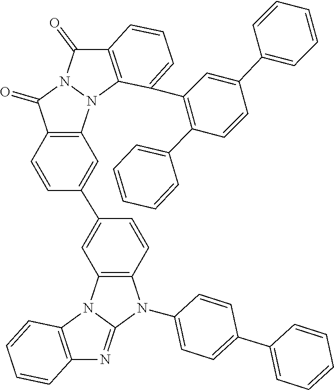 [SMILES: O=c1c2ccc(-c3ccc4c(c3)n3c5ccccc5nc3n4-c3ccc(-c4ccccc4)cc3)cc2n2c3c(-c4cc(-c5ccccc5)ccc4-c4ccccc4)cccc3c(=O)n12] | 37% |
| V214 | 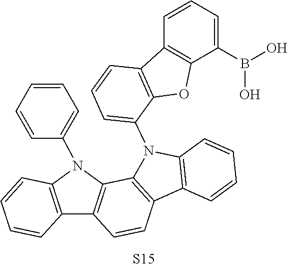 [SMILES: OB(O)c1cccc2c1oc1c(-n3c4ccccc4c4ccc5c6ccccc6n(-c6ccccc6)c5c43)cccc12 ; "S15"] 1609088-39-2 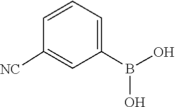 [SMILES: N#Cc1cccc(B(O)O)c1] 150255-96-2 | 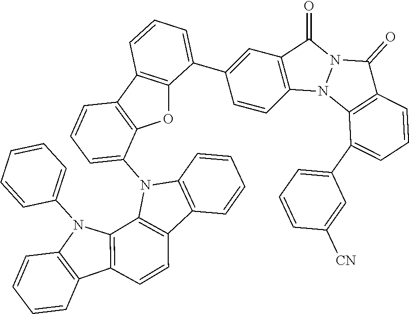 [SMILES: N#Cc1cccc(-c2cccc3c(=O)n4c(=O)c5cc(-c6cccc7c6oc6c(-n8c9ccccc9c9ccc%10c%11ccccc%11n(-c%11ccccc%11)c%10c98)cccc67)ccc5n4c23)c1] | 35% |
| V215 | 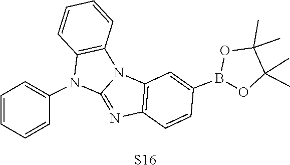 [SMILES: CC1(C)OB(c2ccc3nc4n(-c5ccccc5)c5ccccc5n4c3c2)OC1(C)C ; "S16"] 2093171-71-0 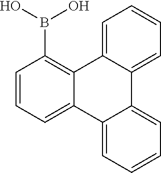 [SMILES: OB(O)c1cccc2c3ccccc3c3ccccc3c12] 1430392-46-3 | 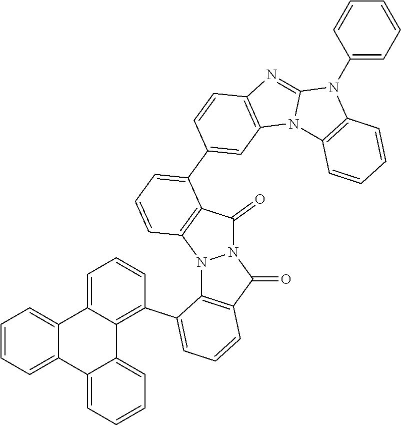 [SMILES: O=c1c2cccc(-c3cccc4c5ccccc5c5ccccc5c34)c2n2c3cccc(-c4ccc5nc6n(-c7ccccc7)c7ccccc7n6c5c4)c3c(=O)n12] | 31% |
| V216 | 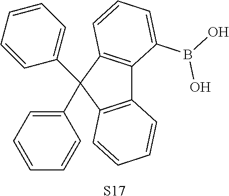 [SMILES: OB(O)c1cccc2c1-c1ccccc1C2(c1ccccc1)c1ccccc1 ; "S17"] 1224976-40-2 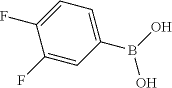 [SMILES: OB(O)c1ccc(F)c(F)c1] 168267-41-2 | 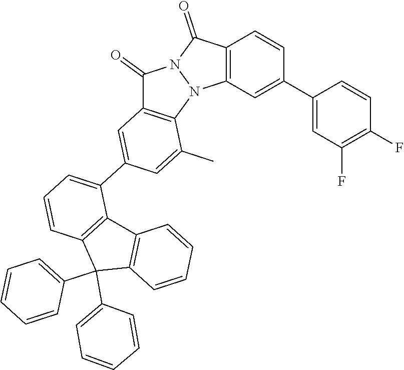 [SMILES: Cc1cc(-c2cccc3c2-c2ccccc2C3(c2ccccc2)c2ccccc2)cc2c(=O)n3c(=O)c4ccc(-c5ccc(F)c(F)c5)cc4n3c12] | 43% |
| V217 | 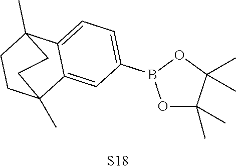 [SMILES: CC12CCC(C)(CC1)c1cc(B3OC(C)(C)C(C)(C)O3)ccc12 ; "S18"] 1801624-61-2 200 mmol 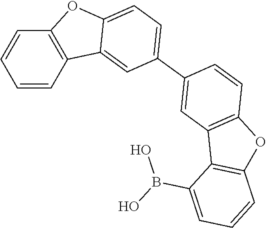 [SMILES: OB(O)c1cccc2oc3ccc(-c4ccc5oc6ccccc6c5c4)cc3c12] 1822320-55-7 | 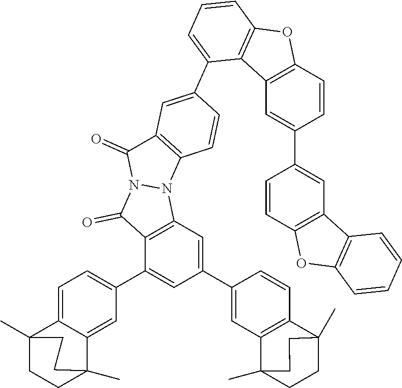 [SMILES: CC12CCC(C)(CC1)c1cc(-c3cc(-c4ccc5c(c4)C4(C)CCC5(C)CC4)c4c(=O)n5c(=O)c6cc(-c7cccc8oc9ccc(-c%10ccc%11oc%12ccccc%12c%11c%10)cc9c78)ccc6n5c4c3)ccc12] | 35% |
| V218 | 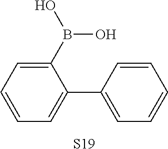 [SMILES: OB(O)c1ccccc1-c1ccccc1 ; "S19"] 4688-76-0 200 mmol 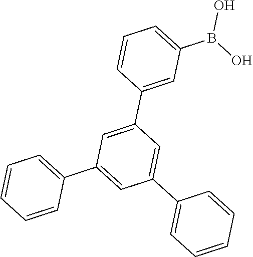 [SMILES: OB(O)c1cccc(-c2cc(-c3ccccc3)cc(-c3ccccc3)c2)c1] 1233200-59-3 | 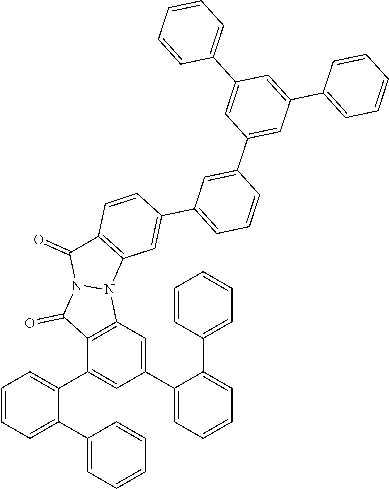 [SMILES: O=c1c2ccc(-c3cccc(-c4cc(-c5ccccc5)cc(-c5ccccc5)c4)c3)cc2n2c3cc(-c4ccccc4-c4ccccc4)cc(-c4ccccc4-c4ccccc4)c3c(=O)n12] | 33% |
| V219 | 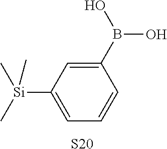 177171-16-3 200 mmol 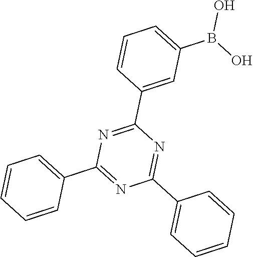 [SMILES: OB(O)c1cccc(-c2nc(-c3ccccc3)nc(-c3ccccc3)n2)c1] 1612243-82-9 | 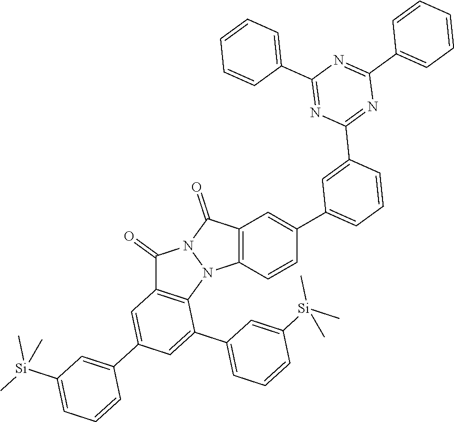 [SMILES: C[Si](C)(C)c1cccc(-c2cc(-c3cccc([Si](C)(C)C)c3)c3c(c2)c(=O)n2c(=O)c4cc(-c5cccc(-c6nc(-c7ccccc7)nc(-c7ccccc7)n6)c5)ccc4n32)c1] | 29% |
| V220 | 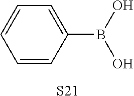 98-80-6 200 mmol 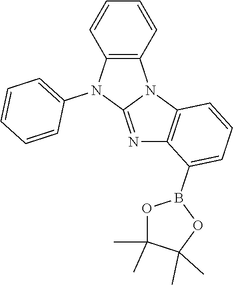 [SMILES: CC1(C)OB(c2cccc3c2nc2n(-c4ccccc4)c4ccccc4n32)OC1(C)C] 2111193-19-0 | 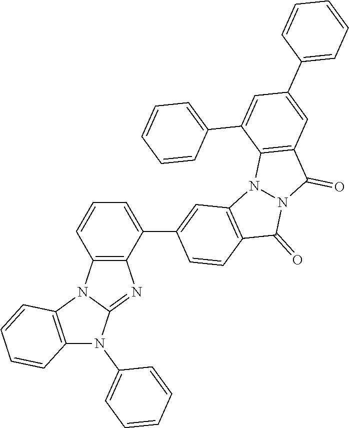 [SMILES: O=c1c2ccc(-c3cccc4c3nc3n(-c5ccccc5)c5ccccc5n43)cc2n2c3c(-c4ccccc4)cc(-c4ccccc4)cc3c(=O)n12] | 37% |
| V221 | 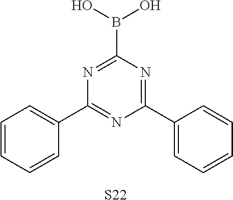 [SMILES: OB(O)c1nc(-c2ccccc2)nc(-c2ccccc2)n1 ; "S22"] 1251825-65-6 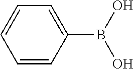 98-80-6 210 mmol | 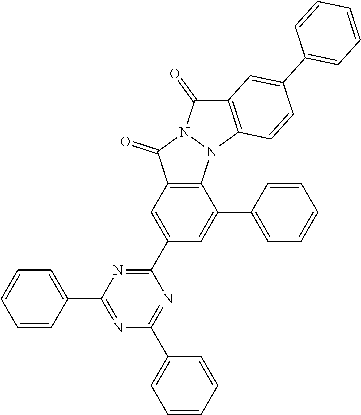 [SMILES: O=c1c2cc(-c3ccccc3)ccc2n2c3c(-c4ccccc4)cc(-c4nc(-c5ccccc5)nc(-c5ccccc5)n4)cc3c(=O)n12] | 35% |
| V222 | 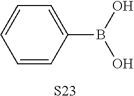 98-80-6 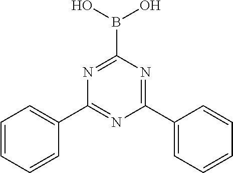 [SMILES: OB(O)c1nc(-c2ccccc2)nc(-c2ccccc2)n1] 1251825-65-6 220 mmol | 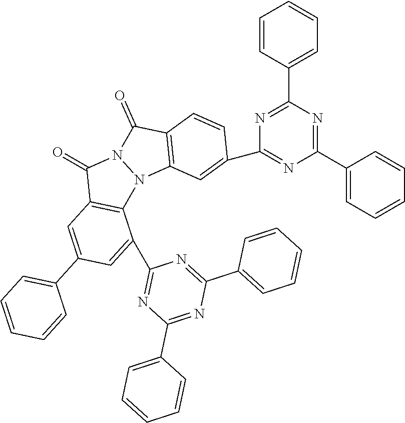 [SMILES: O=c1c2ccc(-c3nc(-c4ccccc4)nc(-c4ccccc4)n3)cc2n2c3c(-c4nc(-c5ccccc5)nc(-c5ccccc5)n4)cc(-c4ccccc4)cc3c(=O)n12] | 31% |
| V223 | 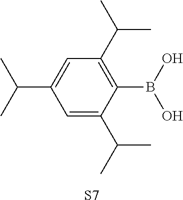 [SMILES: CC(C)c1cc(C(C)C)c(B(O)O)c(C(C)C)c1 ; "S7"] 154549-38-9 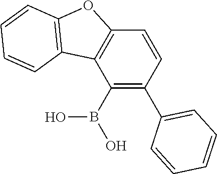 [SMILES: OB(O)c1c(-c2ccccc2)ccc2oc3ccccc3c12] 2402769-87-1 | 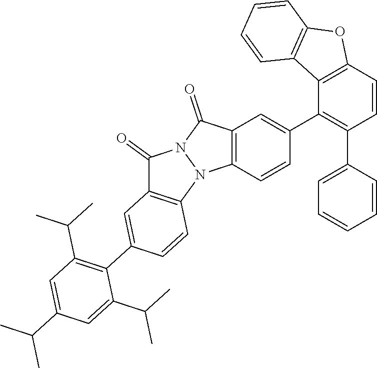 [SMILES: CC(C)c1cc(C(C)C)c(-c2ccc3c(c2)c(=O)n2c(=O)c4cc(-c5c(-c6ccccc6)ccc6oc7ccccc7c56)ccc4n32)c(C(C)C)c1] | 48% |
| V224 | 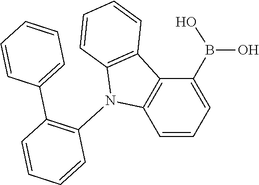 [SMILES: OB(O)c1cccc2c1c1ccccc1n2-c1ccccc1-c1ccccc1] 2361216-20-6 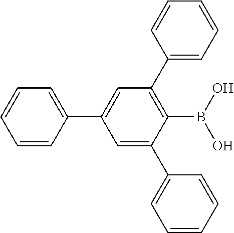 [SMILES: OB(O)c1c(-c2ccccc2)cc(-c2ccccc2)cc1-c1ccccc1] 1004321-87-2 | 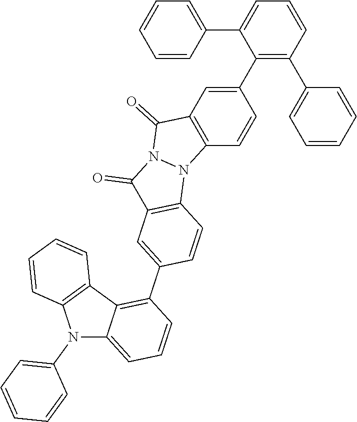 [SMILES: O=c1c2cc(-c3c(-c4ccccc4)cccc3-c3ccccc3)ccc2n2c3ccc(-c4cccc5c4c4ccccc4n5-c4ccccc4)cc3c(=O)n12] | 47% |

### Example V300

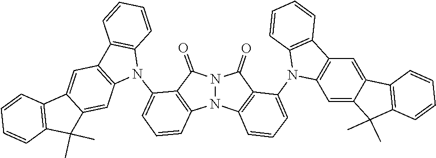 [SMILES: CC1(C)c2ccccc2-c2cc3c4ccccc4n(-c4cccc5c4c(=O)n4c(=O)c6c(-n7c8ccccc8c8cc9c(cc87)C(C)(C)c7ccccc7-9)cccc6n54)c3cc21]

A mixture of 13.6 g (50 mmol) of S4, 31.2 g (110 mmol) of 5,7-dihydro-7,7-dimethylindeno[2,1-b]carbazole [1257220-47-5], 14.4 g (150 mmol) of sodium tert-butoxide, 2.65 g (4 mmol) of rac-BINAP (it is alternatively possible to use S-Phos, X-Phos, tri-tert-butylphosphine), 674 mg (3 mmol) of palladium(II) acetate, 100 g of glass beads (diameter 3 mm) and 300 ml of toluene is stirred at 110° C. for 24 h. While the mixture is still hot, it is filtered with suction through a Celite bed in the form of a hot toluene slurry, the filtrate is concentrated to dryness under reduced pressure, and the solids are extracted by stirring in 300 ml of hot methanol. The solids are filtered off with suction while the mixture is still warm, washed twice with 50 ml of methanol and dried under reduced pressure. Further purification is effected by hot extraction crystallization (solvent mixtures: DCM/acetonitrile 1:3 to 3:1) and fractional sublimation. Yield: 20.1 g (25 mol), 50%. Purity: about 99.9% by HPLC.

The following compounds can be prepared analogously:

| Ex. | Reactants | Product | Yield |
|---|---|---|---|
| V301 | 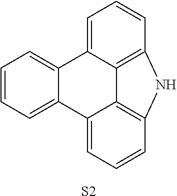 109606-75-9 | 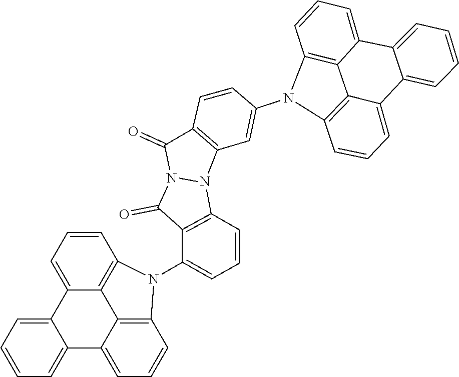 [SMILES: O=c1c2ccc(-n3c4cccc5c6ccccc6c6cccc3c6c54)cc2n2c3cccc(-n4c5cccc6c7ccccc7c7cccc4c7c65)c3c(=O)n12] | 46% |
| V302 | 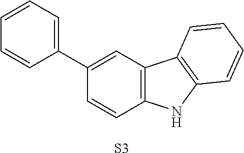 103012-26-6 | 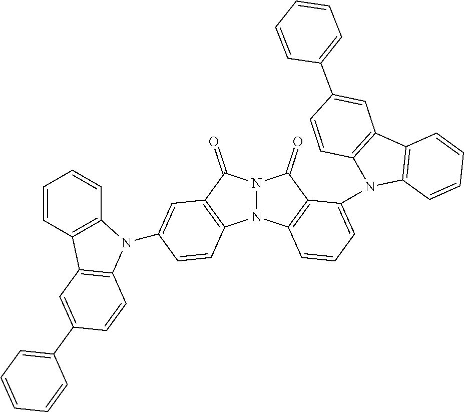 [SMILES: O=c1c2cc(-n3c4ccccc4c4cc(-c5ccccc5)ccc43)ccc2n2c3cccc(-n4c5ccccc5c5cc(-c6ccccc6)ccc54)c3c(=O)n12] | 44% |
| V303 | 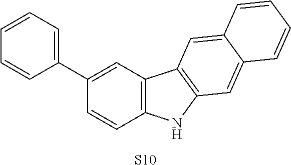 [SMILES: c1ccc(-c2ccc3[nH]c4cc5ccccc5cc4c3c2)cc1 ; "S10"] 1819346-21-8 | 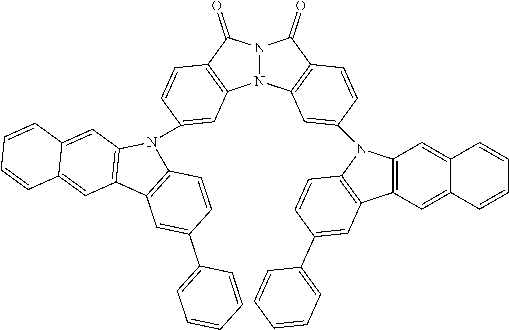 [SMILES: O=c1c2ccc(-n3c4ccc(-c5ccccc5)cc4c4cc5ccccc5cc43)cc2n2c3cc(-n4c5ccc(-c6ccccc6)cc5c5cc6ccccc6cc54)ccc3c(=O)n12] | 40% |
| V304 | 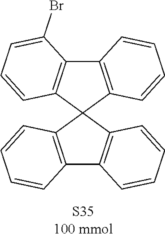 [SMILES: Brc1cccc2c1-c1ccccc1C21c2ccccc2-c2ccccc21] 1161009-88-6 110 mmol | 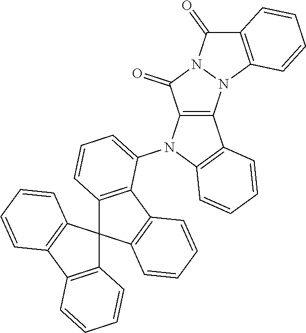 [SMILES: O=c1c2ccccc2n2c3c4ccccc4n(-c4cccc5c4-c4ccccc4C54c5ccccc5-c5ccccc54)c3c(=O)n12] | 57% |
| V305 | 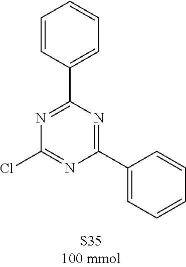 [SMILES: Clc1nc(-c2ccccc2)nc(-c2ccccc2)n1] 3842-55-5 120 mmol 110 mmol NaO-t-Bu | 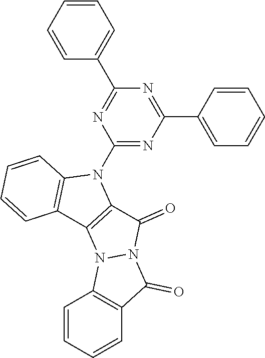 [SMILES: O=c1c2ccccc2n2c3c4ccccc4n(-c4nc(-c5ccccc5)nc(-c5ccccc5)n4)c3c(=O)n12] | 59% |
| V306 | 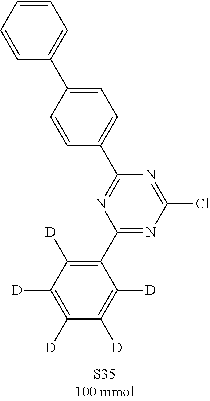 [SMILES: [2H]c1c([2H])c([2H])c(-c2nc(Cl)nc(-c3ccc(-c4ccccc4)cc3)n2)c([2H])c1[2H]] 2401923-63-3 130 mmol 110 mmol NaO-t-Bu dimethylacetamide in place of toluene | 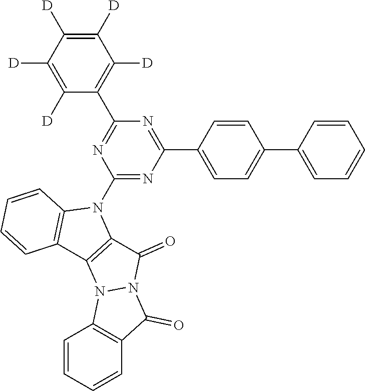 [SMILES: [2H]c1c([2H])c([2H])c(-c2nc(-c3ccc(-c4ccccc4)cc3)nc(-n3c4ccccc4c4c3c(=O)n3c(=O)c5ccccc5n43)n2)c([2H])c1[2H]] | 56% |
| V307 | 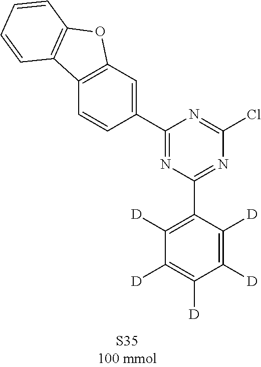 [SMILES: [2H]c1c([2H])c([2H])c(-c2nc(Cl)nc(-c3ccc4c(c3)oc3ccccc34)n2)c([2H])c1[2H]] 2226747-65-3 130 mmol 110 mmol NaO-t-Bu dimethylacetamide in place of toluene | 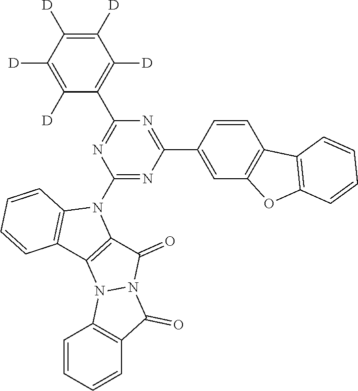 [SMILES: [2H]c1c([2H])c([2H])c(-c2nc(-c3ccc4c(c3)oc3ccccc34)nc(-n3c4ccccc4c4c3c(=O)n3c(=O)c5ccccc5n43)n2)c([2H])c1[2H]] | 54% |
| V308 | 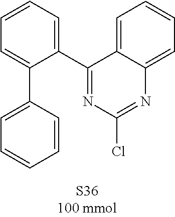 [SMILES: Clc1nc(-c2ccccc2-c2ccccc2)c2ccccc2n1] 1852465-53-2 120 mmol 110 mmol NaO-t-Bu dimethylacetamide in place of toluene | 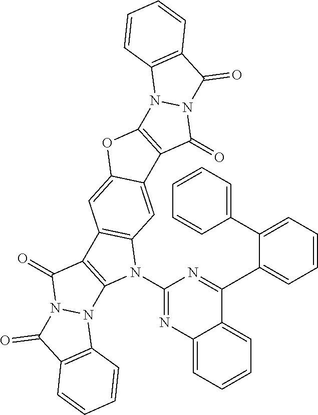 [SMILES: O=c1c2ccccc2n2c3oc4cc5c6c(=O)n7c(=O)c8ccccc8n7c6n(-c6nc(-c7ccccc7-c7ccccc7)c7ccccc7n6)c5cc4c3c(=O)n12] | 60% |
| V309 | 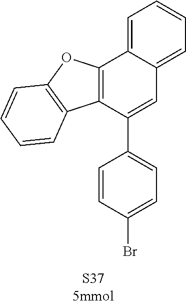 [SMILES: Brc1ccc(-c2cc3ccccc3c3oc4ccccc4c23)cc1] 2541485-90-7 110 mmol | 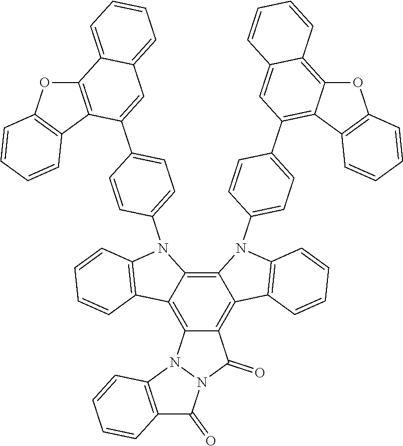 [SMILES: O=c1c2ccccc2n2c3c(c(=O)n12)c1c2ccccc2n(-c2ccc(-c4cc5ccccc5c5oc6ccccc6c45)cc2)c1c1c3c2ccccc2n1-c1ccc(-c2cc3ccccc3c3oc4ccccc4c23)cc1] | 34% |
| V310 | 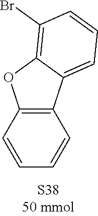 [SMILES: Brc1cccc2c1oc1ccccc12] 89827-45-2 110 mmol | 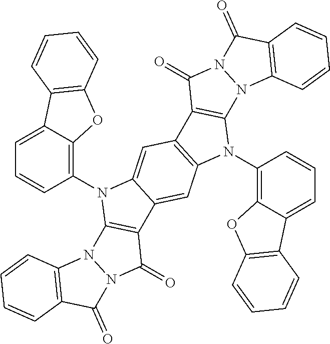 [SMILES: O=c1c2ccccc2n2c3c(c(=O)n12)c1cc2c(cc1n3-c1cccc3c1oc1ccccc13)c1c(=O)n3c(=O)c4ccccc4n3c1n2-c1cccc2c1oc1ccccc12] | 47% |

### Example L1

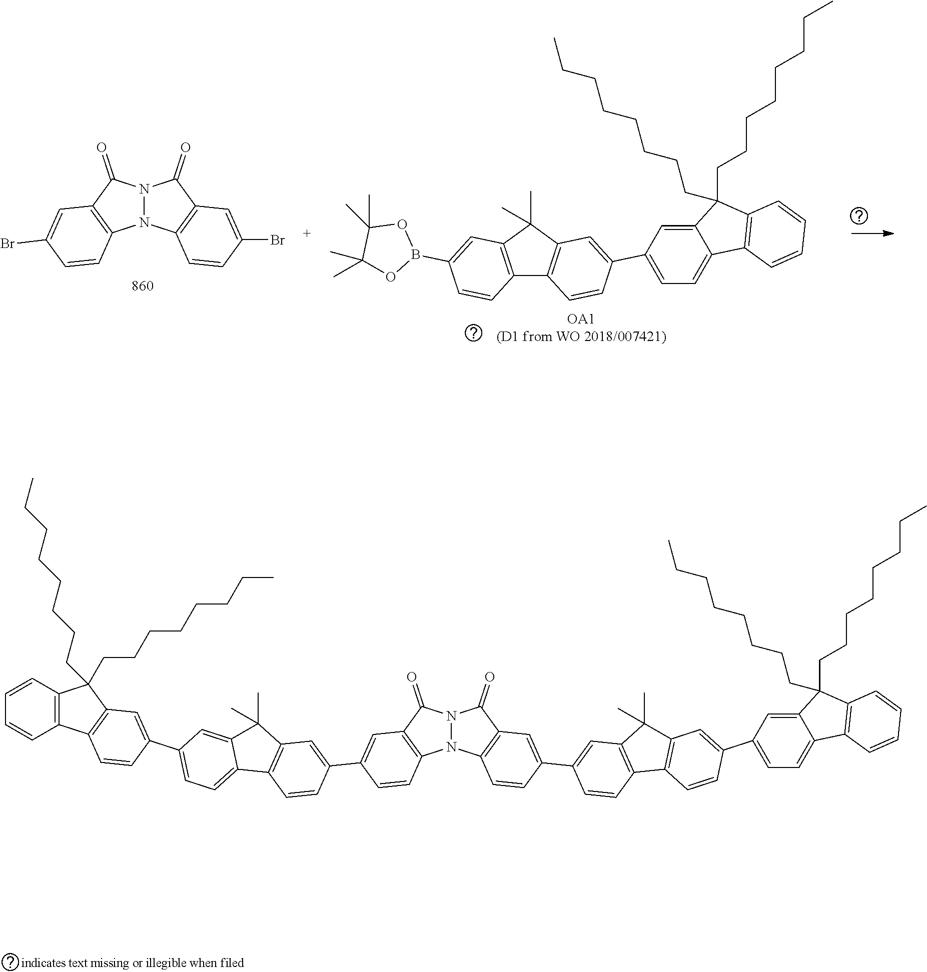 [SMILES: O=c1c2cc(Br)ccc2n2c3ccc(Br)cc3c(=O)n12 ; CCCCCCCCC1(CCCCCCCC)c2ccccc2-c2ccc(-c3ccc4c(c3)C(C)(C)c3cc(B5OC(C)(C)C(C)(C)O5)ccc3-4)cc21 ; CCCCCCCCC1(CCCCCCCC)c2ccccc2-c2ccc(-c3ccc4c(c3)C(C)(C)c3cc(-c5ccc6c(c5)c(=O)n5c(=O)c7cc(-c8ccc9c(c8)C(C)(C)c8cc(-c%10ccc%11c(c%10)C(CCCCCCCC)(CCCCCCCC)c%10ccccc%10-%11)ccc8-9)ccc7n65)ccc3-4)cc21]

8.27 g (21 mmol) of S50, 32.60 g (46 mmol) of OA1 and 4.5 g (42.4 mmol) of sodium carbonate are suspended in 600 ml of a mixture of toluene/ethanol/water (2:1:1) and degassed with argon. After addition of 613 mg (0.53 mmol) of tetrakis(triphenylphosphine)palladium(0), the mixture is stirred at 110° C. After six hours, the reaction mixture is allowed to cool to room temperature and a further 200 ml toluene is added. The organic phase is removed, washed twice with 300 ml each time of water and once with 300 ml of saturated sodium chloride solution, dried over sodium sulfate and filtered through a Celite bed in the form of a toluene slurry. The filtrate is concentrated to dryness, and the residue is dissolved in 300 ml of dichloromethane, 200 ml of methanol is added, and the mixture is concentrated to about 300 ml on a rotary evaporator. The precipitated solids are filtered off with suction and washed three times with 50 ml each time of methanol. The solids obtained are purified by means of column chromatography and repeated recrystallization from toluene/n-heptane up to an HPLC purity of >99.9%, and finally subjected to heat treatment at 10⁻⁶ mbar and 250° C. Yield: 6.85 g (4.9 mmol), 24%.

The following compounds can be synthesized in an analogous manner:

| | Reactant | | |
|---|---|---|---|
| L | 1 | Reactant 2 | Product |
| L2 | S50 | CAS 2171483- 75-1 | 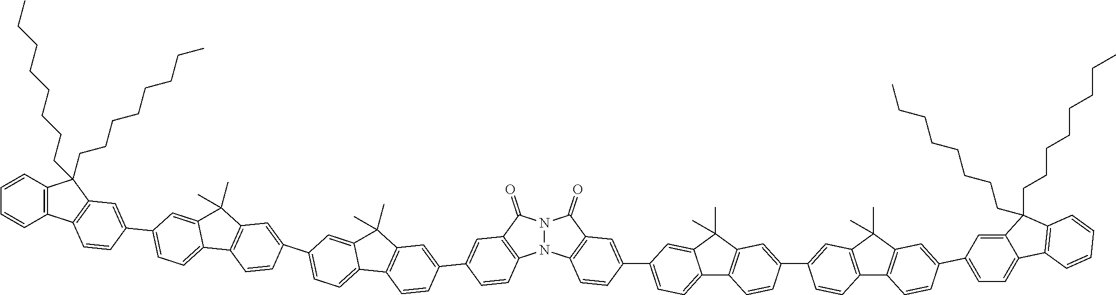 [SMILES: CCCCCCCCC1(CCCCCCCC)c2ccccc2-c2ccc(-c3ccc4c(c3)C(C)(C)c3cc(-c5ccc6c(c5)C(C)(C)c5cc(-c7ccc8c(c7)c(=O)n7c(=O)c9cc(-c%10ccc%11c(c%10)C(C)(C)c%10cc(-c%12ccc%13c(c%12)C(C)(C)c%12cc(-c%14ccc%15c(c%14)C(CCCCCCCC)(CCCCCCCC)c%14ccccc%14-%15)ccc%12-%13)ccc%10-%11)ccc9n87)ccc5-6)ccc3-4)cc21] |
| L3 | S50 | G10 from WO2018/ 07421 | 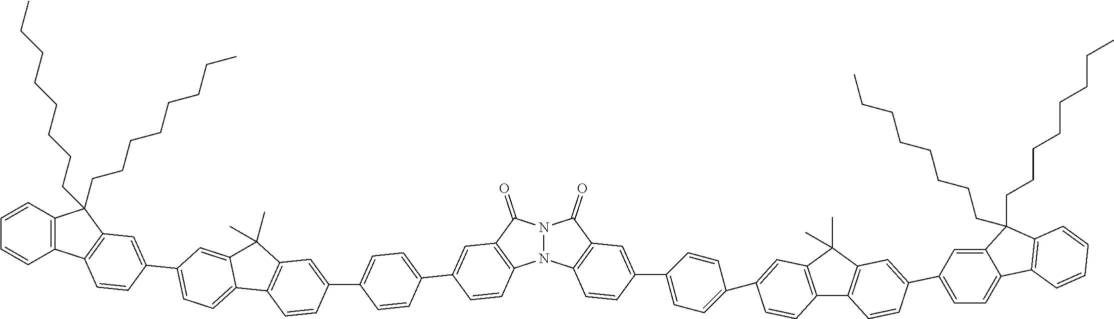 [SMILES: CCCCCCCCC1(CCCCCCCC)c2ccccc2-c2ccc(-c3ccc4c(c3)C(C)(C)c3cc(-c5ccc(-c6ccc7c(c6)c(=O)n6c(=O)c8cc(-c9ccc(-c%10ccc%11c(c%10)C(C)(C)c%10cc(-c%12ccc%13c(c%12)C(CCCCCCCC)(CCCCCCCC)c%12ccccc%12-%13)ccc%10-%11)cc9)ccc8n76)cc5)ccc3-4)cc21] |
| L4 | S50 | Int-D1 | 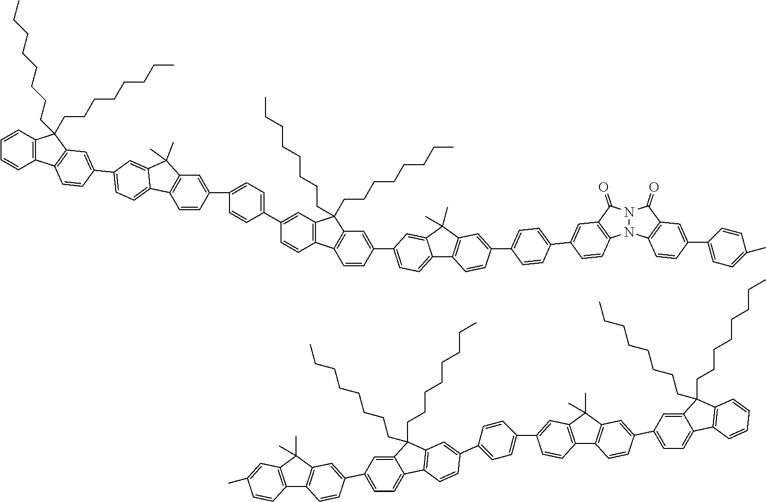 |
| L5 | S51 | Int-D1 | 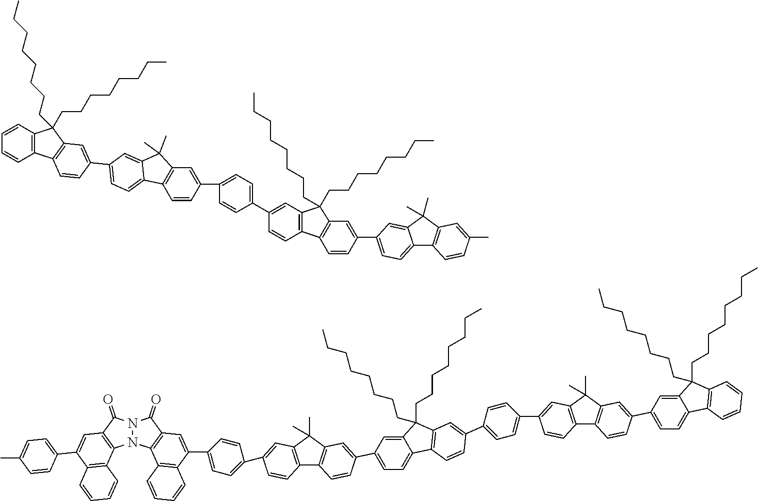 |
| L6 | S52 | Int-D1 | 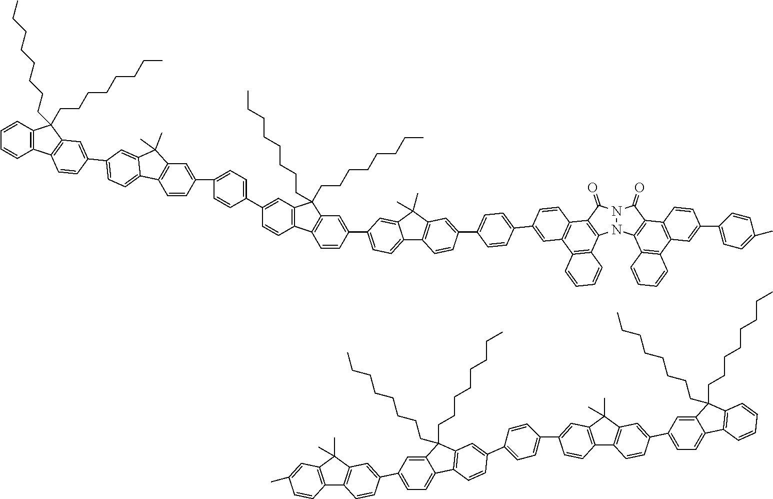 |

### Measurement of Photoluminescence Spectra (PL Spectra):

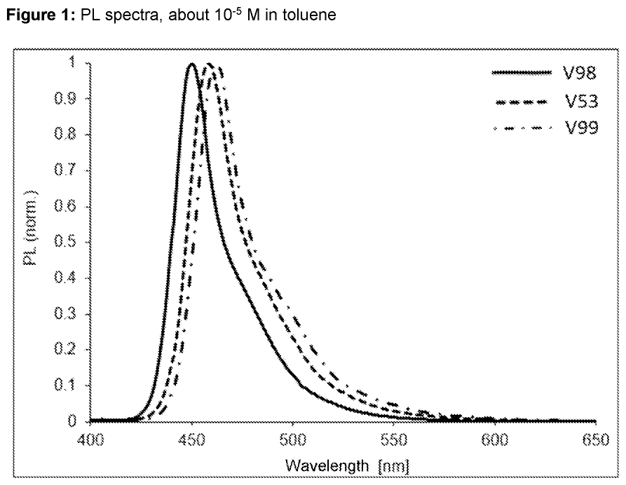

FIG. 1 shows PL spectra of inventive compounds V53, V98 and V99, measured with a Hitachi F-4500 PL spectrometer in about 10⁻⁵ molar degassed toluene solution at room temperature (about 25° C.).

The PL spectra have very narrow emission bands with low FWHM values (<0.18 eV) and lead to particularly pure-colour emission.

### Production of OLED Components

### 1) Vacuum-Processed Devices:

OLEDs of the invention and OLEDs according to the prior art are produced by a general method according to WO 2004/058911, which is adapted to the circumstances described here (variation in layer thickness, materials used).

In the examples which follow, the results for various OLEDs are presented. Cleaned glass plates (cleaning in Miele laboratory glass washer, Merck Extran detergent) coated with structured ITO (indium tin oxide) of thickness 50 nm are pretreated with UV ozone for 25 minutes (PR-100 UV ozone generator from UVP) and, within 30 min, for improved processing, coated with 20 nm of PEDOT:PSS (poly(3,4-ethylenedioxythiophene) poly(styrenesulfonate), purchased as CLEVIOS™ P VP Al 4083 from Heraeus Precious Metals GmbH Deutschland, spun on from aqueous solution) and then baked at 180° C. for 10 min. These coated glass plates form the substrates to which the OLEDs are applied.

### 1a) Blue and Green Fluorescence OLED Components—BF and GF:

All materials are applied by thermal vapour deposition in a vacuum chamber. The emission layer (EML) here always consists of at least one matrix material (host material) SMB (see table 1) and an emitting dopant (dopant, emitter) V, which is added to the matrix material(s) in a particular proportion by volume by co-evaporation. Details given in such a form as SMB:V (97:3%) mean here that the material SMB is present in the layer in a proportion by volume of 97% and the dopant V in a proportion of 3%. Analogously, the inventive compounds V44, V45 and V46 may be used as host for green-fluorescing components. Analogously, the electron transport layer may also consist of a mixture of two materials; see table 1. The materials used for production of the OLEDs are shown in table 7.

The OLEDs are characterized in a standard manner. For this purpose, the electroluminescence spectra, the current efficiency (measured in cd/A), the power efficiency (measured in lm/W) and the external quantum efficiency (EQE, measured in percent) as a function of luminance, calculated from current-voltage-luminance characteristics (IUL characteristics) assuming Lambertian emission characteristics, and also the lifetime are determined. Electroluminescence spectra are determined at a luminance of 1000 cd/m², and these are used to calculate the CIE 1931 x and y colour coordinates and the FWHM (full width at half maximum).

The OLEDs have the Following Layer Structure:

### Substrate

Hole injection layer 1 (HIL1) composed of HTM1 doped with 5%
NDP-9 (commercially available from Novaled), 20 nm
Hole transport layer 1 (HTL1) composed of HTM1, 140 nm
Hole transport layer 2 (HTL2) composed of HTM2, 10 nm
Emission layer (EML), see table 1
Electron transport layer (ETL2), see table 1
Electron transport layer (ETL1), see table 1
Electron injection layer (EIL) composed of ETM2, 1 nm
Cathode composed of aluminium, 100 nm

TABLE 1
Structure of blue BF and green GF fluorescence
OLED
components

| Ex. | EML | ETL2 | ETL1 |
|---|---|---|---|
| BF1 | SMB1:V64 (97:3%) | — | ETM1:ETM2 |
| | 20 nm | | (50:50%) |
| | | | 30 nm |
| BF2 | SMB2:V65 (97:3%) | — | ETM1:ETM2 |
| | 20 nm | | (50:50%) |
| | | | 30 nm |
| BF3 | SMB3:V67 (97:3%) | ETM1 | ETM1:ETM2 |
| | 20 nm | 5 nm | (50:50%) |
| | | | 30 nm |
| BF4 | SMB1:V83 (97:3%) | V5 | V5:ETM2 (50:50%) |
| | 20 nm | 5 nm | 30 nm |
| BF5 | SMB1:V53 (97:3%) | — | ETM1:ETM2 |
| | 20 nm | | (50:50%) |
| | | | 30 nm |
| BF6 | SMB1:V98 (97:3%) | — | ETM1:ETM2 |
| | 20 nm | | (50:50%) |
| | | | 30 nm |
| BF7 | SMB1:V99 (97:3%) | — | ETM1:ETM2 |
| | 20 nm | | (50:50%) |
| | | | 30 nm |
| GF1 | V44:V54 (95:5%) | — | ETM1:ETM2 |
| | 20 nm | | (50:50%) |
| | | | 30 nm |
| GF2 | V45:V57 (95:5%) | V4 | V4:ETM2 (50:50%) |
| | 20 nm | 5 nm | 30 nm |

TABLE 2
Results for blue fluorescence OLED components

| | EQE (%) | Voltage (V) | CIE |
|---|---|---|---|
| Ex. | 1000 cd/m² | 1000 cd/m² | (x/y) |
| BF1 | 6.8 | 3.5 | 0.16/0.06 |
| BF2 | 7.5 | 3.4 | 0.16/0.10 |
| BF3 | 6.7 | 3.3 | 0.15/0.08 |
| BF4 | 7.2 | 3.1 | 0.15/0.09 |
| BF5 | 6.3 | 3.5 | 0.15/0.11 |
| BF6 | 7.4 | 3.6 | 0.15/0.08 |
| BF7 | 7.0 | 3.5 | 0.16/0.13 |
| GF1 | 28.0 | 3.0 | 0.26/0.64 |
| GF2 | 25.6 | 2.8 | 0.24/0.59 |

### 1b) Phosphorescence OLED Components—BP, GP, RP:

Among other uses, the compounds of the invention can be used as electron-conducting host material eTMM in the emission layer EML of a phosphorescent OLED, and as electron transport material in the HBL and ETL.

For this purpose, all the materials are applied by thermal vapour deposition in a vacuum chamber. The emission layer here always consists of at least one or more than one matrix material M (host material) and a phosphorescent dopant Ir which is added to the matrix material(s) in a particular proportion by volume by co-evaporation. Details given in such a form as M1:M2:Ir (55%:35%:10%) mean here that the material M1 is present in the layer in a proportion by volume of 55%, M2 in a proportion by volume of 35% and Ir in a proportion by volume of 10%. Analogously, the electron transport layer may also consist of a mixture of two materials. The exact structure of the OLEDs can be found in table 3. The materials used for production of the OLEDs are shown in Table 7.

The OLEDs are characterized in a standard manner. For this purpose, the electroluminescence spectra, the current efficiency (measured in cd/A), the power efficiency (measured in lm/W) and the external quantum efficiency (EQE, measured in percent) as a function of luminance, calculated from current-voltage-luminance characteristics (IUL characteristics) assuming Lambertian emission characteristics, and also the lifetime are determined. Electroluminescence spectra are determined at a luminance of 1000 cd/m², and these are used to calculate the CIE 1931 x and y colour coordinates.

The OLEDs have the Following Layer Structure:

### Substrate

Hole injection layer 1 (HIL1) composed of HTM1 doped with 5% NDP-9 (commercially available from Novaled), 20 nm
Hole transport layer 1 (HTL1) composed of HTM1, 170 nm for blue, 40 nm for green/yellow and 90 nm for red components
Hole transport layer 2 (HTL2), see table 3
Emission layer (EML), see table 3
Electron transport layer (ETL2), see table 3
Electron transport layer (ETL1), see table 3
Electron injection layer (EIL) composed of ETM2, 1 nm
Cathode composed of aluminium, 100 nm

TABLE 3
Structure of phosphorescence OLED components

| | | | | |
|---|---|---|---|---|
| | HTL2 | EML | ETL2 | ETL1 |
| Ex. | thickness | thickness | thickness | thickness |

Blue OLEDs

| | | | | |
|---|---|---|---|---|
| BP1 | HTM3 | M3:M4:IrB1 | ETM3 | V1:ETM2 |
| | 10 nm | (30%:60%:10%) | 10 nm | (70%:30%) |
| | | 30 nm | | 30 nm |
| BP2 | HTM3 | M3:M4:IrB1 | ETM3 | V6:ETM2 |
| | 10 nm | (30%:60%:10%) | 10 nm | (60%:40%) |
| | | 30 nm | | 30 nm |

Green and yellow OLEDs

| | | | | |
|---|---|---|---|---|
| GP1 | HTM2 | V9:M1:IrG1 | ETM1 | ETM1:ETM2 |
| | 20 nm | (50%:40%:10%) | 5 nm | (50%:50%) |
| | | 40 nm | | 30 nm |
| GP2 | HTM2 | V12:M2:IrG2 | ETM1 | ETM1:ETM2 |
| | 20 nm | (50%:35%:15%) | 5 nm | (50%:50%) |
| | | 40 nm | | 30 nm |
| GP3 | HTM2 | V24:M1:IrG3 | ETM1 | ETM1:ETM2 |
| | 20 nm | (45%:45%:10%) | 5 nm | (50%:50%) |
| | | 40 nm | | 30 nm |
| GP4 | HTM2 | V32:M2:IrG3 | V3 | ETM1:ETM2 |
| | 20 nm | (45%:45%:10%) | 5 nm | (50%:50%) |
| | | 40 nm | | 30 nm |
| GP5 | HTM2 | V35:M1:IrG3 | V5 | V5:ETM2 |
| | 20 nm | (45%:45%:10%) | 5 nm | (60%:40%) |
| | | 40 nm | | 30 nm |

Red OLEDs

| | | | | |
|---|---|---|---|---|
| RP1 | HTM2 | V94:IrR1 | ETM1 | ETM1:ETM2 |
| | 10 nm | (97%:3%) | 10 nm | (50%:50%) |
| | | 35 nm | | 30 nm |

TABLE 4
Results for phosphorescence OLED components

| | | | |
|---|---|---|---|
| | EQE (%) | Voltage (V) | CIE x/y |
| Ex. | 1000 cd/m² | 1000 cd/m² | 1000 cd/m² |

Blue OLEDs

| | | | |
|---|---|---|---|
| BP1 | 20.6 | 3.5 | 0.16/0.36 |
| BP2 | 22.3 | 3.4 | 0.16/0.36 |

Green and yellow OLEDs

| | | | |
|---|---|---|---|
| GP1 | 23.2 | 3.3 | 0.34/0.62 |
| GP2 | 21.9 | 3.2 | 0.35/0.65 |
| GP3 | 27.7 | 3.2 | 0.49/0.51 |
| GP4 | 28.3 | 3.1 | 0.49/0.51 |
| GP5 | 28.5 | 3.0 | 0.50/0.50 |

Red OLEDs

| | | | |
|---|---|---|---|
| RP1 | 18.6 | 2.7 | 0.70/0.30 |

### 2) Solution-Processed Components: 2a) Solution-Processed Phosphorescence OLED Components—Sol-GP and Sol-RP:

The iridium complexes of the invention can also be processed from solution and lead therein to OLEDs which are much simpler in terms of process technology compared to the vacuum-processed OLEDs, but nevertheless have good properties. The production of such components is based on the production of polymeric light-emitting diodes (PLEDs), which has already been described many times in the literature (for example in WO 2004/037887). The structure is composed of:

The construction used is thus as follows:
substrate,
ITO (50 nm),
hole injection layer (60 nm),
interlayer (20 nm), composed of HTL-S1
emission layer (60 nm), see table 5
hole blocker layer (10 nm), composed of ETM-1
electron transport layer (40 nm), composed of ETM-1/ETM-2 (50:50)
cathode (Al) (100 nm)

For this purpose, substrates from Technoprint (soda-lime glass) are used, to which the ITO structure (indium tin oxide, a transparent conductive anode) is applied. The substrates are cleaned in a cleanroom with DI water and a detergent (Deconex 15 PF) and then activated by a UV/ozone plasma treatment. Thereafter, likewise in a cleanroom, a 20 nm hole injection layer (PEDOT:PSS from Clevios™) is applied by spin-coating.

The required spin rate depends on the degree of dilution and the specific spin-coater geometry. In order to remove residual water from the layer, the substrates are baked on a hotplate at 180° C. under air for 10 minutes. The interlayer used serves for hole transport; in this case, HTL-S1 from Merck is used (WO2013156130). The solids content is about 5 g/l, in order to achieve a layer thickness of 20 nm by means of spin-coating. The interlayer is baked in an inert gas atmosphere, argon in the present case, at 220° C. on a hotplate for 30 minutes. The interlayer may alternatively also be replaced by one or more layers which merely have to fulfil the condition of not being leached off again by the subsequent processing step of EML deposition from solution. For production of the emission layer, the triplet emitters Ir are dissolved together with the matrix materials in toluene or chlorobenzene. The typical solids content of such solutions is between 16 and 25 g/l when, as here, the layer thickness of 60 nm which is typical of a device is to be achieved by means of spin-coating. The solution-processed devices contain an emission layer composed of Ma:Mb:Ir (w %:x %:z %); see table 5. The emission layer is spun on in an inert gas atmosphere, argon in the present case, and baked at 160° C. for 10 min. Vapour-deposited atop the latter are the hole blocker layer and the electron transport layer (vapour deposition systems from Lesker or the like, typical vapour deposition pressure 5×10⁻⁶ mbar). Finally, a cathode of aluminium (high-purity metal from Aldrich) is applied by vapour deposition. In order to protect the device from air and air humidity, the device is finally encapsulated and then characterized. The OLED examples cited have not yet been optimized. Table 5 summarizes the data

TABLE 5
Results with materials processed from solution

| | | EQE | Voltage | |
|---|---|---|---|---|
| | | (%) | (V) | CIE x/y |
| | | 1000 | 1000 | 1000 |
| Ex. | Emission layer | cd/m² | cd/m² | cd/m² |
| Sol- | V22:M5:IrG-Sol1 | 22.0 | 4.4 | 0.34/0.62 |
| GP1 | (20%:58%:22%) | | | |
| Sol- | V200:M5:IrG-Sol1 | 22.7 | 4.3 | 0.34/0.63 |
| GP2 | (20%:58%:22%) | | | |
| Sol- | V213:M5:IrG-Sol1 | 22.5 | 4.3 | 0.34/0.62 |
| GP3 | (30%:50%:20%) | | | |
| Sol- | V307:M5:IrR1 | 17.7 | 3.8 | 0.70/0.30 |
| RP1 | (25%:57%:18%) | | | |

### 2b) Solution-Processed Fluorescence OLED Components—Sol-GP

The inventive fluorescence emitters L can likewise be processed from solution and lead to good properties. Such components are produced analogously to the solution-processed phosphorescence OLED components—GP, RP—with the adjustment described below made in the construction:
substrate,
ITO (50 nm),
hole injection layer (20 nm),
interlayer (20 nm), composed of HTL-S1
emission layer (40 nm), composed of fluorescent emitter L and matrix MS
hole blocker layer (10 nm), composed of ETM-1
electron transport layer (40 nm), composed of ETM-1/ETM-2 (50:50)
cathode (Al) (100 nm).

For production of the emission layer, the inventive fluorescence emitters L (5% by weight) are dissolved together with the matrix material MS (95% by weight) in toluene or chlorobenzene. The typical solids content of such solutions is 14 g/I when, as here, the layer thickness of 40 nm which is typical of a device is to be achieved by means of spin-coating. The emission layer is spun on in an inert gas atmosphere, argon in the present case, and baked at 160° C. for 10 min. Table 6 summarizes the data obtained.

TABLE 6
Results with materials processed from solution

| | | | EQE | | |
|---|---|---|---|---|---|
| | | | (%) | Voltage | CIE x/y |
| | Emission layer | | 1000 | (V) | 1000 |
| Ex. | Host | Emitter | cd/m² | 1000 cd/m² | cd/m² |
| Sol- | MS | L1 | 3.9 | 4.3 | 0.14/0.14 |
| BF1 | | | | | |
| Sol- | MS | L2 | 4.3 | 4.3 | 0.14/0.14 |
| BF2 | | | | | |
| Sol- | MS | L3 | 4.5 | 4.4 | 0.14/0.13 |
| BF3 | | | | | |
| Sol- | MS | L4 | 5.9 | 4.4 | 0.14/0.13 |
| BF4 | | | | | |

TABLE 7
Structural formulae of the material used

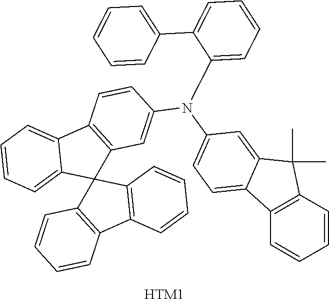

136463-07-5

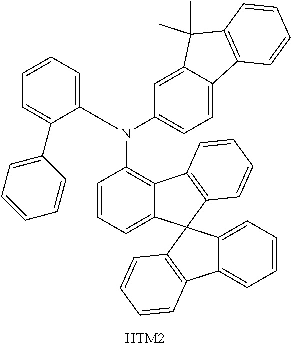

1450933-44-4

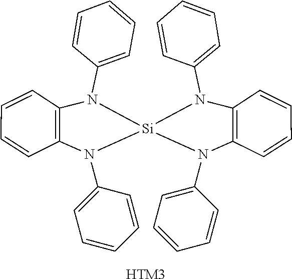

1206465-62-4

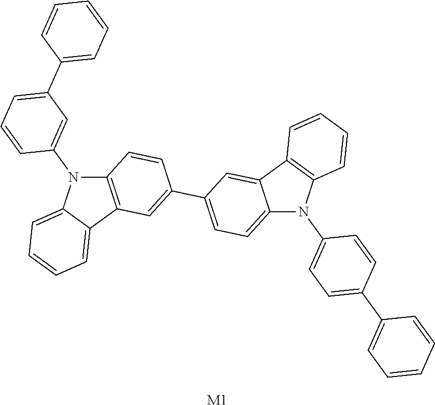 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; "M1"]

1643479-47-3

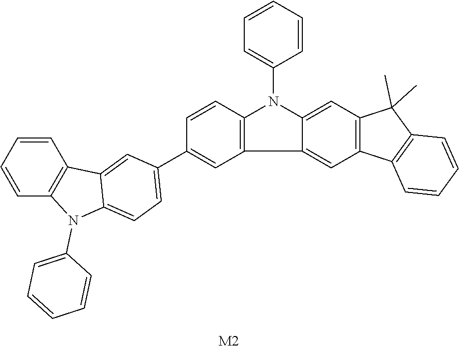 [SMILES: CC1(C)c2ccccc2-c2cc3c4cc(-c5ccc6c(c5)c5ccccc5n6-c5ccccc5)ccc4n(-c4ccccc4)c3cc21 ; "M2"]

1357150-54-9

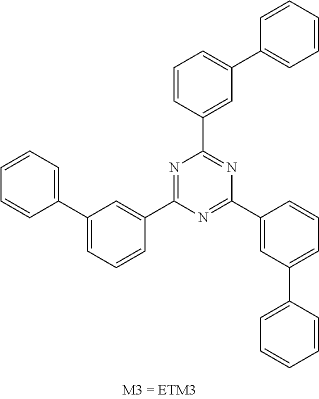 [SMILES: c1ccc(-c2cccc(-c3nc(-c4cccc(-c5ccccc5)c4)nc(-c4cccc(-c5ccccc5)c4)n3)c2)cc1]

1201800-83-0

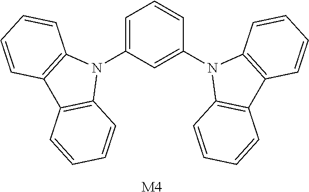 [SMILES: c1cc(-n2c3ccccc3c3ccccc32)cc(-n2c3ccccc3c3ccccc32)c1 ; "M4"]

550378-78-4

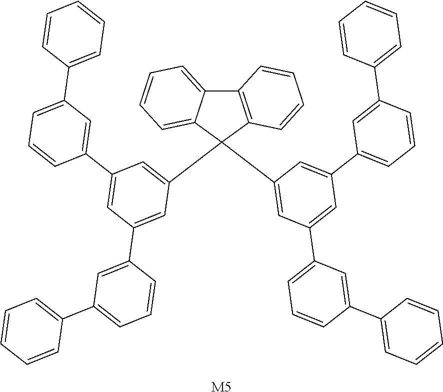 [SMILES: c1ccc(-c2cccc(-c3cc(-c4cccc(-c5ccccc5)c4)cc(C4(c5cc(-c6cccc(-c7ccccc7)c6)cc(-c6cccc(-c7ccccc7)c6)c5)c5ccccc5-c5ccccc54)c3)c2)cc1 ; "M5"]

1246496-85-4

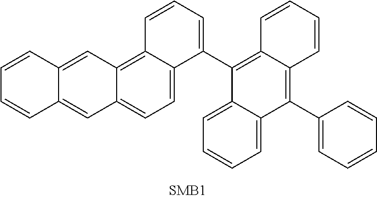 [SMILES: c1ccc(-c2c3ccccc3c(-c3cccc4c3ccc3cc5ccccc5cc34)c3ccccc23)cc1]

[1087346-88-0]

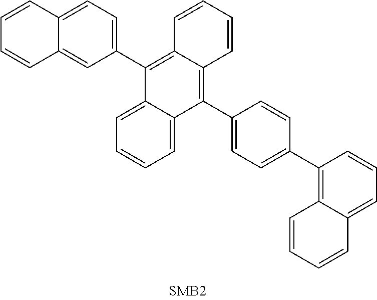 [SMILES: c1ccc2cc(-c3c4ccccc4c(-c4ccc(-c5cccc6ccccc56)cc4)c4ccccc34)ccc2c1]

[667940-34-3]

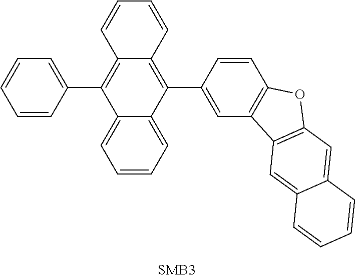 [SMILES: c1ccc(-c2c3ccccc3c(-c3ccc4oc5cc6ccccc6cc5c4c3)c3ccccc23)cc1]

[1627916-48-6]

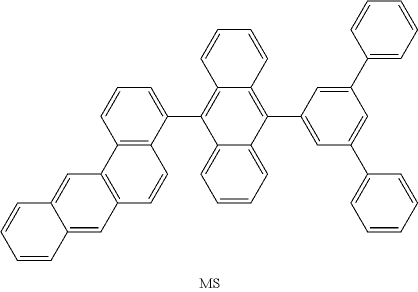 [SMILES: c1ccc(-c2cc(-c3ccccc3)cc(-c3c4ccccc4c(-c4cccc5c4ccc4cc6ccccc6cc45)c4ccccc34)c2)cc1]

[1273319-88-2]

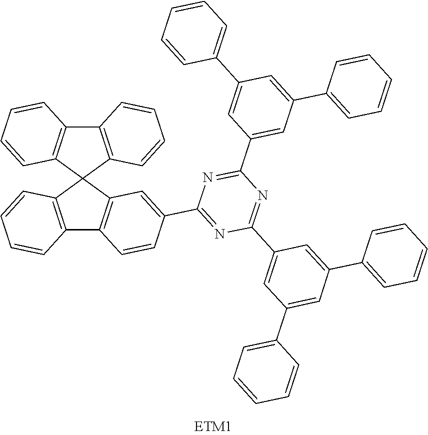 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

1233200-52-6

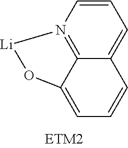

25387-93-3

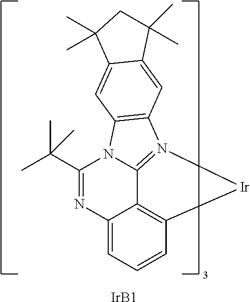

1541114-98-0

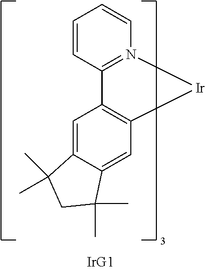

1562420-772

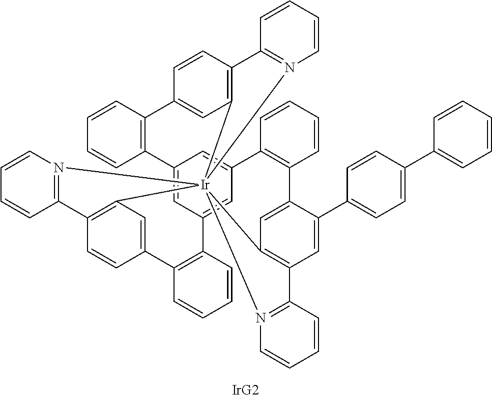

2245866-06-0

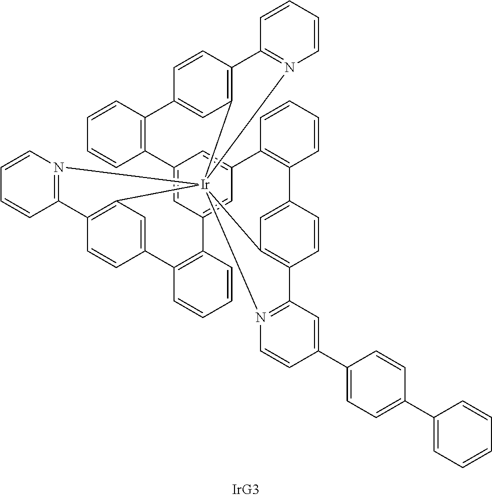

2245945-28-0

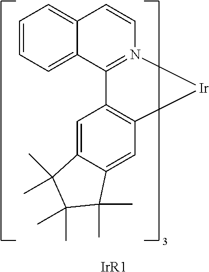

1562420-79-4

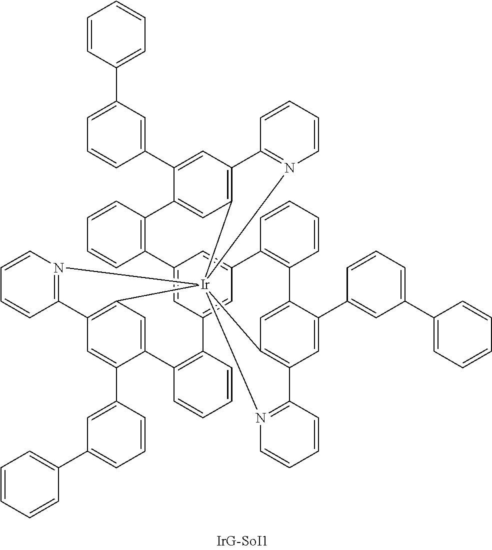

1989601-89-9

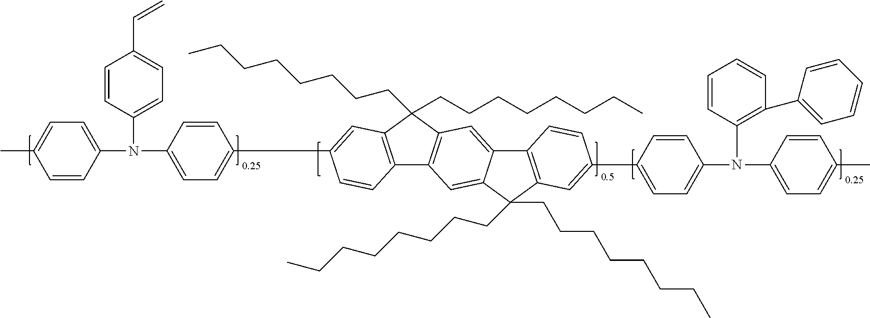

HTL-S1 WO2013156130

The abbreviations shown in tables 1, 3, 5 and 6 in relation to the materials of the invention, for example L1, L2, L3, L4, V1, V4, V5, V6, V9, V12, V22, V24, V32, V35, V44, V45, V54, V57, V64, V65, V67, V83, V94, V200, V213 and V307 etc., relate to the compounds recited in detail above in the synthesis examples.

1.-18. (canceled)

19. A compound including at least one structure of the formula (I) 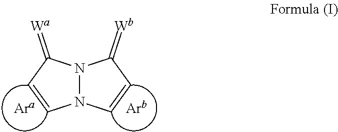
where the ring Ar^{a} is the same or different at each instance and is an aromatic or heteroaromatic ring system which has 5 to 60 aromatic ring atoms and may be substituted by one or more Ar or R^{a} radicals, the ring Ar^{b} is the same or different at each instance and is an aromatic or heteroaromatic ring system which has 5 to 60 aromatic ring atoms and may be substituted by one or more Ar or R^{b} radicals;
and where the further symbols and indices used are as follows:
W^{a}, W^{b} is the same or different at each instance and is O or S;
Ar is the same or different at each instance and is an aromatic or heteroaromatic ring system which has 5 to 60 aromatic ring atoms and may be substituted by one or more R radicals; the Ar group here may form a ring system with at least one Ar, R, R^{a}, R^{b} group or a further group;
R, R^{a}, R^{b} is the same or different at each instance and is H, D, OH, F, Cl, Br, I, CN, NO₂, N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; or heteroarylthio group which has 5 to 60 aromatic ring atoms and may be substituted by one or more R¹ radicals, or a diarylamino, arylheteroarylamino, diheteroarylamino group which has 5 to 60 aromatic ring atoms and may be substituted by one or more R¹ radicals, or an aralkyl or heteroarylalkyl group which has 5 to 60 aromatic ring atoms and 1 to 10 carbon atoms in the alkyl radical and may be substituted by one or more R¹ radicals; at the same time, two R, R^{a}, R^{b} radicals together or with a further group may also form a ring system;
Ar′ is the same or different at each instance and is an aromatic or heteroaromatic ring system which has 5 to 60 aromatic ring atoms and may be substituted by one or more R¹ radicals; at the same time, it is possible for two Ar′ radicals bonded to the same carbon atom, silicon atom, nitrogen atom, phosphorus atom or boron atom also to be joined together via a bridge by a single bond or a bridge selected from B(R¹), C(R¹)₂, Si(R¹)₂, C═O, C═NR¹, C═C(R¹)₂, O, S, S═O, SO₂, N(R¹), P(R¹) and P(═O)R¹;
R¹ is the same or different at each instance and is H, D, F, Cl, Br, I, CN, NO₂, N(Ar″)₂, N(R²)₂, C(═O)Ar″, C(═O)R², P(═O)(Ar″)₂, P(Ar″)₂, B(Ar″)₂, B(R²)₂, C(Ar″)₃, C(R²)₃, Si(Ar″)₃, Si(R²)₃, a straight-chain alkyl, alkoxy or thioalkoxy group having 1 to 40 carbon atoms or a branched or cyclic alkyl, alkoxy or thioalkoxy group having 3 to 40 carbon atoms or an alkenyl group having 2 to 40 carbon atoms, each of which may be substituted by one or more R² radicals, where one or more nonadjacent CH₂ groups may be replaced by —R²C═CR²—, —C≡C—, Si(R²)₂, C═O, C═S, C═Se, C═NR², —C(═O)O—, —C(═O)NR²—, NR², P(═O)(R²), —O—, —S—, SO or SO₂ and where one or more hydrogen atoms may be replaced by D, F, Cl, Br, I, CN or NO₂, or an aromatic or heteroaromatic ring system which has 5 to 60 aromatic ring atoms, each of which may be substituted by one or more R² radicals, or an aryloxy or heteroaryloxy group which has 5 to 60 aromatic ring atoms and may be substituted by one or more R² radicals, or an aralkyl or heteroaralkyl group which has 5 to 60 aromatic ring atoms and may be substituted by one or more R² radicals, or a combination of these systems; at the same time, two or more R¹ radicals together may form a ring system; at the same time, one or more R¹ radicals may form a ring system with a further part of the compound;
Ar″ is the same or different at each instance and is an aromatic or heteroaromatic ring system which has 5 to 30 aromatic ring atoms and may be substituted by one or more R² radicals; at the same time, it is possible for two Ar″ radicals bonded to the same carbon atom, silicon atom, nitrogen atom, phosphorus atom or boron atom also to be joined together via a bridge by a single bond or a bridge selected from B(R²), C(R²)₂, Si(R²)₂, C═O, C═NR², C═C(R²)₂, O, S, S═O, SO₂, N(R²), P(R²) and P(═O)R²;
R² is the same or different at each instance and is selected from the group consisting of H, D, F, CN, an aliphatic hydrocarbyl radical having 1 to 20 carbon atoms or an aromatic or heteroaromatic ring system which has 5 to 30 aromatic ring atoms and in which one or more hydrogen atoms may be replaced by D, F, Cl, Br, I or CN and which may be substituted by one or more alkyl groups each having 1 to 4 carbon atoms; at the same time, two or more substituents R² together may form a ring system;
with exclusion of compounds of the formulae (A), (B) and (C) 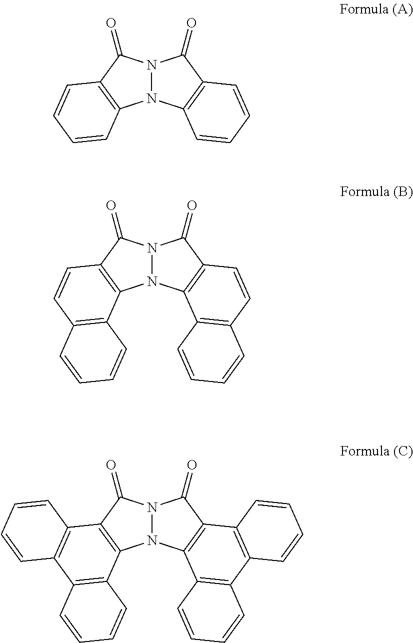 [SMILES: O=c1c2ccccc2n2c3ccccc3c(=O)n12 ; O=c1c2ccc3ccccc3c2n2c3c(ccc4ccccc43)c(=O)n12 ; O=c1c2c3ccccc3c3ccccc3c2n2c3c4ccccc4c4ccccc4c3c(=O)n12]
from protection.

20. The compound according to claim 19, comprising at least one structure of the formula (II-1) to (II-42): 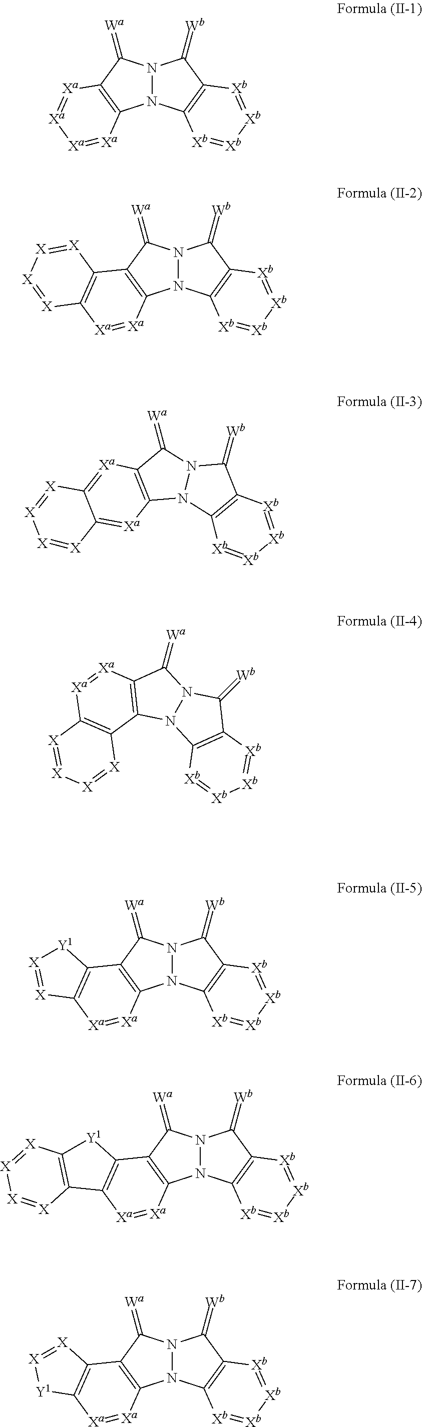 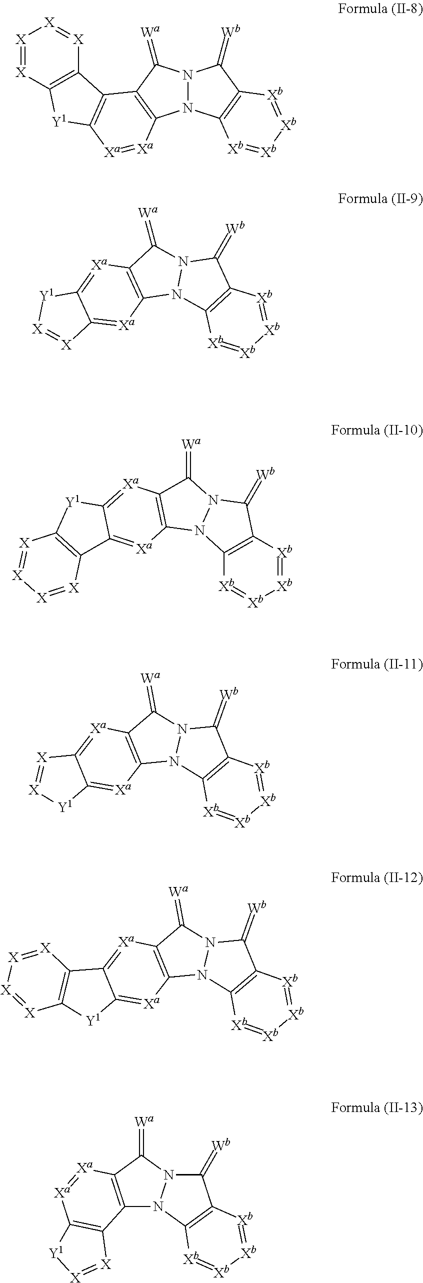 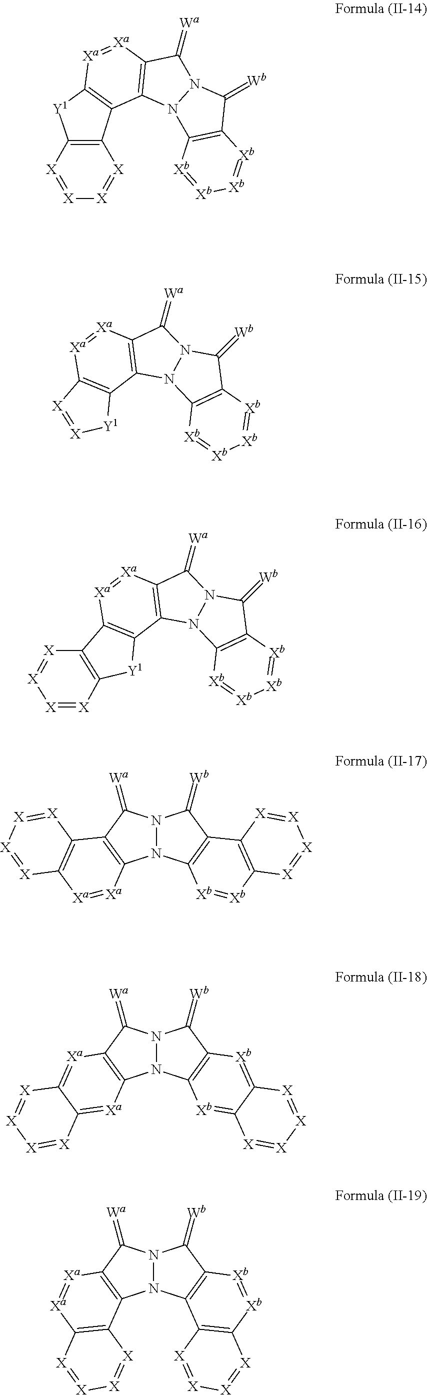 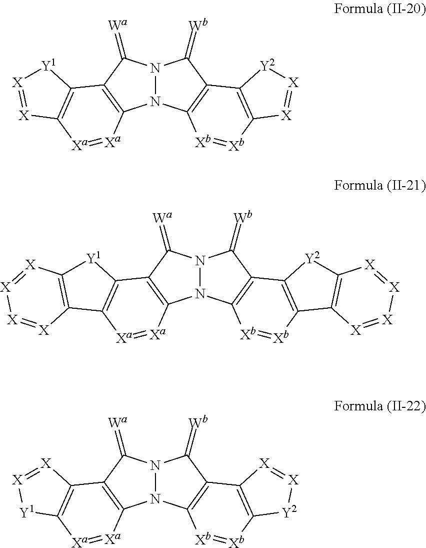 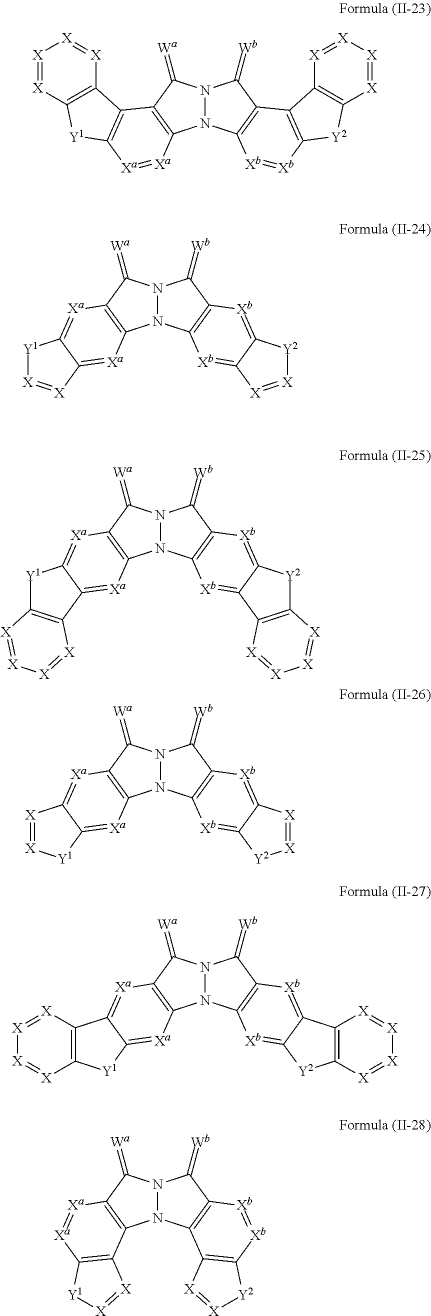 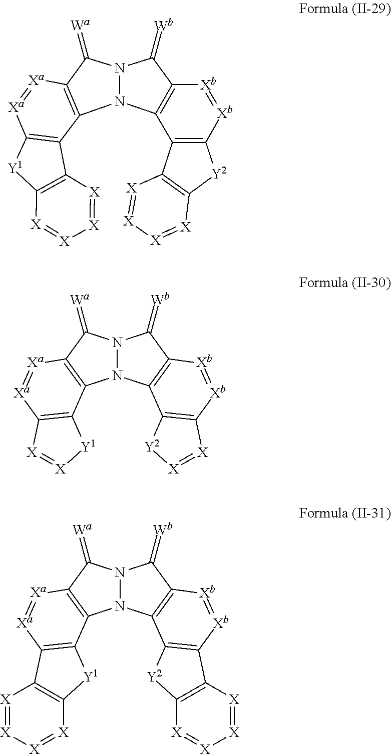 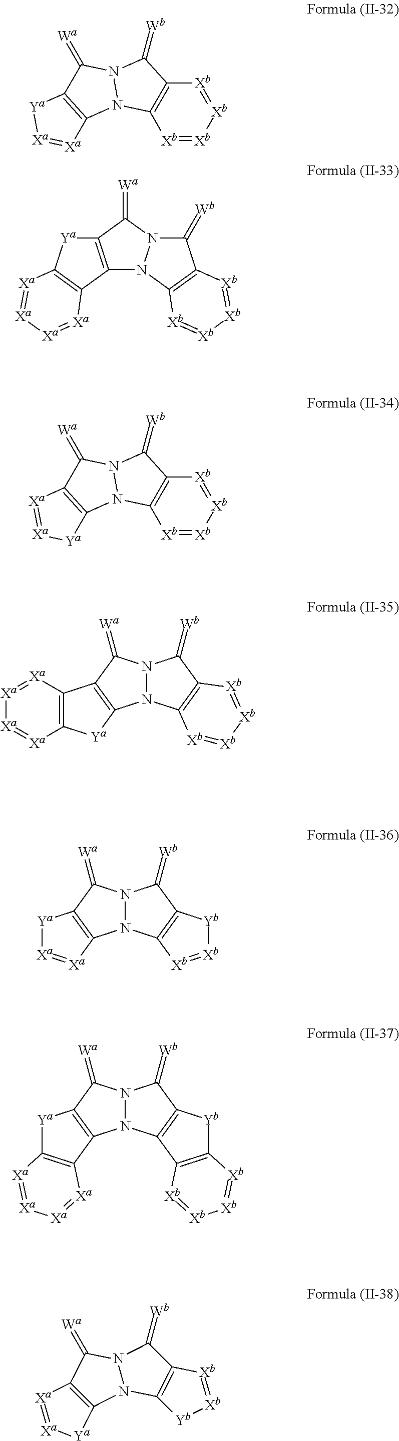 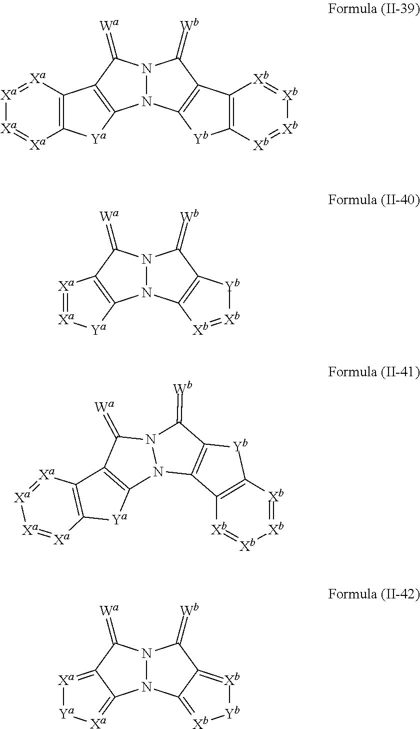
where the symbols W^{a} and W^{b} have the definitions given in claim 19 and the further symbols are as follows:
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, where R^{a} has the definition detailed in claim 19;
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, where R^{b} has the definition detailed in claim 19;
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, where R has the definition detailed in claim 19;
X is the same or different at each instance and is N or CR, with the proviso that not more than two of the X groups in one cycle are N, where R has the definition detailed in claim 19;
X^{a} is the same or different at each instance and is N or CR^{a}, with the proviso that not more than two of the X^{a} groups in one cycle are N, where R^{a} has the definition detailed in claim 19;
X^{b} is the same or different at each instance and is N or CR^{b}, with the proviso that not more than two of the X^{b} groups in one cycle are N, where R^{b} has the definition detailed in claim 19.

21. The compound according to claim 20, wherein
Y^{a} is the same or different at each instance and is N(Ar), C(R^{a})₂, O or S;
Y^{b} is the same or different at each instance and is N(Ar), C(R^{b})₂, O or S; and
Y¹, Y² is the same or different at each instance and is N(Ar), C(R)₂, O or S;

22. The compound according to claim 19, including at least one structure of the formulae (III-1) to (III-41): 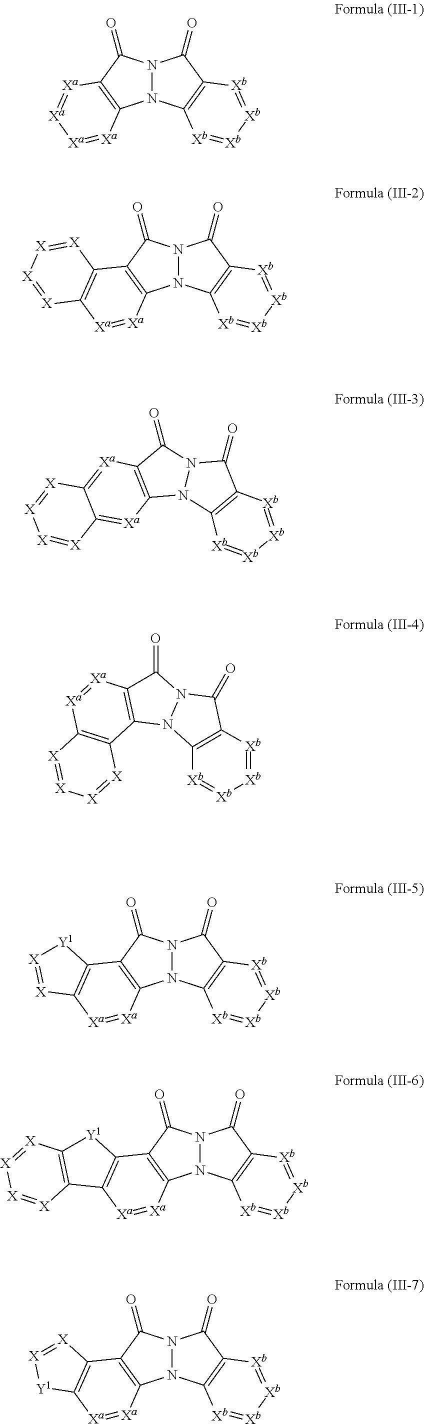 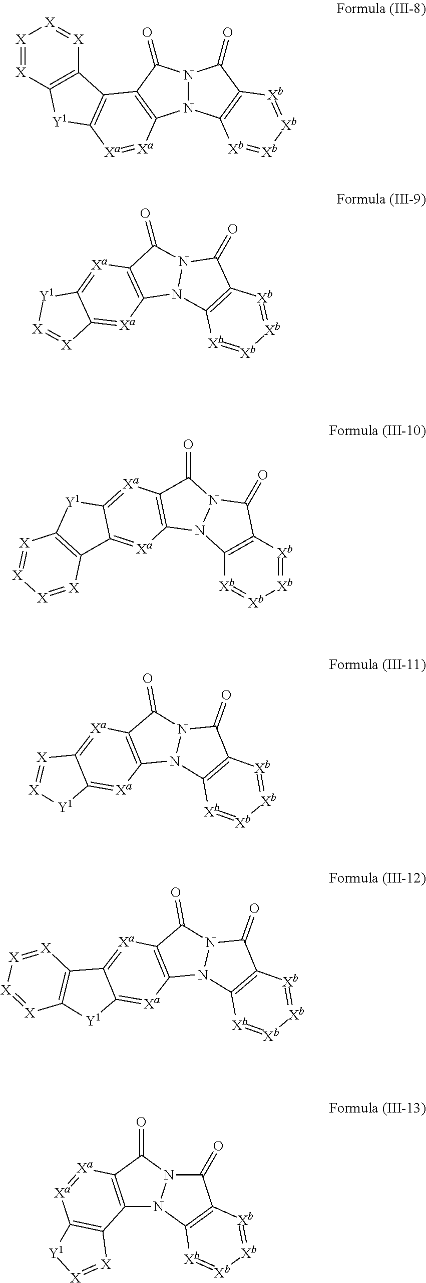 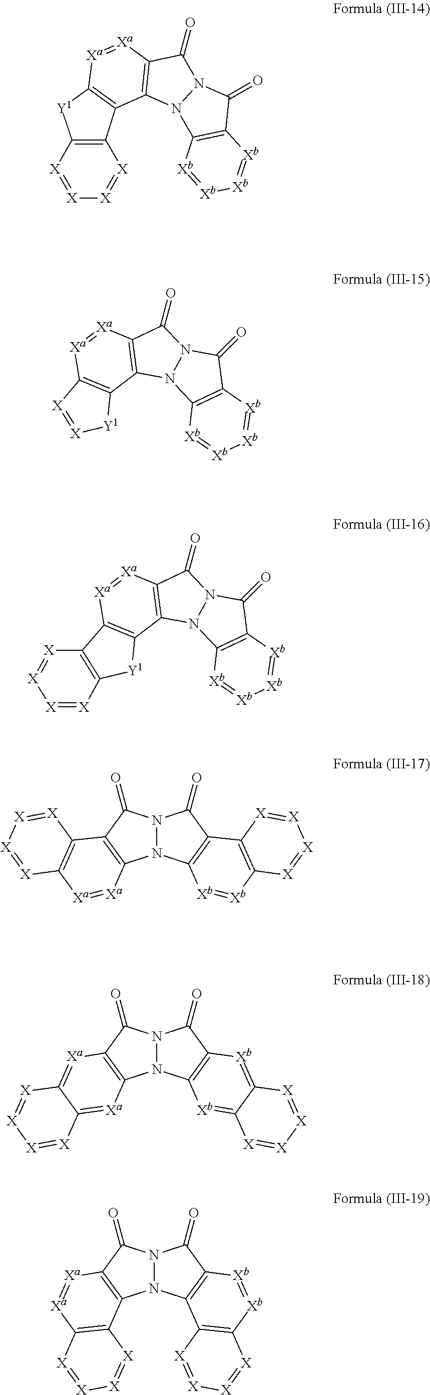 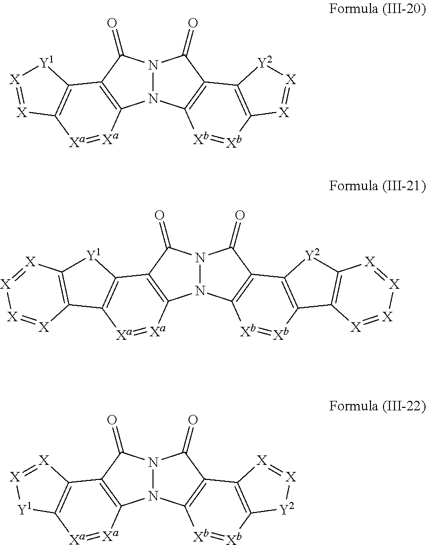 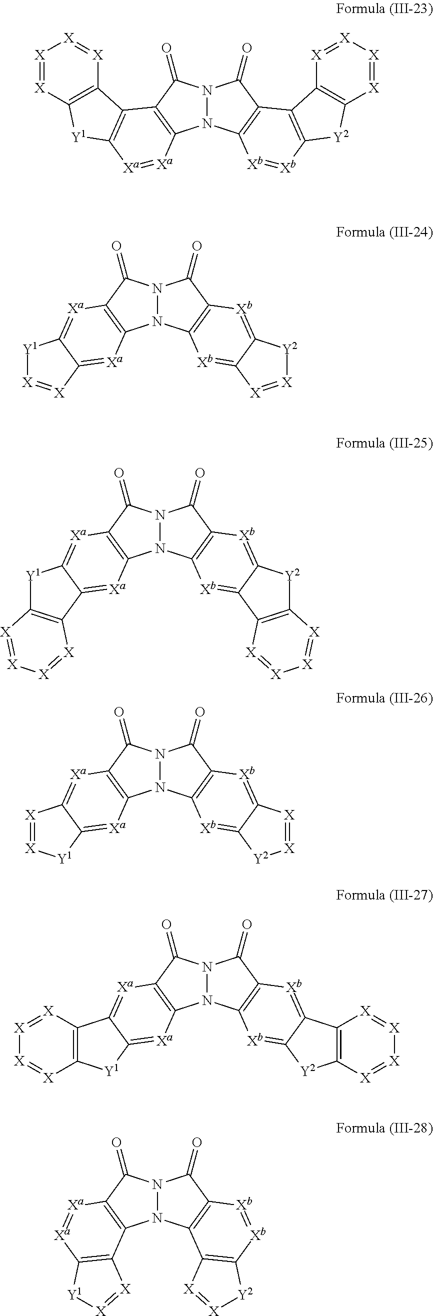 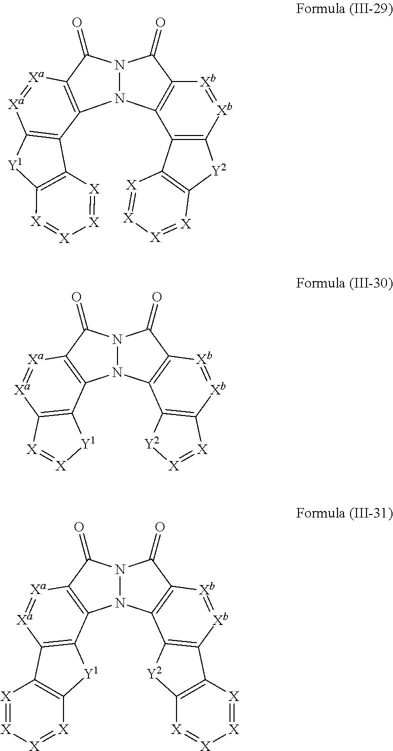 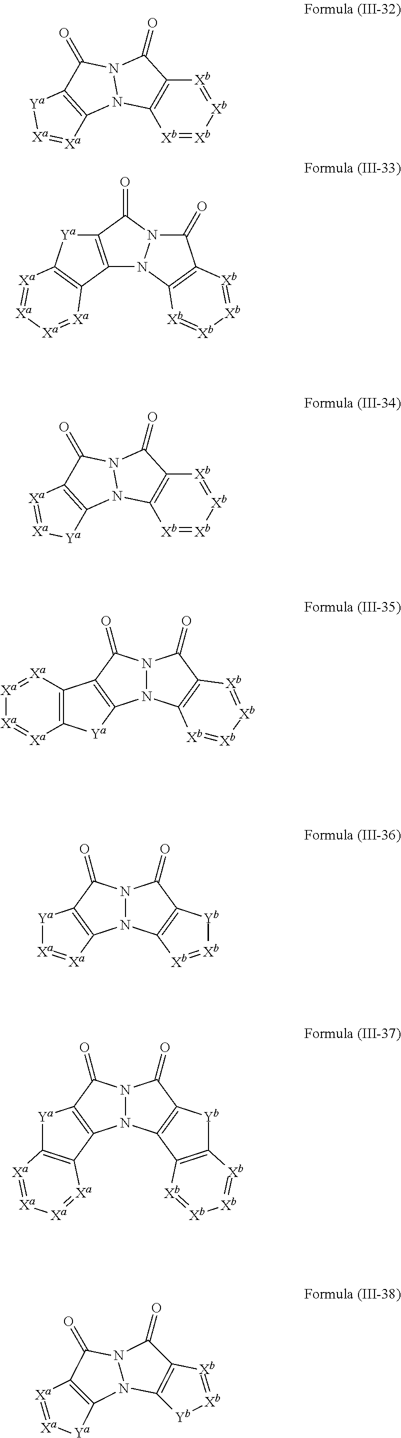 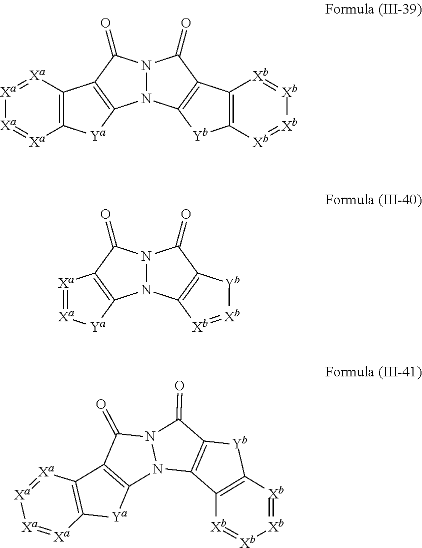
wherein
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, where R^{a} has the definition detailed in claim 19;
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, where R^{b} has the definition detailed in claim 19;
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, where R has the definition detailed in claim 19;
X is the same or different at each instance and is N or CR, with the proviso that not more than two of the X groups in one cycle are N, where R has the definition detailed in claim 19;
X^{a} is the same or different at each instance and is N or CR^{a}, with the proviso that not more than two of the X^{a} groups in one cycle are N, where R^{a} has the definition detailed in claim 19;
X^{b} is the same or different at each instance and is N or CR^{b}, with the proviso that not more than two of the X^{b} groups in one cycle are N, where R^{b} has the definition detailed in claim 19.

23. The compound according to claim 19, including at least one structure of the formulae (IV-1) to (IV-41): 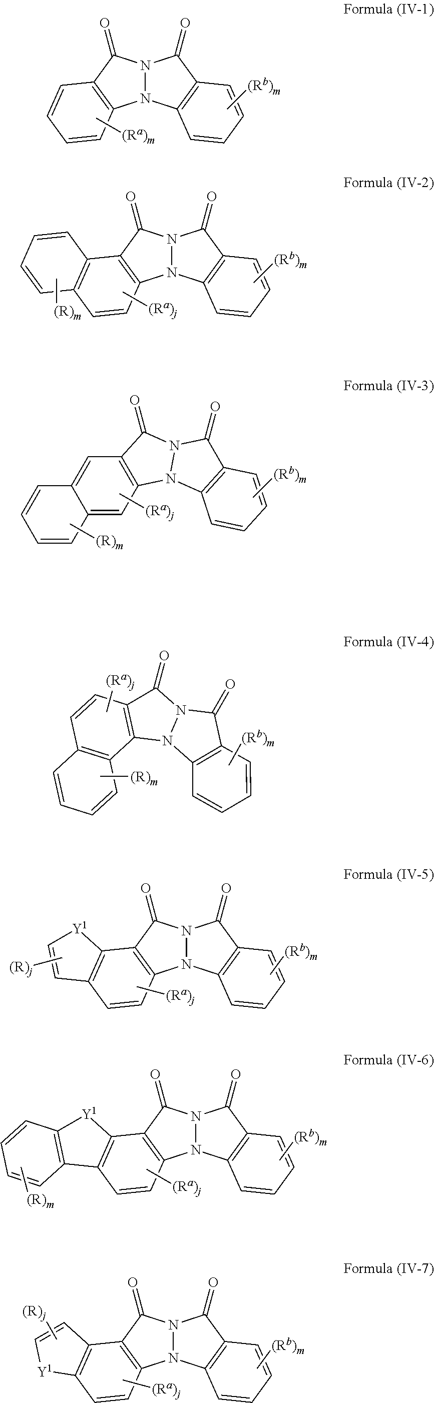 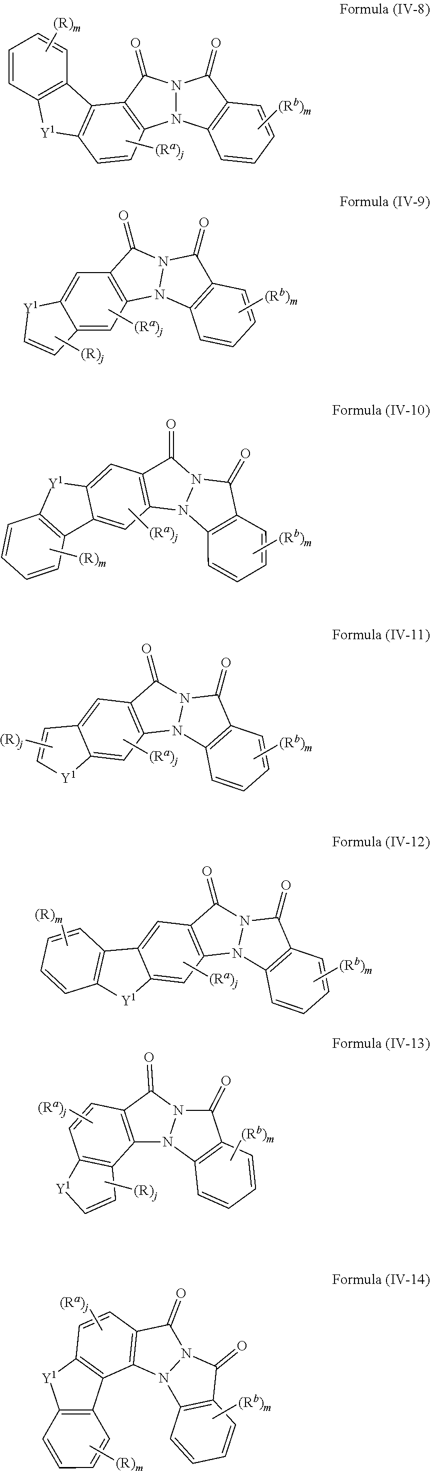 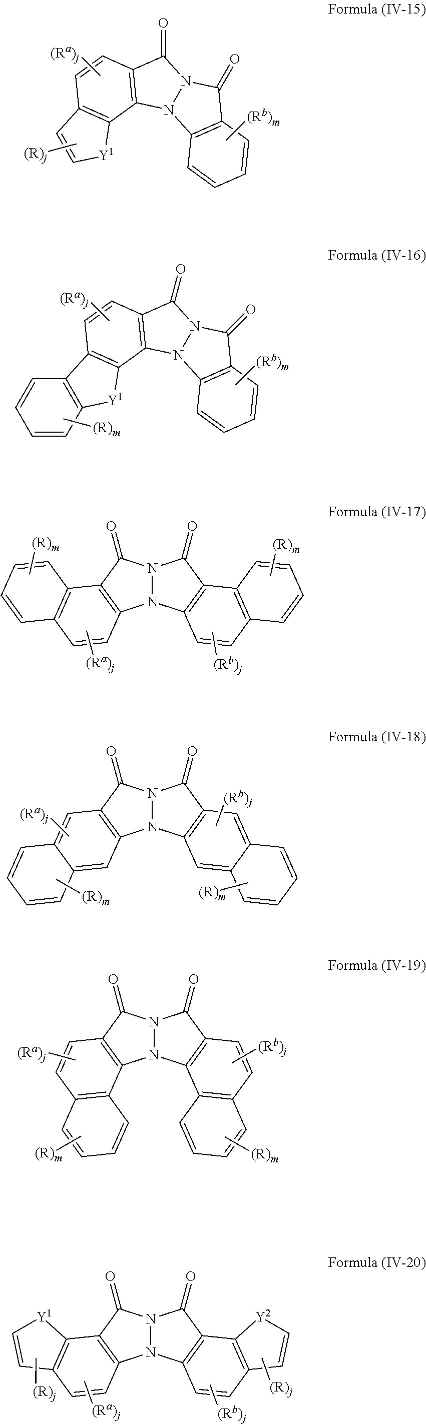 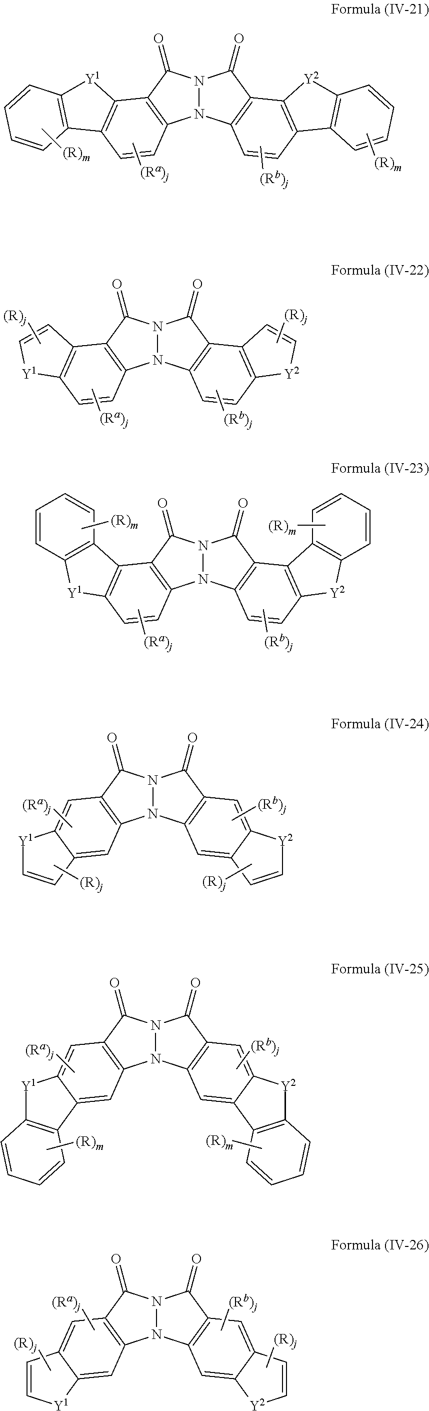 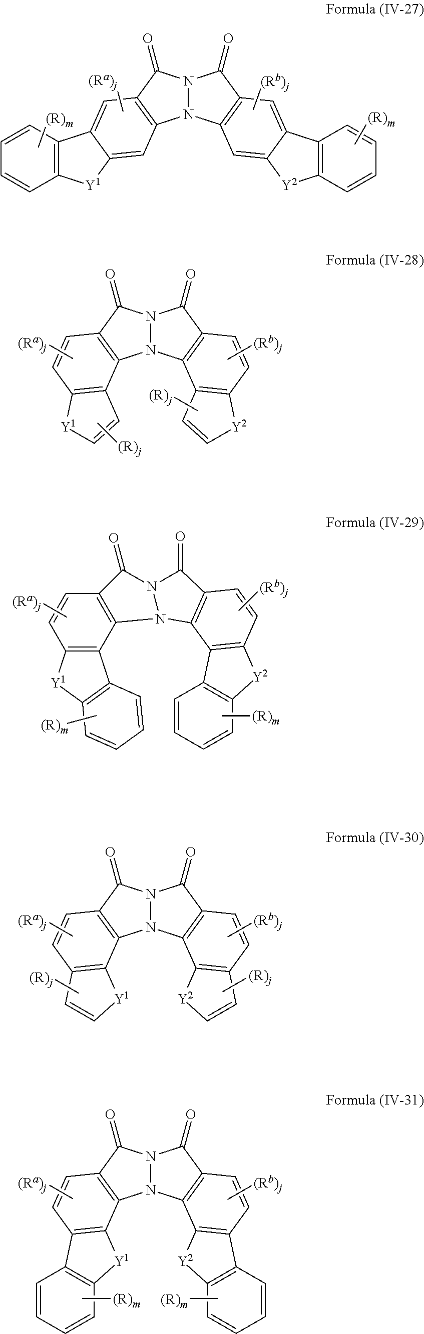 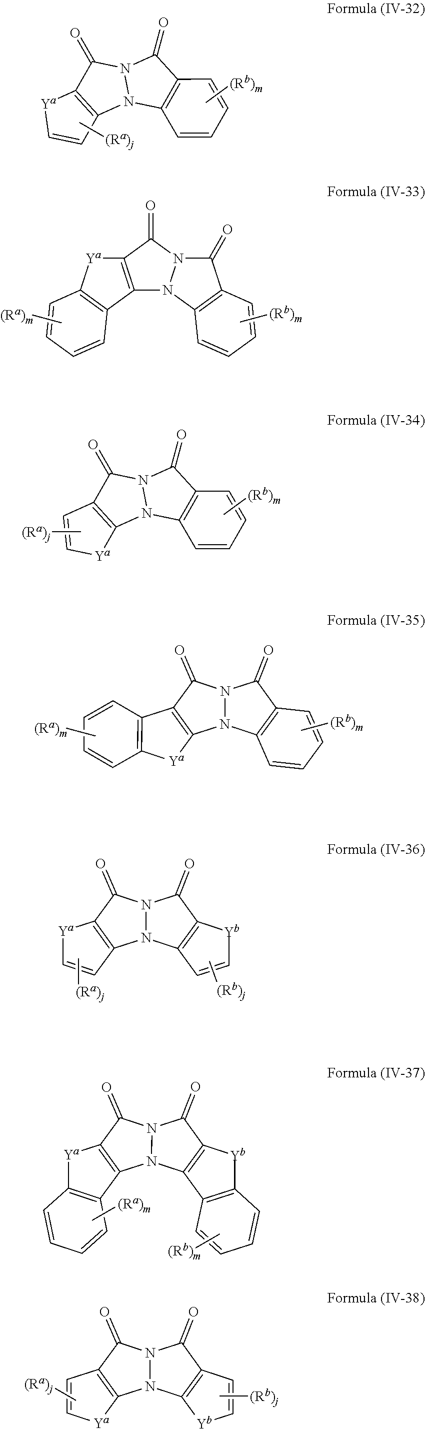 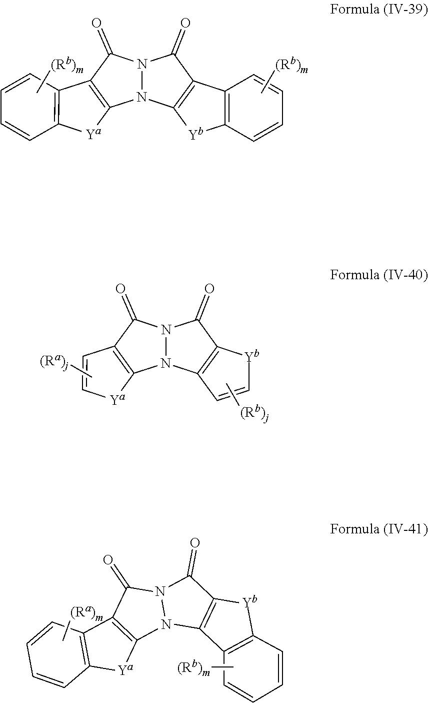
where the symbols R, R^{a} and R^{b} have the definitions given in claim 19, the symbols,
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, where R^{a} has the definition detailed in claim 19;
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, where R^{b} has the definition detailed in claim 19;
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, where R has the definition detailed in claim 19;
and the further symbols have the following definition:
m is 0, 1, 2, 3 or 4;
j is 0, 1 or 2.

24. The compound according to claim 19, including at least one structure of the formulae (V-1) to (V-8): 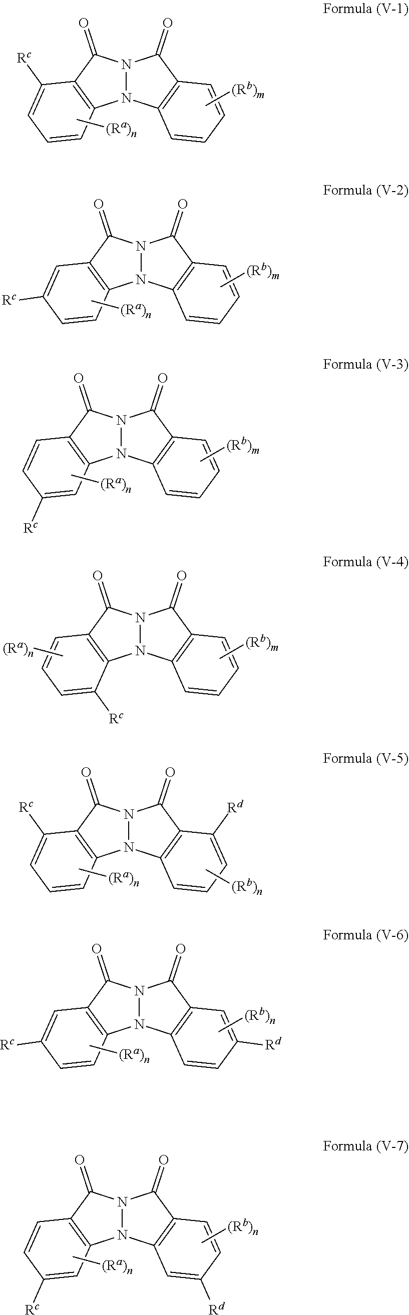 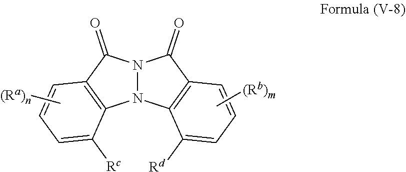
where the symbols R^{a} and R^{b} have the definitions given in claim 19 and the further symbols have the following definition:
R^{c}, R^{d} is the same or different at each instance and is N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; or heteroarylthio group which has 5 to 60 aromatic ring atoms and may be substituted by one or more R¹ radicals, or a diarylamino, arylheteroarylamino, diheteroarylamino group which has 5 to 60 aromatic ring atoms and may be substituted by one or more R¹ radicals, or an arylalkyl or heteroarylalkyl group which has 5 to 60 aromatic ring atoms and 1 to 10 carbon atoms in the alkyl radical and may be substituted by one or more R¹ radicals; at the same time, one R^{c} radical may form a ring system with an R, R^{a}, R^{b}, R^{d} radical or a further group; at the same time, an R^{d} radical may form a ring system with an R, R^{a}, R^{b}, R^{c} radical or a further group;
m is 0, 1, 2, 3 or 4;
n is 0, 1, 2 or 3.

25. The compound according to claim 20, wherein at least one R, R^{a}, R^{b}, R^{c}, R^{d} radical is a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may be substituted in each case by one or more R¹ radicals, where one or more nonadjacent CH₂ groups may be replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals.

26. The compound according to claim 20, wherein at least two R, R^{a}, R^{b}, R^{c}, R^{d} radicals together with the further groups to which the two R, R^{a}, R^{b}, R^{c}, R^{d} radicals bind form a fused ring, where the two R, R^{a}, R^{b}, R^{c}, R^{d} radicals form at least one structure of the formulae (RA-1) to (RA-12): 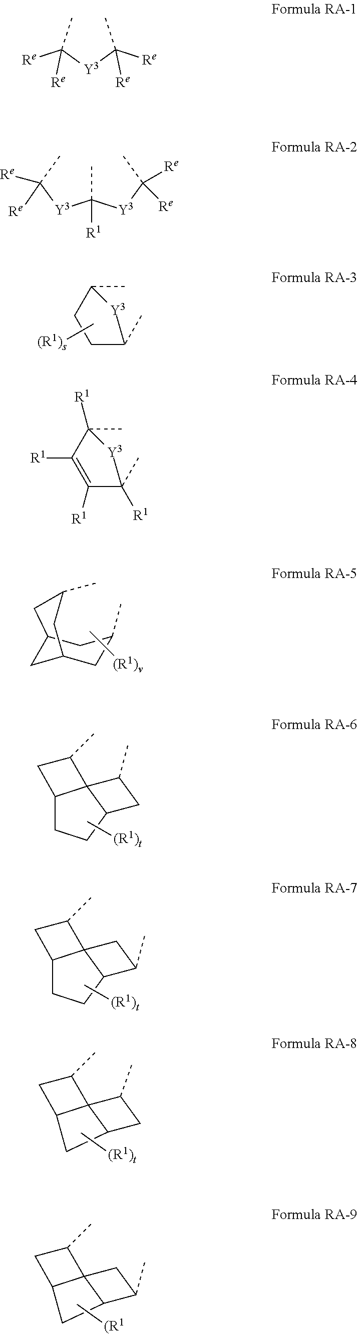 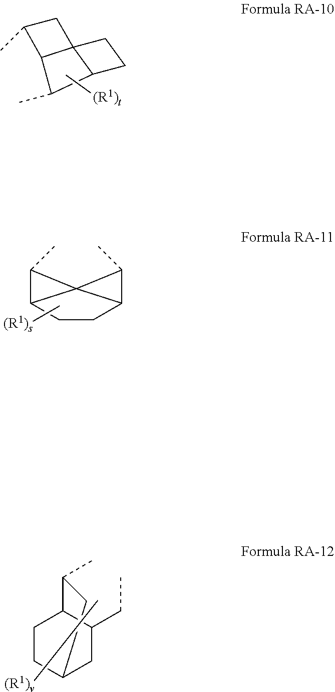
where R¹ has the definition detailed above, the dotted bonds represent the sites of attachment via which the two R, R^{a}, R^{b}, R^{c}, R^{d} radicals bind to the further groups, and the further symbols have the following definition:
Y³ is the same or different at each instance and is C(R¹)₂, (R¹)₂C—C(R¹)₂, (R¹)C═C(R¹), NR¹, NAr′, O or S;
R is the same or different at each instance and is F, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R² radicals, where one or more nonadjacent CH₂ groups may be replaced by R²C═CR², C≡C, Si(R²)₂, C═O, C═S, C═Se, C═NR², —C(═O)O—, —C(═O)NR²—, NR², P(═O)(R¹), —O—, —S—, SO or SO₂, or an aromatic or heteroaromatic ring system which has 5 to 60 aromatic ring atoms and may be substituted in each case by one or more R² radicals, or an aryloxy or heteroaryloxy group which has 5 to 60 aromatic ring atoms and may be substituted by one or more R² radicals; at the same time, two R radicals together or one R radical together with an R¹ radical or with a further group form a ring system;
s is 0, 1, 2, 3, 4, 5 or 6;
t is 0, 1, 2, 3, 4, 5, 6, 7 or 8;
v is 0, 1, 2, 3, 4, 5, 6, 7, 8 or 9.

27. The compound according to claim 20, wherein at least two R, R^{a}, R^{b}, R^{c}, R^{d} radicals together with the further groups to which the two R, R^{a}, R^{b}, R^{c}, R^{d} radicals bind form a fused ring, where the two R, R^{a}, R^{b}, R^{c}, R^{d} radicals form structures of the formula (RB): 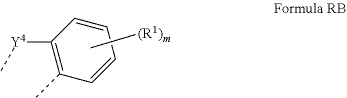
the dotted bonds represent the sites of attachment via which the two R, R^{a}, R^{b}, R^{c}, R^{d} radicals bind to the further groups, the index m is 0, 1, 2, 3 or 4 and Y⁴ is C(R¹)₂, NR¹, NAr′, BR¹, BAr′, O or S.

28. The compound according to claim 19, comprising at least one structure of the formulae (VI-1) to (VI-22), where the compounds have at least one fused ring, 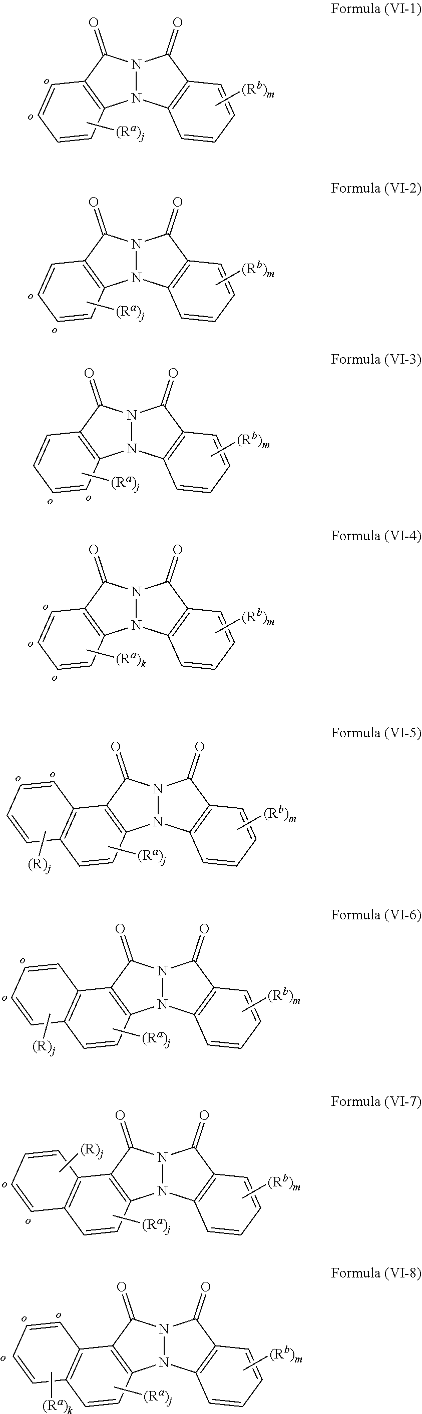 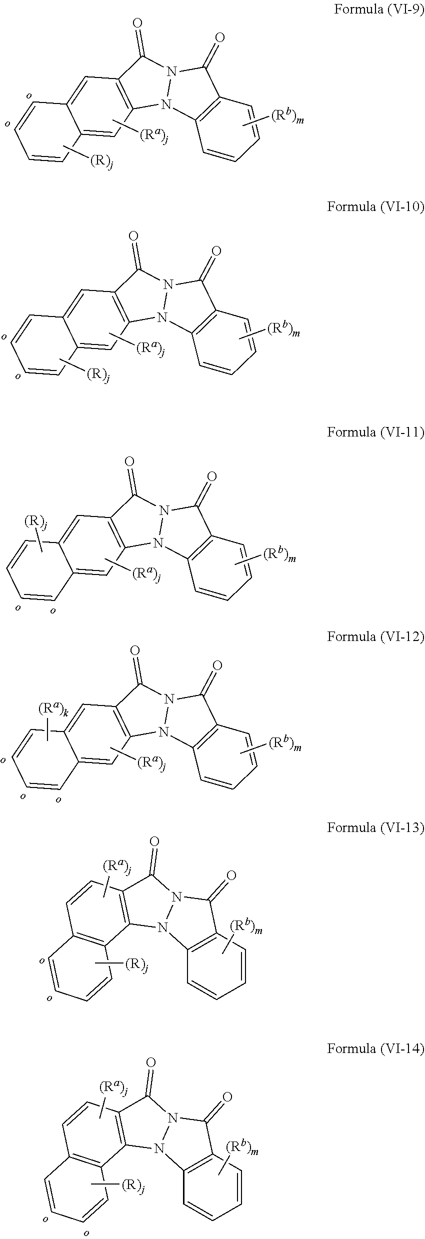 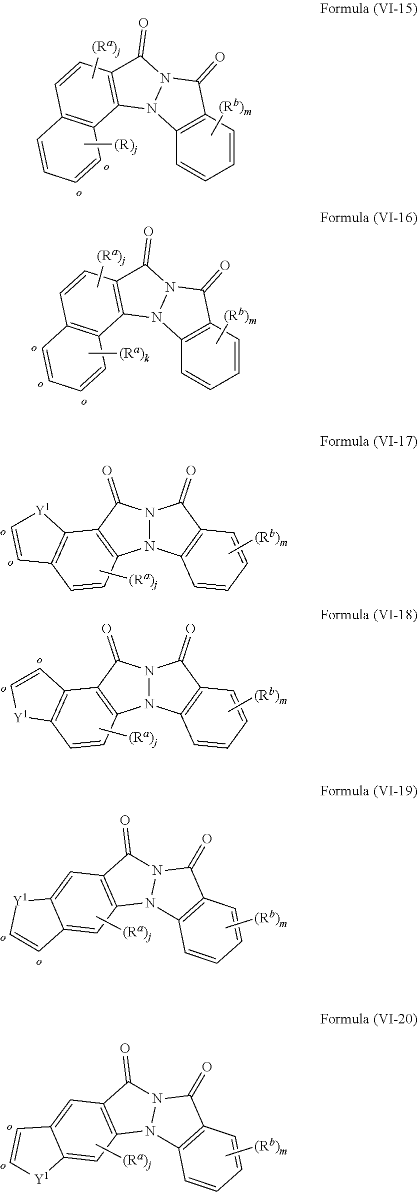 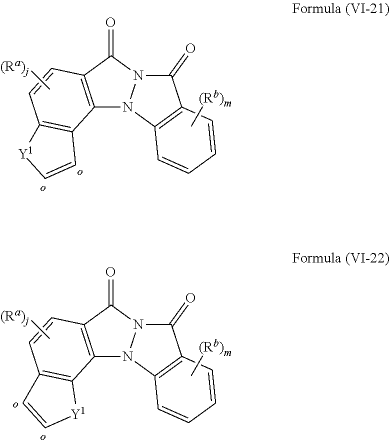
where the symbols R, R^{a}, R^{b}, Y¹ and Y² have the definitions given in claim 19, the symbol o represents the sites of attachment, and the further symbols have the following definition:
m is 0, 1, 2, 3 or 4;
n is 0, 1, 2 or 3;
j is 0, 1 or 2.

29. The compound according to claim 19, wherein at least one substituent R, R^{a}, R^{b} is the same or different at each instance and is selected from the group consisting of H, D, a branched or cyclic alkyl, alkoxy or thioalkoxy group having 3 to 20 carbon atoms or an aromatic or heteroaromatic ring system selected from the groups of the following formulae Ar-1 to Ar-78: 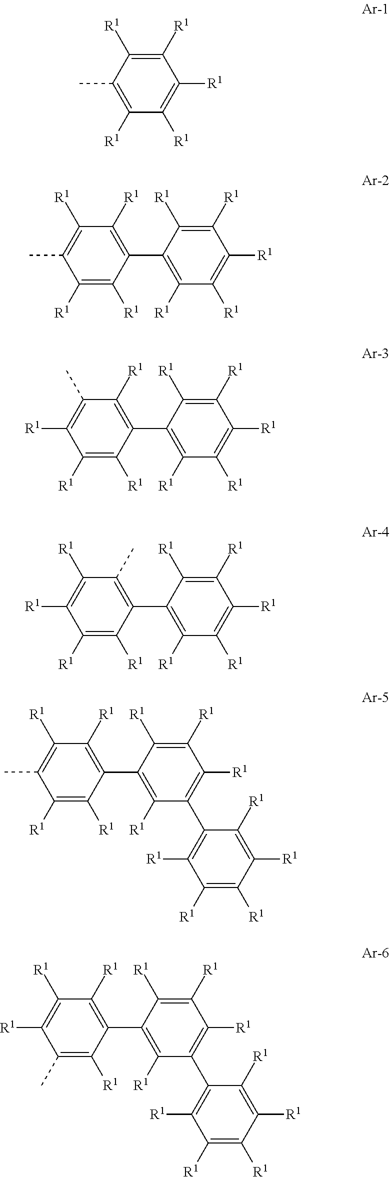 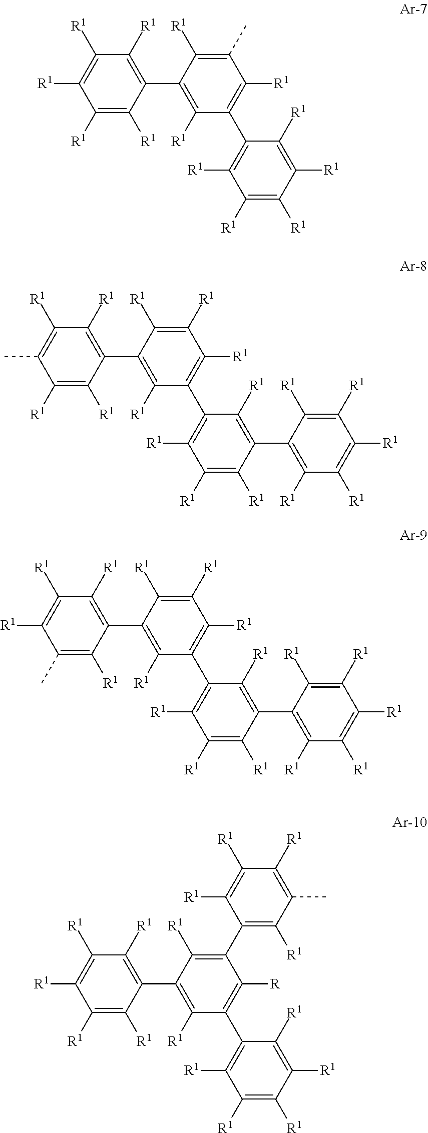 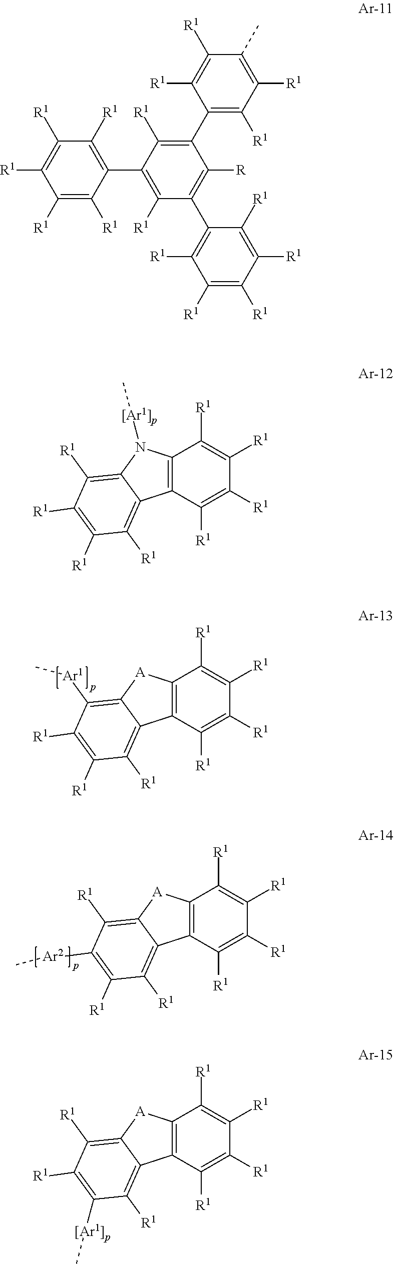 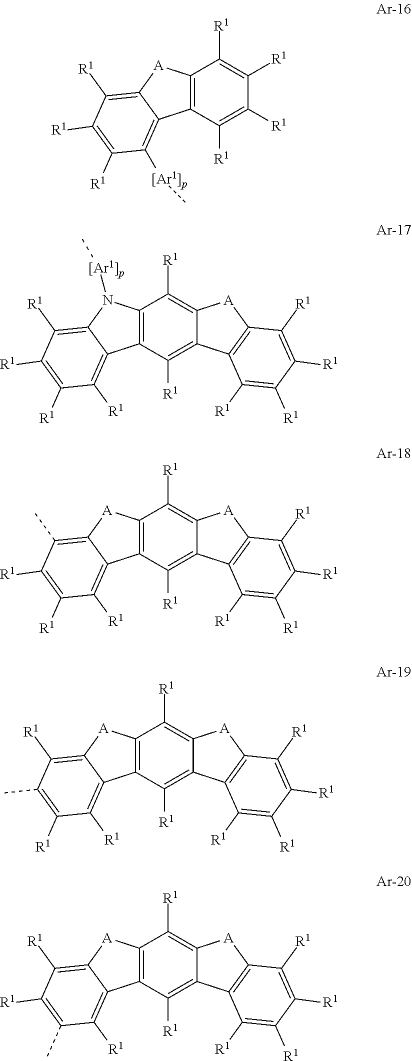 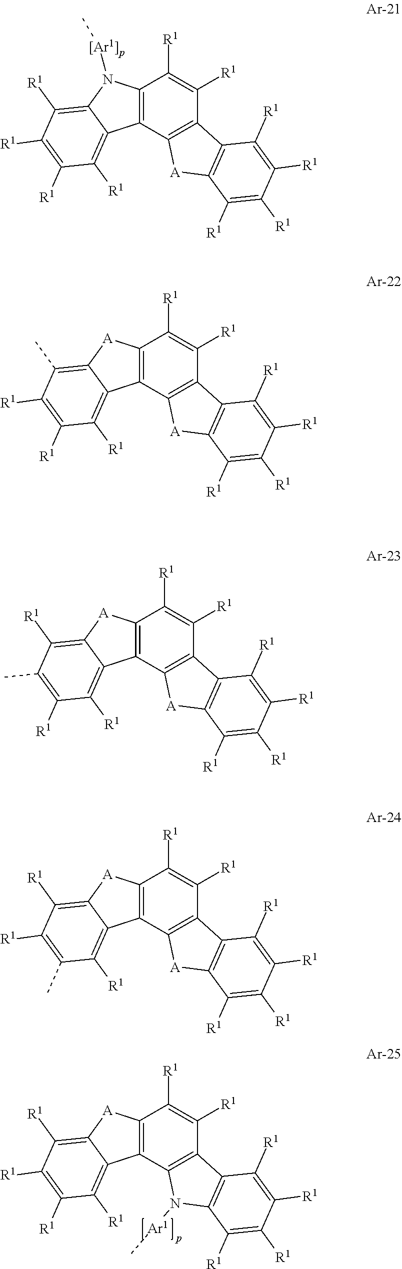 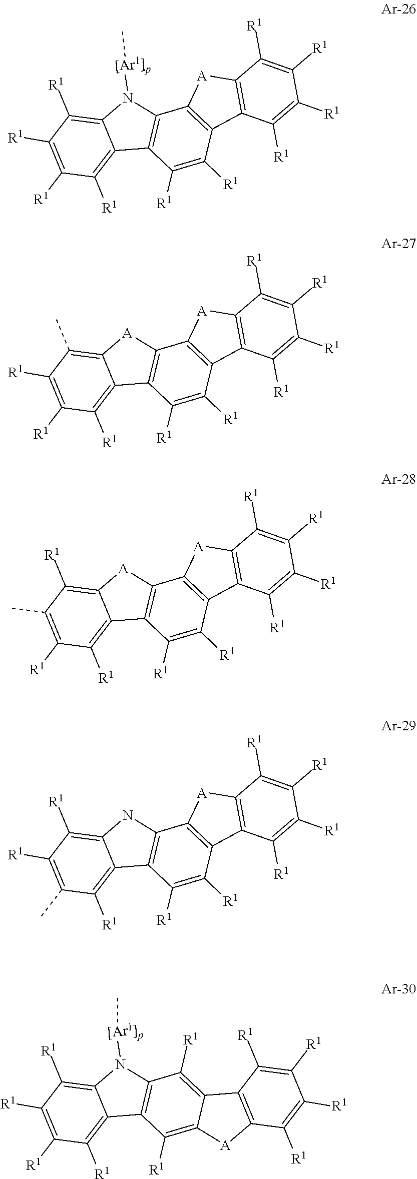 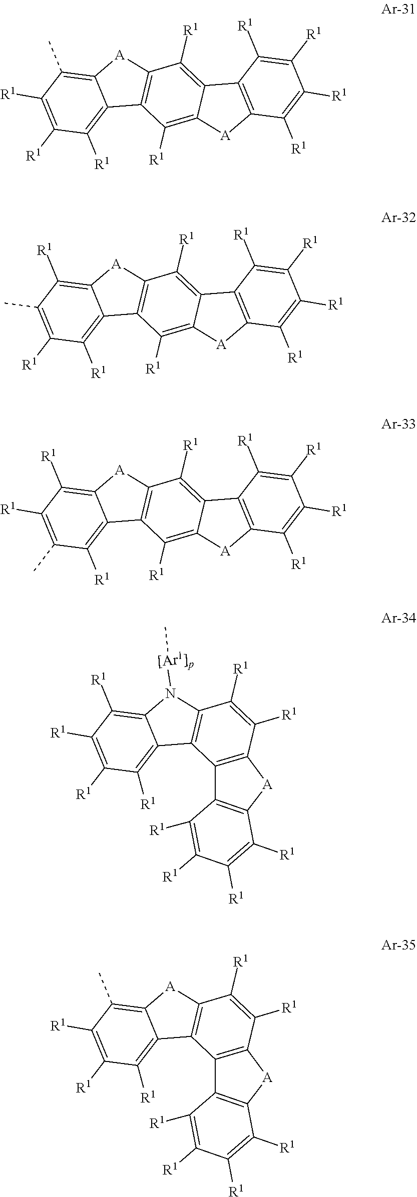 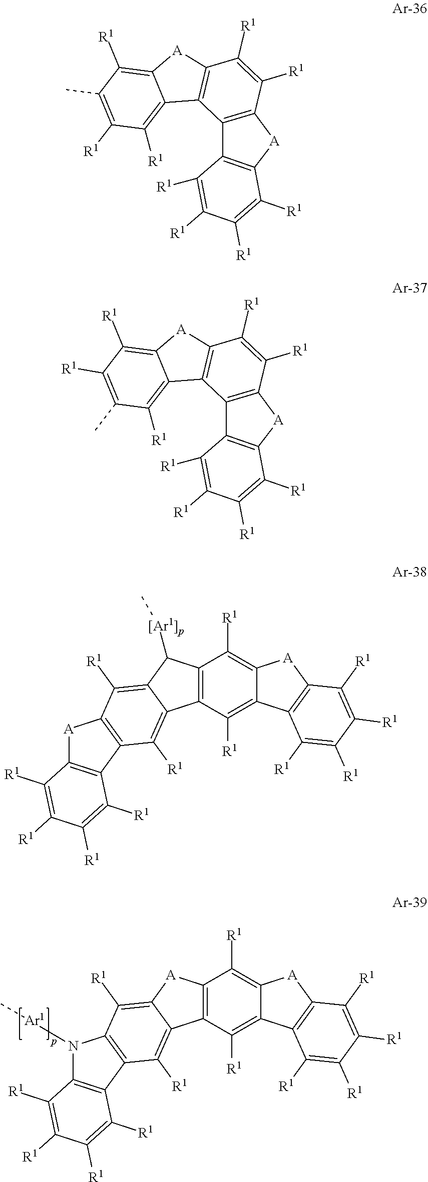 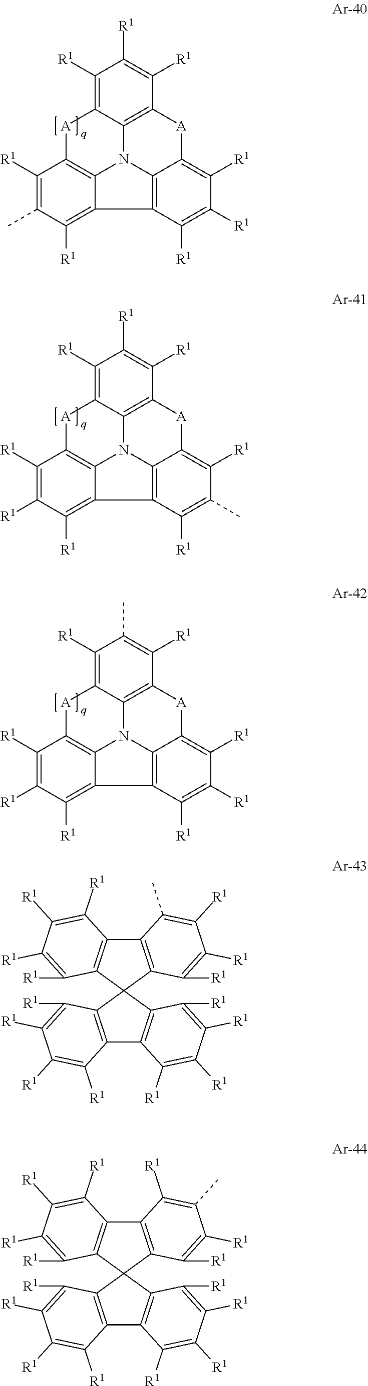 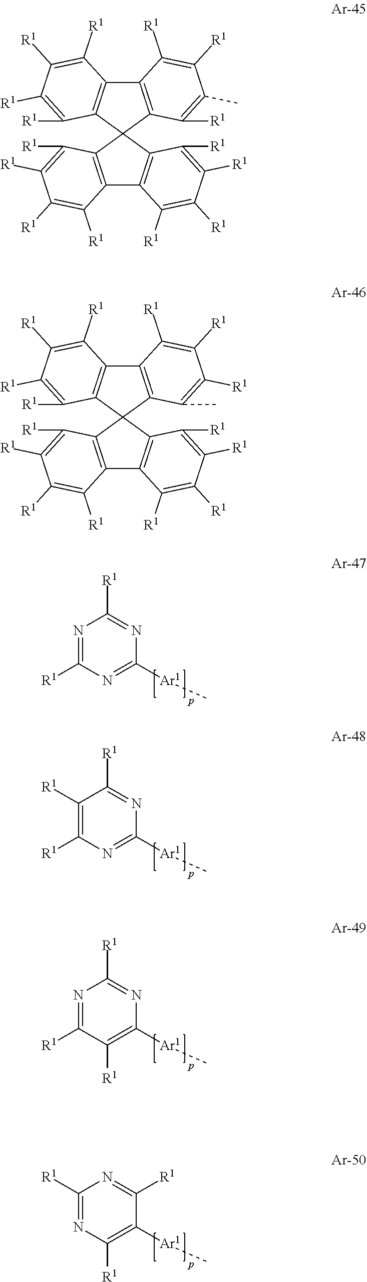 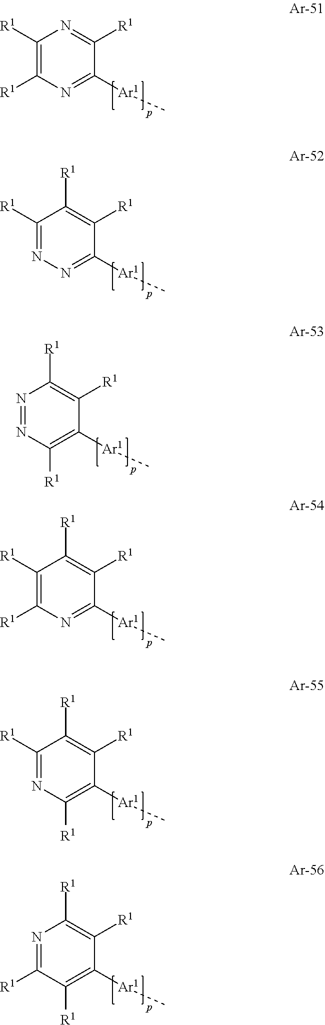 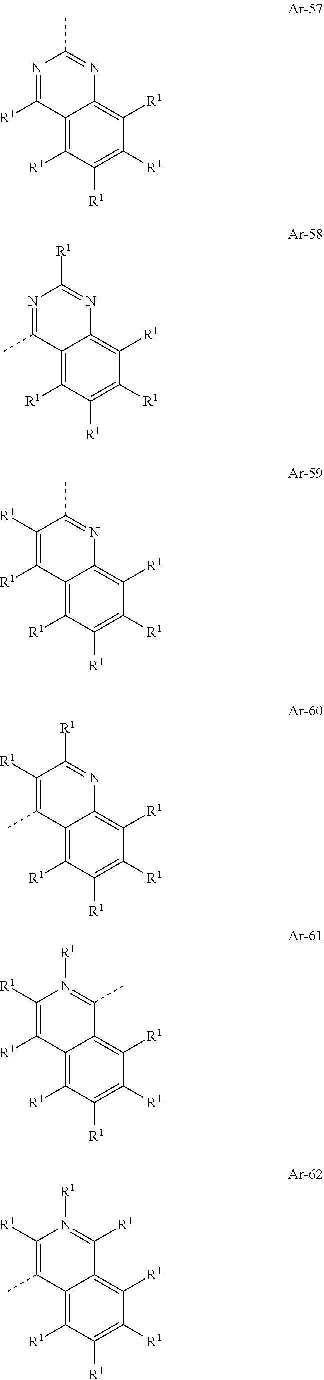 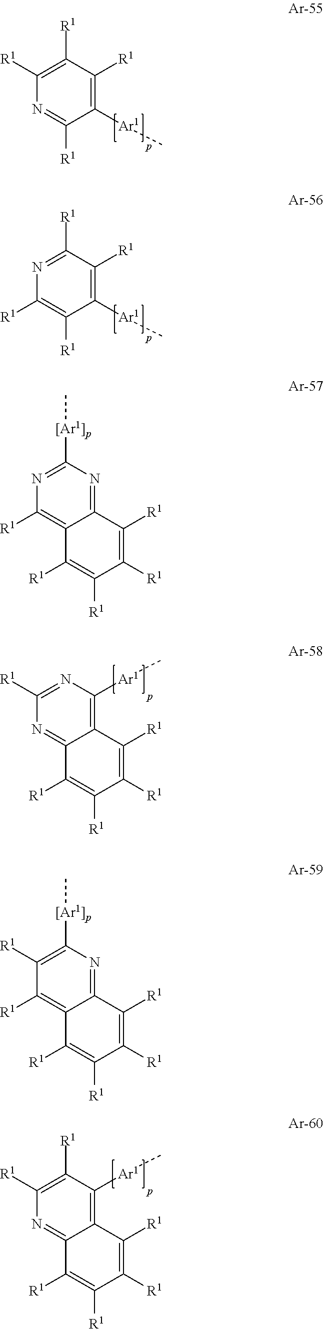 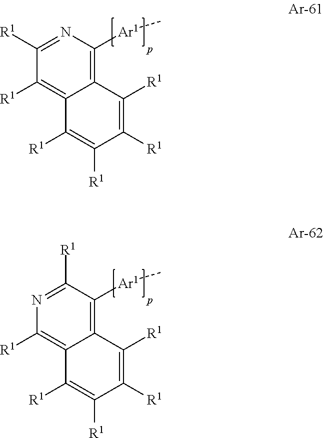 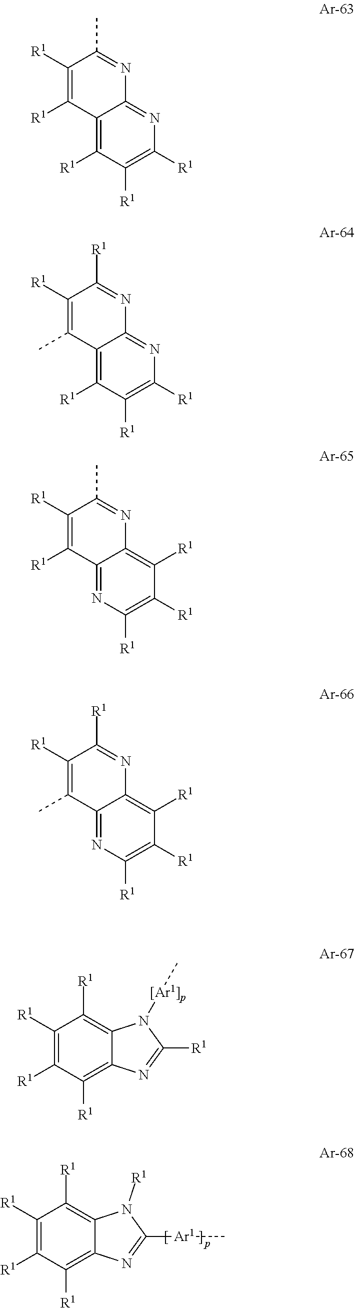 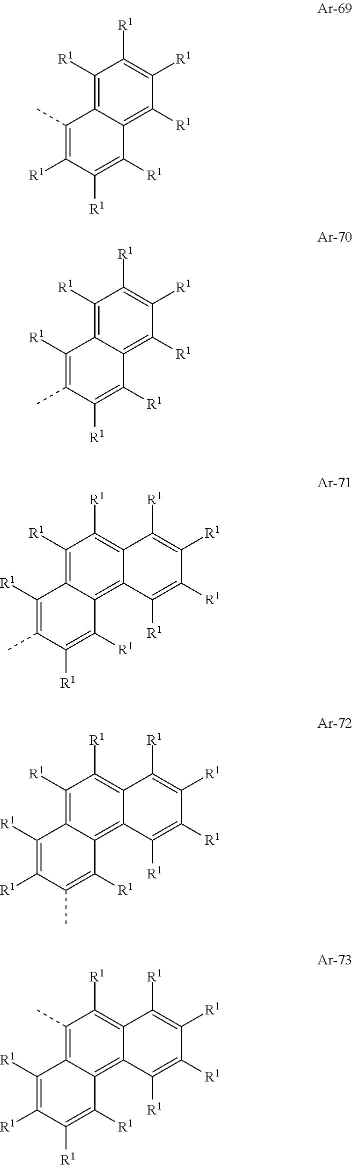 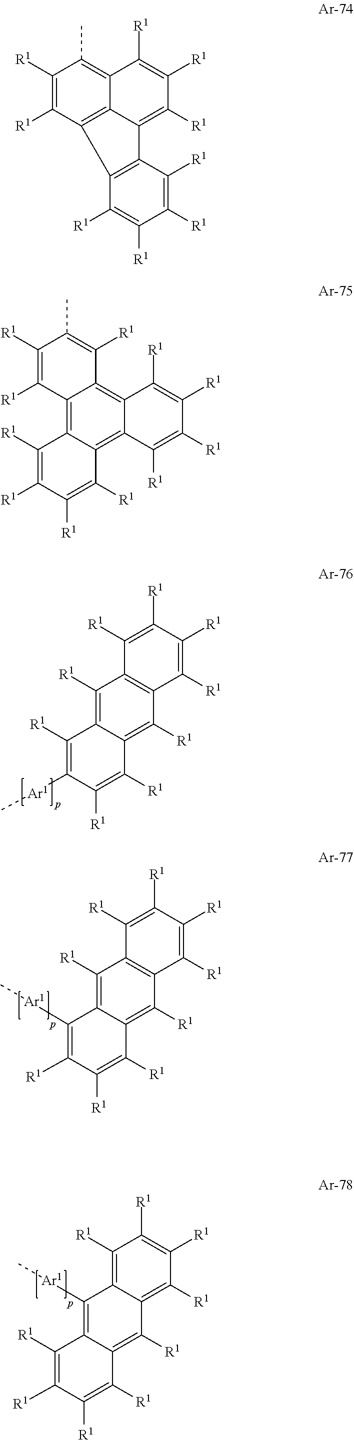
where R¹ has the definitions given above, the dotted bond represents the bond on the corresponding group and in addition:
Ar¹ is the same or different at each instance and is a bivalent aromatic or heteroaromatic ring system which has 6 to 18 aromatic ring atoms and may be substituted in each case by one or more R¹ radicals;
A is the same or different at each instance and is C(R¹)₂, NR¹, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R¹ radicals are bonded to the corresponding carbon atoms instead.

30. The compound according to claim 24, wherein the substituent R^{c}, R^{d} is the same or different at each instance and represents an aromatic or heteroaromatic ring system which has 6 to 30 aromatic ring atoms and may be substituted by one or more R¹ radicals selected from the groups of the formulae (Ar-1) to (Ar-78)

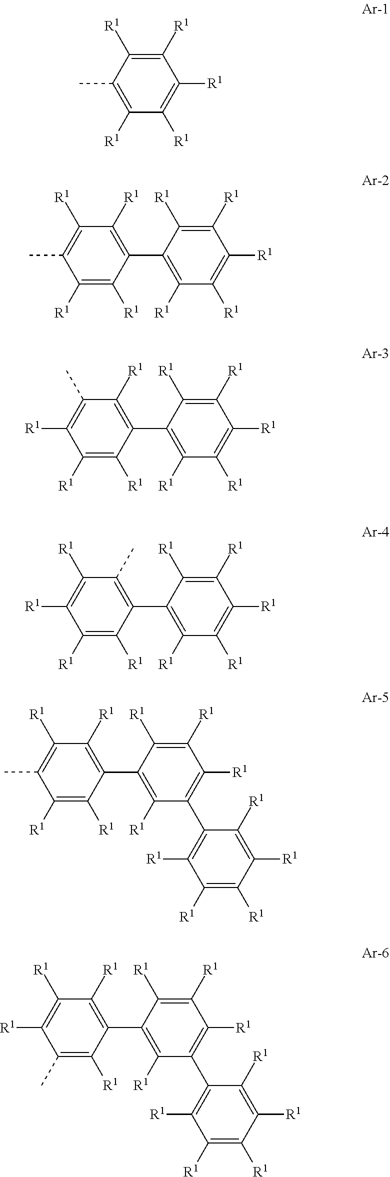

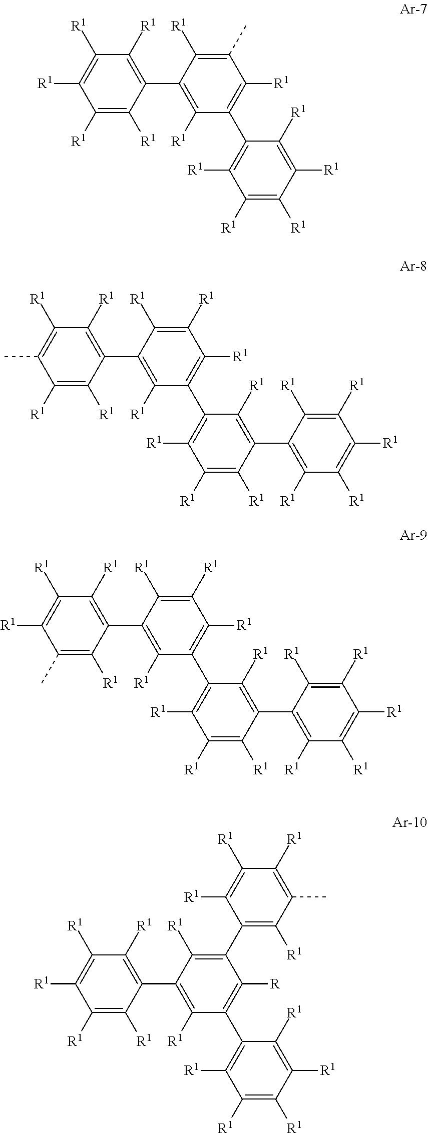

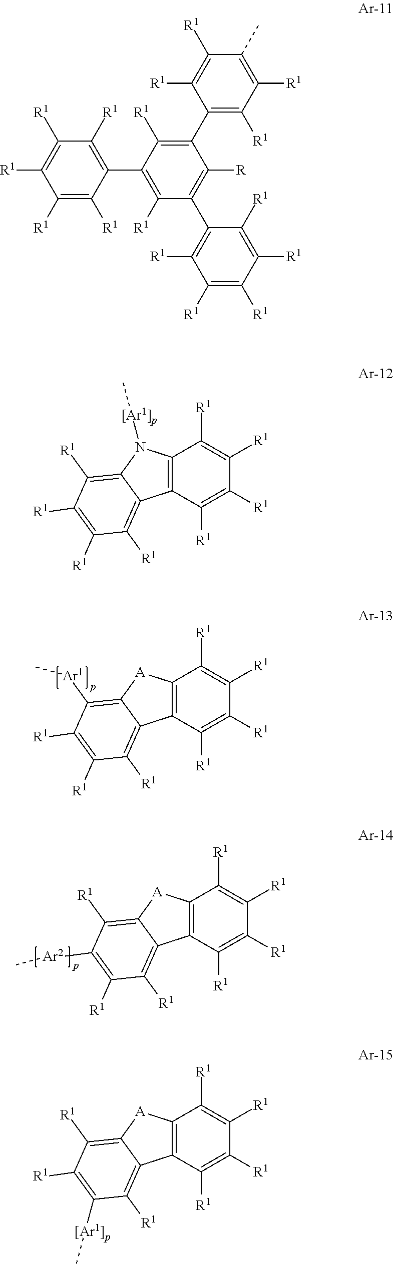

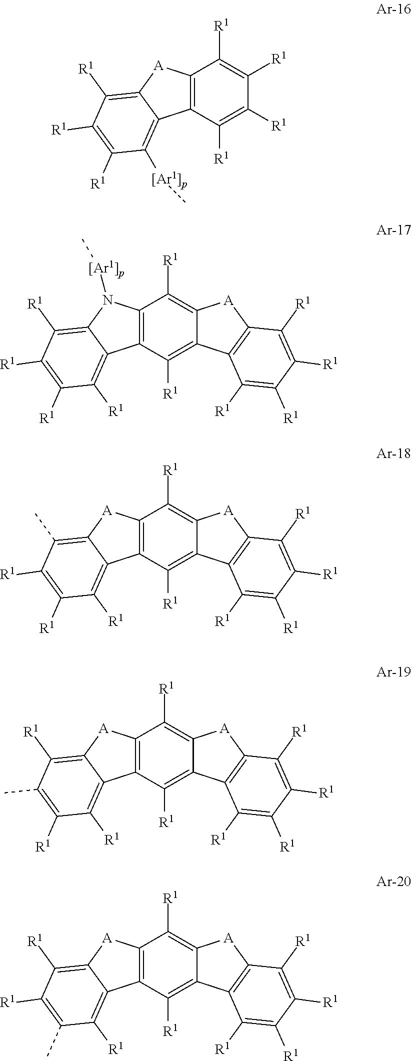

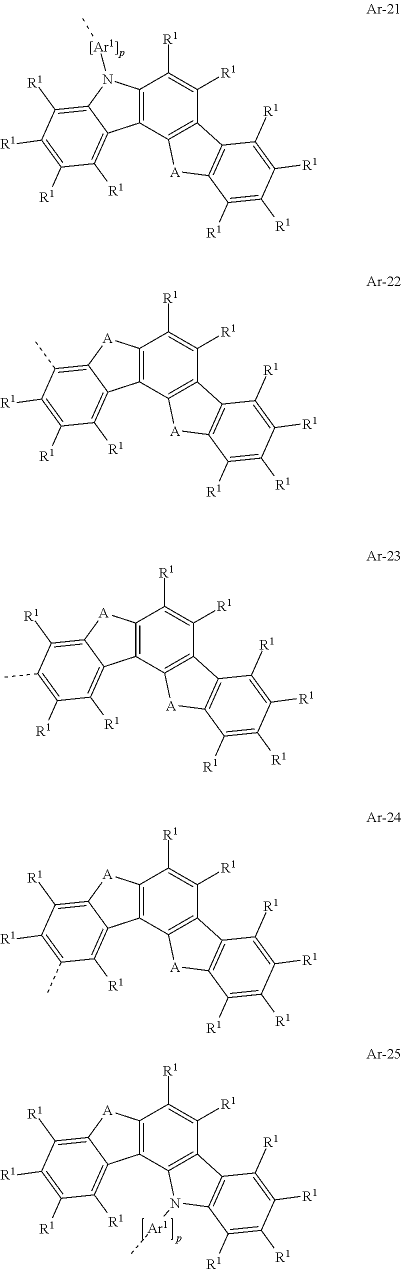

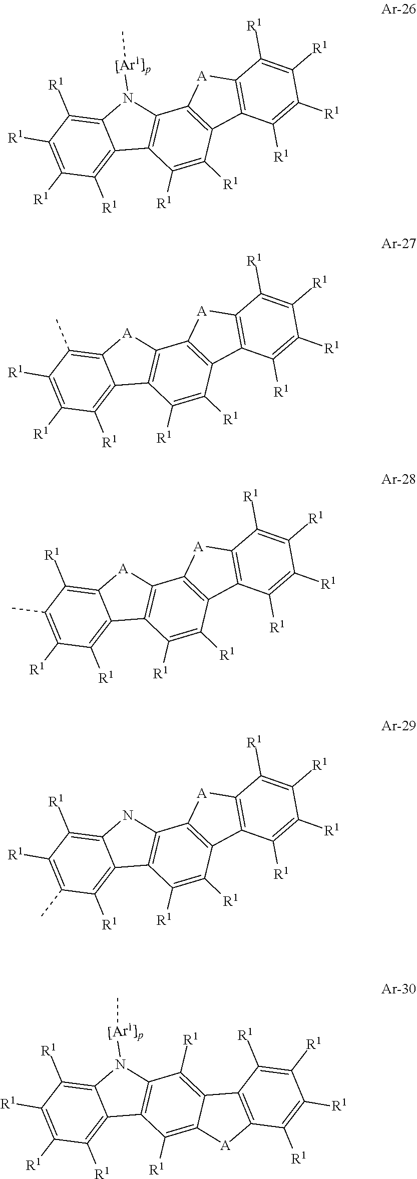

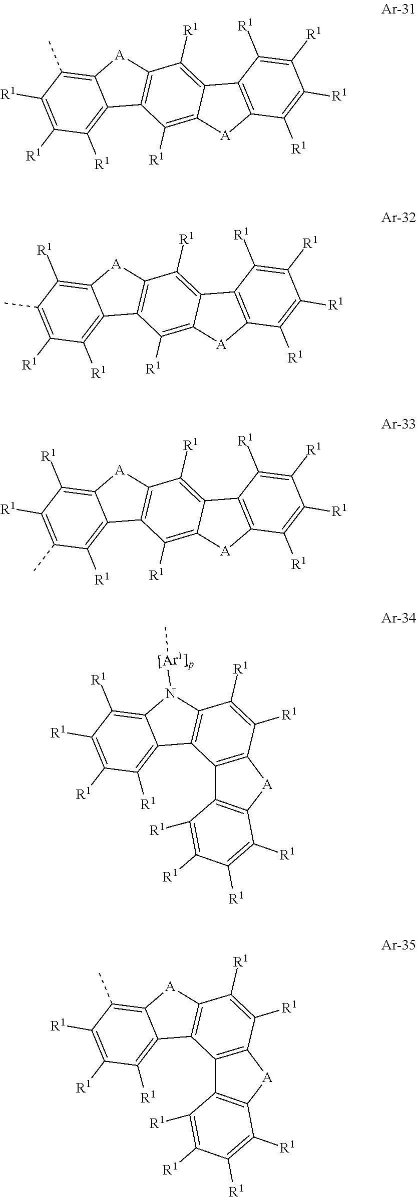

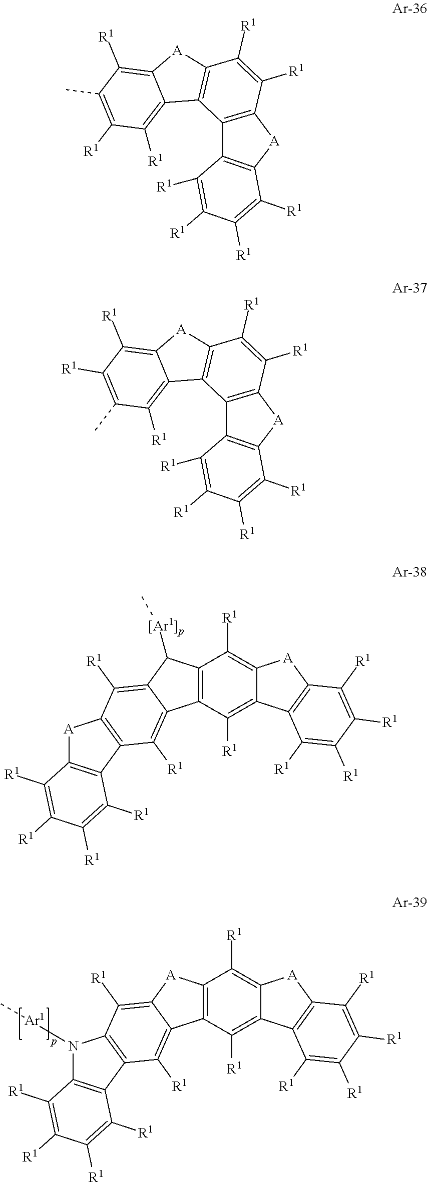

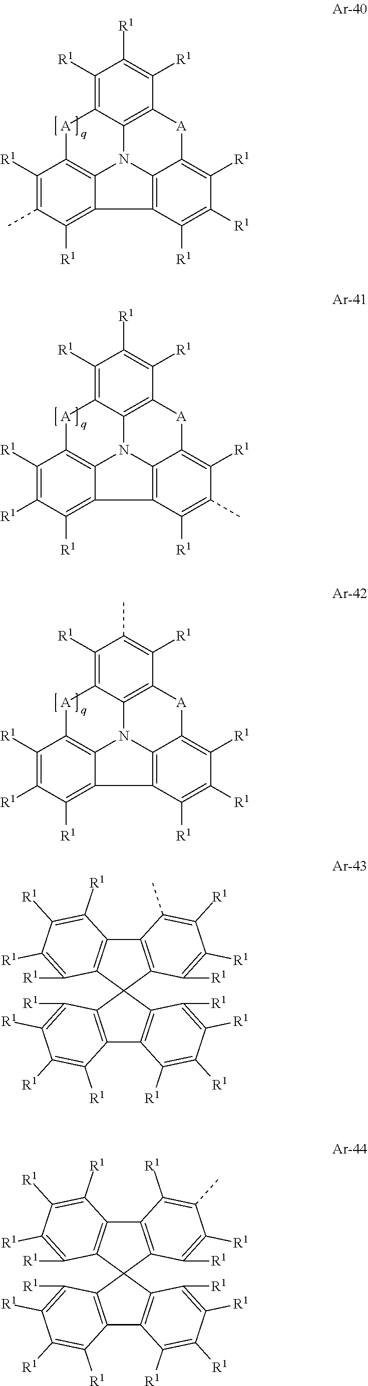

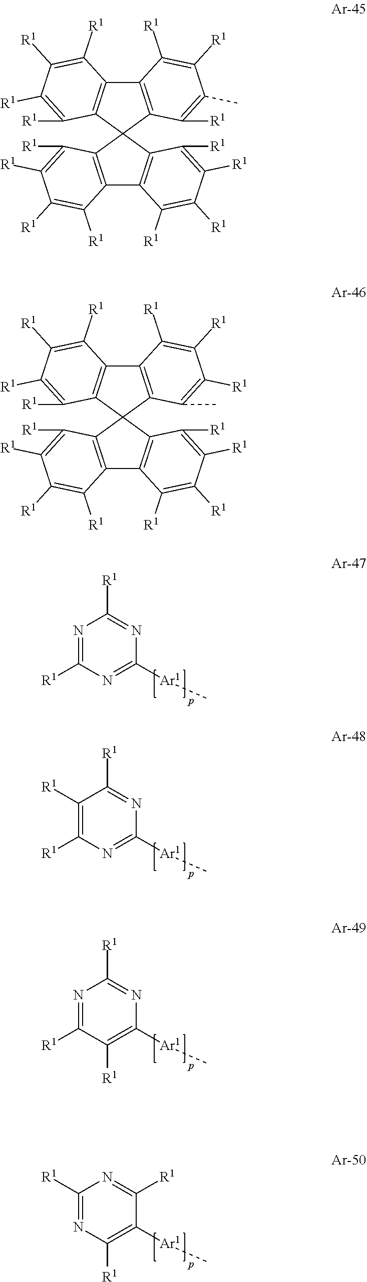

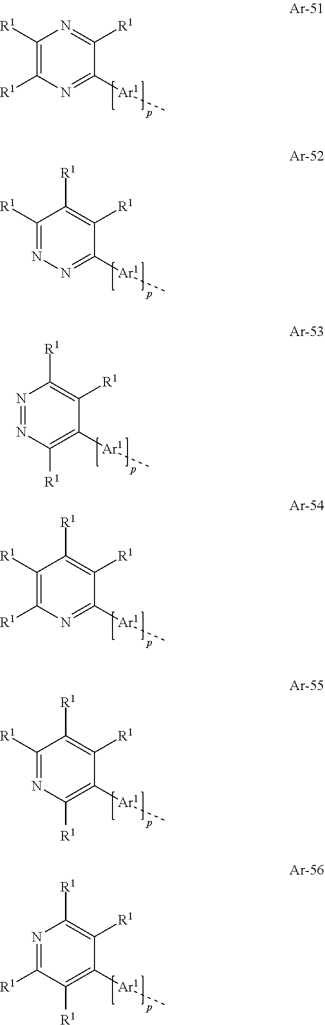

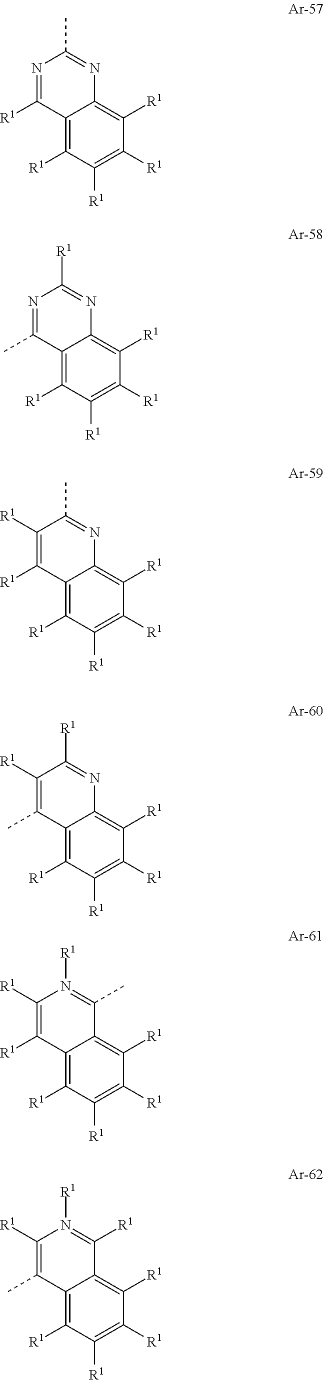

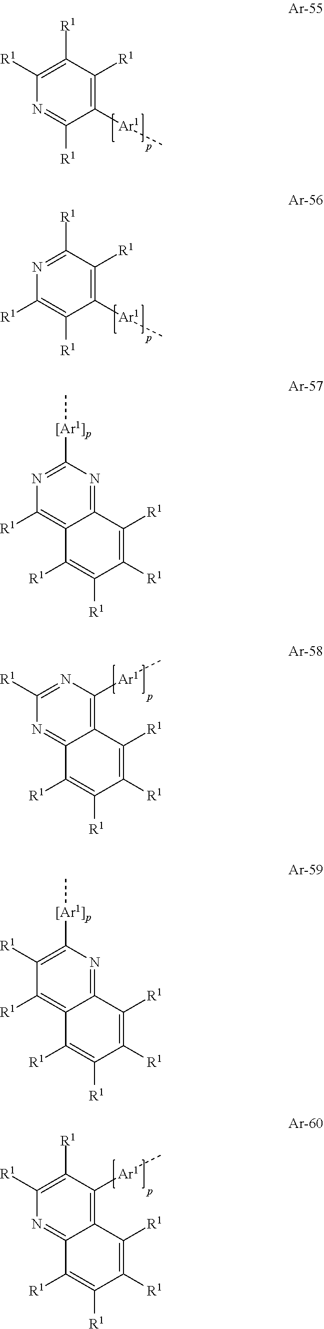

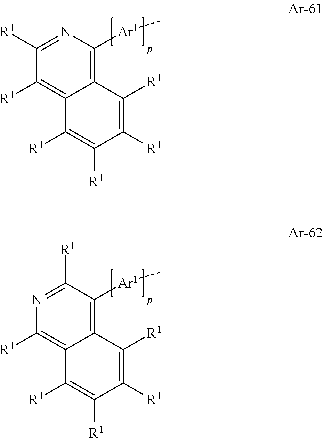

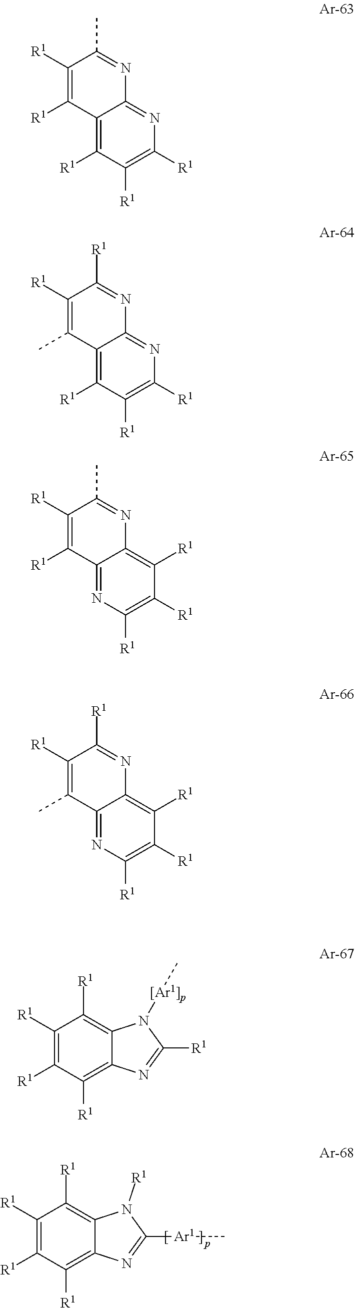

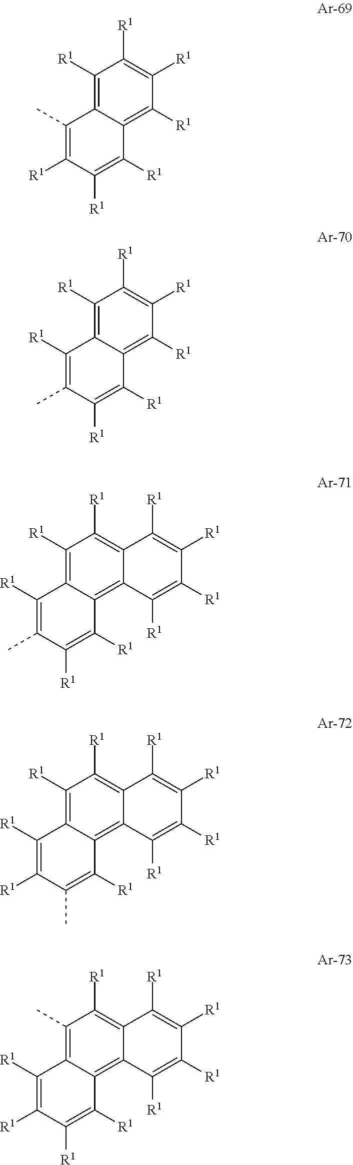

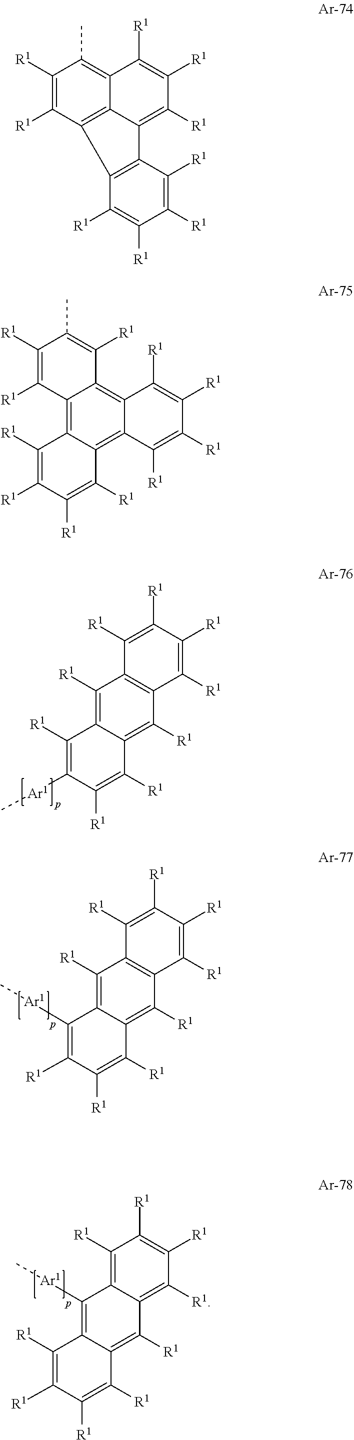

31. The compound according to claim 19, wherein the compound includes exactly two or exactly three structures of formula (I), (II-1) to (II-42), (III-1) to (III-41), (IV-1) to (IV-41), (V-1) to (V-8) and/or (VI-1) to (VI-22). 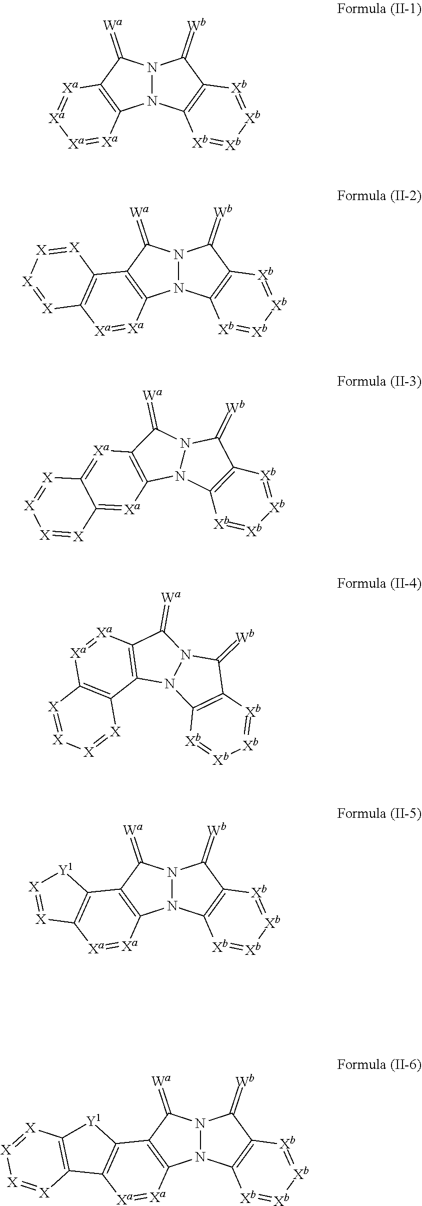 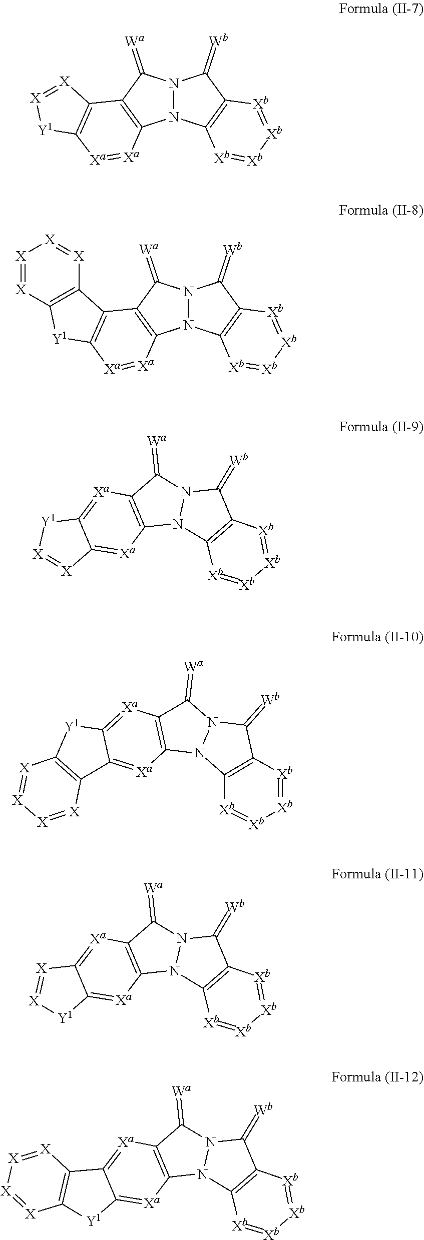 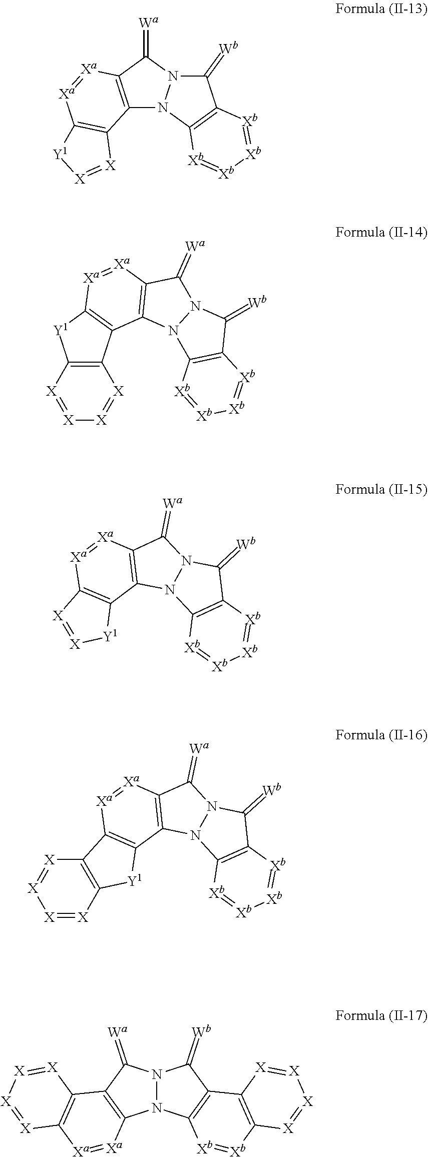 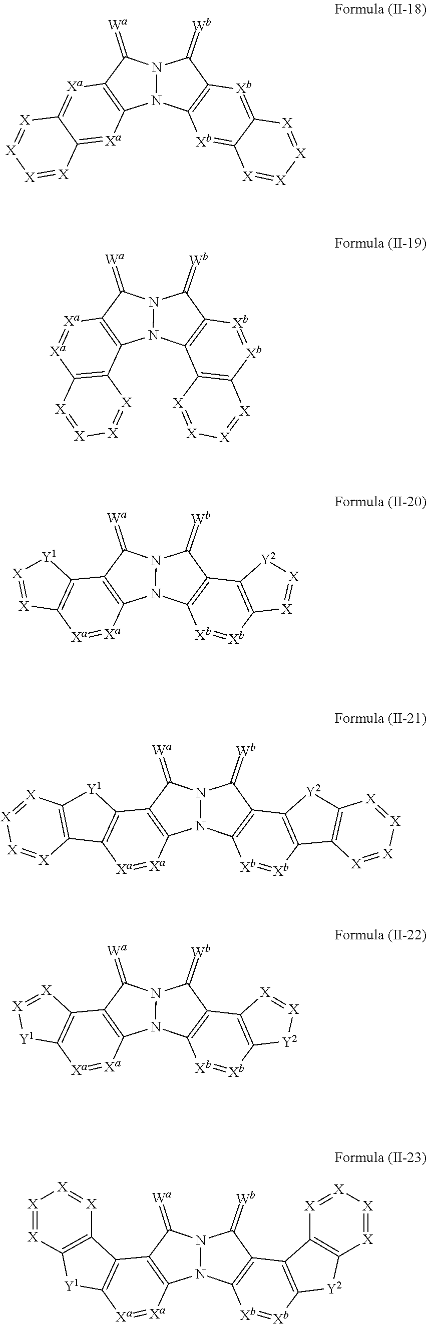 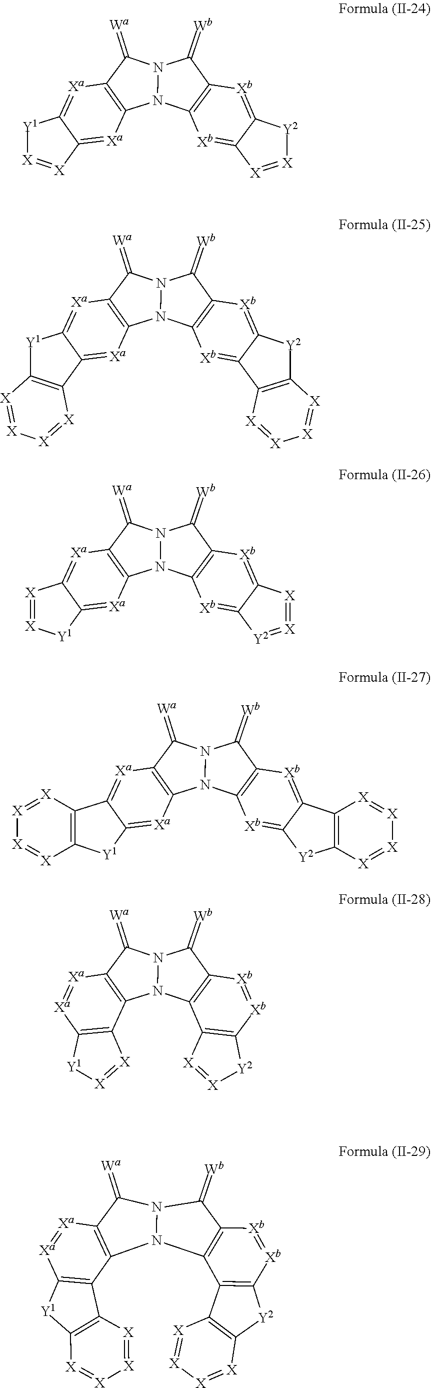 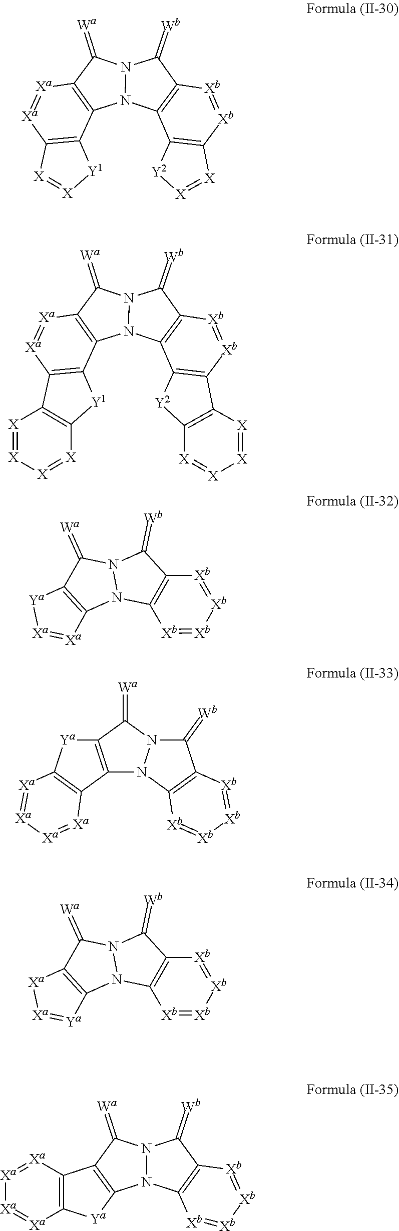 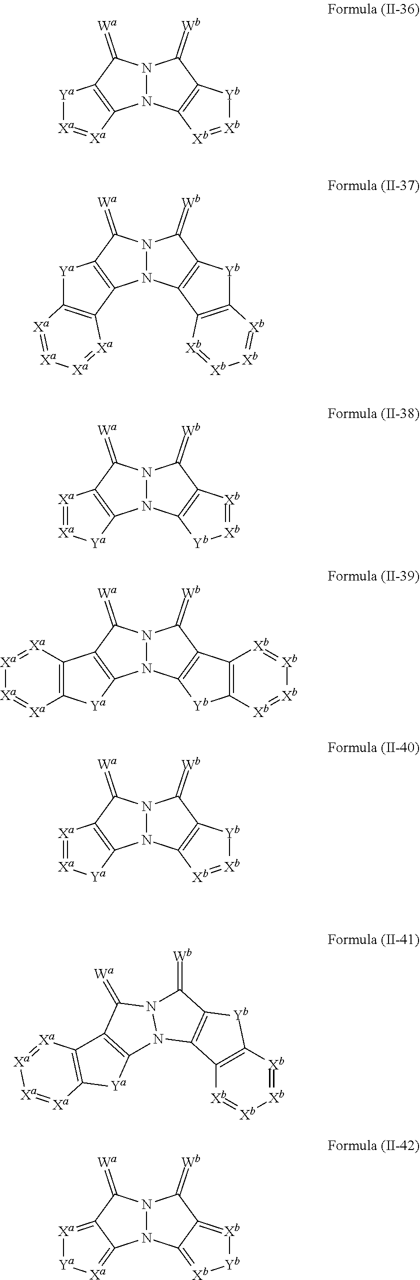
where the symbols W^{a} and W^{b} have the definitions given in claim 19 and the further symbols are as follows:
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, where R^{a} has the definition detailed in claim 19;
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, where R^{b} has the definition detailed in claim 19;
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, where R has the definition detailed in claim 19;
X is the same or different at each instance and is N or CR, with the proviso that not more than two of the X groups in one cycle are N, where R has the definition detailed in claim 19;
X^{a} is the same or different at each instance and is N or CR^{a}, with the proviso that not more than two of the X^{a} groups in one cycle are N, where R^{a} has the definition detailed in claim 19;
X^{b} is the same or different at each instance and is N or CR^{b}, with the proviso that not more than two of the X^{b} groups in one cycle are N, where R^{b} has the definition detailed in claim 19; 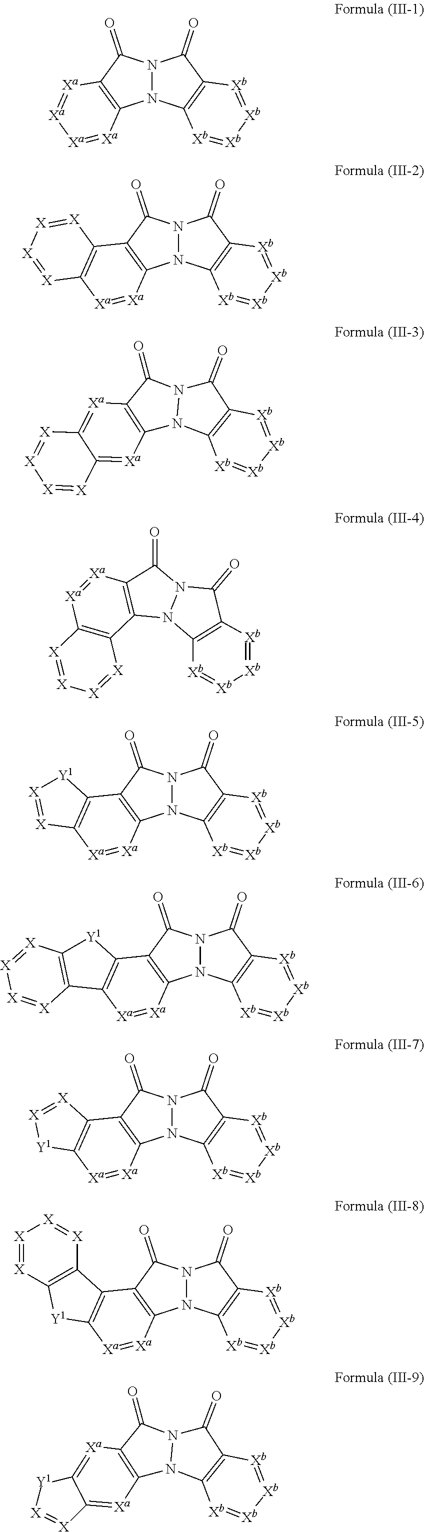 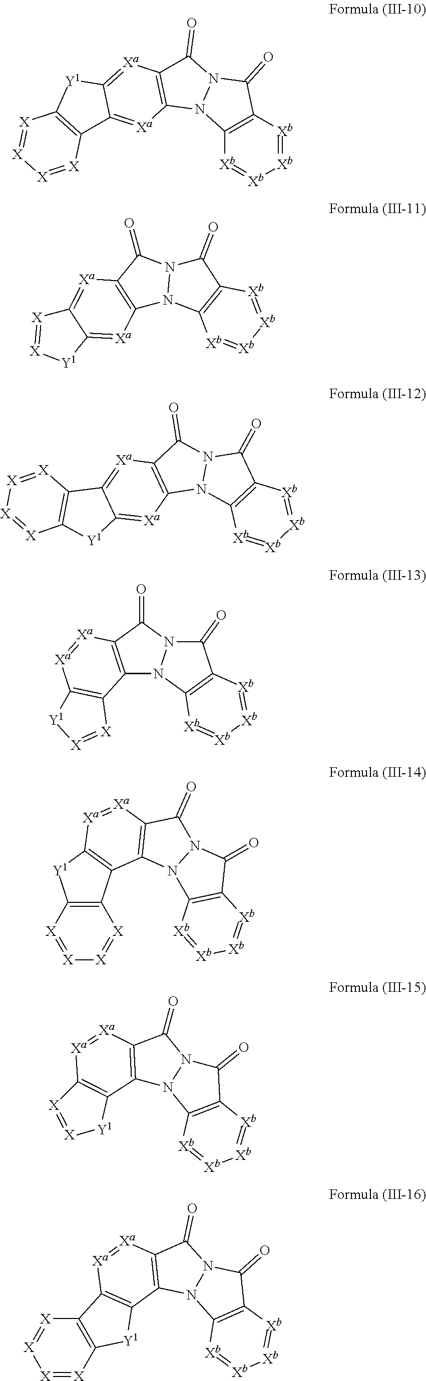 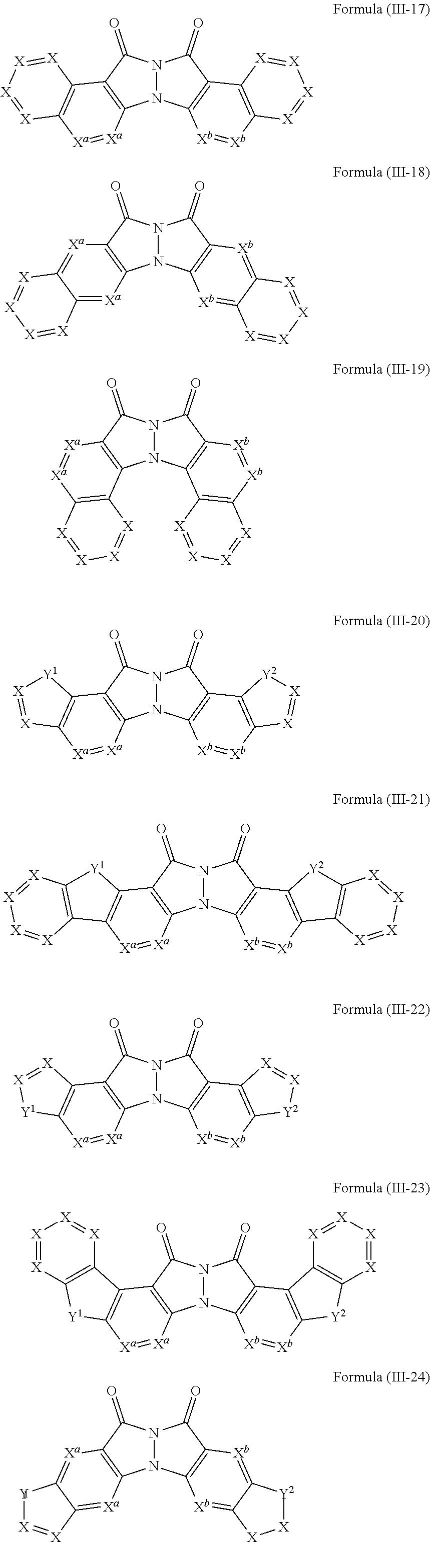 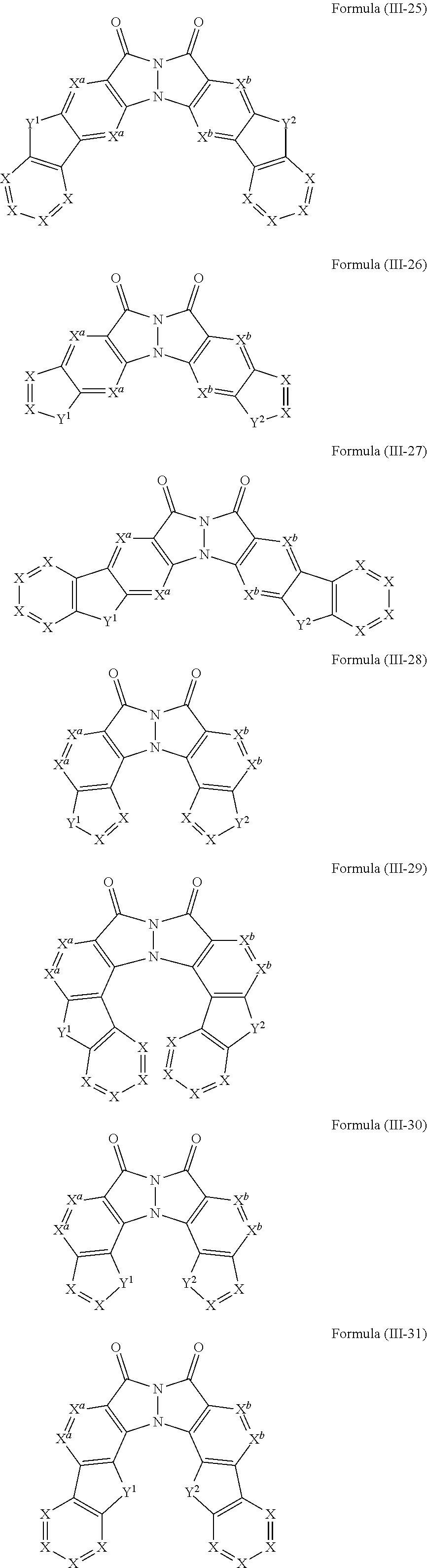 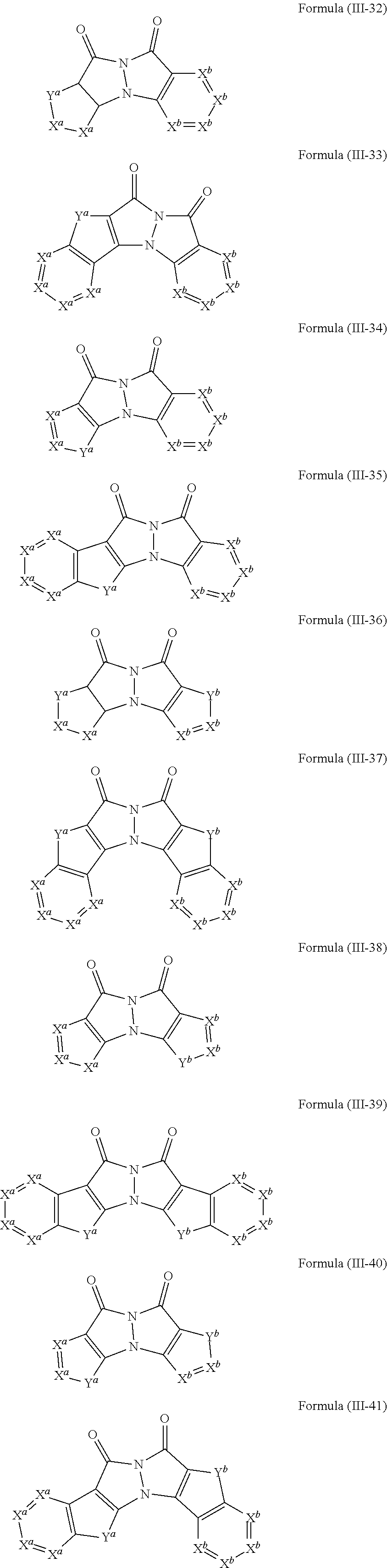
wherein
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, where R^{a} has the definition detailed in claim 19;
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, where R^{b} has the definition detailed in claim 19;
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, where R has the definition detailed in claim 19;
X is the same or different at each instance and is N or CR, with the proviso that not more than two of the X groups in one cycle are N, where R has the definition detailed in claim 19;
X^{a} is the same or different at each instance and is N or CR^{a}, with the proviso that not more than two of the X^{a} groups in one cycle are N, where R^{a} has the definition detailed in claim 19;
X^{b} is the same or different at each instance and is N or CR^{b}, with the proviso that not more than two of the X^{b} groups in one cycle are N, where R^{b} has the definition detailed in claim 19; 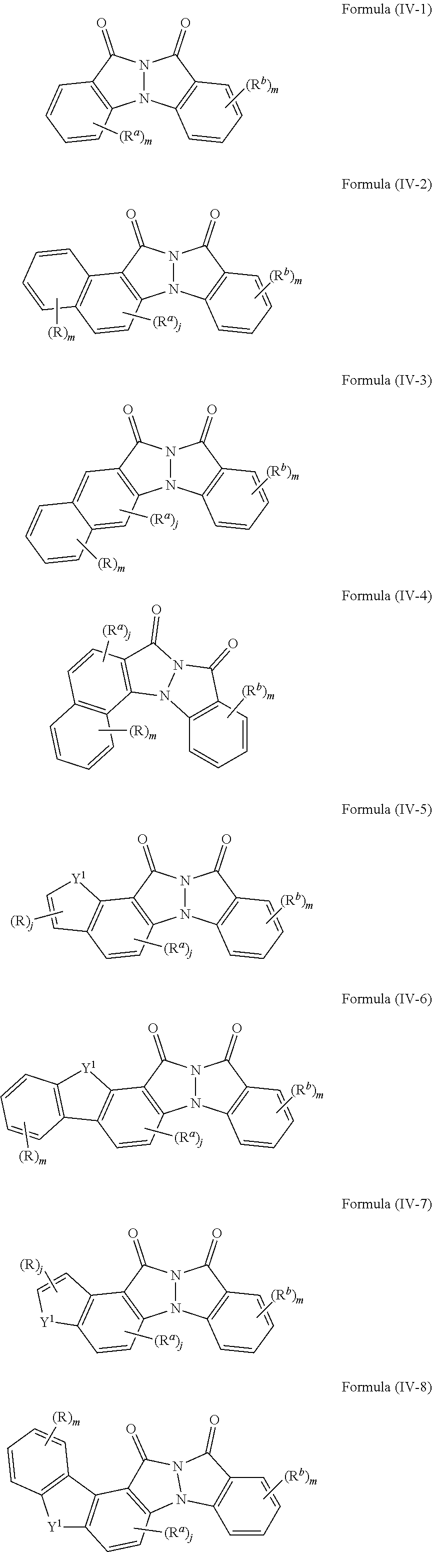 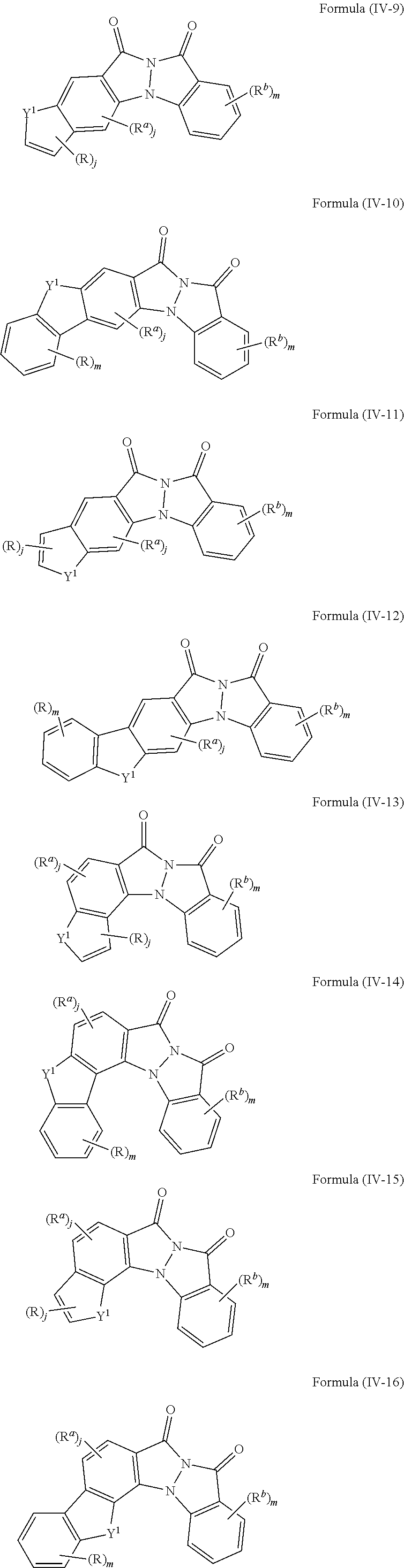 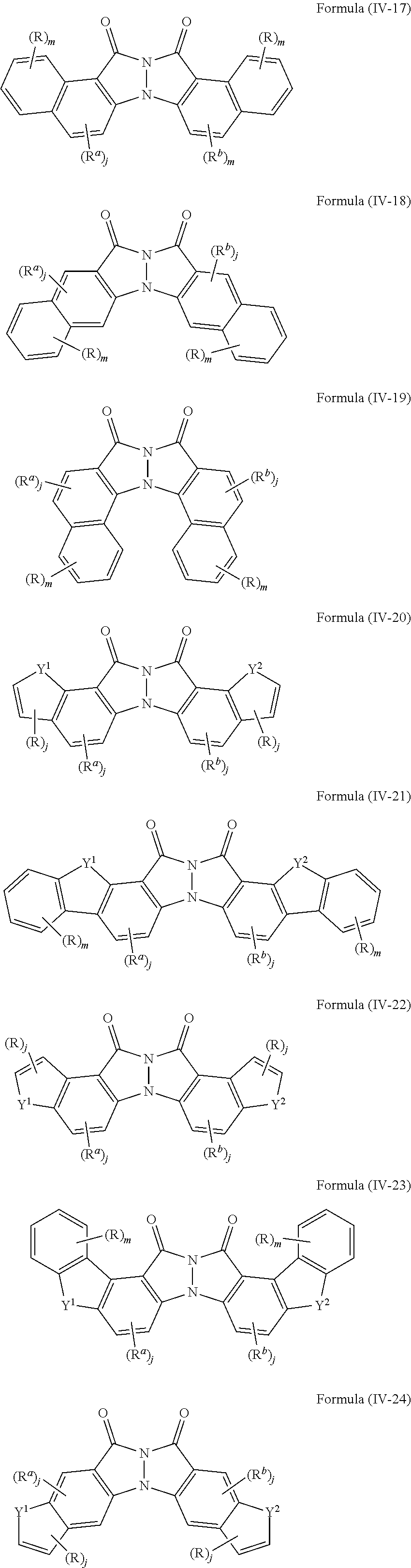 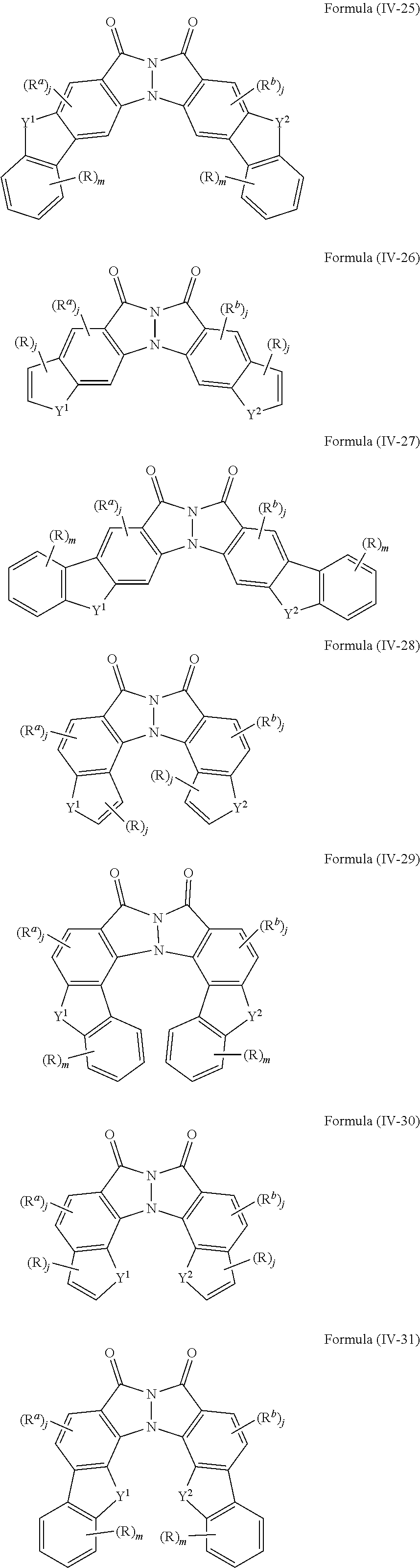 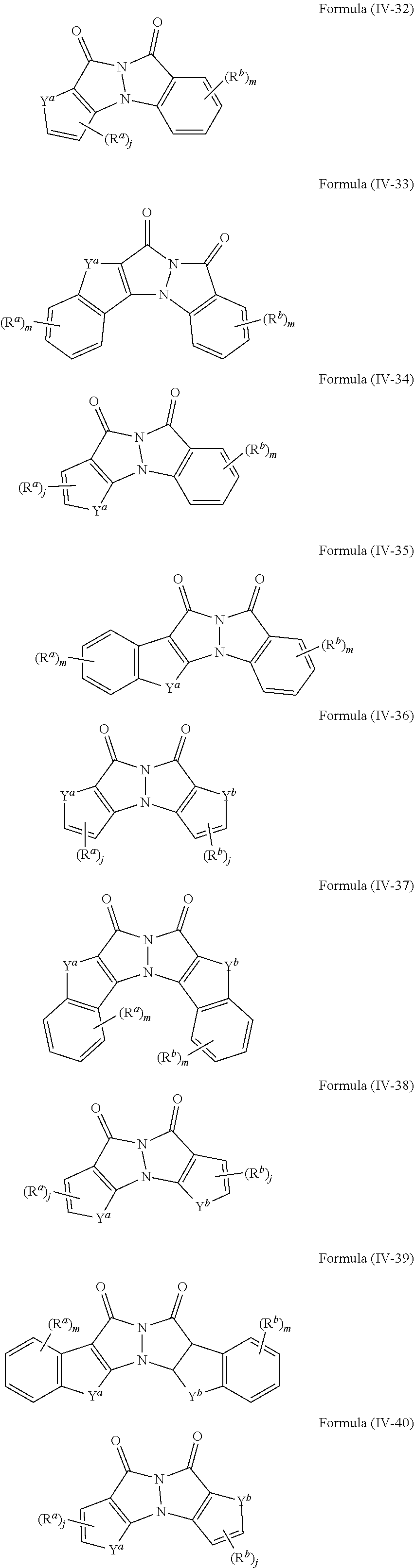 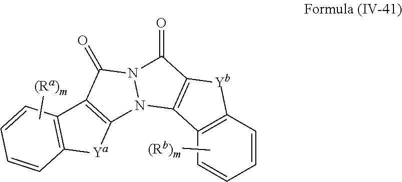
where the symbols R, R^{a} and R^{b} have the definitions given in claim 19, the symbols,
Y^{a} is the same or different at each instance and is N(Ar), N(R^{a}), P(Ar), P(R^{a}), P(═O)Ar, P(═O)R^{a}, P(═S)Ar, P(═S)R^{a}, B(Ar), B(R^{a}), Al(Ar), Al(R^{a}), Ga(Ar), Ga(R^{a}), C═O, C(R^{a})₂, Si(R^{a})₂, Ge(R^{a})₂, C═NR^{a}, C═NAr, C═C(R^{a})₂, C═C(R^{a})(Ar), O, S, Se, S═O or SO₂, where R^{a} has the definition detailed in claim 19;
Y^{b} is the same or different at each instance and is N(Ar), N(R^{b}), P(Ar), P(R^{b}), P(═O)Ar, P(═O)R^{b}, P(═S)Ar, P(═S)R^{b}, B(Ar), B(R^{b}), Al(Ar), Al(R^{a}), Ga(Ar), Ga(R^{b}), C═O, C(R^{b})₂, Si(R^{b})₂, Ge(R^{b})₂, C═NR^{b}, C═NAr, C═C(R^{b})₂, C═C(R^{b})(Ar), O, S, Se, S═O or SO₂, where R^{b} has the definition detailed in claim 19;
Y¹, Y² is the same or different at each instance and is N(Ar), N(R), P(Ar), P(R), P(═O)Ar, P(═O)R, P(═S)Ar, P(═S)R, B(Ar), B(R), Al(Ar), Al(R), Ga(Ar), Ga(R), C═O, C(R)₂, Si(R)₂, Ge(R)₂, C═NR, C═NAr, C═C(R)₂, C═C(R)(Ar), O, S, Se, S═O or SO₂, where R has the definition detailed in claim 19;
and the further symbols have the following definition:
m is 0, 1, 2, 3 or 4;
j is 0, 1 or 2; 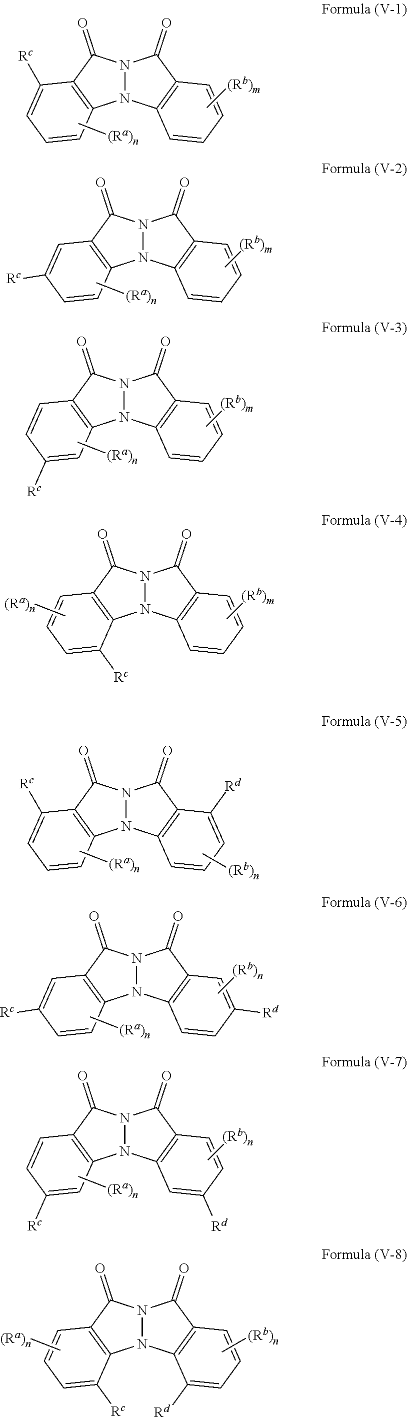
where the symbols R^{a} and R^{b} have the definitions given in claim 19 and the further symbols have the following definition:
R^{c}, R^{d} is the same or different at each instance and is N(Ar′)₂, N(R¹)₂, C(═O)N(Ar′)₂, C(═O)N(R¹)₂, C(Ar′)₃, C(R¹)₃, Si(Ar′)₃, Si(R¹)₃, B(Ar′)₂, B(R¹)₂, C(═O)Ar′, C(═O)R¹, P(═O)(Ar′)₂, P(═O)(R¹)₂, P(Ar′)₂, P(R¹)₂, S(═O)Ar′, S(═O)R¹, S(═O)₂Ar′, S(═O)₂R¹, OSO₂Ar′, OSO₂R¹, a straight-chain alkyl, alkoxy or thioalkoxy group having 1 to 40 carbon atoms or an alkenyl or alkynyl group having 2 to 40 carbon atoms or a branched or cyclic alkyl, alkoxy or thioalkoxy group having 3 to 20 carbon atoms, where the alkyl, alkoxy, thioalkoxy, alkenyl or alkynyl group may in each case be substituted by one or more R¹ radicals, where one or more nonadjacent CH₂ groups may replaced by R¹C═CR¹, C≡C, Si(R¹)₂, C═O, C═S, C═Se, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms and may be substituted in each case by one or more R¹ radicals, or an aryloxy or heteroaryloxy group which has 5 to 60 aromatic ring atoms and may be substituted by one or more R¹ radicals; or heteroarylthio group which has 5 to 60 aromatic ring atoms and may be substituted by one or more R¹ radicals, or a diarylamino, arylheteroarylamino, diheteroarylamino group which has 5 to 60 aromatic ring atoms and may be substituted by one or more R¹ radicals, or an arylalkyl or heteroarylalkyl group which has 5 to 60 aromatic ring atoms and 1 to 10 carbon atoms in the alkyl radical and may be substituted by one or more R¹ radicals; at the same time, one R^{c} radical may form a ring system with an R, R^{a}, R^{b}, R^{d} radical or a further group; at the same time, an R^{d} radical may form a ring system with an R, R^{a}, R^{b}, R^{c} radical or a further group;
m is 0, 1, 2, 3 or 4;
n is 0, 1, 2 or 3; 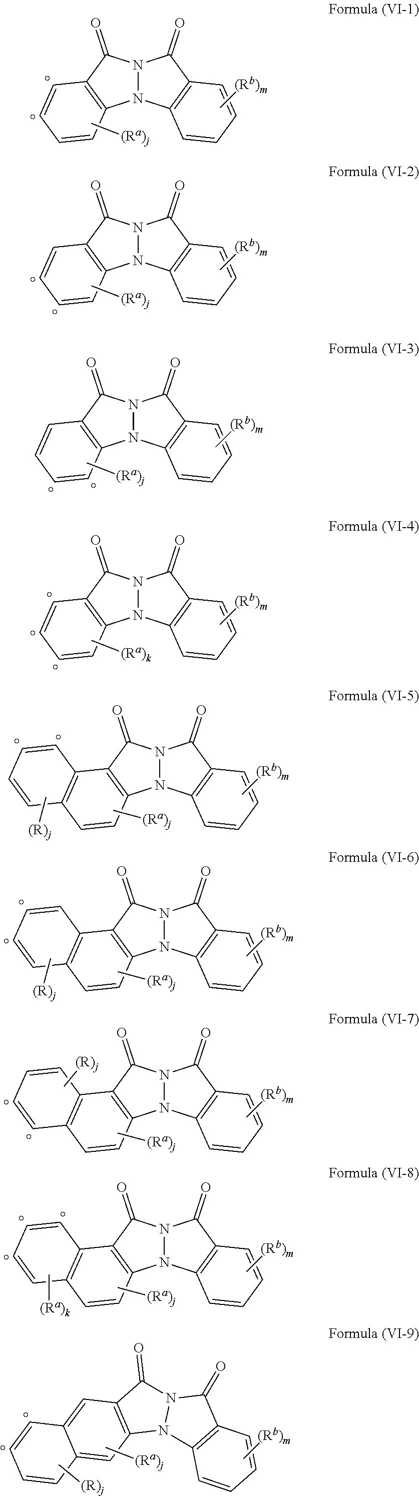 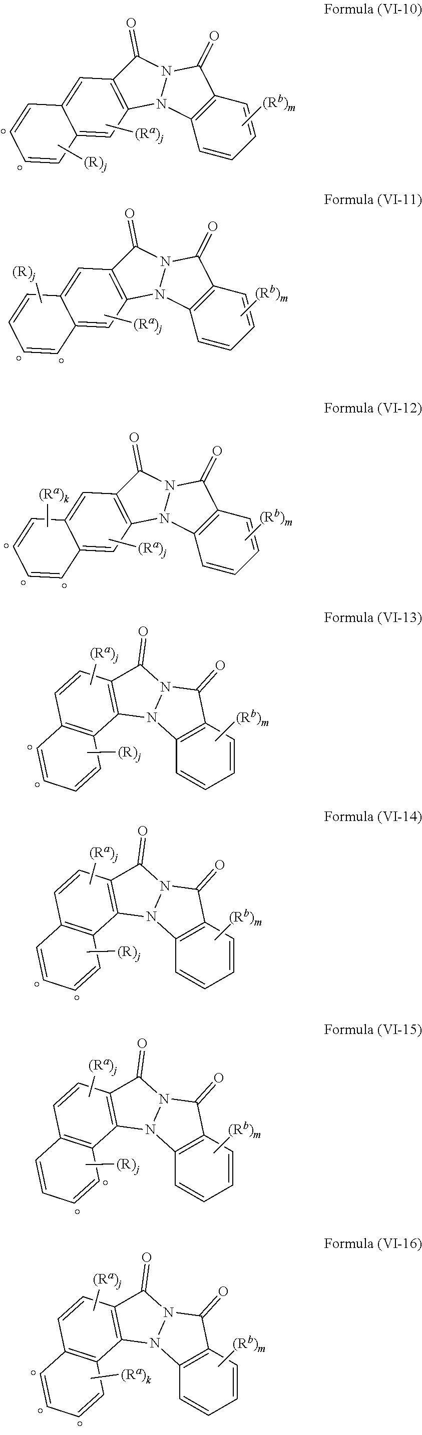 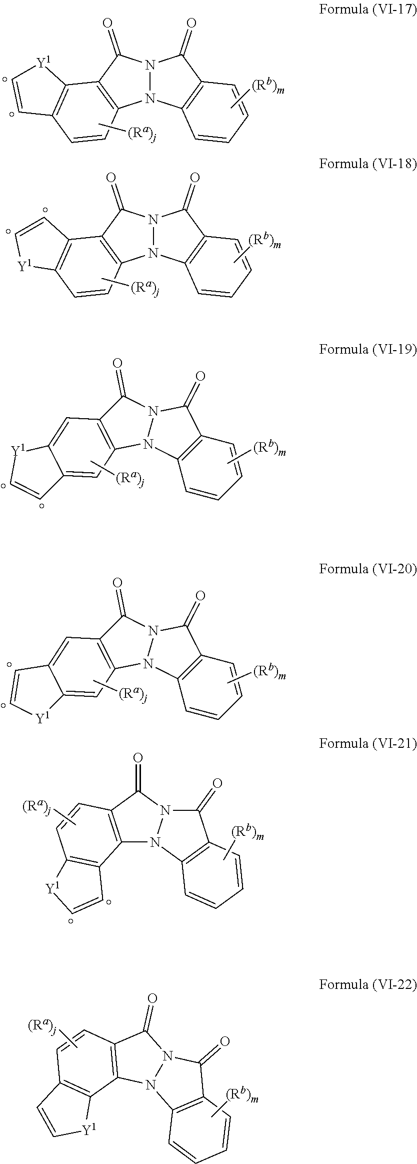
where the symbols R, R^{a}, R^{b}, Y¹ and Y² have the definitions given in claim 19, the symbol o represents the sites of attachment, and the further symbols have the following definition:
m is 0, 1, 2, 3 or 4;
n is 0, 1, 2 or 3;
j is 0, 1 or 2.

32. An oligomer, polymer or dendrimer containing one or more compounds according to claim 19, wherein, rather than a hydrogen atom or a substituent, there are one or more bonds of the compounds to the polymer, oligomer or dendrimer.

33. A formulation comprising at least one the compound according to claim 19 and at least one further compound.

34. A formulation comprising the oligomer, polymer or dendrimer according to claim 32 and at least one further compound.

35. A composition comprising at least one compound according to claim 19 and at least one further compound selected from the group consisting of fluorescent emitters, phosphorescent emitters, emitters that exhibit TADF, host materials, electron transport materials, electron injection materials, hole conductor materials, hole injection materials, electron blocker materials and hole blocker materials.

36. A composition comprising the oligomer, polymer or dendrimer as claimed in claim 31 and at least one further compound selected from the group consisting of fluorescent emitters, phosphorescent emitters, emitters that exhibit TADF, host materials, electron transport materials, electron injection materials, hole conductor materials, hole injection materials, electron blocker materials and hole blocker materials.

37. A process for preparing the compound according to claim 19, which comprises synthesizing a base skeleton having at least one of the W^{a} groups or a precursor of one of the W^{a} groups, and an aromatic or heteroaromatic radical is introduced by means of a nucleophilic aromatic substitution reaction or a coupling reaction.

38. An electronic device comprising at least one compound according to claim 19.

39. An electronic device comprising the oligomer, polymer or dendrimer according to claim 32.
